(19) United States
(12) Patent Application Publication
(10) Pub. No.: US 2016/0186208 A1
(43) Pub. Date: Jun. 30, 2016

(54) Methods of Mutating, Modifying or Modulating Nucleic Acid in a Cell or Nonhuman Mammal

(71) Applicant: Whitehead Institute for Biomedical Research, Cambridge, MA (US)

(72) Inventors: Rudolf Jaenisch, Cambridge, MA (US); Haoyi Wang, Cambridge, MA (US); Hui Yang, Cambridge, MA (US); Chikdu Shivalila, Cambridge, MA (US); Wu Albert Cheng, Cambridge, MA (US)

(21) Appl. No.: 14/785,031

(22) PCT Filed: Apr. 16, 2014
(86) PCT No.: PCT/US2014/034387

§ 371 Date: Oct. 16, 2015

Related U.S. Application Data
(60) Provisional application No. 61/812,720, filed on Apr. 16, 2013.
(60) Provisional application No. 61/824,920, filed on May 17, 2013.
(60) Provisional application No. 61/858,437, filed on Jul. 25, 2013.
(60) Provisional application No. 61/865,888, filed on Aug. 14, 2013.

(51) Int. Cl. C12N 15/85; C12N 15/90; A01K 67/027

(52) U.S. Cl. CPC C12N 15/8509; A01K 67/0276; C12N 15/907; A01K 2207/05; A01K 2217/15; A01K 2227/105

(57) ABSTRACT

The invention is directed to a method of mutating one or more target nucleic acid sequences in a stem cell or a zygote comprising introducing into the stem cell or zygote (i) ribonucleic acid (RNA) sequences that comprise a portion that is complementary to a portion of each of the target nucleic acid sequences and comprise a binding site for a CRISPR associated (Cas) protein; and a Cas nucleic acid sequence or a variant thereof that encodes a Cas protein having nuclease activity. The stem cell or zygote is maintained under conditions in which the target nucleic acid sequences are mutated in the stem cell or zygote. The invention is also directed to methods of producing a non human mammal carrying mutations and methods of modulating the expression and/or activity target nucleic acid sequences and cells or zygotes.

### RELATED APPLICATIONS

This application claims the benefit of U.S. Provisional Application No. 61/812,720, filed on Apr. 16, 2013; U.S. Provisional Application No. 61/824,920, filed on May 17, 2013; U.S. Provisional Application No. 61/858,437, filed on Jul. 25, 2013; and U.S. Provisional Application No. 61/865,888, filed on Aug. 14, 2013.

The entire teachings of the above applications are incorporated herein by reference.

### GOVERNMENT SUPPORT

This invention was made with government support under HD 045022 and R37CA084198 from the National Institutes of Health. The government has certain rights in the invention.

### BACKGROUND OF THE INVENTION

Genetically modified mice represent a crucial tool for understanding gene function in development and disease. Mutant mice are conventionally generated by insertional mutagenesis (Copeland and Jenkins, 2010; Kool and Berns, 2009) or by gene targeting methods (Capecchi, 2005). In conventional gene targeting methods, mutations are introduced through homologous recombination in mouse embryonic stem (ES) cells. Targeted ES cells injected into wild-type blastocysts can contribute to the germline of chimeric animals, generating mice containing the targeted gene modification (Capecchi, 2005). It is costly and time-consuming to produce single gene knockout mice, and even more so to make double mutant mice. Moreover, in most other mammalian species no established ES cell lines are available that contribute efficiently to chimeric animals, which greatly limits the genetic studies in many species.

Alternative methods have been developed to accelerate the process of genome modification by directly injecting DNA or mRNA of site-specific nucleases into the one cell embryo to generate DNA double strand break (DSB) at a specified locus in various species (Bogdanove and Voytas, 2011; Carroll et al., 2008; Urnov et al., 2010). DSBs induced by these site-specific nucleases can then be repaired by either error-prone non-homologous end joining (NHEJ) resulting in mutant mice and rats carrying deletions or insertions at the cut site (Carbery et al., 2010; Geurts et al., 2009; Sung et al., 2013; Tesson et al., 2011). If a donor plasmid with homology to the ends flanking the DSB is co-injected, high-fidelity homologous recombination can produce animals with targeted integrations (Cui et al., 2011; Meyer et al., 2010). Because these methods require the complex designs of zinc finger nucleases (ZNFs) or Transcription activator-like effector nucleases (TALENs) for each target gene and because the efficiency of targeting may vary substantially, no multiplexed gene targeting has been reported to date.

Thus, improved methods for producing genetically modified non-human mammals, such as mice, are needed.

### SUMMARY OF THE INVENTION

Described herein is the use of the Clustered Regularly Interspaced Short Palindromic Repeats (CRISPR) and CRISPR associated (Cas) proteins (CRISPR/Cas) system to drive both non-homologous end joining (NHEJ) based gene disruption and homology directed repair (HDR) based precise gene editing to achieve highly efficient and simultaneous targeting of multiple nucleic acid sequences in cells and nonhuman mammals.

Accordingly, in one aspect, the invention is directed to a method of mutating one or more target nucleic acid sequences in a (one or more) stem cell or a zygote comprising introducing into the stem cell or zygote (i) one or more ribonucleic acid (RNA) sequences that comprise a portion that is complementary to a portion of each of the one or more target nucleic acid sequences and comprise a binding site for a CRISPR associated (Cas) protein; and a Cas nucleic acid sequence or a variant thereof that encodes a Cas protein having nuclease activity. The stem cell or zygote is maintained under conditions in which the one or more RNA sequences hybridize to the portion of each of the one or more target nucleic acid sequences, and the Cas protein cleaves each of the one or more target nucleic acid sequences upon hybridization of the one or more RNA sequences to the portion of the target nucleic acid sequence, thereby mutating one or more target nucleic acid sequences in the stem cell or zygote.

In some aspects, the invention is directed to a method of producing a nonhuman mammal carrying mutations in one or more target nucleic acid sequences comprising introducing into a zygote or an embryo (i) one or more ribonucleic acid (RNA) sequences that comprise a portion that is complementary to a portion of each of the one or more target nucleic acid sequences and comprise a binding site for a CRISPR associated (Cas) protein; and ii) a Cas nucleic acid sequence or a variant thereof that encodes a Cas protein having nuclease activity. The zygote or the embryo is maintained under conditions in which RNA hybridizes to the portion of each of the one or more target nucleic acid sequences, and the Cas protein cleaves each of the one or more target nucleic acid sequences upon hybridization of the RNA to the portion of the target nucleic acid sequence, thereby producing an embryo having one or more mutated nucleic acid sequences. The embryo having one or more mutated nucleic acid sequences may be transferred into a foster nonhuman mammalian mother. The foster nonhuman mammalian mother is maintained under conditions in which one or more offspring carrying the one or more mutated nucleic acid sequences are produced, thereby producing a nonhuman mammal carrying mutations in one or more target nucleic acid sequences.

In some aspects, the invention is directed to a method of modulating the expression and/or activity of one or more target nucleic acid sequences in one or more cells or zygotes comprising introducing into the cell or zygote (i) one or more ribonucleic acid (RNA) sequences that comprise a portion that is complementary to each of the one or more target nucleic acid sequences and comprise a binding site for a CRISPR associate (Cas) protein; (ii) a Cas nucleic acid sequence or a variant thereof that encodes the Cas protein that targets but does not cleave the target nucleic acid sequence; and (iii) an effector domain. The method further comprises maintaining the cell under conditions in which the one or more RNA sequences hybridize to the portion of each of the one or more target nucleic acid sequences, the Cas protein binds to each of the one or more RNA sequences and the effector domain modulates the expression and/or activity of the target nucleic acid, thereby modulating the expression and/or activity of the one or more target nucleic acid sequences in the cell or zygote.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1E show multiplexed gene targeting in mESCs. FIG. 1A shows a schematic of the Cas9/sgRNA targeting sites in Tet1, 2, and 3. The sgRNA targeting sequence is underlined, and the protospacer-adjacent motif (PAM) sequence is labeled in green. The restriction sites at the target regions are bold and capitalized. Restriction enzymes used for restriction fragment length polymorphism (RFLP) and Southern blot analysis are shown, and the Southern blot probes are shown as orange boxes (SEQ ID NOs: 27-29). FIG. 1B shows a surveyor assay for Cas9-mediated cleavage at Tet1, 2, 3 loci in mESCs. FIG. 1C shows the genotyping of triple targeted mESCs, clone #51, #52, and #53 are shown. The upper panels in FIG. 1C is the RFLP analysis. Tet1 PCR products were digested with SacI, Tet2 PCR products were digested with EcoRV, and Tet3 PCR products were digested with XhoI. Lower panel, Southern blot analysis. For the Tet1 locus, SacI digested genomic DNA was hybridized with a 5′ probe. Expected fragment size: WT (wild type)=5.8 kb, TM (Targeted mutation)=6.4 kb. For the Tet2 locus, SacI and EcoRV double digested genomic DNA was hybridized with a 3′ probe. Expected fragment size: WT=4.3 kb, TM=5.6 kb. For the Tet3 locus, BamHI and XhoI double digested genomic DNA was hybridized with a 5′ probe. Expected fragment size: WT=3.2 kb, TM=8.1 kb. FIG. 1D shows the sequence of six mutant alleles in triple targeted mESC clone #14 and #41 (SEQ ID NOs: 30-45). Protospacer-Adjacent Motif (PAM) sequence is labeled in red. FIG. 1E shows the analysis of 5hmC levels in DNA isolated from triple targeted mES clones by dot blot assay using anti-5hmC antibody. A previously characterized DKO clone derived using traditional method is used as a control. See also FIGS. 5A-D, Tables 1 and 4.

FIGS. 2A-2F show single and double gene targeting in vivo by injection into fertilized eggs. (2A) Genotyping of Tet1 single targeted mice. FIG. 2B shows genotyping of Tet2 single targeted mice. RFLP analysis is shown in the upper panel, and Southern blot analysis is shown in the lower panel of FIG. 2B. FIG. 2C is the sequence of both alleles of targeted gene in Tet1 biallelic mutant mouse #2 and Tet2 biallelic mutant mouse #4 (SEQ ID NOs: 30, 32-34, 46, 47). FIG. 2D is the genotyping of Tet1/Tet2 double mutant mice. Analysis of mice #1 to #12 is shown. RFLP analysis is shown in the upper panel, and Southern blot analysis is shown in the lower panel of FIG. 2D. The Tet1 locus is displayed in the left panel and the Tet2 locus in the right panel. FIG. 2E is the sequence of four mutant alleles from double mutant mouse #9 and #10 (SEQ ID NOs: 30, 33, 42, 46, 48-53). PAM sequences are labeled in red. FIG. 2F is a picture of three-week-old double mutant mice. All RFLP and Southern digestions and probes are the same as those used in FIGS. 1A-E. See also FIGS. 6A-6F, Tables 2 and 3.

FIGS. 3A-3C show multiplexed HR-mediated genome editing in vivo. FIG. 3A shows a schematic of the oligo targeting sites at Tet1 and Tet2 loci (SEQ ID NOs: 54-57). The sgRNA targeting sequence is underlined, and the PAM sequence is labeled in green. Oligo targeting each gene is shown under the target site, with 2 bp changes labeled in red. Restriction enzyme sites used for RFLP analysis are bold and capitalized. FIG. 3B is RFLP analysis of double oligo injection mice with HDR-mediated targeting at the Tet1 and Tet2 loci. FIG. 3C shows the sequences of both alleles of Tet1 and Tet2 in mouse #5 and #7 show simultaneously HDR-mediated targeting at one allele or two alleles of each gene, and NHEJ-mediated disruption at the other alleles (SEQ ID NOs: 30, 33, 40, 58-61). See also FIGS. 8A-8C.

FIGS. 4A-4B show multiplexed genome editing in mES cells and mouse. FIG. 4A is a diagram representing multiple gene targeting in mES cells. FIG. 4B shows one step generation of mice with multiple mutations. Upper panel, multiple targeted mutations with random indels introduced through NHEJ. Lower panel, multiple predefined mutations introduced through HDR-mediated repair.

FIGS. 5A-5D show single, triple, and quintuple gene targeting in mES cells. FIG. 5A is RFLP analysis of clones from each single targeting experiment (#1 to #17 are shown). FIG. 5B is RFLP analysis of triple gene targeted clones (#37 to #53 are shown). Tet1 PCR products were digested with SacI, Tet2 PCR products were digested with EcoRV, and Tet3 PCR products were digested with XhoI. WT control is shown in the last lane. Genotyping of clone #51, #52, and #53 are also shown in FIG. 1C. FIG. 5C is a schematic of the Cas9/sgRNA targeting sites in Sry and Uty (SEQ ID NOs: 62-64). The sgRNA targeting sequence is underlined, and the protospacer-adjacent motif (PAM) sequence is labeled in green. The restriction sites at the target regions are bold and capitalized. Restriction enzymes used for RFLP analysis are shown. FIG. 5D is RFLP analysis of quintuple gene targeted clones (#1 to #10 are shown). Sry PCR products were digested with BsaJI, Uty PCR products were digested with AvrII. WT control is shown in the last lane. RFLP analysis of Tet1, 2, 3 loci are not shown. FIGS. 5A-5D are related to FIG. 1A-1E, Tables 1 and 4.

FIGS. 6A-6F show one step generation of single gene mutant mice by zygote injection FIG. 6A is RFLP analysis of blastocysts injected with different concentration of Cas9 mRNA and Tet1 sgRNA at 20 ng/μl. Tet1 PCR products were digested with SacI. FIG. 6B shows commonly recovered Tet1 and Tet2 alleles resulted from MMEJ (SEQ ID NOs: 30, 33, 34, 40, 46, 52). PAM sequence of each targeting sequence is labeled in green. Microhomology flanking the DSB is bold and underlined in WT sequence. FIG. 6C is RFLP analysis of eight Tet3 targeted blastocysts demonstrated high targeting efficiency (embryo #3 and #5 failed to amplify). Tet3 PCR products were digested with XhoI. FIG. 6D is a picture of how some Tet3 targeted mice show smaller size and all homozygous mutants died within one day after birth. FIG. 6E is RFLP analysis of Tet3 single targeted new born mice. Mouse #8 and #14 survived after birth. Sample #2 and #6 failed to amplify. FIG. 6F are sequences of both Tet3 alleles of surviving Tet3 targeted mouse #14. PAM sequences are labeled in red. FIGS. 6A-6F are related to FIGS. 2A-2F and Table 2.

FIGS. 7A-7B show off target analysis of double mutant mice. FIG. 7A shows three potential off targets of Tet1 sgRNA and four potential off targets of Tet2 sgRNA are shown (SEQ ID NOs: 66-74). The 12 bp perfect matching seed sequence is labeled in blue, and NGG PAM sequence is labeled in red. FIG. 7B shows a surveyor assay of all seven potential off target loci in seven double mutant mice derived with high concentration of Cas9 mRNA (100 ng/μl) injection. WT control is included as the eighth sample. The weak cleavage activity at Ubr1 locus is not due to off target effect, since sequences of these PCR products show no mutations. FIGS. 7A-7B are related to FIGS. 2A-2F and Table 5.

FIGS. 8A-8C show multiplexed precise HDR-mediated genome editing in vivo. FIG. 8A is RFLP analysis of single oligo injection embryos with HDR-mediated targeting at Tet1 and Tet2 locus. FIG. 8B is RFLP analysis of double oligo injection embryos with multiplexed HDR-mediated targeting at both Tet1 and Tet2 loci. FIG. 8C shows the sequences of both alleles of Tet1 and Tet2 in embryo #2 and show simultaneously HDR-mediated targeting at one allele of both genes, and NHEJ-mediated gene disruption at the other allele of each gene (SEQ ID NOs: 30, 33, 53, 58, 59, 75). FIGS. 8A-8C are related to FIGS. 3A-3C.

FIG. 9 shows 20 bp sequences of Tet1, Tet2, Tet3, Sry and Uty and the full length sequences of the RNA sequences (SEQ ID NOs: 76-85).

FIGS. 10A-10C: dCas9ta guided by sgRNA targeting tet binding site activates TetO promoter in HeLa cell. (10A) Schematic of dCas9ta fusion protein generated by mutation of two amino acids of Cas9 protein and fusion to 3×VP16 minimal transactivation domain (10B) Schematic of a TetO::tdTomato reporter system to test dCas9ta fusion protein. (10C) Phase contrast, fluorescent microscopy and fluorescent activated cell sorting (FACS) profile of HeLa/TetO::tdTomato; EF1a::NLSM2rtTA cells transfected by pmaxGFP (i), under dox exposure for 2 days (ii), transfected with dCas9ta without sgRNA (iii), and transfected with dCas9ta with sgRNA (sgTetO) complementary to tet binding sites (iv).

FIGS. 11A-11C dCas9ta guided by sgRNAs targeting Nanog promoter activates a NanogGFP reporter and endogenous Nanog gene in NIH3T3 cells. (11A) Schematic of the experiment showing the target sites of the sgRNAs relative to Nanog locus and the NanogGFP reporter. (11B) qRT-PCR analysis of endogenous Nanog. The fold change of (i) NanogGFP-only, (ii) NanogGFP+dCas9ta, (iii) NanogGFP+dCas9ta+sgmNanog were expressed relative to NanogGFP-only control. (11C) Microscope pictures of cells transfected with (i) NanogGFP-only, (ii) NanogGFP+dCas9ta, (iii) NanogGFP+dCas9ta+sgmNanog.

FIGS. 12A-12D: dCas9ta guided by sgRNA targeting tet binding site activates TetO promoter in NIH3T3 cell. Phase contrast, fluorescent microscopy and fluorescent activated cell sorting (FACS) profile of NIH3T3/TetO::tdTomato; EF1a::NLSM2rtTA cells transfected by pmaxGFP (12A), under dox exposure for 2 days (12B), transfected with dCas9ta without sgRNA (12C), and transfected with dCas9ta with sgRNA complementary to tet binding sites (12D).

FIGS. 13A-13D: Microscope pictures and FACS analysis of HeLa/TetO::tdTomato cells (13A) No transfection. (13B) Transfected with dCas9ta+sgTetO. (13C) Transfected with dCas9Cdk9+dCas9CycT+sgTetO. (13D) Transfected with dCas9ta+dCas9Cdk9+dCas9CycT+sgTetO.

FIG. 14: the wild type (Wt) Cas9 (S. pyogenes) nucleotide sequence (SEQ ID NO: 485).

FIG. 15A: Alignment of HMG box sequences of Sry proteins from different mammalian species (SEQ ID NOs: 86-91). Position 94 (shown in red) is highly conserved in different species (h: human; m: mouse; c: Chimpanzee; pc: Pygmy Chimpanzee; g: gorilla; py: Pongo; hl: Hylobates; b: Baboon and cj: Calitrix jaccus). (From Shahid et al. BMC Medical Genetics 2010 11:131 doi:10.1186/1471-2350-11-131).

FIG. 15B: Genetic modification of Sry using TALENs. Schematic of Sry TALEN pair 2 and its recognition sequence within high mobility group (HMG) domain of Sry (SEQ ID NOs: 86-96). TAL repeats are color-coded to represent each of four repeat variable di-residues (RVDs); each RVD recognizes one corresponding DNA base (NI=A, NG=T, HD=C, NN=G). Nucleotides bound by TALENs are capitalized. Shown below are clones (targeted mutation [TM] alleles 1-3) where Sry deletions induced by TALENs were indicated in dashed lines. Srytm4 (540-bp deletion) and Srytm5 (440-bp deletion) clones are not shown.

FIGS. 16A-16F: One step generation of the Sox2-V5 allele. (16A) Schematic of the Cas9/sgRNA/oligo targeting site at the Sox2 stop codon (SEQ ID NOs: 97 and 98). The sgRNA coding sequence is underlined, capitalized, and labeled in red. The protospacer-adjacent motif (PAM) sequence is labeled in green. The stop codon of Sox2 is labeled in orange. The oligo contained 60 bp homologies flanking the DSB. In the oligo donor sequence, the V5 tag sequence is labeled as a green box. PCR primers (SF, V5F, and SR) used for PCR genotyping are shown as red arrowheads. (16B) Upper panel, PCR genotyping using primers V5F and SR produced bands with correct size in targeted ES samples T1 to T5, but not in WT sample. Lower panel, PCR genotyping using primers SF and SR produced slightly larger products, indicating the 42 bp V5 tag sequence was integrated. T1 only contain larger product, suggesting either both alleles were targeted, or one allele failed to amplify. (16C) PCR products using primers SF and SR were cloned into plasmid and sequenced. Sequence across the targeting region confirmed correct fusion of V5 tag to the last codon of Sox2. (16D) Western blot analysis identified Sox2-V5 protein using V5 antibody in ES cells containing Sox2-V5 allele. Beta-actin was shown as the loading control. (16E) Immunostaining of targeted blastocyst using V5 antibody showed signal in ICM. Scale bar, 50 μm. (16F) Immunostaining of targeted ES cells using V5 antibody showed uniform Sox2 expression. Scale bar, 100 μm.

FIGS. 17A-17F: One step generation of an endogenous reporter allele (SEQ ID NOs: 99 and 100). (17A) Schematic overview of strategy to generate a Nanog-mCherry knock-in allele. The sgRNA coding sequence is underlined, capitalized, and labeled in red. The protospacer-adjacent motif (PAM) sequence is labeled in green. The stop codon of Nanog is labeled in orange. The homologous arms of the donor vector are indicated as HA-L (2 kb) and HA-R (3 kb). The restriction enzyme used for Southern blot analysis is shown, and the Southern blot probes are shown as red boxes. (17B) Southern analysis of Nanog-mCherry targeted allele. NcoI-digested genomic DNA was hybridized with 3′external probe. Expected fragment size: WT (wild type)=11.5 kb, T (Targeted)=5.6 kb. The blot was then stripped and hybridized with mCherry internal probe. Expected fragment size: WT=N/A, T=6.6 kb. (17C) Nanog-mCherry targeted blastocysts showed expression in ICM. Mouse ES cell lines derived from targeted blastocysts remain mCherry positive, and the mCherry expression disappear upon differentiation. Scale bar, 100 μm. (17D) Schematic overview of strategy to generate an Oct4-eGFP knock-in allele (SEQ ID NO: 101). The sgRNA coding sequence is underlined, capitalized, and labeled in red. The protospacer-adjacent motif (PAM) sequence is labeled in green. The homologous arms of the donor vector are indicated as HA-L (4.5 kb) and HA-R (2 kb). The IRES-eGFP transgene is indicated as a green box, and the PGK-Neo cassette is indicated as a grey box. The restriction enzyme used for Southern blot analysis is shown, and the Southern blot probes are shown as red boxes. (17E) Oct4-eGFP targeted blastocysts showed expression in ICM. Scale bar, 50 μm. Mouse ES cell lines derived from targeted blastocysts remain GFP positive. Scale bar, 100 μm. (17F) Southern analysis of Oct4-eGFP targeted allele. Southern analysis of Oct4-eGFP targeted allele. HindIII-digested genomic DNA was hybridized with 3′external probe. Expected fragment size: WT=9 kb, Targeted=7.2 kb. The blot was then stripped and hybridized with eGFP internal probe. Expected fragment size: WT=N/A, Targeted=7.2 kb.

FIGS. 18A-18E: One step generation of a Mecp2 floxed allele. (18A) Schematic of the Cas9/sgRNA/oligo targeting sites in Mecp2 intron 2 and intron 3 (SEQ ID NOs: 102-106). The sgRNA coding sequence is underlined, capitalized, and labeled in red. The protospacer-adjacent motif (PAM) sequence is labeled in green. In the oligo donor sequence, the loxP site is indicated as an orange box, and the restriction site sequences are in bold and capitalized. Restriction enzymes used for RFLP and Southern blot analysis are shown, and the Southern blot probes are shown as red boxes. (18B) Southern analysis of targeted alleles. Data of five mice are shown. EcoRI/NheI-digested genomic DNA was hybridized with the exon3 probe. Expected fragment size: WT=5.2 kb, 2loxP=0.7 kb, L2-loxP=3.9 kb, R1-loxP=2 kb. The blot was then stripped and hybridized with the exon4 probe. Expected fragment size: WT=5.2 kb, 2loxP=3.2 kb. L2-loxP=3.9 kb, R1-loxP=3.2 kb. The sequence of the floxed allele is shown in FIG. 26B. (18C) In vitro Cre-mediated recombination of the floxed Mecp2 allele. The genomic DNA of targeted mice #1 and #3 was incubated with Cre recombinase, and used as PCR template. Primers DF and DR flanking the floxed allele produce shorter products upon Cre-dependent excision. Primers CF and CR detect the circular molecule, which only form upon Cre-loxP recombination. The position of each primer is shown at the bottom cartoon. The deletion and circular PCR products were sequenced and the sequences are shown in FIG. 26C. (18D) Injection of Cas9 mRNA and both L2 and R1 sgRNA generated Mecp2 mutant allele with deletion of exon 3. PCR genotyping using primers DF and DR identified defined deletion events in mice #1, #6, and #8 (indicated by stars). (18E) Sequences of three mutant alleles with exon 3 deletions in three mice (SEQ ID NOs: 107-111). R2 and L1 sgRNA coding sequences were underlined, capitalized, and labeled in red. The protospacer-adjacent motif (PAM) sequence is labeled in green.

FIGS. 19A-19C: Integration of loxP sites at Tet1 and Tet2 loci. (19A) Schematic of the Cas9/sgRNA/oligo targeting sites in Ted exon 4 and Tet2 exon 3 (SEQ ID NOs: 112-115). The sgRNA coding sequence is underlined, capitalized, and labeled in red. The protospacer-adjacent motif (PAM) sequence is labeled in green. In oligo donor sequence, the loxP site is indicated by an orange box, and the restriction site sequence is in bold and capitalized. (19B) RFLP analysis of double sgRNA/oligo injection mice with HDR-mediated targeting at the Ted and Tet2 loci. About 500 bp regions around the targeting sites at Tet1 and Tet2 were amplified from 16 embryos and digested with EcoRI. A corrected targeted allele is identified as a cleaved fragment. Samples containing targeted alleles are indicated by stars. (19C) The sequences of targeted alleles of Ted and Tet2 in sample #2 and #9 confirmed precise integration of loxP sites at both loci.

FIGS. 20A-20C: Characterization of Nanog-mCherry alleles. (20A) ES clone with mosaic expression of mCherry. The mCherry negative colony is indicated by the arrow. (20B) Southern analysis of Nanog-mCherry targeted allele identified mosaic animal. NcoI-digested genomic DNA was hybridized with 3′external probe. Expected fragment size: WT=11.5 kb, T=5.6 kb. Mouse #6 is identified as mosaic, because the targeted band (indicated by arrow) is weaker than WT band. (20C) The blot was then stripped and hybridized with mCherry internal probe. Expected fragment size: WT=NA, Targeted=6.6 kb. In addition to the targeted allele, one extra band (indicated by arrow) is present in mouse #3, indicating a ramdon insertion of the donor vector.

FIGS. 21A-21B: Integration of loxP sites at Mecp2 intron 2 and 3. (21A) Schematic of the Cas9/sgRNA/oligo targeting sites (SEQ ID NOs: 116-124). The sgRNA coding sequence is underlined, capitalized, and labeled in red. The protospacer-adjacent motif (PAM) sequence is labeled in green. In oligo donor sequence, the loxP site is labeled as an orange box, and the restriction site sequence is in bold and capitalized. PCR primers used for RFLP analysis are shown as red arrows. For intron 2, two sgRNA coding sequences L1 and L2 are shown, and their corresponding oligos are named accordingly. For intron 3, R1, R2 and their targeting oligos are shown. PCR primers LF and LR are used to amplify the intron 2 region, while RF and RR are used to amplify the intron 3 region. (21B) RFLP analysis of single sgRNA/oligo injection mice with HDR-mediated targeting at Mecp2 intron 2 or intron 3. Cleavage of PCR product upon NheI or EcoRI digestion indicates loxP integration at intron 2 or intron 3 respectively. LoxP integration efficiency at L1, L2, R1, and R2 sites are compared. Samples containing loxP site are labeled by stars. Three out of eight samples contained a loxP site at the L1 site, and four out of eight contained a loxP site at the L2 site. Two out of six samples contained loxP site at R1 site, while none was detected at the R2 site. Primers used for each PCR are labeled.

FIGS. 22A-22C: Analysis of Mecp2 floxed allele. (22A) RFLP analysis detected loxP integration at intron 2 (Mecp2-L2) and intron 3 (Mecp2-R1) in mice derived from L2 and R1 double sgRNA/oligo injections. Primers LF and LR were used to amplify intron 2 region, and RF and RR were used to amplify intron 3 region. Mice containing loxP sites in both introns are marked by stars. (22B) Partial chromatograph from one single sequencing file crossing both loxP sites, exon 3, and flanking intron sequences. (22C) Partial chromatograph from sequences of Cre-mediated recombination PCR products (deletion and circular products from FIG. 18C).

FIGS. 23A-23D: CRISPR-on activates exogenous transgenes. (23A) Schematic of the dCas9VP48 mediated transgene activation in HeLa cells. dCas9VP48 was generated by fusing dCas9 (indicated by black circle) to VP48 domain (indicated by green diamond). sgRNA complementary to rtTA binding site is indicated by small hairpin labeled sgTetO. (23B) dCas9VP48 activates TetO::tdTomato transgene in HeLa cells. Upper (top) panel, phase contrast picture of transfected cells; middle panel, tdTomato signal using fluorescent microscopy; bottom panel, FACS analysis of transfected cells. Column i, cells transfected with GFP plasmid; Column ii, cells treated with doxycycline; Column iii, cells transfected with dCas9VP48 only; Column iv, cells transfected with dCas9VP48 and sgTetO. Cells were transfected with the indicated plasmids and 48 hr later were analyzed by flow cytometry for tdTomato expression. (23C) Schematic of the dCas9VP48 mediated reporter activation in early mouse embryos. dCas9VP48, Nanog::EGFP vector, and 7 sgRNAs targeted on Nanog promoter were co-injected into mouse zygotes and cultured into blastocyst stage. (23D) dCas9VP48/sgRNA can activate gene in vivo. Left panel, embryos injected with dCas9VP48 and Nanog::EGFP vector; right panel, embryos injected with dCas9VP48, Nanog::EGFP vector and sgRNAs targeting Nanog promoter. Embryos two, three, four days post-injection were shown.

FIGS. 24A-24G: dCas9VP160 activated multiple endogenous genes simultaneously. (24A) Protein architecture of dCas9VP160 compared to VP48. (24B) Schematic of the human IL1RN promoter region. Locations of transcription start site (TSS) and start codon (ATG) are indicated. Short lines with number indicate targeting sites of the sgRNAs. (24C) Activation of human IL1RN expression in HEK293T cells. Cells transfected with dCas9VP160 and indicated sgRNAs were analyzed by qRT-PCR 2 days later. sgTetO-mut, negative control sgRNA. Error bars show standard deviation (SD) among triplicates. (24D) Schematic of the human SOX2 promoter region. Locations of TSS and start codon (ATG) are indicated. Short lines with number indicate locations of sgRNAs. (24E) Activation of SOX2. Cells transfected with dCas9VP160 and indicated sgRNAs were analyzed by qRT-PCR 2 days later. sgTetO-mut, negative control sgRNA. Error bars show SD among triplicates. (24F) Schematic of the human OCT4 promoter region. Locations of transcription start site (TSS) and start codon (ATG) are indicated. Short lines with number indicate locations of sgRNAs. (24G) Activation of OCT4. Cells transfected with dCas9VP160 and indicated sgRNAs were analyzed by qRT-PCR 2 days later. sgTetO-mut, negative control sgRNA. Error bars show SD among triplicates.

FIGS. 25A-25B: Multiple exogenous and endogenous genes were simultaneously activated by CRISPR-on. (25A) One exogenous and two endogenous genes were simultaneously activated by CRISPR-on. Cells transfected with dCas9VP160 and indicated sgRNAs were analyzed by qRT-PCR 2 days later. sgTetO-mut, negative control sgRNA. Error bars show SD among triplicates. (25B) Three endogenous genes SOX2, IL1RN, and OCT4, can be simultaneously activated by dCas9VP160/sgRNAs. Cells were transfected with dCas9VP160 and indicated sgRNAs and were analyzed by qRT-PCR 2 days later. sgTetO-mut, negative control sgRNA. The last three sets of bars represent triple activation experiments using sgSOX2, sgOCT4 and sgIL1RN with three different ratios of sgSOX2:sgIL1RN, keeping the amount of sgOCT4 constant, as indicated by numbers above line. Error bars show SD among triplicates.

FIGS. 26A-26D: CRISPR-on is specific. (26A) The histogram showing distribution of Log₂ fold changes of gene expression in sample transfected with dCas9VP160/sgTetO over dCas9VP160/sgTetO-mut control. (26B) A histogram showing distribution of Log₂ fold changes of gene expression in sample transfected with dCas9VP160/sgIL1RN1˜3 over dCas9VP160/sgTetO-mut control. The vertical line marks the fold change of the target gene IL1RN. (26C) Column graph showing the log₂ fold changes of genes up-regulated by at least two fold in cells transfected with dCas9VP160/sgTetO over dCas9VP160/sgTetO-mut. The dotted line indicates the 2 fold cut-off. (26D) Column graph showing the log₂ fold changes of genes up-regulated by more than two fold in cells transfected with dCas9VP160/sgIL1RN1˜3 over dCas9VP160/sgTetO-mut.

FIG. 27: The persistence of CRISPR-on mediated transgene expression. Cells were transfected with dCas9VP48/sgTetO in HeLa/TetO::tdTomato; EF1a::rtTA-M2 cells and mean fluorescence measured by FACS was shown as fold relative to non-transfected control for the indicated samples 2 days, 12 days and 18 days after transfection.

FIGS. 28A-28B: CRISPR-on activates transgene in mouse cells. dCas9VP48 guided by sgRNA targeting rtTA binding site activates TetO promoter in NIH3T3/TetO::tdTomato; EF1a::rtTA-M2 cells. (28A) Schematic of the dCas9VP48 mediated transgene activation in NIH3T3 cells. dCas9VP48 was generated by fusion of dCas9 to VP48 and then co-transfected with sgRNA complementary to tet binding site in NIH3T3/TetO::tdTomato; EF1a::rtTA-M2 cells. (28B) dCas9VP48 depends on sgRNA to bind to the target tetO promoter to activate TetO::tdTomato transgene in NIH3T3 cells. Cells were transfected with the indicated plasmids or sgRNAs and were analyzed by flow cytometry for tdTomato expression 48 hours later.

FIG. 29: CRISPR-on activated a single-copy transgene in ESCs. Cells were transfected with the indicated plasmids into a Tet-inducible MSI1 over-expression mouse embryonic stem cell (mESC) line and were analyzed by western blot for MSI1 expression 48 hours later.

FIGS. 30A-30B: Tunable gene activation can be achieved by titration of sgRNA. (30A) Schematic of CRISPR-on-mediated transgene activation with titration of sgRNA in Hela cells. (30B) Fold changes of mean fluorescence of tdTomato under the control of TetO promoter. Cells were transfected with dCas9 activator and indicated amount of sgTetO and mean fluorescence measured by FACS were analyzed 2 days later. NT=not transfected; C=negative control sgRNA.

FIGS. 31A-31B: dCas9VP48 with 6 sgRNAs failed to activate the IL1RN gene. (31A) Schematic of the human IL1RN promoter region. Locations of transcription start site (TSS) and start codon (ATG) are indicated. Short lines with number indicate locations of sgRNAs. (31B) Activation of human IL1RN expression in HEK293T cells by dCas9VP48/sgRNAs. Cells were transfected with dCas9VP48 and six sgRNAs and 2 days later were analyzed by qRT-PCR. sgTetO-mut, negative control sgRNA. Error bars show SD among triplicates.

FIGS. 32A-32C: Nucleotide sequences of dCas9VP64 on pmax expression vector (SEQ ID NO: 486), dCas9Vp96 on pmax expression vector (SEQ ID NO: 487), and dCas9Vp160 on pmax expression vector (SEQ ID NO: 488).

### DETAILED DESCRIPTION OF THE INVENTION

A description of example embodiments of the invention follows.

Mice carrying mutations in multiple genes are traditionally generated by sequential recombination in embryonic stem cells and/or time-consuming intercrossing of mice with single mutants. Described herein is the development of an efficient technology for the generation of animals carrying multiple mutated genes. Specifically, the clustered regularly interspaced short palindromic repeats (CRISPR) and CRISPR associated genes (Cas genes), referred to herein as the CRISPR/Cas system, has been adapted as an efficient gene targeting technology e.g., for multiplexed genome editing. Demonstrated herein is that CRISPR/Cas mediated gene editing allows the simultaneous disruption of five genes (Tet1, Tet2, Tet3, Sry, Uty—8 alleles) in mouse embryonic stem cells (mESCs) with high efficiency. Co-injection of Cas9 mRNA and single guide RNA (sgRNA) targeting Tet1 and Tet2 into zygotes generated mice with biallelic mutations in both genes with an efficiency of 80%. In addition, co-injection of Cas9 mRNA/sgRNAs with mutant oligos generated precise point mutations in target genes. Thus, shown herein is that the CRISPR/Cas system allows the one step generation of animals carrying mutations in multiple genes, an approach that will greatly accelerate the in vivo study of, for example, functionally redundant genes and of epistatic gene interactions. In certain embodiments a method described herein generates non-human mammals, e.g., mice, with biallelic mutations in 1, 2, 3, 4, 5, or more genes with an efficiency of between 20% and 95%, or even more, e.g., at least 20%, 30%, 40%, 50%, 60%, 70%, 80%, 85%, 90%, 95%, or more, e.g., up to 96%, 97%, 98%, 99%, or more. For example, in certain embodiments a method described herein generates non-human mammals, e.g., mice, with biallelic mutations in 2, 3, 4, 5, or more genes with an efficiency of at least 70%, 80%, 85%, 90%, 95%, or more, e.g., between 70% and 85%, 90%, 95%, 96%, 97%, 98%, 99%, or more.

Accordingly, in one aspect, the invention is directed to a method of mutating or modulating one or more target nucleic acid sequences in a (one or more) stem cell or a zygote comprising introducing into the stem cell or zygote (i) one or more ribonucleic acid (RNA) sequences that comprise a portion that is complementary to a portion of each of the one or more target nucleic acid sequences and comprise a binding site for a CRISPR associated (Cas) protein; and a Cas nucleic acid sequence or a variant thereof that encodes a Cas protein having nuclease activity. The stem cell or zygote is maintained under conditions in which the one or more RNA sequences hybridize to the portion of each of the one or more target nucleic acid sequences, and the Cas protein cleaves each of the one or more target nucleic acid sequences upon hybridization of the one or more RNA sequences to the portion of the target nucleic acid sequence, thereby mutating one or more target nucleic acid sequences in the stem cell or zygote. In a particular aspect, the stem cell or zygote into which the one or more RNA sequences and Cas nucleic acid sequence are introduced is an isolated stem cell or isolated zygote. The method can also further comprise introducing the stem cell or zygote into a nonhuman mammal.

The methods described herein can be used to mutate or modulate one or more nucleic acid sequences in a variety of stem cells which include totipotent, pluripotent, multipotent, oligipotent and unipotent stem cells. Specific examples of stem cells include embryonic stem cells, fetal stem cells, adult stem cells, and induced pluripotent stem cells (iPSCs) (e.g., see U.S. Published Application Nos. 20100144031, 20110076678, 20110088107, 20120028821 all of which are incorporated herein by reference).

In some embodiments a stem cell is a pluripotent cell. A “pluripotent” cell has the ability to self-renew and to differentiate into cells of all three embryonic germ layers (endoderm, mesoderm and ectoderm) and, typically, has the potential to divide in vitro for a long period of time, e.g., at least 20, at least 25, or at least 30 passages, or more (e.g., up to 80 passages, or up to 1 year, or more), without losing its self-renewal and differentiation properties. A pluripotent cell is said to exhibit or be in a “pluripotent state”. A pluripotent cell line or cell culture is often characterized in that the cells can differentiate into a wide variety of cell types in vitro and in vivo. Cells that are able to form teratomas containing cells having characteristics of endoderm, mesoderm, and ectoderm when injected into SCID mice are considered pluripotent. Cells that possess ability to participate in formation of chimeras (upon injection into a blastocyst of the same species that is transferred to a suitable foster mother of the same species) that survive to term are pluripotent. If the germ line of the chimeric animal contains cells derived from the introduced cell, the cell is considered germline-competent in addition to being pluripotent.

ES cells are examples of pluripotent cells. ES cells have been derived from mice, primates (including humans), and some other species. ES cells are often derived from cells obtained from the inner cell mass (ICM) of a vertebrate blastocyst but can also be derived from single blastomeres (e.g., removed from a morula). Pluripotent cells can also be obtained using somatic cell nuclear transfer in at least some species, e.g., mice and various non-human primates. Pluripotent cells can also be obtained using parthenogenesis, e.g., from germ cells, e.g., oocytes. Other pluripotent cells include embryonic carcinoma (EC) and embryonic germ (EG) cells. See, e.g., Yu J, Thomson J A, Pluripotent stem cell lines. 22(15):1987-97, 2008.

“Reprogramming”, as used herein, refers to a process that alters the differentiation state or identity of a cell. Induced pluripotent stem (iPS) cells are pluripotent, ES-like cells derived from somatic cells (e.g., fibroblasts, keratinocytes, hematopoietic cells, neural precursor cells) by reprogramming. Reprogramming can be performed using a variety of different methods. As used herein, “reprogramming protocol” refers to any treatment or combination of treatments that causes at least some cells to become reprogrammed. In some embodiments “reprogramming protocol” refers to a set of manipulations (e.g., introduction of nucleic acid(s), e.g., vector(s), carrying particular genes) and/or culture conditions (e.g., culture in medium containing particular compounds) that generates pluripotent cells from somatic cells, e.g., in vitro. As used herein, the term “reprogramming factor” encompasses genes, RNAs, or proteins that promote or contribute to cell reprogramming, e.g., in vitro. Many useful reprogramming factors are transcription factors. In some aspects the terms “reprogramming”, “reprogramming to a pluripotent state”, “reprogramming to pluripotency”, refer to in vitro reprogramming methods that do not require and typically do not include nuclear or cytoplasmic transfer or cell fusion, e.g., with oocytes, embryos, germ cells, or pluripotent cells. Any embodiment or claim may specifically exclude compositions or methods relating to or involving nuclear or cytoplasmic transfer or cell fusion, e.g., fusion of a somatic cell with oocytes, embryos, germ cells, or pluripotent cells or transfer of a somatic cell nucleus to oocytes, embryos, germ cells, or pluripotent cells.

Differentiated cells can be reprogrammed to a pluripotent state by overexpress of the four transcription factors Oct4, Sox2, Klf4, and c-Myc (Takahashi, K. & Yamanaka, S. Cell 126, 663-676, 2006). Fully reprogrammed induced pluripotent stem cells (iPSCs) can contribute to the three germ layers and give rise to fertile mice by tetraploid complementation (Wernig, M., et al. (2007). In vitro reprogramming of fibroblasts into a pluripotent ES-cell-like state. Nature 448, 318-324); Hanna, J., et al. (2009). Direct cell reprogramming is a stochastic process amenable to acceleration. Nature 462, 595-601). The reprogramming process is characterized by widespread epigenetic changes that generate iPSCs that are functionally and molecularly similar to embryonic stem (ES) cells (Carey, B. W. et al. Reprogramming factor stoichiometry influences the epigenetic state and biological properties of induced pluripotent stem cells. Cell Stem Cell 9, 588-598, (2011)).

Reprogramming somatic cells to a pluripotent state can be achieved by infecting cells with retroviruses that encode the transcription factors Oct4, Sox2, Klf4, and c-Myc (termed “OSKM factors”) under control of a viral LTR. Oct4, Sox2 and Klf4 (“OSK factors”) are also sufficient to reprogram mammalian, e.g., rodent or human, somatic cells to pluripotency. Other sets of reprogramming factors, e.g., Oct4, Sox2, Nanog, and Lin28 (OSNL factors) can be used to reprogram mammalian cells, e.g., rodent or human cells, with Lin28 being dispensable. The ectopically expressed factors induce expression of endogenous pluripotency genes such as Oct4 and Nanog. Since the retroviral vectors in iPS cells derived by this approach are silenced, maintenance of pluripotency relies on expression of such endogenous genes and establishment of an appropriate transcriptional network in the reprogrammed cells. Furthermore, reprogramming factors that are members of the same gene family may be used in place of one another in certain embodiments. For example, Klf2 and Klf5 can substitute for Klf4, Sox1 for Sox2 and N-Myc for c-Myc. It has recently been discovered that reprogramming can be achieved using Sall4, Nanog, Esrrb, and Lin28 as reprogramming factors (SNEL factors) or using Sal4, Lin28, Essrb, and Dppa2 (SLED factors) (Buganim Y, et al., Cell. 2012 Sep. 14; 150(6):1209-22). Thus, examples of reprogramming factors of interest for reprogramming somatic cells to pluripotency in vitro include Oct4, Sall4, Nanog, Esrrb, Lin28, Klf4, c-Myc, Dppa2, and any gene/RNA/protein that can substitute for one or more of these in a method of reprogramming somatic cells in vitro.

Exogenous reprogramming factors may be introduced into somatic cells in any form that is capable of maintaining exogenous reprogramming factors for a period of time and at levels sufficient to activate endogenous pluripotency genes and for reprogramming of at least some of the somatic cells into which the exogenous reprogramming factors are introduced to occur. As used herein, “exogenous” refers to a substance present in a cell or organism other than its native source. For example, the terms “exogenous nucleic acid” or “exogenous protein” refer to a nucleic acid or protein that has been introduced by a process involving the hand of man into a biological system such as a cell or organism in which it is not normally found or in which it is found in lower amounts. A substance will be considered exogenous if it is introduced into a cell or an ancestor of the cell that inherits the substance. In contrast, the term “endogenous” refers to a substance that is native to the biological system.

Somatic cells of use in aspects of the invention may be primary cells (non-immortalized cells), such as those freshly isolated from an animal, or may be derived from a cell line capable of prolonged proliferation in culture (e.g., for longer than 3 months) or indefinite proliferation (immortalized cells). Adult somatic cells may be obtained from individuals, e.g., human subjects, and cultured according to standard cell culture protocols available to those of ordinary skill in the art. Cells may be maintained in cell culture following their isolation from a subject. In certain embodiments, the cells are passaged once or more following their isolation from the individual (e.g., between 2-5, 5-10, 10-20, 20-50, 50-100 times, or more) prior to their use in a method of the invention. In some embodiments, cells may be frozen and subsequently thawed prior to use. In some embodiments, cells will have been passaged no more than 1, 2, 5, 10, 20, or 50 times following their isolation from an individual prior to their use in a method of the invention. Somatic cells of use in aspects of the invention include mammalian cells, such as, for example, human cells, non-human primate cells, or rodent (e.g., mouse, rat) cells. They may be obtained by well-known methods from various organs, e.g., skin, lung, pancreas, liver, stomach, intestine, heart, breast, reproductive organs, muscle, blood, bladder, kidney, urethra and other urinary organs, etc., generally from any organ or tissue containing live somatic cells. Mammalian somatic cells useful in various embodiments include, for example, fibroblasts, Sertoli cells, granulosa cells, neurons, pancreatic cells, epidermal cells, epithelial cells, endothelial cells, hepatocytes, hair follicle cells, keratinocytes, hematopoietic cells, melanocytes, chondrocytes, lymphocytes (B and T lymphocytes), macrophages, monocytes, mononuclear cells, cardiac muscle cells, skeletal muscle cells, etc. In some embodiments a somatic cell is a terminally differentiated somatic cell. In some embodiments a somatic cell is a progenitor (precursor) cell, which has not terminally differentiated.

In some embodiments, reprogramming factors are introduced into somatic cells in the form of one or more nucleic acid sequences encoding the reprogramming factors. In some embodiments, reprogramming factors are introduced into somatic cells in the form of one or more nucleic acid sequences encoding the reprogramming factors. In some embodiments, the one or more nucleic acid sequences comprise DNA. In some embodiments, the one or more nucleic acid sequences comprise RNA. In some embodiments, the one or more nucleic acid sequences comprise a nucleic acid construct. In some embodiments, the one or more nucleic acid sequences comprise a vector for delivery of the reprogramming factors into a target cell (e.g., a mammalian somatic cell, e.g., a human or mouse fibroblast cell). Any suitable vector may be used. Examples of suitable vectors are described by Stadtfeld and Hochedlinger (Genes Dev. 24:2239-2263, 2010, incorporated herein by reference in its entirety). Other suitable vectors are apparent to those skilled in the art.

In some embodiments, a vector comprises an inducible vector. In some embodiments, the inducible vector is a doxycycline inducible vector (i.e., a vector activates expression of said reprogramming factors in the presence of doxycycline in a culture medium). “Expression” refers to the cellular processes involved in producing RNA and proteins as applicable, for example, transcription, translation, folding, modification and processing. “Expression products” include RNA transcribed from a gene and polypeptides obtained by translation of mRNA transcribed from a gene. In some embodiments, the inducible vector is a tamoxifen inducible vector or encodes a tamoxifen-inducible protein. In some embodiments, a vector is an integrating vector that integrates into a genome of a host cell (e.g., a mammalian somatic cell). In some embodiments, a vector comprises a viral vector, e.g., a retroviral vector, e.g., a lentiviral vector. In some embodiments, a vector comprises an excisable vector. In some embodiments, the excisable vector comprises a transposon, wherein said excisable vector is excisable from said genome by transient expression of a transposase. In certain embodiments, the transposon comprises a piggyback transposon (See, e.g., Woltjen et al. Nature 458:766-770, 2009; Yusa et al. Nat Methods 6:363-369, 2009, incorporated herein by reference in its entirety). In some embodiments, the excisable vector comprises one or more loxP site incorporated into said vector, wherein said vector can be excised from said genome by transient expression of a Cre recombinase (See, e.g., Kaji et al. Nature 458:771-775, 2009; Soldner et al. Cell 136:964-977, 2009, each of which is incorporated herein by reference in its entirety). In some embodiments, the excisable vector comprises a floxed lentiviral vector.

In some embodiments, the vector does not integrate into the genome of said somatic cell. In some embodiments, the vector comprises an adenoviral vector (See, e.g., Zhou and Freed. Stem Cells 27:2667-2674, 2009, the teachings of which are incorporated herein by reference). In some embodiments, the vector comprises a Sendai viral vector (See, e.g., Fusaki et al. Proc Jpn Acad 85:348-362, 2009, the teachings of which are incorporated herein by reference). In some embodiments, the vector comprises a plasmid. In some embodiments, the vector comprises an episome (Yu et al. Science 324(5928):797-801, 2009, the teachings of which are incorporated herein by reference).

In some embodiments, to minimize the number of independent proviral integrations required for reprogramming, a nucleic acid construct comprises a polycistronic vector that can transduce any combination of reprogramming factors with a goal of reducing the number of proviral integrations. Such polycistronic nucleic acid constructs, expression cassettes, and vectors that employ internal ribosomal entry sites and self-cleaving peptides and are capable of transducing any combination of reprogramming factors are described in PCT Application Publication No. WO 2009/152529, incorporated herein by reference in its entirety.

In certain embodiments reprogramming factors are provided by polycistronic nucleic acid constructs (e.g., expression cassettes, and vectors comprising such constructs). In certain embodiments the polycistronic nucleic acid constructs comprise a portion that encodes a self-cleaving peptide. In certain embodiments a polycistronic nucleic acid construct comprises at least two, three, or four, coding regions, wherein the coding regions are linked to each by a nucleic acid that encodes a self-cleaving peptide so as to form a single open reading frame, and wherein the coding regions encode at least first and second reprogramming factors capable, either alone or in combination with one or more additional reprogramming factors, of reprogramming a mammalian somatic cell to pluripotency. In some embodiments of the invention the construct comprises two coding regions separated by a self-cleaving peptide. In some embodiments constructs encode a polyprotein that comprises 2, 3, or 4 reprogramming factors, separated by self-cleaving peptides. In some embodiments the construct comprises expression control element(s), e.g., a promoter, suitable to direct expression in mammalian cells, wherein the portion of the construct that encodes the polyprotein is operably linked to the expression control element(s). The promoter drives transcription of a polycistronic message that encodes the reprogramming factors, each reprogramming factor being linked to at least one other reprogramming factor by a self-cleaving peptide. The promoter can be a viral promoter (e.g., a CMV promoter) or a mammalian promoter (e.g., a PGK promoter). The expression cassette or construct can comprise other genetic elements, e.g., to enhance expression or stability of a transcript. In some embodiments of the invention any of the foregoing constructs or expression cassettes may further include a coding region that does not encode a reprogramming factor, wherein the coding region is separated from adjacent coding region(s) by a self-cleaving peptide. In some embodiments the additional coding region encodes a selectable marker. In some embodiments, the self-cleaving peptide is a viral 2A peptide. In some embodiments, the self-cleaving peptide is an aphthovirus 2A peptide.

In some embodiments a construct comprises sites for a recombinase that is functional in mammalian cells, wherein the sites flank at least the portion of the construct that comprises the coding regions for the factors (i.e., one site is positioned 5′ and a second site is positioned 3′ to the portion of the construct that encodes the polyprotein), so that the sequence encoding the factors can be excised from the genome after reprogramming. The recombinase can be, e.g., Cre or Flp, where the corresponding recombinase sites are LoxP sites and Frt sites. In some embodiments the recombinase is a transposase. It will be understood that the recombinase sites need not be directly adjacent to the region encoding the polyprotein but will be positioned such that a region whose eventual removal from the genome is desired is located between the sites. In some embodiments the recombinase sites are on the 5′ and 3′ ends of an expression cassette. Excision may result in a residual copy of the recombinase site remaining in the genome, which in some embodiments is the only genetic change resulting from the reprogramming process.

In some embodiments, one or more nucleic acids for introducing reprogramming factors comprise mRNA that is translatable in a mammalian somatic cell. In some embodiments, the mRNA can be introduced in vitro into somatic cells to be reprogrammed and translated by endogenous enzymes into proteins that can activate one or more endogenous pluripotency genes in the cell. As used herein, “pluripotency gene”, refers to a gene whose expression under normal conditions (e.g., in the absence of genetic engineering or other manipulation designed to alter gene expression) occurs in and is typically restricted to pluripotent stem cells, and is crucial for their functional identity as such. It will be appreciated that the polypeptide encoded by a pluripotency gene may be present as a maternal factor in the oocyte. The gene may be expressed by at least some cells of the embryo, e.g., throughout at least a portion of the preimplantation period and/or in germ cell precursors of the adult. The gene may be expressed in ES cells and/or in embryonic carcinoma cells. The pluripotency gene is typically substantially not expressed in somatic cell types that constitute the body of an adult animal under normal conditions (with the exception of germ cells or precursors thereof, or possibly in certain disease states such as cancer). For example, the pluripotency gene may be one whose average expression level (based on RNA or protein) in ES cells is at least 50-fold or 100-fold greater than its average level in those terminally differentiated cell types present in the body of an adult mammal. In some embodiments, the pluripotency gene is one that encodes multiple splice variants or isoforms of a protein, wherein one or more such variants or isoforms is expressed in at least some adult somatic cell types, while one or more other variants or isoforms is not substantially expressed in adult somatic cells under normal conditions. In some embodiments, expression of the pluripotency gene is essential to maintain the viability or pluripotent state of iPSCs. Thus if the gene is knocked out or its expression is inhibited (i.e., its expression is eliminated or substantially reduced, e.g., such that the average steady state level of RNA transcript and/or protein encoded by the gene is decreased by at least 50%, 60%, 70%, 80%, 90%, 95%, or more), the iPSCs are not formed, die or, in some embodiments, differentiate or cease to be pluripotent. In some embodiments the pluripotency gene is characterized in that its expression in an ES cell or iPS cell decreases (resulting in, e.g., a reduction in the average steady state level of RNA transcript and/or protein encoded by the gene by at least 50%, 60%, 70%, 80%, 90%, 95%, or more) when the cell differentiates into a terminally differentiated cell. Oct4 and Nanog are exemplary pluripotency genes. In some embodiments, the mRNA is in vitro transcribed mRNA. Non-limiting examples of producing in vitro transcribed mRNA are described by Warren et al. (Cell Stem Cell 7(5):618-30, 2010, Mandal P K, Rossi D J. Nat Protoc. 2013 March; 8(3):568-82, and/or PCT/US2011/032679 (WO/2011/130624) the teachings of each of which are incorporated herein by reference). The protocols described may be adapted to produce one or more mRNAs of interest in the present invention. In some embodiments, mRNA, e.g., in vitro transcribed mRNA, comprises a sequence encoding SV40 large T (LT). In some embodiments, mRNA, e.g., in vitro transcribed mRNA, comprises one or more modifications that increase stability or translatability of said mRNA. In some embodiments, mRNA, e.g., in vitro transcribed mRNA comprises a 5′ cap. The cap may be wild-type or modified. Examples of suitable caps and methods of synthesizing mRNA containing such caps are apparent to those skilled in the art.

In some embodiments, mRNA, e.g., in vitro transcribed mRNA, comprises an open reading frame flanked by a 5′ untranslated region and a 3′ untranslated region that enhance translation of said open reading frame, e.g., a 5′ untranslated region that comprises a strong Kozak translation initiation signal, and/or a 3′ untranslated region comprises an alpha-globin 3′ untranslated region.

In some embodiments, mRNA, e.g., in vitro transcribed mRNA comprises a polyA tail. Methods of adding a polyA tail to mRNA are known in the art, e.g., enzymatic addition via polyA polymerase or ligation with a suitable ligase.

The methods provided herein can also be used to mutate or modulate one or more nucleic acids in stem cells that are present in cell compositions such as embryos, zygotes, fetuses, and post-natal mammals. In some embodiments, a stem cell (e.g., an ES or iPS cell), zygote, embryo, or post-natal mammal is already genetically modified (already harbors one or more genetic modifications) prior to being subjected to the methods described herein. For example, the stem cell (e.g., an ES or iPS cell), zygote, embryo, or post-natal mammal may be one into which an exogenous nucleic acid has been introduced by a process involving the hand of man (or may be descended at least in part from a cell or organism into which an exogenous nucleic acid has been introduced by a process involving the hand of man). The nucleic acid may for example contain a sequence that is exogenous to the cell, it may contain native sequences (i.e., sequences naturally found in the cells) but in a non-naturally occurring arrangement (e.g., a coding region linked to a promoter from a different gene), or altered versions of native sequences, etc. In some embodiments, a stem cell (e.g., an ES or iPS cell), zygote, embryo, or post-natal mammal is not already genetically modified (does not already harbor one or more genetic modifications) prior to being subjected to the methods described herein.

The stem cell, zygote, embryo, or post-natal mammal can be of vertebrate (e.g., mammalian) origin. In some aspects, the vertebrates are mammals or avians. Particular examples include primate (e.g., human), rodent (e.g., mouse, rat), canine, feline, bovine, equine, caprine, porcine, or avian (e.g., chickens, ducks, geese, turkeys) stem cells, zygotes, embryos, or post-natal mammals. In some embodiments, the stem cell, zygote, embryo, or post-natal mammal is isolated (e.g., an isolated stem cell; an isolated zygote; an isolated embryo). In some embodiments, a mouse stem cell, mouse zygote, mouse embryo, or mouse post-natal mammal is used. In some embodiments, a rat stem cell, rat zygote, rat embryo, or rat post-natal mammal is used. In some embodiments, a human stem cell, human zygote or human embryo is used.

In some aspects, the invention is directed to a method of producing a nonhuman mammal carrying mutations in one or more target nucleic acid sequences comprising introducing into a zygote or an embryo (i) one or more ribonucleic acid (RNA) sequences that comprise a portion that is complementary to a portion of each of the one or more target nucleic acid sequences and comprise a binding site for a CRISPR associated (Cas) protein; and ii) a Cas nucleic acid sequence or a variant thereof that encodes a Cas protein having nuclease activity. The zygote or the embryo is maintained under conditions in which RNA hybridizes to the portion of each of the one or more target nucleic acid sequences, and the Cas protein cleaves each of the one or more target nucleic acid sequences upon hybridization of the RNA to the portion of the target nucleic acid sequence, thereby producing an embryo having one or more mutated nucleic acid sequences. The embryo having one or more mutated nucleic acid sequences may be transferred into a foster nonhuman mammalian mother. The foster nonhuman mammalian mother is maintained under conditions in which one or more offspring carrying the one or more mutated nucleic acid sequences are produced, thereby producing a nonhuman mammal carrying mutations in one or more target nucleic acid sequences.

As will be apparent to those of skill in the art, the nonhuman mammals can also be produced using methods described herein and/or with conventional methods, see for example, U.S. Published Application No. 20110302665. A method of producing a non-human mammalian embryo can comprise injecting non-human mammalian ES cells (e.g., iPSCs) genetically modified according to an inventive method of the present invention into non-human tetraploid blastocysts and maintaining said resulting tetraploid blastocysts under conditions that result in formation of embryos, thereby producing a non-human mammalian embryo. In some embodiments, said non-human mammalian cells are mouse cells and said non-human mammalian embryo is a mouse. In some embodiments, said mouse cells are mutant mouse cells and are injected into said non-human tetraploid blastocysts by microinjection. In some embodiments laser-assisted micromanipulation or piezo injection is used. In some embodiments, a non-human mammalian embryo comprises a mouse embryo.

Another example of such conventional techniques is two step cloning which involves introducing embryonic stem (ES) and/or induced pluripotent stem (iPS) cells comprising the one or more mutations into a blastocyst (e.g., a tetraploid blastocyst) and maintaining the blastocyst under conditions that result in development of an embryo. The embryo is then transferred (impregnated) into an appropriate foster mother, such as a pseudopregnant female (e.g., of the same species as the embryo). The foster mother is then maintained under conditions that result in development of live offspring that harbor the one or more mutations.

Another example is the use of the tetraploid complementation assay in which cells of two mammalian embryos are combined to form a new embryo (Tam and Rossant, Develop, 130:6156-6163 (2003)). The assay involves producing a tetraploid cell in which every chromosome exists fourfold. This is done by taking an embryo at the two-cell stage and fusing the two cells by applying an electrical current. The resulting tetraploid cell continues to divide, and all daughter cells will also be tetraploid. Such a tetraploid embryo develops normally to the blastocyst stage and will implant in the wall of the uterus. In the tetraploid complementation assay, a tetraploid embryo (either at the morula or blastocyst stage) is combined with normal diploid embryonic stem cells (ES) from a different organism. The embryo develops normally; the fetus is exclusively derived from the ES cell, while the extra-embryonic tissues are exclusively derived from the tetraploid cells.

Another conventional method used to produce nonhuman mammals includes pronuclear microinjection. DNA is introduced directly into the male pronucleus of a nonhuman mammal egg just after fertilization. Similar to the two-step cloning described above, the egg is implanted into a pseudopregnant female. Offspring are screened for the integrated transgene. Heterozygous offspring can be subsequently mated to generate homozygous animals.

A variety of nonhuman mammals can be used in the methods described herein. For example, the nonhuman mammal can be a rodent (e.g., mouse, rat, guinea pig, hamster), a nonhuman primate, a canine, a feline, a bovine, an equine, a porcine or a caprine.

In some aspects, various mouse strains and mouse models of human disease are used in conjunction with the methods of producing a nonhuman mammal carrying mutations in one or more target nucleic acid sequences described herein. One of ordinary skill in the art appreciates the thousands of commercially and non-commercially available strains of laboratory mice for modeling human disease. Mice models exist for diseases such as cancer, cardiovascular disease, autoimmune diseases and disorders, inflammatory diseases, diabetes (type 1 and 2), neurological diseases, and other diseases. Examples of commercially available research strains include, and is not limited to, 11BHSD2 Mouse, GSK3B Mouse, 129-E Mouse HSD11B1 Mouse, AKR Mouse Immortomouse®, Athymic Nude Mouse, LCAT Mouse, B6 Albino Mouse, Lox-1 Mouse, B6C3F1 Mouse, Ly5 Mouse, B6D2F1 (BDF1) Mouse, MMP9 Mouse, BALB/c Mouse, NIH-III Nude Mouse, BALB/c Nude Mouse, NOD Mouse, NOD SCID Mouse, Black Swiss Mouse, NSE-p25 Mouse, C3H Mouse, NU/NU Nude Mouse, C57BL/6-E Mouse, PCSK9 Mouse, C57BL/6N Mouse, PGP Mouse (P-glycoprotein Deficient), CB6F1 Mouse, repTOP™ ERE-Luc Mouse, CD-1® Mouse, repTOP™ mitoIRE Mouse, CD-1® Nude Mouse, repTOP™ PPRE-Luc Mouse, CD1-E Mouse, Rip-HAT Mouse, CD2F1 (CDF1) Mouse, SCID Hairless Congenic (SHC™) Mouse, CF-1™ Mouse, SCID Hairless Outbred (SHO™) Mouse, DBA/2 Mouse, SJL-E Mouse, Fox Chase CB17™ Mouse, SKH1-E Mouse, Fox Chase SCID® Beige Mouse, Swiss Webster (CFW®) Mouse, Fox Chase SCID® Mouse, TARGATT™ Mouse, FVB Mouse, THE POUND MOUSE™, and GLUT 4 Mouse. Other mouse strains include BALB/c, C57BL/6, C57BL/10, C3H, ICR, CBA, A/J, NOD, DBA/1, DBA/2, MOLD, 129, HRS, MRL, NZB, NIH, AKR, SJL, NZW, CAST, KK, SENCAR, C57L, SAMR1, SAMP1, C57BR, and NZO.

In some aspects, the method of producing a nonhuman mammal carrying mutations in one or more target nucleic acid sequences further comprises mating one or more commercially and/or non-commercially available nonhuman mammal with the nonhuman mammal carrying mutations in one or more target nucleic acid sequences produced by the methods described herein. The invention is also directed to nonhuman mammals produced by the methods described herein.

In the methods provided herein, one or more ribonucleic acid (RNA) sequences comprise a portion that is complementary to a portion of each of the one or more target nucleic acid sequences and comprise a binding site for a CRISPR associated (Cas) protein is introduced into the stem cell, zygote and/or embryo, etc. In some embodiments, the RNA sequence is referred to as guide RNA (gRNA) or single guide RNA (sgRNA).

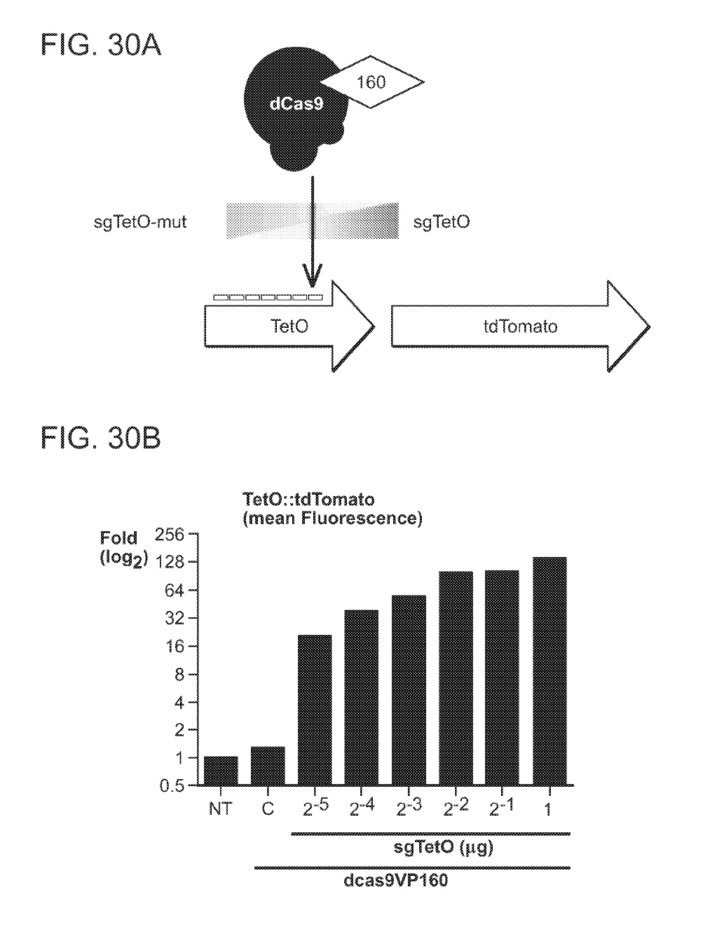

In some aspects, a single RNA sequence can be complementary to one or more (e.g., all) of the target nucleic acid sequences that are being modulated or mutated. In one aspect, a single RNA is complementary to a single target nucleic acid sequence. In a particular aspect in which two or more target nucleic acid sequences are to be modulated or mutated, multiple (e.g., 2, 3, 4, 5, 6, 7, 8, 9, 10, or more) RNA sequences are introduced wherein each RNA sequence is complementary to (specific for) one target nucleic acid sequence. In some aspects, two or more, three or more, four or more, five or more, or six or more RNA sequences are complementary to (specific for) different parts of the same target sequence. In one aspect, two or more RNA sequences bind to different sequences of the same region (e.g. promoter) of DNA (see e.g., FIG. 30A). In some aspects, a single RNA sequence is complementary to at least two target or more (all) of the target nucleic acid sequences. It will also be apparent to those of skill in the art that the portion of the RNA sequence that is complementary to one or more of the target nucleic acid sequences and the portion of the RNA sequence that binds to Cas protein can be introduced as a single sequence or as 2 (or more) separate sequences into a cell, zygote, embryo or nonhuman animal. In some embodiments the sequence that binds to Cas protein comprises a stem-loop.

In some embodiments, the RNA sequence used to modify gene expression in a nonhuman mammal is a naturally occurring RNA sequence, a modified RNA sequence (e.g., a RNA sequence comprising one or more modified bases), a synthetic RNA sequence, or a combination thereof. As used herein a “modified RNA” is an RNA comprising one or more modifications (e.g., RNA comprising one or more non-standard and/or non-naturally occurring bases) to the RNA sequence (e.g., modifications to the backbone and or sugar). Methods of modifying bases of RNA are well known in the art. Examples of such modified bases include those contained in the nucleosides 5-methylcytidine (5 mC), pseudouridine (Ψ), 5-methyluridine, 2′O-methyluridine, 2-thiouridine, N-6 methyladenosine, hypoxanthine, dihydrouridine (D), inosine (I), and 7-methylguanosine (m7G). It should be noted that any number of bases in a RNA sequence can be substituted in various embodiments. It should further be understood that combinations of different modifications may be used.

In some aspects, the RNA sequence is a morpholino. Morpholinos are typically synthetic molecules, of about 25 bases in length and bind to complementary sequences of RNA by standard nucleic acid base-pairing. Morpholinos have standard nucleic acid bases, but those bases are bound to morpholine rings instead of deoxyribose rings and are linked through phosphorodiamidate groups instead of phosphates. Morpholinos do not degrade their target RNA molecules, unlike many antisense structural types (e.g., phosphorothioates, siRNA). Instead, morpholinos act by steric blocking and bind to a target sequence within a RNA and block molecules that might otherwise interact with the RNA.

Each RNA sequence can vary in length from about 8 base pairs (bp) to about 200 bp. In some embodiments, the RNA sequence can be about 9 to about 190 bp; about 10 to about 150 bp; about 15 to about 120 bp; about 20 to about 100 bp; about 30 to about 90 bp; about 40 to about 80 bp; about 50 to about 70 bp in length.

The portion of each target nucleic acid sequence to which each RNA sequence is complementary can also vary in size. In particular aspects, the portion of each target nucleic acid sequence to which the RNA is complementary can be about 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38 39, 40, 41, 42, 43, 44, 45, 46 47, 48, 49, 50, 51, 52, 53, 54, 55, 56, 57, 58, 59 60, 61, 62, 63, 64, 65, 66, 67, 68, 69, 70, 71, 72, 73, 74, 75, 76, 77, 78, 79, 80 81, 82, 83, 84, 85, 86, 87 88, 89, 90, 81, 92, 93, 94, 95, 96, 97, 98, or 100 nucleotides (contiguous nucleotides) in length. In some embodiments, each RNA sequence can be at least about 70%, 75%, 80%, 85%, 90%, 95%, 100%, etc. identical or similar to the portion of each target nucleic acid sequence. In some embodiments, each RNA sequence is completely or partially identical or similar to each target nucleic acid sequence. For example, each RNA sequence can differ from perfect complementarity to the portion of the target sequence by about 1, 2, 3, 4, 5, 6, 7, 8, 9, 10, 11, 12, 13, 14, 15, 16, 17, 18, 19, 20, etc. nucleotides. In some embodiments, one or more RNA sequences are perfectly complementary (100%) across at least about 10 to about 25 (e.g., about 20) nucleotides of the target nucleic acid.

As will be apparent to those of ordinary skill in the art, the one or more RNA sequences can further comprise one or more expression control elements. For example, in some embodiments the RNA sequences comprises a promoter, suitable to direct expression in cells, wherein the portion of the RNA sequence is operably linked to the expression control element(s). The promoter can be a viral promoter (e.g., a CMV promoter) or a mammalian promoter (e.g., a PGK promoter). The RNA sequence can comprise other genetic elements, e.g., to enhance expression or stability of a transcript. In some embodiments the additional coding region encodes a selectable marker (e.g., a reporter gene such as green fluorescent protein (GFP)).

As described herein, the one or more RNA sequences also comprise a (one or more) binding site for a (one or more) CRISPR associated (Cas) protein, and, upon hybridization of the one or more RNA sequences to the one or more target sequences, a (one or more) Cas protein or variant thereof cleaves or nicks each of the target nucleic acid sequences. In a particular aspect, upon hybridization of the one or more RNA sequences to the one or more target nucleic acid sequences, the Cas protein or variants thereof binds to the one or more RNA sequences and cleaves the one or more target nucleic acids sequences. Bacteria and Archaea have evolved an RNA-based adaptive immune system that uses CRISPR (clustered regularly interspaced short palindromic repeat) and Cas (CRISPR-associated) proteins to detect and destroy invading viruses and plasmids (Horvath and Barrangou, Science, 327(5962):167-170 (2010); Wiedenheft et al., Nature, 482(7385):331-338 (2012)). Cas proteins, CRISPR RNAs (crRNAs) and trans-activating crRNA (tracrRNA) form ribonucleoprotein complexes, which target and degrade foreign nucleic acids, guided by crRNAs (Gasiunas et al., Proc. Natl. Acad. Sci, 109(39):E2579-86 (2012); Jinek et al., Science, 337:816-821 (2012)).

In one aspect, the method further comprises introducing one or more Cas nucleic acid or variant thereof into the cell, embryo, zygote, or non-human mammal. In some aspects, a Cas protein or variant thereof is introduced into the cell, embryo, zygote, or non-human mammal. In some aspects, a cell, e.g., stem cell (ES or iPS cell), zygote, embryo, or animal may already harbor a nucleic acid that encodes Cas (may be constitutive or inducible) and/or may already contain Cas protein. For example, in some embodiments a cell, e.g., stem cell (ES or iPS cell), zygote, embryo, or animal, may be descended from a cell or organism into which a nucleic acid encoding a Cas protein has been introduced by a process involving the hand of man.

A variety of CRISPR associated (Cas) genes or proteins which are known in the art can be used in the methods of the invention and the choice of Cas protein will depend upon the particular conditions of the method (e.g., www.ncbi.nlm.nih.gov/gene/?term=cas9). Specific examples of Cas proteins include Cas1, Cas2, Cas3, Cas4, Cas5, Cas6, Cas7, Cas8, Cas9 and Cas10. In a particular aspect, the Cas nucleic acid or protein used in the methods is Cas9. In some embodiments a Cas protein, e.g., a Cas9 protein, may be from any of a variety of prokaryotic species. In some embodiments a particular Cas protein, e.g., a particular Cas9 protein, may be selected to recognize a particular protospacer-adjacent motif (PAM) sequence. In certain embodiments a Cas protein, e.g., a Cas9 protein, may be obtained from a bacteria or archaea or synthesized using known methods. In certain embodiments, a Cas protein may be from a gram positive bacteria or a gram negative bacteria. In certain embodiments, a Cas protein may be from a Streptococcus, (e.g., a S. pyogenes, a S. thermophilus) a Crptococcus, a Corynebacterium, a Haemophilus, a Eubacterium, a Pasteurella, a Prevotella, a Veillonella, or a Marinobacter. In some embodiments nucleic acids encoding two or more different Cas proteins, or two or more Cas proteins, may be introduced into a cell, zygote, embryo, or animal, e.g., to allow for recognition and modification of sites comprising the same, similar or different PAM motifs.

The Cas protein can cleave one strand or both strands (e.g., of a double stranded target nucleic acid), or alternatively, nick one strand or both strands (e.g., of a double stranded target nucleic acid). In some embodiments a Cas9 nickase may be generated by inactivating one or more of the Cas9 nuclease domains. In some embodiments, an amino acid substitution at residue 10 in the RuvC I domain of Cas9 converts the nuclease into a DNA nickase. For example, the aspartate at amino acid residue 10 can be substituted for alanine (Cong et al., Science, 339:819-823). Other amino acids mutations that create a catalytically inactive Cas9 protein includes mutating at residue 10 and/or residue 840. Mutations at both residue 10 and residue 840 can create a catalytically inactive Cas9 protein, sometimes referred herein as dCas9. For example, a D10A and a H840A Cas9 mutant is catalytically inactive.

As shown herein, fusions of a catalytically inactive (D10A; H840A) Cas9 protein (dCas9) tethered with all or a portion of (e.g., biologically active portion of) an (one or more) effector domain create chimeric proteins that can be guided to specific DNA sites by one or more RNA sequences (sgRNA) to modulate activity and/or expression of one or more target nucleic acids sequences (e.g., exert certain effects on transcription or chromatin organization, or bring specific kind of molecules into specific DNA loci, or act as sensor of local histone or DNA state). As used herein, a “biologically active portion of an effector domain” is a portion that maintains the function (e.g. completely, partially, minimally) of an effector domain (e.g., a “minimal” or “core” domain). Specifically, shown herein is that fusion of the Cas9 (e.g., dCas9) with all or a portion of one or more effector domains (e.g., transcriptional activation domains) created a chimeric protein. In one aspect, fusion of a dCas9 with one or more effector domains created a chimeric protein dCas9TA. In some aspects, the one or more effector domains are the same (e.g., VP16 transcriptional activation domains). In other aspects, the one or more effector (e.g., transcriptional activation) domains are different. In some aspects, dCas9TA is guided to specific nucleic acid sites by one or more RNA (e.g. sgRNA). In some aspects, dCas9TA is guided to specific nucleic acid sites by RNA (e.g. sgRNA) to modulate gene expression. In some aspects, all or a portion of one or more VP16 effector domains are fused with Cas9 (e.g., dCas9). In other aspects, 1, 2, 3, 4, 5, 6, 7, 8, 9, 10, 11, 12, 13, 14, 15, 16, 17, 18, 19, 20, or more VP16 effector domains (all or a biologically active portion) are fused with dCas9. In some aspects, a chimeric protein comprising a fusion of a catalytically inactive Cas to all or a portion of one or more effector domains is referred to herein as “CRISPRzyme” or “CRISPR-on”.

In one aspect, fusion of Cas9 with all or a portion of one or more effector domains comprise one or more linkers. As used herein, a “linker” is something that connects or fuses two or more effector domains (e.g see Hermanson, Bioconjugate Techniques, 2^{nd} Edition, which is hereby incorporated by reference in its entirety). As will be appreciated by one of ordinary skill in the art, a variety of linkers can be used. In one aspect, a linker comprises one or more amino acids. In some aspects, a linker comprises 2 or more amino acids. In one aspect, a linker comprises the amino acid sequence GS. In some aspects, fusion of Cas9 (e.g., dCas9) with two or more effector domains (e.g., VP16 core domain such as DALDDFDLDML) comprises one or more interspersed linkers (e.g., GS linkers) between the domains. In some aspects, dCas9 is fused with 3 VP16 core domains with interspersed linkers, referred to herein as dCas9VP48. In other aspects, dCas9 is fused with 4 VP16 core domains with interspersed GS linkers between the core domains, referred herein as dCas9VP48 (SEQ ID NO:14). In other aspects, dCas9 is fused with 6 VP16 core domains with interspersed GS linkers between the core domains, referred herein as dCas9VP96 (SEQ ID NO:15). In other aspects, fusion of dCas9 with 10 VP16 core domains with interspersed GS linkers between the core domains, referred herein as dCas9VP160 (SEQ ID NO:16).

Accordingly, in some aspects, the invention is directed to a method of modulating the expression and/or activity of one or more target nucleic acid sequences in a cell or zygote comprising introducing into the cell or zygote (i) one or more ribonucleic acid (RNA) sequences that comprise a portion that is complementary to each of the one or more target nucleic acid sequences and comprise a binding site for a CRISPR associate (Cas) protein; (ii) a Cas nucleic acid sequence or a variant thereof that encodes the Cas protein that targets but does not cleave the target nucleic acid sequence; and (iii) an (one or more) effector domain. The method further comprises maintaining the cell or zygote under conditions in which the one or more RNA sequences hybridize to the portion of each of the one or more target nucleic acid sequences. The Cas protein binds to each of the one or more RNA sequences and the effector domain modulates the expression and/or activity of the target nucleic acid, thereby modulating the expression and/or activity of a target nucleic acid sequence. As with some aspects of the invention, one or more RNA sequences, Cas nucleic acid sequences and effector domains can be introduced into a cell, zygote, embryo or non-human mammal.

In some aspects, the method of modulating the expression and/or activation of one or more target nucleic acids in a cell is used to reprogram a cell's potency. Cells can be reprogrammed, e.g., by the methods described herein. In one aspect, the invention is directed to a method of modulating the expression and/or activity of one or more target nucleic acid sequences in a cell wherein the cell or cell's potency (e.g., totipotency, pluripotency, multipotency, oligopotency and unipotency) is reprogrammed (e.g., a differentiated cell; a non-differentiated cell). In one aspect, the method results in differentiation of a cell (e.g., a totipotent or pluripotent cell differentiates into a unipotent cell or differentiated cell). In another aspect, the methods results in dedifferentiation of a cell (e.g. a differentiated cell reverts to an earlier developmental stage). For example, the invention is directed to reprogramming a differentiated cell to a totipotent, pluripotent, or multipotent state. In other aspects the method results in transdifferentiation of the cell (e.g. a fibroblast is reprogrammed to a fat cell or a fat cell is reprogrammed to a fibroblast). In one aspect, the one or more target nucleic acid sequences in a cell are overexpressed causing the cell to be reprogrammed. In another aspect, one or more transcription factors are modulated altering cell potency or dedifferentiation. In another aspect, one or transcription factors such as Oct4, Sox2, Klf4, and c-Myc are modulated (e.g. overexpressed) in a cell. (Takahashi, K. & Yamanaka, S. Cell 126, 663-676, 2006).

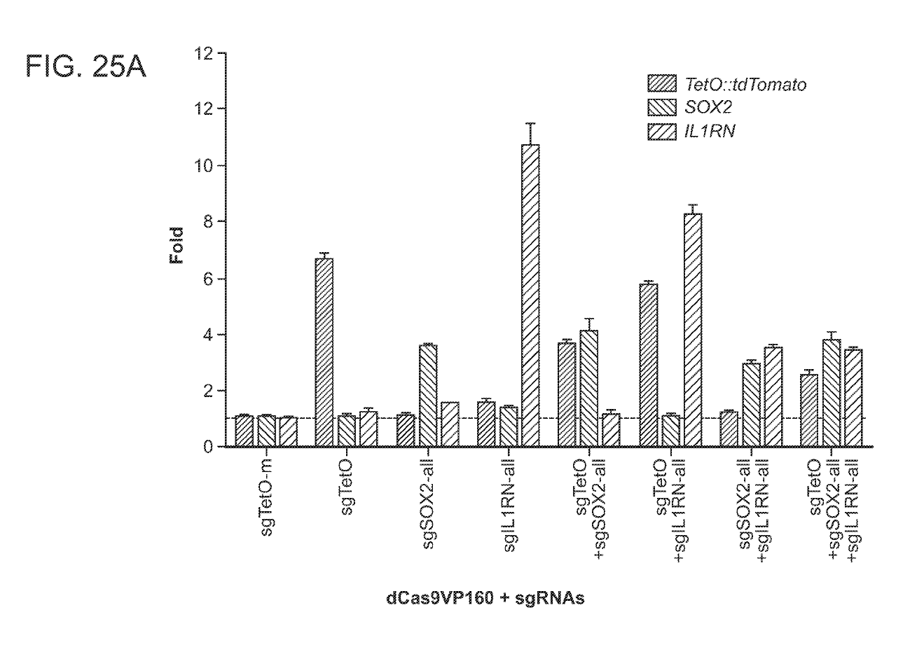

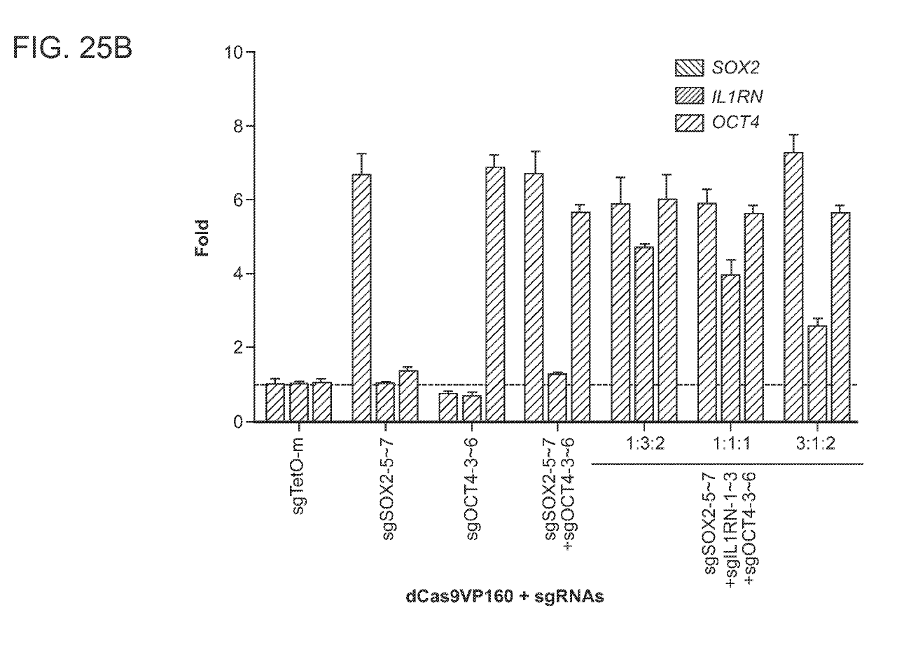

In some aspects, the invention is directed to a method of modulating one or more target nucleic acid sequences comprising simultaneous activation of the one or more target nucleic acid sequences. In another aspect, the method of modulating one or more target nucleic acid sequences comprises adjusting the level of modulation of one or more target nucleic acid sequences by adjusting the amount (e.g. grams, milligrams, micrograms, nanograms, moles, millimoles, micromoles, nanomoles, stoichiometric amount, molar ratio) of the one or more ribonucleic acid sequences introduced into the cell or zygote (FIG. 30B). In some aspects, the level of modulation of one target nucleic acid sequence is the same or different compared to the level of modulation of another target nucleic acid sequence in the same cell or zygote (FIG. 25B). In one aspect, multiple target nucleic acid sequences are modulated (e.g. multiplexed activation).

In some aspects the invention is directed to (e.g., a composition comprising, consisting essentially of, consisting of) a nucleic acid sequence that encodes a fusion protein (chimeric protein) comprising all or a portion of a Cas protein fused to all or a portion of an effector domain. In some aspects, the invention is directed to (e.g., a composition comprising, consisting essentially of, consisting of) a fusion protein comprising all or a portion of a cas protein fused to all or a portion of an effector domain. In some aspects, all or a portion of the cas protein has endonuclease activity (e.g., can cleave and/or nick a target nucleic acid sequence) and/or targeting activity. In some aspects all or a portion of the Cas protein targets but does not cleave a nucleic acid sequence. In some aspects, the Cas protein can be fused to the N-terminus or C-terminus of the effector domain. In some aspects, the portion of the effector domain modulates the expression and/or activation of a target nucleic acid sequence (e.g., gene).

In some aspects, the nucleic acid sequence encoding the fusion protein and/or the fusion protein are isolated. An “isolated,” “substantially pure,” or “substantially pure and isolated” nucleic acid sequence, as used herein, is one that is separated from nucleic acids that normally flank the gene or nucleotide sequence (as in genomic sequences) and/or has been completely or partially purified from other transcribed sequences (e.g., as in an RNA or cDNA library). For example, an isolated nucleic acid of the invention may be substantially isolated with respect to the complex cellular milieu in which it naturally occurs, or culture medium when produced by recombinant techniques, or chemical precursors or other chemicals when chemically synthesized. An “isolated,” “substantially pure,” or “substantially pure and isolated” protein (e.g., chimeric protein; fusion protein), as used herein, is one that is separated from or substantially isolated with respect to the complex cellular milieu in which it naturally occurs, or culture medium when produced by recombinant techniques, or chemical precursors or other chemicals when chemically synthesized. In some instances, the isolated material will form part of a composition (for example, a crude extract containing other substances), buffer system, or reagent mix. In other circumstances, the material may be purified to essential homogeneity, for example, as determined by agarose gel electrophoresis or column chromatography such as HPLC. Preferably, an isolated nucleic acid molecule comprises at least about 50%, 80%, 90%, 95%, 98% or 99% (on a molar basis) of all macromolecular species present.

“Modulate” is used consistently with its use in the art, i.e., meaning to cause or facilitate a qualitative or quantitative change, alteration, or modification in a process, pathway, or phenomenon of interest. Without limitation, such change may be an increase, decrease, or change in relative strength or activity of different components or branches of the process, pathway, or phenomenon. A “modulator” is an agent that causes or facilitates a qualitative or quantitative change, alteration, or modification in a process, pathway, or phenomenon of interest.

In some aspects, “modulating” (“modulates”; “modulation”) the expression and/or activity of a target nucleic acid sequence refers to any of a variety of alterations to the expression and/or activation of the one or more target nucleic acid sequences. For example, the method of modulating the expression and/or activity of the one or more target nucleic acid sequences includes activating, increasing, decreasing, coactivating, regulating, repressing, organizing, remodeling, modifying, and/or fusing the expression and/or activity of one or more target nucleic acid sequences.

Thus, the one or more RNA sequences can be complementary to any of a variety of all or a portion of a target nucleic acid sequence that is to be modulated. In some aspects of the invention, the method of modulating one or more target nucleic acid sequences comprises introducing one or more RNA sequences that are complementary to all or a portion of a (one or more) regulatory region, an open reading frame (ORF; a splicing factor), an intronic sequence, a chromosomal region (e.g., telomere, centromere) of the one or more target nucleic acid sequences into a cell. In some aspects, the target nucleic acid sequence is all or a portion of a plasmid or linear double stranded DNA (dsDNA). In some aspects, the regulatory region targeted by the one or more target nucleic acid sequences is a promoter, enhancer, and/or operator region. In some aspects, all or a portion of the regulatory region is targeted by the one or more target nucleic acid sequences. In some aspects, the regulatory region targeted by the one or more target nucleic acid sequences is exactly or within about 25 bases, 50 bases, 100 bases, 200 bases, 300 bases, 400 bases, 500 bases, 600 bases, 700 bases, 800 bases, 900 bases, 1000 bases, 1500 bases, 2000 bases, or more upstream to the one or more genes (e.g., endogenous genes; exogenous genes) or a (one or more) transcription start site (TSS). In some aspects, the one or more target nucleic acid sequences is exactly or within about 25 bases, 50 bases, 100 bases, 200 bases, 300 bases, 400 bases, 500 bases, 600 bases, 700 bases, 800 bases, 900 bases, 1000 bases, 1500 bases, 2000 bases, or more downstream to the one or more genes (e.g., endogenous genes; exogenous genes) or a TSS. As will be appreciated by one of ordinary skill in the art, the regulatory region targeted by one or more target nucleic acid sequences can be entirely or partially found at or about the 5′ end of the gene (e.g., endogenous or exogenous) or a TSS. The 5′ end of a gene can include untranscribed (flanking) regions (e.g., all or a portion of a promoter) and a portion of the transcribed region.

As used herein, a “regulatory region” is any segment of a nucleic acid sequence capable of modulating (e.g. increasing, decreasing) expression and/or activity of one or more target nucleic acid sequences (e.g. genes). Examples of regulatory regions include a promoter, enhancer, telomere, locus control region, insulator, centromere, repeat sequence, transposable element, synthetic sequence, and operator. Specific examples of regulatory regions include CAAT box, CCAAT box, Pribnow box, TATA box, SECIS element, Polyadenylation signals, A-box, Z-box, C-box, E-box, and/or G-box.

The method of modulating one or more target nucleic acid sequences comprises introducing a Cas nucleic acid sequence or a variant thereof that encodes the Cas protein that targets but does not cleave the target nucleic acid sequence into the cell. In some aspects, a Cas protein or variant thereof is introduced into the cell. In some aspects, the Cas nucleic acid sequence encodes a Cas protein that does not have endonuclease activity. In some aspects, the Cas nucleic acid sequence encodes a Cas protein that does not have nickase activity. In some aspects, the Cas nucleic acid sequence encodes a Cas protein that does not have endonuclease and nickase activity. In some aspects, the Cas nucleic acid sequence encodes a Cas protein that does not have enzymatic activity or is catalytically inactive.

In some aspects of the invention, the method of modulating one or more target nucleic acid sequences comprises introducing a Cas nucleic acid sequence or a variant thereof that encodes a Cas9 protein. In some aspects, the Cas nucleic acid sequence encodes a Cas9 protein that comprises one or more mutations. In some aspects, the Cas nucleic acid sequence encodes a Cas9 protein that comprises a mutation at amino acid position 10, 840, or a combination thereof. In some aspects, the Cas nucleic acid sequence encodes a Cas9 protein wherein the amino acid at position 10 is mutated from aspartate (D) to alanine (A) and the amino acid at position 840 is mutated from histidine (H) to alanine (A).

The method of modulating one or more target nucleic acid sequences also comprises introducing one or more effector domains. As used herein an “effector domain” is a molecule (e.g., protein) that modulates the expression and/or activation of a target nucleic acid sequence (e.g., gene). In some aspects, the effector domain targets one or both alleles of a gene. The effector domain can be introduced as a nucleic acid sequence and/or as a protein. In some aspects, the effector domain can be a constitutive or an inducible effector domain. In some aspects, a Cas nucleic acid sequence or variant thereof and an effector domain nucleic acid sequence are introduced into the cell as a chimeric sequence. In some aspects, the effector domain is fused to a molecule that associates with (e.g., binds to) Cas protein (e.g., the effector molecule is fused to an antibody or antigen binding fragment thereof that binds to Cas protein). In some aspects, a Cas protein or variant thereof and an effector domain are fused or tethered creating a chimeric protein and are introduced into the cell as the chimeric protein. In some aspects, the Cas protein and effector domain bind as a protein-protein interaction. In some aspects, the Cas protein and effector domain are covalently linked. In some aspects, the effector domain associates non-covelently with the Cas protein. In some aspects, a Cas nucleic acid sequence and an effector domain nucleic acid sequence are introduced as separate sequences and/or proteins. In some aspects, the Cas protein and effector domain are not fused or tethered.

Examples of effector domains include a transcription(al) activating domain (e.g., VP16, VP48, VP64, VP96 and VP160), a coactivator domain, a transcription factor, a transcriptional pause release factor domain, a negative regulator of transcriptional elongation domain, a transcriptional repressor domain, a chromatin organizer domain, a remodeler domain, a histone modifier domain, a DNA modification domain, a RNA binding domain, a protein interaction input devices domain (Grünberg and Serrano, Nucleic Acids Research, 38(8):2663-2675 (2010)), and a protein interaction output device domain (Grünberg and Serrano, Nucleic Acids Research, 38(8):2663-2675 (2010)). As used herein a “protein interaction input device” and a “protein interaction output device” refers to a protein-protein interaction (PPI). In some embodiments the PPI is regulatable, e.g., by a small molecule or by light. In some aspect, binding partners are targeted to different sites in the genome using the inactive Cas protein. The binding partners interact, thereby bringing the targeted loci into proximity. A protein interaction output device is a system for detecting/monitoring occurrence of a PPI, generally by producing a detectable signal when the PPI occurs (e.g., by reconstituting a fluorescent protein) or to trigger specific cellular responses (e.g., by reconstituting a caspase protein to induce apoptosis). The idea in this context is to target different sites in the genome with the components of the “output device”. If the interaction occurs, the “output device” generates a signal. This can be used to determine or monitor the proximity of the targeted loci. In some aspects, cells are treated with an agent and the effect of the agent on the cell is determined. Other examples of effector domains include histone marks readers/interactors (http://www.cell.com/abstract/S0092-8674(10)00951-7) and DNA modification readers/interactors.

In some aspects, the effector domain is a VP16 effector domain. In some aspects, the effector domain is a VP48 effector domain. In some aspects, the effector domain is a VP64 effector domain. In some aspects, the effector domain is a VP96 effector domain. In some aspects, the effector domain is a VP160 effector domain.

In one aspect of the invention, fusion of the Cas9 to an effector domain can be to that of a single copy or multiple/tandem copies of full-length or partial-length effectors. Other fusions can be with split (functionally complementary) versions of the effector domains. Effector domains for use in the methods include any one of the following classes of proteins: proteins that mediate drug inducible looping of DNA and/or contacts of genomic loci, proteins that aid in the three-dimensional proximity of genomic loci bound by dCas9 with different sgRNA.

Specific examples of transcription activators or coactivators include VP16, tandem copies comprising all or a biologically active portion of the activation peptide from VP16 (e.g. minimal transactivation domain), such as ADALDDFDLDMLP (SEQ ID NO: 125) and DALDDFDLDML (SEQ ID NO: 126), VP48 (e.g, 3 copies of VP16 minimal transactivation domain), VP64 (e.g., 4 copies of VP16 minimal TA), VP96 (e.g., 6 copies of VP16 minimal TA), VP160 (e.g, 10 copies of VP16 minimal TA), Brd4, and p65.

A specific example of a transcription factor is MYC.

Specific examples of transcriptional pause release factors include proteins in the PTEFb complex, such as Cyclin T1, Cyclin T2, Cyclin T3, Cdk9.

Specific examples of negative regulators of transcriptional elongation include negative elongation factor (NELF) components.

Specific examples of transcriptional repressors include engrailed (EnR), KRAB, Sin3-interaction domain (SID) and EMSY.

Specific examples of chromatin organizers and remodelers include insulator proteins, such as CTCF (transcriptional repressor CTCF or CCCTC-binding factor) to disrupt interactions between enhancers and promoters, cohesin complex and mediator complex Med1 to activate gene expression, switch/sucrose nonfermentable (SWI/SNF) complex—INI1, BAF155b, BAF170, BRG1, hBRM to open up chromatin, and polycomb repressive complex to induce repressive domains on chromatin.

Specific examples of histone modifiers include histone acetyltransferases such as p300/EP300 (p300HAT), CBP/CREBBP (CBPHAT), MGEA5, CDYL, CLOCK, ELP3, GTF3C4, KAT2A, KAT2B, KAT5, MYST2, MYST3, MYST4, HAT1, NAT10, NCOA1, NCOA3, MYST1, CDY1B, CDY1; histone methyltransferases such as SET7, PRMT1, PRMT2, PRMT5, PRMT6, PRMT7, PRMT8, G9a, CARM1, MLL, Set2/SET1A, Ash2, Wdr5, Rbbp5, EZH1, EZH2, MLL2, MLL3, MLL4, MLL5, WHSC1L1, PRDM9, SETD1A, SETD1B, SETD2, SETD7, SETD8, SETDB1, SETDB2, SETMAR, SUV39H1, SUV39H2, SUV420H1, SUV420H2, NSD1, DOT1L, EHMT2, EHMT1, SMYD2, PRDM2, ASH1L, WHSC1, SMYD3; histone Deiminases such as PADI4; histone biotinases such as HLCS; histone ribosylases such as PARP1; histone ubiquitinases such as RNF20, RNF40, DTX3L, HUWE1, RBX1, RING1, RNF2, RNF168, RNF8, UBR2, UHRF1, RAG1; histone kinases such as CDK17, CDK3, CDK5, DAPK3, PRKDC, GSK3B, CHUK, LIMK2, MASTL, MAP3K8, MLT, BUB1, PRKCB, PRKCD, RPS6KA4, RPS6KA5, ATM, STK10, AURKB, STK4, ATR, GSG2, PKN1, NEK6, NEK9, PAK2, TLK1, BAZ1B, JAK2; histone demethylases such as Jarid1, Rbr-2, JMJD6, PHF8, KDM2A, KDM2B, KDM3A, KDM3B, KDM4A, KDM4B, KDM4C, KDM4D, KDM5A, KDM5B, KDM5C, KDM5D, KDM6A, KDM6B, JHDM1D, JMJD5, C14orf189, KDM1A, KDM1B; histone deribosylases such as PARG; histone deubiquitinases such as MSYM1, BRCC3, USP16, USP22, USP3; histone phosphatases such as DUSP1, EYA1, EYA2, EYA3, PPM1D, PPP2CA, PPP2CB, PPP4C, PPP5C, PPP1CC; histone deacetylases such as HDAC1, HDAC10, HDAC11, HDAC2, HDAC3, HDAC4, HDAC5, HDAC6, HDAC7, HDAC8, HDAC9, SIRT1, SIRT2, SIRT3, SIRT6.

Specific examples of DNA modifiers include 5hmc conversion from 5mC such as Tet1 (Tet1CD); DNA demethylation by Tet1, ACIDA, MBD4, Apobec1, Apobec2, Apobec3, Tdg, Gadd45a, Gadd45b, ROS1; DNA methylation by Dnmt1, Dnmt3a, Dnmt3b, CpG Methyltransferase M.SssI, and/or M.EcoHK31I.

Specific examples of RNA binding domains to bring RNA molecules to specific genomic loci include Rbfox2, CUG-BP, MBNL1, MBNL2, MBNL3, MS2 coat protein (MS2 hairpin), and engineered Pumilio.

Specific examples of protein interaction input devices (Grünberg and Serrano, Nucleic Acids Research, 38(8):2663-2675 (2010)) to mediate drug inducible looping of DNA and/or contact of genomic loci include rapamycin induced FKBP:FRB interaction, Jun:Fos, engineered variants of constitutive leuzine zipper interaction, and light-inducible PIF3:PhyB interaction.

Specific examples of protein interaction output devices (Grünberg and Serrano, Nucleic Acids Research, 38(8):2663-2675 (2010)) to report three-dimensional proximity of genomic loci bound by dCas9 with different sgRNA targeting different genomic loci includes split green fluorescent protein (GFP), Fluorescent Resonance Energy Transfer (FRET), split lactamase (antibiotic resistance-based selection) and split capase. These proteins can also be extended to a screening platform for proximal domains in chromatin with a library of sgRNA expression constructs.

Specific examples of histone marks readers/interactors include Sgf29, BPTF, C17orf49/BAP18, GATAD1, TRRAP, PHF8, N-PAC, MSH-6, and NSD1, NSD2, CBX1, CBX3, CBX5, CDYL, and CDYL2.

Specific examples of DNA modification readers/interactors include MeCP2, MBD1, MBD2, MBD3 MBD4, ZBTB4, ZBTB33, ZBTB38, UHRF1, and UHRF2.

In some aspects of the invention, the method of modulating one or more target nucleic acid sequences in a cell can further comprise introducing an effector molecule. As used herein, an “effector molecule” is a molecule (e.g., nucleic acid sequence; protein; organic molecule; inorganic molecule, small molecule) or physical trigger that associates with (e.g., binds to; specifically binds to) the effector domain to modulate the expression and/or activity of a target nucleic acid sequence (e.g., an inducer molecule; a trigger molecule). In some aspects, the effector molecule is a physical signal such as light (e.g., at one or more specific wavelengths; temperature (e.g., temperature-sensitivity); magnetism; stressor and the like. The effector molecule can be contacted with the cell and/or introduced into the cell (e.g., as a nucleic acid sequence or as protein sequence). In some embodiments, the effector molecule is endogenous. In other embodiments, the effector molecule is exogenous. For example, an exogenous effector molecule can be introduced to the cell. In some aspects, the effector molecule binds to the effector domain. In some aspects, the effector molecule is a nucleic acid, protein, drug, small organic molecule and derivatives/variants thereof. In some aspects of the invention, the effector molecule is an antibiotic or derivatives/variants thereof. For example, the antibiotic is doxycycline. One of ordinary skill in the art can appreciate other types of antibiotics used, including but not limited to, tetracycline, ampicillin, puromycin, and neomycin. In some aspects, the effector molecule is rapamycin, tamoxifen and/or derivative/variants thereof (e.g., (Z)-4-hydroxytamoxifen).

As will be appreciated by those of skill in the art, the effector molecule can also associate with one or more domains (e.g., binding domains) that are fused to or associated with the effector domain. For example, the effector domain can be fused to or associated with a receptor domain and/or an antigen binding domain, and the effector molecule (e.g., a ligand specific to the receptor domain; an antibody specific to the antigen binding domain) can bind to the receptor domain and/or antigen binding domain which activates the effector domain, thereby modulating the expression and/or activity of the one or more target nucleic acid sequences.

As will be apparent to those of skill in the art, the method can further comprise introducing other molecules or factors into the cell to facilitate modulation of the activation and/or expression of the target nucleic acid sequence. Examples of such molecules include coactivators, chromatin remodelers, histone acetylases, deacetylases, kinases, and methylases. The methods described herein can also be used to silence expression of a nucleic acid sequence (e.g., a gene) by guiding a repressor to a target nucleic acid sequence.

A variety of target nucleic acid sequences can be mutated or modulated using the methods described herein and will depend upon the desired results. In one aspect, the target nucleic acid sequence is a gene sequence. In particular aspects, the methods described herein can be used to genetically modify two or more different genes in the same gene family, two or more genes that have a redundant function (e.g., redundant may mean that one needs to inactivate at least two of the genes to produce a particular phenotype, e.g., a detectable phenotype), two or more genes of which at least one gene does not or is believed not to produce detectable phenotype when inactivated (e.g., in the strain background used), two or more genes at least 80%, 85%, 90%, 95%, 96%, 97%, 98%, 99%, or more identical, two or more copies of the same gene, two or more genes in same biological pathway (e.g., signaling pathway, metabolic pathway), two or more genes that share at least one biological activity and/or act on at least one common substrate and/or are part of the same protein or protein-nucleic acid complex (e.g., a heteroligomeric protein, spliceosome, proteasome, RISC, transcription complex, replication complex, kinetochore, channel, transporter).

In some aspects, the target nucleic acid sequence is associated with a disease or condition (e.g., see van der Weyden et al., Genome Biol, 12:224 (2011)). Specific examples of genetic modifications of interest include modifying sequence(s), (e.g., gene(s)) to match sequence in different species (e.g., change mouse sequence to human sequence for any gene(s) of interest), alter sites of potential or known post-translational modification of proteins (e.g., phosphorylation, glycosylation, lipidation, acylation, acetylation), alter sites of potential or known epigenetic modification, alter sites of potential or known protein-protein or protein-nucleic acid interaction, inserting tag, e.g., epitope tag, and/or inserting or deleting splice sites. Other examples, include mutating a cell or nonhuman mammal to insert an epitope tag or transgene at an endogenous locus, make a reporter mouse, introduce loxP sites or FlpRT sites flanking certain genomic regions, and/or insert a cassette (e.g., a loxP-stop-loxP or FRT-stop-FRT cassette) in front of a gene to produce conditional alleles (e.g., see Frese and Tuveson, Nature Rev, 7:645-658 (2007); Nern et al., PNAS, 108(34):14198-14203 (2011); Freidal et al., Meth Molec Biol, 693:205-231 (2011)).

In some aspects, one copy of the one or more target nucleic acid sequences is mutated. In some aspects, both copies of one or more of the target nucleic acid sequences in the stem cell or zygote are mutated. In some aspects, the one or more target nucleic acid sequences that are mutated are endogenous to the stem cell or zygote.

In particular aspects, at least two of the target nucleic acid sequences are endogenous nucleic acid sequences. In some aspects, at least two of the target nucleic acid sequences are exogenous nucleic acid sequences. In some aspects where there are at least two target nucleic acid sequences, at least one of the target nucleic acid sequences is an endogenous nucleic acid sequence and at least one of the target nucleic acid sequences is an exogenous nucleic acid sequence. In some aspects, at least two of the target nucleic acid sequences are endogenous genes. In some aspects, at least two of the target nucleic acid sequences are exogenous genes. In some aspects where there are at least two target nucleic acid sequences, at least one of the target nucleic acid sequences is an endogenous gene and at least one of the target nucleic acid sequences is an exogenous gene. In some aspects, at least two of the target nucleic acid sequences are at least 1 kB apart. In some aspects, at least two of the target nucleic acid sequences are on different chromosomes.

As used herein “mutate”, “mutated” or “mutation” and the like refers to alteration of a sequence (a target sequence). For example, in some aspects, a target sequence that has been mutated refers to the replacement, introduction, and/or deletion of one or more nucleotides in the target sequence. In some aspects, a target sequence has been mutated to replace one or more nucleotides in the sequence with one or more nucleotides that occur in one or more natural states of the sequence (e.g., target sequence that is mutated with respect to a wild type sequence has been mutated to replace one or more nucleotides in the sequence with one or more nucleotides that occur in a wild type sequence). In some aspects, a target sequence has been mutated to replace one or more nucleotides that occurs in one or more natural states of the sequence (wild type) with one or more other nucleotides.

In particular aspects, at least one mutation comprises an insertion of a tag (e.g., an epitope tag such as a V5 tag; a fluorescent tag), a transgene (e.g, a reporter gene such as p2A-mCherry, GFP), a translation initiation site (e.g., IRES sequence), a transcription initiation site (e.g., TATA box) and/or an insertion of a site recognized by a recombinase (e.g., Cre). In some aspects, at least one mutation renders expression of an endogenous gene conditional. In yet some aspects, at least one mutation renders expression of an endogenous gene inducible, repressible, or tissue-specific. In still some aspects, the mutations comprise inserting recombination sites (e.g., loxP sites or FRT sites) flanking a selected genomic region, wherein the selected genomic region is optionally within a gene. The mutations can also comprise inserting a recombination-site-STOP-recombination site cassette (e.g., a loxP-STOP-loxP or FRT-STOP-FRT cassette) in a gene, between a promoter and a coding region of a gene, or in a regulatory region of a gene. In this aspect, the recombination-site-STOP-recombination site cassette is positioned so as to disrupt expression of the gene and wherein excision of the cassette by a recombinase renders the gene expressible.

The methods provided herein provide for multiplexed genome editing in cells, embryos, zygotes and nonhuman mammals. As shown herein, cells, embryos, zygotes and non-human mammals carrying mutations in multiple genes can be generated in a single step. In some aspects, the methods described herein allow for the mutation of 1, 2, 3, 4, 5, 6, 7, 8, 9, 10, 11, 12, 13, 14, 15, 16, 17, 18, 19, 20 21, 22, 23, 24, 25, 26, 27, 28, 29, 30, etc. nucleic acid sequences (e.g., genes) in a (single) cell, zygote, embryo or nonhuman mammal using the methods described herein. In a particular aspect, 1 nucleic acid sequence is mutated in a (single) cell, zygote, embryo or nonhuman mammal. In some aspects, 2 nucleic acid sequences are mutated in a (single) cell, zygote, embryo or nonhuman mammal. In some aspects, 3 nucleic acid sequences are mutated in a (single) cell, zygote, embryo or nonhuman mammal. In some aspects, 4 nucleic acid sequences are mutated in a (single) cell, zygote, embryo or nonhuman mammal. In some aspects, 5 nucleic acid sequences are mutated in a (single) cell, zygote, embryo or nonhuman mammal, etc.

The methods described herein can further comprising introducing one or more additional nucleic acid sequences that are complementary to a portion of the one or more target nucleic acid sequences cleaved by the Cas protein. A variety of nucleic acid sequences can be introduced, and include a single stranded oligonucleotide, a double stranded oligonucleotide, a plasmid, a cDNA, a gene block (e.g., gBlocks™ Gene Fragments (IDT)), a PCR product and the like. Thus, the size of the nucleic acid sequences can vary and will depend upon the reason for introducing the nucleic acid sequence. For example, the one or more nucleic acid sequences can be used to replace one or more nucleotides, introduce one or more additional nucleotides, delete one or more nucleotides or a combination thereof in the one or more target nucleic acid sequences. In a particular aspect, the one or more nucleic acid sequences introduce a point mutation in one or more of the target sequences. In some aspects, the one or more nucleic acid sequences replace one or more mutant nucleotides with one or more wild type nucleotides in one or more of the target sequences. In some aspects, the one or more nucleic acid sequences replace one or more wild type nucleotides with one or more (mutant) nucleotides in one or more of the target sequences. In some aspects, the one or more nucleic acids introduce a tag (e.g., a fluorescent protein such as green fluorescent protein), label and/or cleavage site. Thus, the nucleic acid sequence can be from about 10 nucleotides to about 5000 nucleotides, about 20 to 4500 nucleotides, about 30 to 4000 nucleotides, about 50 to 3500 nucleotides, about 60 to about 3000 nucleotides, about 70 to about 2500 nucleotides, about 80 to about 2000 nucleotides, about 90 to about 1500 nucleotides, about 100 to about 1000 nucleotides, etc. In a particular aspect, the nucleic acid sequence is about 10 to about 500 nucleotides.

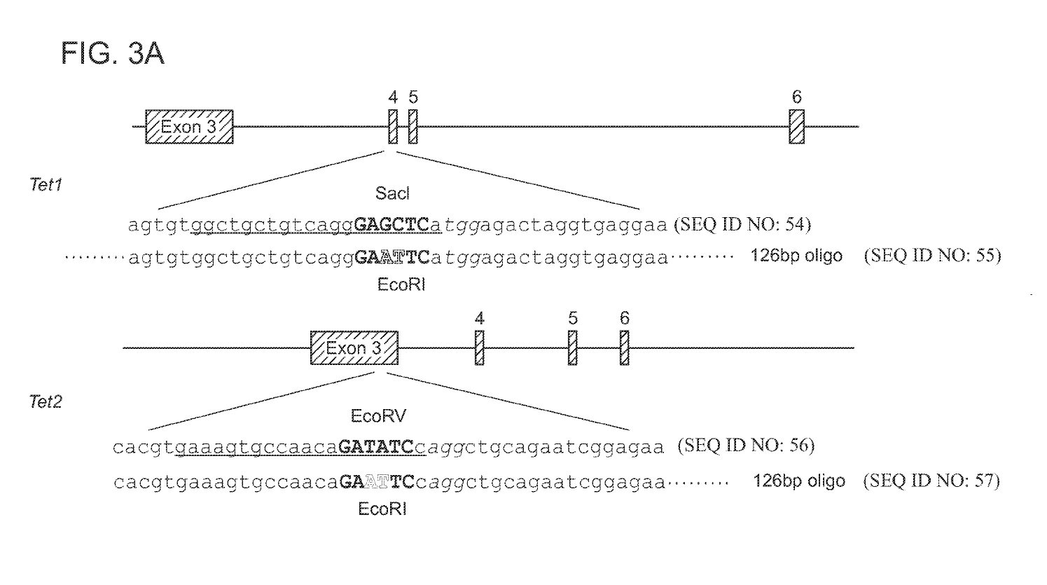

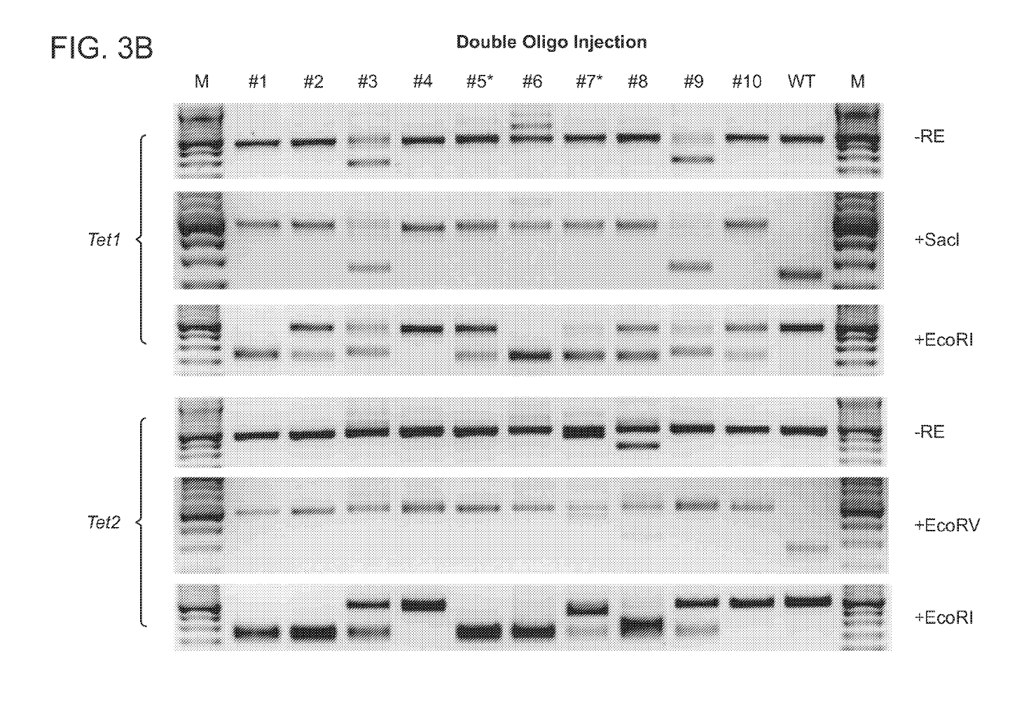

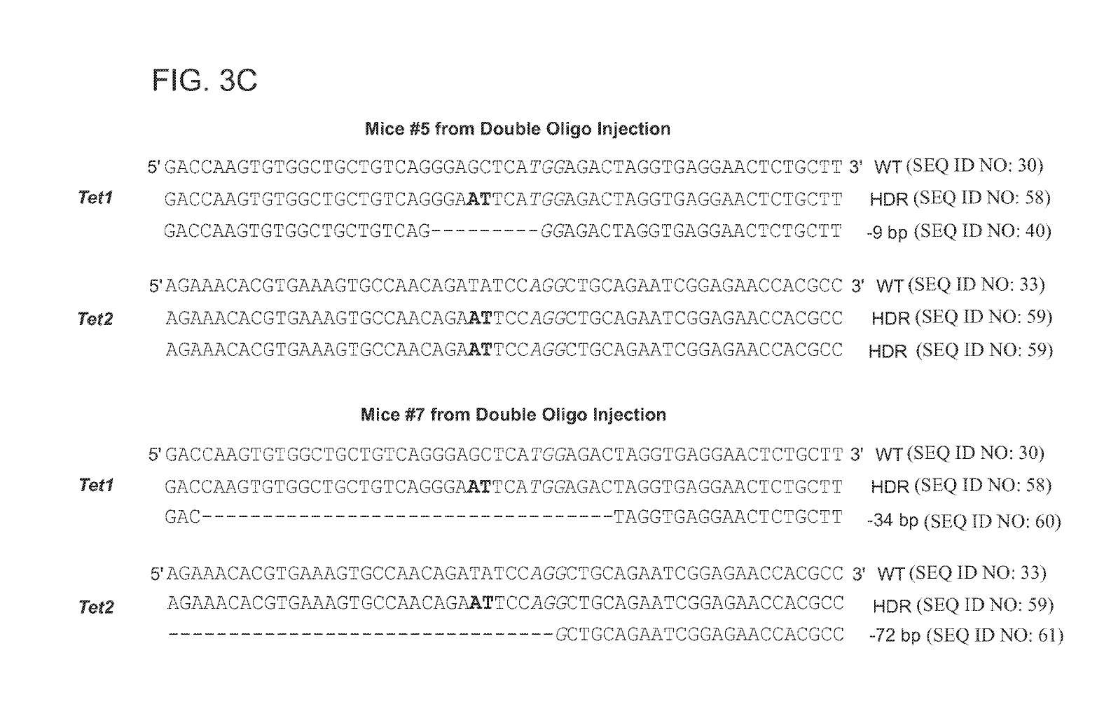

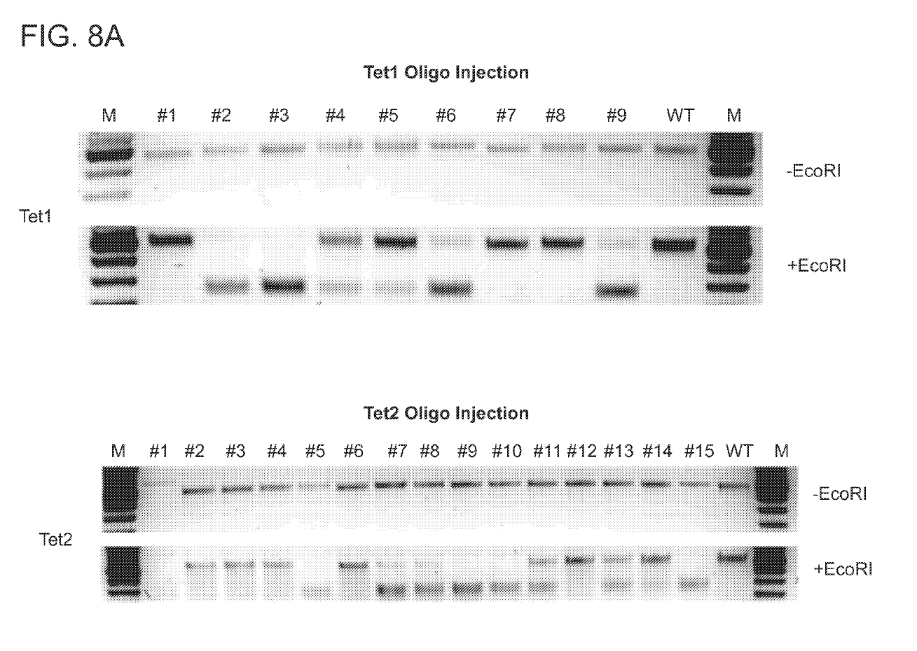

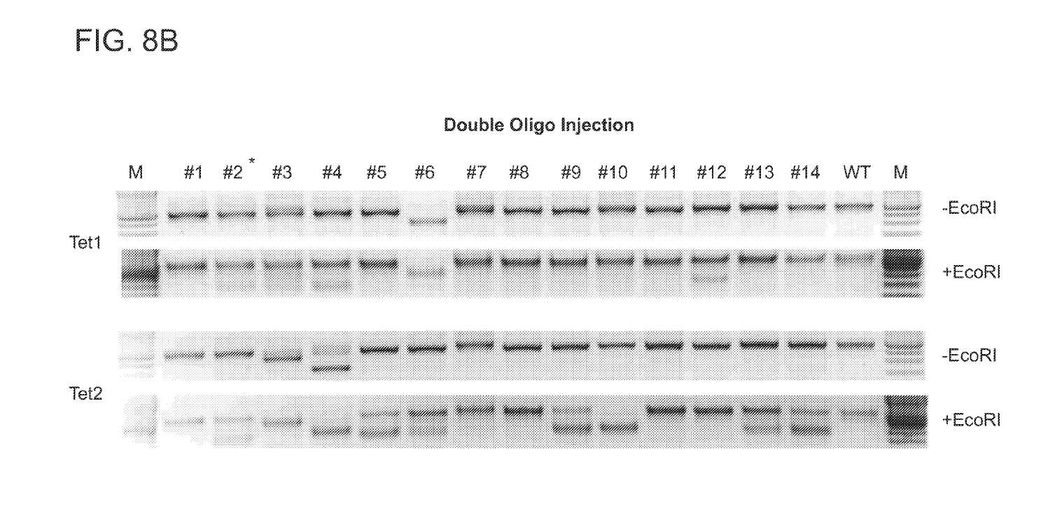

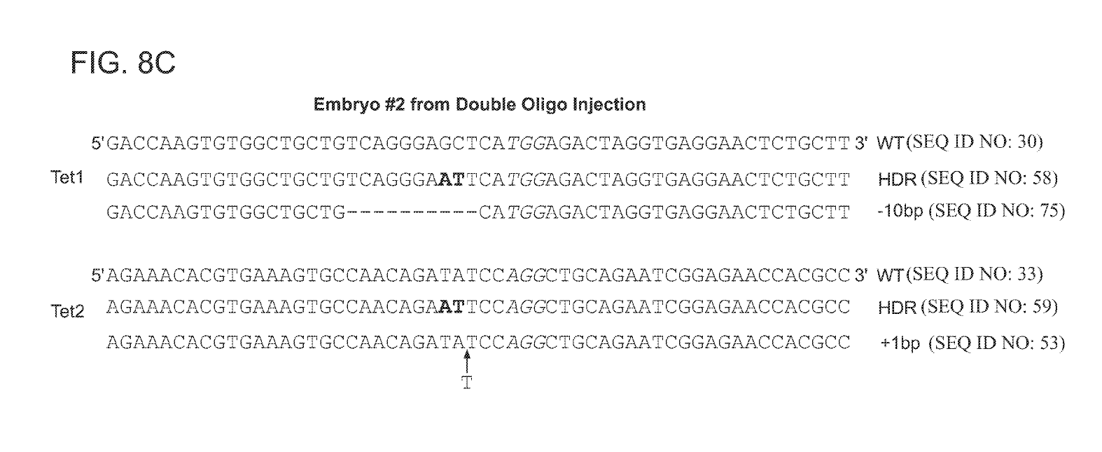

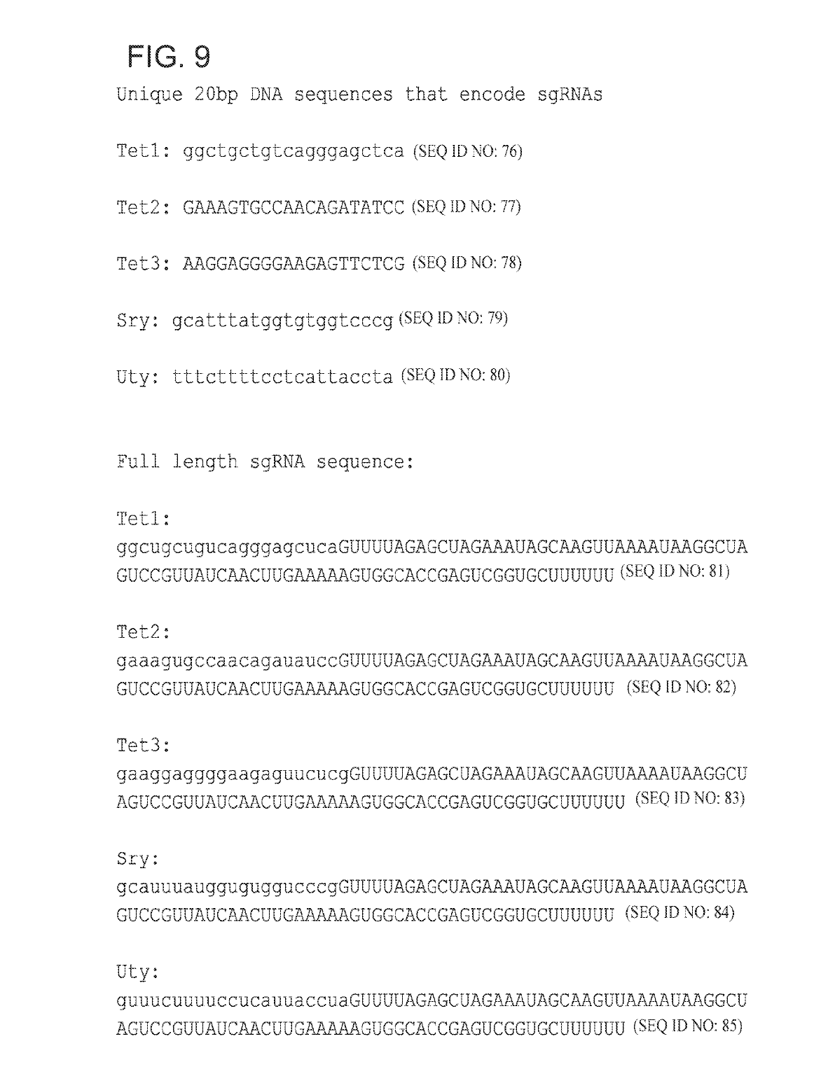

In a particular aspect, the nucleic acid sequence (e.g., oligonucleotide) is used to further modify (alter, edit, mutate) the cleaved target nucleic acid sequence (e.g., such oligo-mediated repair allows for precise genome editing). Thus, this aspect allows for genome editing, however as shown herein the other allele is often mutated through nonhomologous end joining (NHEJ, see FIGS. 3B, 3C, and 8C. Shown herein is that using lower Cas mRNA concentration generates more mice with heterozygous mutations. Therefore, the methods provided herein allow for optimization of the system for more efficient generation of nonhuman mammals with only one oligo-modified allele. In some embodiments, employment of Cas nickase is desirable, since it mainly induces DNA single strand breaks, which is typically repaired through HDR (Cong et al., Science 339:819-823 (2013); Mali et al., Science, 339:823-826 (2013)).

As will be apparent to those of skill in the art, a variety of methods can be used to introduce nucleic acid and/or protein into a stem cell, zygote, embryo, and or mammal. Suitable methods include calcium phosphate or lipid-mediated transfection, electroporation, injection, and transduction or infection using a vector (e.g., a viral vector such as an adenoviral vector). In some aspects, the nucleic acid and/or protein is complexed with a vehicle, e.g., a cationic vehicle, that facilitates uptake of the nucleic acid and/or protein, e.g., via endocytosis.

The method described herein can further comprise isolating the stem cell or zygote produced by the methods. Thus, in some aspects, the invention is directed to a stem cell or zygote (an isolated stem cell or zygote) produced by the methods described herein. In some aspects, the disclosure provides a clonal population of cells harboring the mutation(s), replicating cultures comprising cells harboring the mutation(s) and cells isolated from the generated animals.

The methods described herein can further comprise crossing the generated animals with other animals harboring genetic modifications (optionally in same strain background) and/or having one or more phenotypes of interest (e.g., disease susceptibility—such as NOD mice). In addition, the methods may comprise modifying a stem cell, zygote, and/or animal from a strain that harbors one or more genetic modifications and/or has one or more phenotypes of interest (e.g., disease susceptibility).

In some aspects, various mouse strains and mouse models of human disease are used. One of ordinary skill in the art appreciates the thousands of commercially and non-commercially available strains of laboratory mice for modeling human disease. Mice models exist for diseases such as cancer, cardiovascular disease, autoimmune, inflammatory, diabetes (type 1 and 2), neurobiology, and other diseases. Examples of commercially available research strains include, and is not limited to, 11BHSD2 Mouse, GSK3B Mouse, 129-E Mouse HSD11B1 Mouse, AKR Mouse Immortomouse®, Athymic Nude Mouse, LCAT Mouse, B6 Albino Mouse, Lox-1 Mouse, B6C3F1 Mouse, Ly5 Mouse, B6D2F1 (BDF1) Mouse, MMP9 Mouse, BALB/c Mouse, NIH-III Nude Mouse, BALB/c Nude Mouse, NOD Mouse, NOD SCID Mouse, Black Swiss Mouse, NSE-p25 Mouse, C3H Mouse, NU/NU Nude Mouse, C57BL/6-E Mouse, PCSK9 Mouse, C57BL/6N Mouse, PGP Mouse (P-glycoprotein Deficient), CB6F1 Mouse, repTOP™ ERE-Luc Mouse, CD-1® Mouse, repTOP™ mitoIRE Mouse, CD-1® Nude Mouse, repTOP™ PPRE-Luc Mouse, CD1-E Mouse, Rip-HAT Mouse, CD2F1 (CDF1) Mouse, SCID Hairless Congenic (SHC™) Mouse, CF-1™ Mouse, SCID Hairless Outbred (SHO™) Mouse, DBA/2 Mouse, SJL-E Mouse, Fox Chase CB17™ Mouse, SKH1-E Mouse, Fox Chase SCID® Beige Mouse, Swiss Webster (CFW®) Mouse, Fox Chase SCID® Mouse, TARGATT™ Mouse, FVB Mouse, THE POUND MOUSE™, and GLUT 4 Mouse. Other mouse strains include BALB/c, C57BL/6, C57BL/10, C3H, ICR, CBA, A/J, NOD, DBA/1, DBA/2, MOLD, 129, HRS, MRL, NZB, NIH, AKR, SJL, NZW, CAST, KK, SENCAR, C57L, SAMR1, SAMP1, C57BR, and NZO.

The methods described herein can further comprise assessing whether the one or more target nucleic acids have been mutated and/or modulated using a variety of known methods.

In some embodiments methods described herein are used to produce multiple genetic modifications in a stem cell, zygote, embryo, or animal, wherein at least one of the genetic modifications knocks out (functionally inactivates completely or partially) a gene whose knockout does not produce a detectable phenotype, and at least one of the genetic modifications is in a different gene or genomic location. The resulting stem cell, zygote, embryo, or animal, or a cell, zygote, embryo, or animal generated therefrom, is analyzed for the presence of one or more detectable phenotypes. Such methods may be used to identify genes or genomic locations that have synthetic effects (e.g., effects that are greater in degree or different in kind from the sum of the effects caused by either mutation alone). In some embodiments an effect is synthetic lethality. In some embodiments at least one of the genetic modifications may be conditional (e.g., the effect of the modification, such as gene knockout, only becomes manifest under certain conditions, which are typically under control of the artisan). In some embodiments animals are permitted to develop at least to post-natal stage, e.g., to adult stage. The appropriate conditions for the modification to produce an effect (sometimes termed “inducing conditions”) are imposed, and the phenotype of the animal is subsequently analyzed. A phenotype may be compared to that of an unmodified animal or to the phenotype prior to the imposition of the inducing conditions.

In any aspect or embodiment herein, analysis may comprise any type of phenotypic analysis known in the art, e.g., examination of the structure, size, development, weight, or function, of any tissue, organ, or organ system (or the entire organism), analysis of behavior, activity of any biological pathway or process, level of any particular substance or gene product, etc. In some embodiments analysis comprises gene expression analysis, e.g., at the level of mRNA or protein. In some embodiments such analysis may comprise, e.g., use of microarrays (e.g., oligonucleotide microarrays, sometimes termed “chips”), high throughput sequencing (e.g., RNASeq), ChIP on Chip analysis, ChIPSeq analysis, etc. In some embodiments high content screening may be used, in which elements of high throughput screening may applied to the analysis of individual cells through the use of automated microscopy and image analysis (see, e.g., Zanella et al., (2010). High content screening: seeing is believing. Trends Biotechnol. 28:237-245). In some embodiments analysis comprises quantitative analyses of components of cells such as spatio-temporal distributions of individual proteins, cytoskeletal structures, vesicles, and organelles, e.g., when contacted with test agents, e.g., chemical compounds. In some embodiments activation or inhibition of individual proteins and protein-protein interactions and/or changes in biological processes and cell functions may be assessed. A range of fluorescent probes for biological processes, functions, and cell components are available and may be used, e.g., with fluorescence microscopy. In some embodiments cells or animals generated according to methods herein may comprise a reporter, e.g., a fluorescent reporter or enzyme (e.g., a luciferase such as Gaussia, Renilla, or firefly luciferase) that, for example, reports on the expression or activity of particular genes. Such reporter may be fused to a protein, so that the protein or its activity is rendered detectable, optionally using a non-invasive detection means, e.g., an imaging or detection means such as PET imaging, MRI, fluorescence detection. Multiplexed genome editing according to the invention may allow installation of reporters for detection of multiple proteins, e.g., 2-20 different proteins, e.g., in a cell, tissue, organ, or animal, e.g., in a living animal.

Multiplexed genome editing according to the present invention may be useful to determine or examine the biological role(s) and/or roles in disease of genes of unknown function (e.g., genes whose complete knockout does not produce a detectable phenotype). For example, discovery of synthetic effects caused by mutations in first and second genes may pinpoint a genetic or biochemical pathway in which such gene(s) or encoded gene product(s) is involved. In some embodiments mutations may be generated in stem cells or zygotes from any existing knockout or deletion strain or animals produced according to methods described herein may be crossed with animals from such strain. In some embodiments one or more gain-of-function and/or loss-of-function alleles are generated.

In some embodiments it is contemplated to use, in methods described herein, cells or zygotes generated in or derived from animals produced in projects such as the International Knockout Mouse Consortium (IKMC), the website of which is http://www.knockoutmouse.org). In some embodiments it is contemplated to cross animals generated as described herein with animals generated by or available through the IKMC. For example, in some embodiments a mouse gene to be modified according to methods described herein is any gene from the Mouse Genome Informatics (MGI) database for which sequences and genome coordinates are available, e.g., any gene predicted by the NCBI, Ensembl, and Vega (Vertebrate Genome Annotation) pipelines for mouse Genome Build 37 (NCBI) or Genome Reference Consortium GRCm38.

In some embodiments a gene or genomic location to be modified is included in genome of a species for which a fully sequenced genome exists. Genome sequences may be obtained, e.g., from the UCSC Genome Browser (http://genome.ucsc.edu/index.html). For example, in some embodiments a human gene or sequence to be modified according to methods described herein may be found in Human Genome Build hg19 (Genome Reference Consortium). In some embodiments a gene is any gene for which a Gene ID has been assigned in the Gene Database of the NCBI (http://www.ncbi.nlm.nih.gov/gene). In some embodiments a gene is any gene for which a genomic, cDNA, mRNA, or encoded gene product (e.g., protein) sequence is available in a database such as any of those available at the National Center for Biotechnology Information (www.ncbi.nih.gov) or Universal Protein Resource (www.uniprot.org). Databases include, e.g., GenBank, RefSeq, Gene, UniProtKB/SwissProt, UniProtKB/Trembl, and the like.

In some embodiments a gene encodes a polypeptide. In some embodiments a gene may not encode a polypeptide. A gene may, for example, comprise a template for transcription of a functional RNA, i.e., an RNA that has at least one function other than providing a messenger RNA (mRNA) to be translated into protein. Examples, include, e.g., long non-coding RNA (e.g., greater than 200 bases in length, e.g., 200-5,000 bases), small RNA (e.g., small nuclear RNA), transfer RNA, ribosomal RNA, microRNA precursor, Piwi-interacting RNAs (piRNAs), small nucleolar RNAs (snoRNAs). In some embodiments a small RNA is 25 bases or less, 50 bases or less, 100 bases or less, 200 bases or less in length. Sequences of functional RNAs are available, e.g., from databases such as miRBase (website is http://www.mirbase.org/) (Kozomara A, et al., miRBase: integrating microRNA annotation and deep-sequencing data. NAR 2011 39 (Database Issue):D152-D157), or the Long Non-Coding RNA Database, also called lncRNAdb (website is http://www.lncrnadb.org/), (Amaral P P, et al. (2011) lncRNAdb: a reference database for long noncoding RNAs. Nucleic Acids Res 39: D146-151). In some embodiments a genomic sequence may be suspected of potentially comprising a template for transcription of a functional RNA. A genetic modification may be made in the sequence to determine whether such genetic modification alters the phenotype of a cell or animal or affects production of an RNA or protein or alters susceptibility to a disease.

In some embodiments it is of interest to genetically modify a known or suspected regulatory region, e.g., a known or suspected enhancer region or a known or suspected promoter region. The effect on expression of one or more genes in (e.g., within up to about 1, 2, 5, 10, 20, 50, 100, 500 kB or within about 1, 2, 5, or 10 MB from the modification) may be assessed. A genetic modification may be made in the sequence to determine whether such genetic modification alters the phenotype of a cell or animal or affects production of an RNA or protein or alters susceptibility to a disease.

In some embodiments any method described herein may comprise isolating one or more cells, samples, or substances from an animal generated according to methods described herein, e.g., any genetically modified animal generated as described herein. In some embodiments a method may further comprise analyzing the one or more cells, samples, or substances. Such analysis may, for example assess the effect of a genetic medication(s) introduced according to the methods.

In some embodiments animals generated according to methods described herein may be useful in the identification of candidate agents for treatment of disease and/or for testing agents for potential toxicity or side effects. In some embodiments any method described herein may comprise contacting an animal generated according to methods described herein, e.g., any genetically modified animal generated as described herein, with a test agent (e.g., a small molecule, nucleic acid, polypeptide, lipid, etc.). In some embodiments contacting comprises administering the test agent. Administration may be by any route (e.g., oral, intravenous, intraperitoneal, gavage, topical, transdermal, intramuscular, enteral, subcutaneous), may be systemic or local, may include any dose (e.g., from about 0.01 mg/kg to about 500 mg/kg), may involve a single dose or multiple doses. In some embodiments a method may further comprise analyzing the animal. Such analysis may, for example assess the effect of the test agent in an animal having a genetic medication(s) introduced according to the methods. In some embodiments a test agent that reduces or enhances an effect of one or more genetic modification(s) may be identified. In some embodiments if a test agent reduces or inhibits development of a disease associated with or produced by the genetic modification(s), (or reduces or inhibits one or more symptoms or signs of such a disease) the test agent may be identified as a candidate agent for treatment of a disease associated with or produced by the genetic modification(s) or associated with or produced by naturally occurring mutations in a gene or genomic location harboring the genetic modification.

In some embodiments a cell (e.g., a somatic cell to be used to generate an iPS cell) may be a diseased cell or may originate from a subject suffering from a disease, e.g., a disease affecting the cell or organ from which the cell was obtained. In some embodiments a mutation is introduced into a genomic region of the iPS cell that is associated with a disease (e.g., any disease of interest, such as diseases mentioned herein). For example, in some embodiments it is of interest to knock out or otherwise modify a gene or genomic location that is known or suspected to be involved in disease pathogenesis and/or known or suspected to be associated with increased or decreased risk of developing a disease or particular manifestation(s) of a disease. In some embodiments it is of interest to knock out or otherwise modify a gene or genomic location and determine whether such knockout or modification alters the risk of developing a disease or one or more manifestations of a disease, alters progression of the disease, or alters the response of a subject to therapy or candidate therapy for a disease. In some embodiments it is of interest to modify an abnormal or disease-associated nucleotide or sequence to one that is normal or not associated with disease. In some embodiments this may allow production of genetically matched cells or cell lines (e.g., iPS cells or cell lines) that differ only at one or more selected sites of genetic modification. Multiplexed genome editing as described herein may allow for production of cells or cell lines that are isogenic except with regard to, e.g., between 2 and 20 selected sites or genetic alterations. This may allow for the study of the combined effect of multiple mutations that are suspected of or known to play a role in disease risk, development or progression.

The methods of modulating the expression and/or activity of one or more target nucleic acid sequences in a cell have a variety of uses (e.g., therapeutic, pharmaceutical and/or academic uses). For example, CRISPRzymes can be designed to target specific chromatin loci to exert modification (e.g., methylation or demethylation) on causative genes of diseases due to aberrant chromatin state to correct the chromatin states. In addition, CRISPRzymes can be used to detect/sense certain sequence variation or chromatin states at defined loci guided by sgRNA, or interactions between genomic loci guided by pairs or set of sgRNAs and to exert specific therapeutic outcomes dependent on chromatin state or the interaction of genomic loci.

For example, split fragments of Caspase can be fused to dCas9 and only reconstitute apoptosis-inducing activity when two genomic loci targeted by specific sgRNAs are proximal due to looping under certain disease conditions or cell types, e.g., cancer stem cells. [http://www.ncbi.nlm.nih.gov/pubmed/22070901]. CRISPRzymes can be coupled with biosensors to kill cells on detecting specific histone or DNA modifications at specific loci, e.g., DNA methylation (http://www.ncbi.nlm.nih.gov/pubmed/21797230). A pair of fusions: CRISPR-CaspaseA, MBD1-CaspaseB fusion. MBD1-CaspaseB binds to mCpG, CRISPR-CaspaseA binds to a genomic loci (e.g., hypermethylated genes in cancer) guided by an sgRNA. Only at that defined loci and when the loci is methylated is the Caspase reconstituted, and triggering the killing of cancer cells but not in normal cells. CRISPRzymes can be used to detect chromosomal translocation events resulting in fusion of DNA fragments. dCas9 can be fused to split fragments of fluorescent marker, or luciferase gene and sgRNA targeting the fused genes are used and only when the two specific gene fragments are fused is the reporter reconstituted. This strategy can be used to screen for/detection of subtypes of cancer cells in patient samples/biopsy, at single cell resolution. Similarly fusion with split caspase will allow specific killing/depletion of aberrant cells characteristic of specific chromosomal translocation events. Conversely, CRISPRzymes can be used to restore DNA looping in patients with deficient DNA looping, e.g., Cornelia de Lange patients (defeats in cohesin complex.)

CRISPRzymes can also be used in pharmaceutical and/or academic research. For example, a screen can be used by a library of sgRNA sequences in combination with a CRISPRzyme or a set of CRISPRzymes. The screen can be in the format of library, where each samples (cells, embryos, or tissues) are treated with known and predefined sgRNA or a set of sgRNA. Alternatively, the screen can be pooled whereby vectors expressing different sgRNAs are mixed and introduced to the target (cells, embryos, tissues, etc.) and cells with appropriate phenotype are selected or enriched and the sgRNA harboring the specific phenotype identified by sequencing. CRISPRzymes can be used to elicit chromatin state changes, or transcription activation of specific gene or specific sets of genes in somatic cell, adult stem cells or embryonic stem cells to induce them to reprogram into pluripotent states, to differentiate or transdifferentiate.

In some aspects, methods described herein may be used to produce non-human mammals that have a mutation in the SRY (sex determining region Y) gene. The SRY gene is an intronless gene located on the Y chromosome in therian mammals that encodes a transcription factor that is a member of the SOX (SRY-like box) gene family of DNA-binding proteins. Since a functional Sry protein is required for male development, a mammal that has an X and Y chromosome, wherein the Y chromosome harbors a loss-of-function mutation in SRY, is an anatomic female. An anatomic female may be recognized, e.g., by the presence of a uterus and ovaries and the absence of testes.

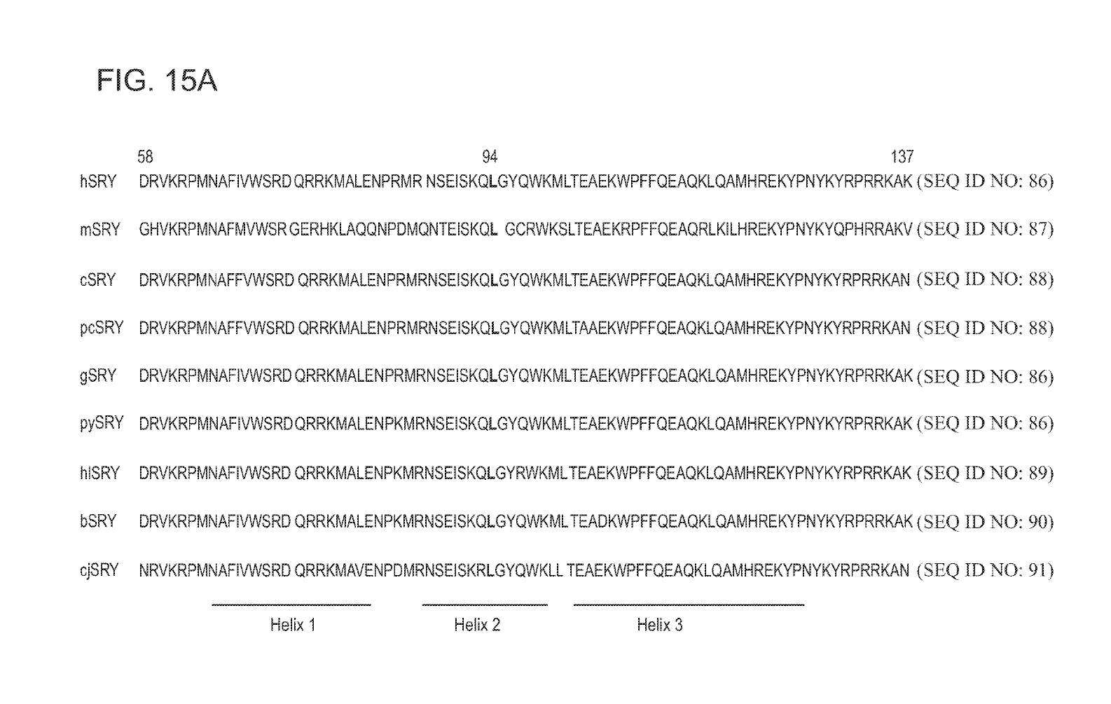

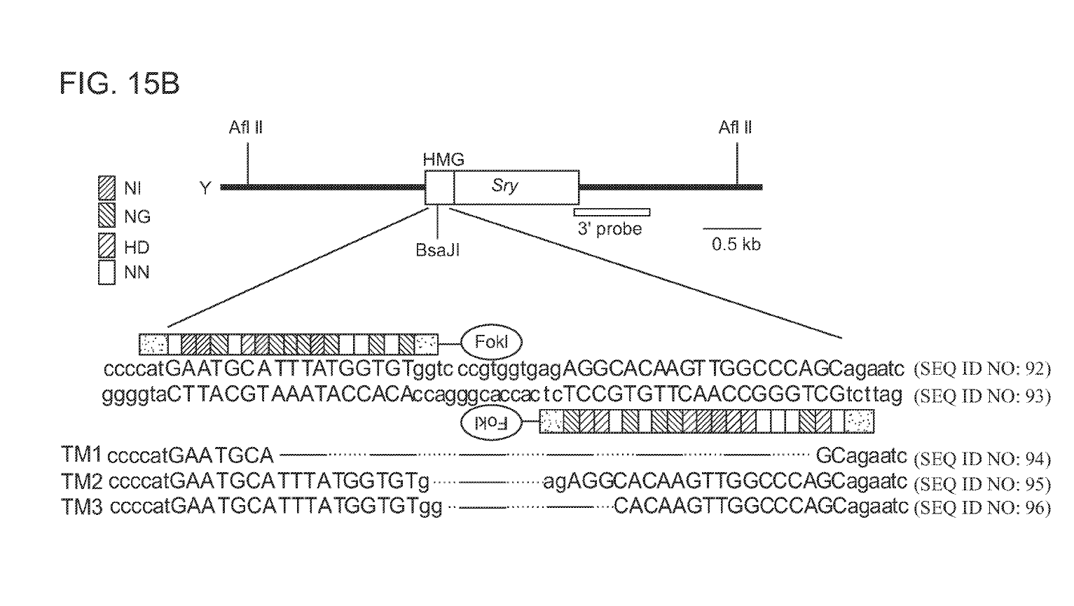

As described herein, the CRISPR/Cas system may be used to generate mutations in SRY, e.g., in a stem cell, zygote, or embryo. Thus in some embodiments, a target nucleic acid sequence mutated according to methods described herein is the SRY gene or a portion thereof. In some embodiments the mutation is a loss-of-function mutation. In some embodiments the loss-of-function mutation is a deletion of part or all of the SRY gene. In some embodiments the mutation, e.g., deletion, is in a portion of the gene that is essential for its function. In some embodiments a mutation is in the portion of the SRY gene that encodes the high mobility group (HMG) DNA binding domain of Sry, termed the HMG box. The HMG box (Nasrin, Nature, 354, 317-320 (1991)). is the characteristic domain of the SOX (SRY-type HMG box) family of transcription factors. It is a 79 amino acid domain that is highly conserved among SRY proteins (at least 50% identical to the human Sry HMG box). In humans, the HMG box extends from amino acid 58 to amino acid 137 of Sry. The corresponding sequences in other species are immediately evident upon aligning the Sry protein sequences with the human sequence (see, e.g., FIG. 15A). For example, in mouse the Sry HMG box extends from amino acid 3 to amino acid 82). The HMG domain is essential for the function of SRY proteins.

In some aspects, the present disclosure relates to the recognition that targeted mutations in SRY cause anatomic sex reversal, resulting in non-human mammals that have X and Y chromosomes but are anatomic females. For example, Applicants have generated XY mice having a variety of deletions or insertion in the SRY gene (Wang H, et al., TALEN-mediated editing of the mouse Y chromosome. Nat Biotechnol. 2013; May 12, doi: 10.1038/nbt.2595. ePub ahead of print, incorporated herein by reference). The mice were generated using transcription activator-like effector nuclease (TALEN) technology to mutate the Sry gene in mouse ES cells. Two pairs of TALENs were generated to target the high mobility group (HMG) DNA binding domain of Sry and were transfected into mouse embryonic stem (ES) cells to generate deletions. TALEN pairs 1 and 2 showed gene modification efficiencies of 15% and 20%, respectively, based on a Surveyor assay. The deletions ranged in size from 11 to 540 bp (Wang, H., supra). Three of the generated deletions are depicted schematically in FIG. 15B. The TALEN cleavage site is in the middle between the binding of the TALEN2 pair as depicted in FIG. 15B. The mutated ES cells were used to produce living mice by tetraploid complementation using standard methods. The resulting mice were found to be anatomic females. In addition, the insertion of a sequence encoding GFP at the same site lead to sex reversal. Adult Sry-targeted mice (anatomic females) showed reduced fertility, but they were fertile and transmitted the Sry-mutated Y chromosome to offspring.

| | TALEN recognition sequences |
|---|---|
| | (bold and underlined) |
| TALEN | & Amino acid sequences of the RVDs |
| Sry | 5′ TGGCCCAGCAGAATCCCAGCATGCAAAATA |
| pair 1 | CAGAGATCAGCAAGC (SEQ ID NO: 127) |
| | 3′ ACCGGGTCGTCTTAGGGTCGTACGTTTTA |
| | TGTCTCTAGTCGTTCG (SEQ ID NO: 128) |
| | NG NN NN HD HD HD NI NN HD NI NN |
| | NI NI NG (Left TALEN) (SEQ ID NO: 129) |
| | NN HD NG NGNN HD NG NN NI NG HD NG HD |
| | NG NN NG (Right TALEN) (SEQ ID NO: 130) |
| Sry | 5′ GAATGCATTTATGGTGTGGTCCCGTGGTGAG |
| pair 2 | AGGCACAAGTTGGCCCAGC (SEQ ID NO: 131) |
| | 3′ CTTACGTAAATACCACACCAGGGCACCACTC |
| | TCCGTGTTCAACCGGGTCG (SEQ ID NO: 132) |
| | NN NI NI NG NN HD NI NG NG NG NI |
| | NG NN NN NG NN NG (SEQ ID NO: 133) |
| | NN HD NG NN NN NN HD HD NI NI HD NG |
| | NG NN NG NN HD HD NG (SEQ ID NO: 134) |

The distributions of genotypes and anatomic sexual phenotypes in progeny from six litters.

| | | Parents | | | | | | |
|---|---|---|---|---|---|---|---|---|
| | | XY^{Sry(tm1)} x XY^{Sry(dl1Rlb )}; Tg(Sry)2Ei | | | | | | |
| Progeny | Genotype | XY^{Sry(dl1Rlb)}; | XY^{Sry(tm1)}; | XX; | XY^{Sry(dl1Rlb)} | XY^{Sry(tm1)} | XX | Y^{Sry(tm1)} |
| | | Tg(Sry)2Ei | Tg(Sry)2Ei | Tg(Sry)2Ei | | | | Y^{Sry(dl1Rlb)}, |
| | | | | | | | | OY^{Sry(dl1Rlb)}, |
| | | | | | | | | Y^{Sry(tm1)} |
| | | | | | | | | Y^{Sry(dl1Rlb)}; |
| | | | | | | | | Tg(Sry)2Ei, |
| | | | | | | | | or |
| | | | | | | | | OY^{Sry(dl1Rlb))}; |
| | | | | | | | | Tg(Sry)2Ei |
| | Anatomic | Male | Male | Male | Female | Female | Female | N/A |
| | Sex | | | | | | | |
| | Number | 5 | 5^{a,b} | 1^{a} | 4 | 2^{a,b} | 5^{a} | Not Viable |
| | (total = 22) | | | | | | | |

From the age of ˜2 months, each of seven XYSry(tm1) females was housed with a single XYSry(dl1Rlb); Tg(Sry)2Ei male for 5-7 months. The result was that three XYSry(tm1) females gave birth to a total of eight litters (two eaten at birth). It has been reported that, in XY female meiosis, the X and Y chromosomes do not pair efficiently and segregate randomly, leading to sex chromosome aneuploidy in the offspring of XY females1, 2. aThese mice may carry either one or two X chromosomes. bThese mice may also carry YSry(dl1Rlb).

In some embodiments the portion of the SRY gene that is targeted is within or overlaps with the portion of the gene that encodes the HMG box. In some embodiments the mutation removes at least 5, 6, 7, 8, 9, 10, 11, 12, 13, 14, 15, 20, 25, 40, 50, 100, or more nucleotides from the gene, e.g., at least 5, 6, 7, 8, 9, 10, 11, 12, 13, 14, 15, 20, 25, 40, 50, 100, or more nucleotides from the portion of the gene that encodes the HMG box. In some embodiments the mutation is in a portion of the gene upstream (5′) of the region that encodes the HMG box, e.g., k encoding a portion of the Sry protein that lies N-terminal to the HMG box. In some embodiments a mutation is an insertion upstream of or within the sequence that encodes the HMG box, wherein the insertion results in a frameshift or stop codon. For example, insertion of 1 or 2 amino acids or a longer sequence not divisible by 3 would result in a frameshift. Insertion of a stop codon in the region located 5′ of the sequence encoding the HMG box would result in a truncated and nonfunctional Sry protein. In some embodiments a mutation may be located in a portion of the SRY gene that encodes a portion of Sry that is C-terminal to the HMG box. In some embodiments a mutation may be in a regulatory region, e.g., a promoter. In some embodiments a mutation may be upstream of the start codon, e.g., in a promoter.

In some embodiments the SRY gene is mutated in a zygote, and the zygote is transferred to the uterus of a foster mother (e.g., a pseudopregnant female) to develop to birth. It will be understood that the zygote may be maintained in culture after mutation of the SRY gene, e.g., to an early embryonic stage (e.g., a blastocyst) and then transferred to the uterus of a foster mother. In some embodiments, the invention provides a zygote having an X and Y chromosome, wherein the Y chromosome has an engineered mutation in the SRY gene, wherein the zygote is capable of developing to an anatomic female. The mammal may be any non-human mammal. In some embodiments a method comprises generating a non-human mammal that has an X chromosome and a Y chromosome (i.e., somatic cells of which contain an X and a Y chromosome).

Methods of creating anatomic females may be useful in any context in which it is desired to reduce the number or proportion of male offspring and/or increase the number of proportion of anatomically female offspring. In some embodiments, methods of generating anatomic females are useful in animal husbandry, which generally refers to the breeding and raising of non-human animals for any of a variety of purposes, e.g., for meat, as sources of animal products (e.g., milk, wool, hair, leather, skin, horn, eggs, or meat), for performing work, or providing companionship, e.g., as pets. In some embodiments it may be of interest to generate anatomic females which may be capable of producing offspring or serving as foster mothers for offspring of that species or producing a product of interest. In some embodiments the non-human mammal is allowed to develop at least until adulthood. In some embodiments the adult non-human mammal gives rise to offspring, which inherit the mutation. In some embodiments a useful product, e.g., milk, wool, hair, leather, skin, horn, or meat, is obtained from the anatomically female non-human mammal.

In the context of dairy farming there is considerable interest in reducing the number of male offspring, as they are not useful for producing milk. In some embodiments, a non-human mammal useful in dairy farming is a cow, goat, sheep, or camel, or other non-human animal useful for the production of milk. In some embodiments a cow is of any of the following breeds: a Holstein (also referred to as Holstein-Friesian), Brown Swiss, Canadienne, Dutch Belted, Guernsey, Ayrshire, Jersey, Kerry, Milking Shorthorn, Milking Devon, or Norwegian Red.

In some embodiments methods of creating anatomic females may be useful in the context of managing species at risk of extinction, e.g., in programs that attempt to maintain or increase the number of individuals of a particular species. In some embodiments a species at risk of extinction may be any species recognized as near threatened, threatened (vulnerable, endangered, or critically endangered), or extinct in the wild by the International Union of Conservation (IUCN). Such species are listed, e.g., on the IUCN Red List of Threatened Species (also known as the IUCN Red List or Red Data List), e.g., the 2012 version (available at the IUCN website at http://www.iucnredlist.org/). In some embodiments the population of a species at risk of extinction may be declining. In some embodiments a species, e.g., a species at risk of extinction, may be, e.g., a bear, canine, caprine, elephant, feline, non-human primate, ovine, rodent, or ungulate species. In some embodiments a species, e.g., a species at risk of extinction, may be a marsupial, e.g., a Tasmanian Devil.

In some embodiments, methods of generating non-human mammals may comprise mutating one or more genes whose mutation results in a phenotype of interest. In some embodiments both copies of the gene are mutated. A phenotype of interest may be any phenotype, e.g., any property of interest. In some embodiments the non-human mammal is a source of food (e.g., milk or meat) or other products useful for humans. In some embodiments at least some humans may be allergic to a component, e.g., a protein, found in the food. A phenotype of interest may comprise reduced or absent production of an allergenic component, or alteration in an allergenic component so as to reduce its allergenicity. For example, in some embodiments the gene encoding a whey protein, e.g., the whey protein beta-lactoglobulin (BLG), a component found in the milk of cows, sheep, and a variety of other species (but not humans) that constitutes a major milk allergen, is mutated. In some embodiments a gene is mutated so as to remove an allergenic epitope or alter it to a non-allergenic form, e.g., by changing or deleting one or more amino acids. The protein may still be produced and able to fulfill its normal function but is no longer allergenic or has reduced allergenicity to humans. In some embodiments a gene is mutated so as to reduce or eliminate production of the protein. In some embodiments a mutation is insertion of a stop codon or deletion or alteration of a start codon or at least a portion of a promoter.

In some embodiments a phenotype of interest may comprise any alteration that qualitatively or quantitatively alters one or more characteristics of a product that is obtained from the non-human mammal, e.g., in a way that makes the product more useful, easier to manipulate, less allergenic, or improved in any way. In some embodiments a characteristic may be color, texture, flavor, consistency, viscosity, thickness, roughness, toughness, tenderness, stringiness, fat content, protein content, sugar content, etc. In some embodiments a phenotype of interest may comprise any alteration that increases the yield of a product (e.g., on a per animal basis, per month or year basis); increases the growth rate; reduces the amount of food, resources, or care consumed or required by the animal; renders the animal more resistant to disease; renders the animal more tolerant of high or low temperature, or reduces the environmental impact of the animal (e.g., reduces methane production). In some embodiments, a phenotype may comprise increased milk production.

In some embodiments a polymorphism, e.g., a single nucleotide polymorphism, may be identified as being associated with a phenotype of interest using methods known in the art (e.g., genetic association studies). Methods described herein may be used to generate non-human mammals having a polymorphism that is associated with the phenotype. The animal may be compared with an otherwise isogenic animal that has not been genetically modified. The effect specifically due to variation at the polymorphic position may be determined. If a mutation or polymorphism confers a phenotype of interest, the non-human mammal may be used as a source of additional animals having the mutation or polymorphism and/or additional mammals having the mutation or polymorphism may be produced using methods described herein.

In some embodiments, methods of generating anatomically female non-human mammals may comprise mutating one or more additional nucleic acids in addition to the SRY gene. For example, any gene the mutation of which results in a phenotype of interest (e.g., reduced allergen content), may be mutated.

The terms “disease”, “disorder” or “condition” are used interchangeably and may refer to any alteration from a state of health and/or normal functioning of an organism, e.g., an abnormality of the body or mind that causes pain, discomfort, dysfunction, distress, degeneration, or death to the individual afflicted. Diseases include any disease known to those of ordinary skill in the art. In some embodiments a disease is a chronic disease, e.g., it typically lasts or has lasted for at least 3-6 months, or more, e.g., 1, 2, 3, 5, 10 or more years, or indefinitely. Disease may have a characteristic set of symptoms and/or signs that occur commonly in individuals suffering from the disease. Diseases and methods of diagnosis and treatment thereof are described in standard medical textbooks such as Longo, D., et al. (eds.), Harrison's Principles of Internal Medicine, 18th Edition; McGraw-Hill Professional, 2011 and/or Goldman's Cecil Medicine, Saunders; 24 edition (Aug. 5, 2011). In certain embodiments a disease is a multigenic disorder (also referred to as complex, multifactorial, or polygenic disorder). Such diseases may be associated with the effects of multiple genes, sometimes in combination with environmental factors (e.g., exposure to particular physical or chemical agents or biological agents such as viruses, lifestyle factors such as diet, smoking, etc.). A multigenic disorder may be any disease for which it is known or suspected that multiple genes (e.g., particular alleles of such genes, particular polymorphisms in such genes) may contribute to risk of developing the disease and/or may contribute to the way the disease manifests (e.g., its severity, age of onset, rate of progression, etc.) In some embodiments a multigenic disease is a disease that has a genetic component as shown by familial aggregation (occurs more commonly in certain families than in the general population) but does not follow Mendelian laws of inheritance, e.g., the disease does not clearly follow a dominant, recessive, X-linked, or Y-linked inheritance pattern. In some embodiments a multigenic disease is one that is not typically controlled by variants of large effect in a single gene (as is the case with Mendelian disorders). In some embodiments a multigenic disease may occur in familial form and sporadically. Examples include, e.g., Parkinson's disease, Alzheimer's disease, and various types of cancer. Examples of multigenic diseases include many common diseases such as hypertension, diabetes mellitus (e.g., type II diabetes mellitus), cardiovascular disease, cancer, and stroke (ischemic, hemorrhagic). In some embodiments a disease, e.g., a multigenic disease is a psychiatric, neurological, neurodevelopmental disease, neurodegenerative disease, cardiovascular disease, autoimmune disease, cancer, metabolic disease, or respiratory disease. In some embodiments at least one gene is implicated in a familial form of a multigenic disease.

In some embodiments a disease is cancer, which term is generally used interchangeably to refer to a disease characterized by one or more tumors, e.g., one or more malignant or potentially malignant tumors. The term “tumor” as used herein encompasses abnormal growths comprising aberrantly proliferating cells. As known in the art, tumors are typically characterized by excessive cell proliferation that is not appropriately regulated (e.g., that does not respond normally to physiological influences and signals that would ordinarily constrain proliferation) and may exhibit one or more of the following properties: dysplasia (e.g., lack of normal cell differentiation, resulting in an increased number or proportion of immature cells); anaplasia (e.g., greater loss of differentiation, more loss of structural organization, cellular pleomorphism, abnormalities such as large, hyperchromatic nuclei, high nuclear:cytoplasmic ratio, atypical mitoses, etc.); invasion of adjacent tissues (e.g., breaching a basement membrane); and/or metastasis. Malignant tumors have a tendency for sustained growth and an ability to spread, e.g., to invade locally and/or metastasize regionally and/or to distant locations, whereas benign tumors often remain localized at the site of origin and are often self-limiting in terms of growth. The term “tumor” includes malignant solid tumors, e.g., carcinomas (cancers arising from epithelial cells), sarcomas (cancers arising from cells of mesenchymal origin), and malignant growths in which there may be no detectable solid tumor mass (e.g., certain hematologic malignancies). Cancer includes, but is not limited to: breast cancer; biliary tract cancer; bladder cancer; brain cancer (e.g., glioblastomas, medulloblastomas); cervical cancer; choriocarcinoma; colon cancer; endometrial cancer; esophageal cancer; gastric cancer; hematological neoplasms including acute lymphocytic leukemia and acute myelogenous leukemia; T-cell acute lymphoblastic leukemia/lymphoma; hairy cell leukemia; chronic lymphocytic leukemia, chronic myelogenous leukemia, multiple myeloma; adult T-cell leukemia/lymphoma; intraepithelial neoplasms including Bowen's disease and Paget's disease; liver cancer; lung cancer; lymphomas including Hodgkin's disease and lymphocytic lymphomas; neuroblastoma; melanoma, oral cancer including squamous cell carcinoma; ovarian cancer including ovarian cancer arising from epithelial cells, stromal cells, germ cells and mesenchymal cells; neuroblastoma, pancreatic cancer; prostate cancer; rectal cancer; sarcomas including angiosarcoma, gastrointestinal stromal tumors, leiomyosarcoma, rhabdomyosarcoma, liposarcoma, fibrosarcoma, and osteosarcoma; renal cancer including renal cell carcinoma and Wilms tumor; skin cancer including basal cell carcinoma and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullary carcinoma. It will be appreciated that a variety of different tumor types can arise in certain organs, which may differ with regard to, e.g., clinical and/or pathological features and/or molecular markers. Tumors arising in a variety of different organs are discussed, e.g., the WHO Classification of Tumours series, 4^{th} ed, or 3^{rd} ed (Pathology and Genetics of Tumours series), by the International Agency for Research on Cancer (IARC), WHO Press, Geneva, Switzerland, all volumes of which are incorporated herein by reference. In some embodiments a cancer is one for which mutation or overexpression of particular genes is known or suspected to play a role in development, progression, recurrence, etc., of a cancer. In some embodiments such genes are targets for genetic modification according to methods described herein. In some embodiments a gene is an oncogene, proto-oncogene, or tumor suppressor gene. The term “oncogene” encompasses nucleic acids that, when expressed, can increase the likelihood of or contribute to cancer initiation or progression. Normal cellular sequences (“proto-oncogenes”) can be activated to become oncogenes (sometimes termed “activated oncogenes”) by mutation and/or aberrant expression. In various embodiments an oncogene can comprise a complete coding sequence for a gene product or a portion that maintains at least in part the oncogenic potential of the complete sequence or a sequence that encodes a fusion protein. Oncogenic mutations can result, e.g., in altered (e.g., increased) protein activity, loss of proper regulation, or an alteration (e.g., an increase) in RNA or protein level. Aberrant expression may occur, e.g., due to chromosomal rearrangement resulting in juxtaposition to regulatory elements such as enhancers, epigenetic mechanisms, or due to amplification, and may result in an increased amount of proto-oncogene product or production in an inappropriate cell type. Proto-oncogenes often encode proteins that control or participate in cell proliferation, differentiation, and/or apoptosis. These proteins include, e.g., various transcription factors, chromatin remodelers, growth factors, growth factor receptors, signal transducers, and apoptosis regulators. A TSG may be any gene wherein a loss or reduction in function of an expression product of the gene can increase the likelihood of or contribute to cancer initiation or progression. Loss or reduction in function can occur, e.g., due to mutation or epigenetic mechanisms. Many TSGs encode proteins that normally function to restrain or negatively regulate cell proliferation and/or to promote apoptosis. Exemplary oncogenes include, e.g., MYC, SRC, FOS, JUN, MYB, RAS, RAF, ABL, ALK, AKT, TRK, BCL2, WNT, HER2/NEU, EGFR, MAPK, ERK, MDM2, CDK4, GLI1, GLI2, IGF2, TP53, etc. Exemplary TSGs include, e.g., RB, TP53, APC, NF1, BRCA1, BRCA2, PTEN, CDK inhibitory proteins (e.g., p16, p21), PTCH, WT1, etc. It will be understood that a number of these oncogene and TSG names encompass multiple family members and that many other TSGs are known. In some embodiments any such gene may be genetically modified, e.g., to generate a cancer model, which may be used, e.g., to determine effect of particular alterations on development of cancer, to determine effect of particular alterations on efficacy of or resistance to treatment, to identify or characterize existing or potential candidate therapeutic agents, etc. Similar methods are envisioned for genes associated with other diseases.

In some embodiments a disease is a cardiovascular disease, e.g., atherosclerotic heart disease or vessel disease, congestive heart failure, myocardial infarction, cerebrovascular disease, peripheral artery disease, cardiomyopathy.

In some embodiments a disease is a psychiatric, neurological, or neurodevelopmental disease, e.g., schizophrenia, depression, bipolar disorder, epilepsy, autism, addiction. Neurodegenerative diseases include, e.g., Alzheimer's disease, Parkinson's disease, amyotrophic lateral sclerosis, frontotemporal dementia.

In some embodiments a disease is an autoimmune diseases e.g., acute disseminated encephalomyelitis, alopecia areata, antiphospholipid syndrome, autoimmune hepatitis, autoimmune myocarditis, autoimmune pancreatitis, autoimmune polyendocrine syndromesautoimmune uveitis, inflammatory bowel disease (Crohn's disease, ulcerative colitis), type I diabetes mellitus (e.g., juvenile onset diabetes), multiple sclerosis, scleroderma, ankylosing spondylitis, sarcoid, pemphigus vulgaris, pemphigoid, psoriasis, myasthenia gravis, systemic lupus erythemotasus, rheumatoid arthritis, juvenile arthritis, psoriatic arthritis, Behcet's syndrome, Reiter's disease, Berger's disease, dermatomyositis, polymyositis, antineutrophil cytoplasmic antibody-associated vasculitides (e.g., granulomatosis with polyangiitis (also known as Wegener's granulomatosis), microscopic polyangiitis, and Churg-Strauss syndrome), scleroderma, Sjögren's syndrome, anti-glomerular basement membrane disease (including Goodpasture's syndrome), dilated cardiomyopathy, primary biliary cirrhosis, thyroiditis (e.g., Hashimoto's thyroiditis, Graves' disease), transverse myelitis, and Guillane-Barre syndrome.

In some embodiments a disease is a respiratory disease, e.g., allergy affecting the respiratory system, asthma, chronic obstructive pulmonary disease, pulmonary hypertension, pulmonary fibrosis, and sarcoidosis.

In some embodiments a disease is a renal disease, e.g., polycystic kidney disease, lupus, nephropathy (nephrosis or nephritis) or glomerulonephritis (of any kind).

In some embodiments a disease is vision loss or hearing loss, e.g., associated with advanced age.

In some embodiments a disease is an infectious disease, e.g., any disease caused by a virus, bacteria, fungus, or parasite. In some embodiments it is of interest to modify genes that may be involved in susceptibility to the disease.

It will be understood that classification of diseases herein is not intended to be limiting. One of ordinary skill in the art will appreciate that various diseases may be appropriately classified in multiple different groups.

In some embodiments a disease is one for which at least one genome-wide association (GWA) study (GWAS) has been performed. In some embodiments a GWAS types multiple “cases” (subjects having a disease of interest or particular manifestations thereof) and “controls” (subjects not having the disease or manifestations) for several thousand to millions, e.g., 1 million or more, e.g., 1-5 million or more, alleles (e.g., single nucleotide polymorphisms) positioned throughout the genome or a substantial portion thereof (e.g., at least 80%, 90%, 95%, or more of the genome). It will be understood that control data may be obtained from historical data. Genotyping may be performed using microarrays or other methods. Alleles associated (e.g., in a statistically significant manner) with increased (or decreased) risk of a disease (or particular manifestations) may thereby be identified. It will be appreciated that statistical results may be corrected for multiple hypothesis testing, e.g., using methods known in the art. In some embodiments a p value of less than about 10⁻⁷, 10⁻⁸, or 10⁻⁹ is considered evidence of association. In some embodiments a gene or allele or polymorphism has been identified as contributing to disease risk or severity in at least one GWAS. See, e.g., http://www.genome.gov/gwastudies for examples of GWAS studies and genetic variants (alleles, polymorphisms) associated with various diseases. In some embodiments a gene (or any sequence) is one for which an allele or polymorphism is associated with an increased or decreased risk of developing a disease of at least 1.1, 1.2, 1.5, 2, 3, 4, 5, 7.5, 10, or more, relative to individuals not having the allele or polymorphism. In some embodiments an allele or polymorphism is associated with an increased or decreased risk of developing a disease of at least 1.1, 1.2, 1.5, 2, 3, 4, 5, 7.5, 10, or more, relative to individuals not having the allele or polymorphism. Genes, alleles, polymorphisms, or genetic loci that may contribute to any phenotypic trait of interest such as longevity, weight, resistance to infection, response or lack thereof to various therapeutic agents, resistance or susceptibility to potentially harmful substances such as toxins or infectious agents (e.g., viruses, bacteria, fungi, parasites), are of interest. A phenotypic trait may be a physical sign (such as blood pressure), a biochemical marker, which in some embodiments may be detectable in a body fluid such as blood, saliva, urine, tears, etc., such as level of a metabolite, LDL, etc., wherein an abnormally low or high level of the marker may correlate with having or not having the disease or with susceptibility to or protection from a disease.

In some embodiments a sequence to be inserted into a genome encodes a tag. The sequence may be inserted into a gene in an appropriate position such that a fusion protein comprising the tag is produced. The term “tag” is used in a broad sense to encompass any of a wide variety of polypeptides. In some embodiments, a tag comprises a sequence useful for purifying, expressing, solubilizing, and/or detecting a polypeptide. In some embodiments a tag may serve multiple functions. In some embodiments a tag is a relatively small polypeptide, e.g., ranging from a few amino acids up to about 100 amino acids long. In some embodiments a tag is more than 100 amino acids long, e.g., up to about 500 amino acids long, or more. In some embodiments, a tag comprises an HA, TAP, Myc, 6×His, Flag, V5, or GST tag, to name few examples. A tag (e.g., any of the afore-mentioned tags) that comprises an epitope against which an antibody, e.g., a monoclonal antibody, is available (e.g., commercially available) or known in the art may be referred to as an “epitope tag”. In some embodiments a tag comprises a solubility-enhancing tag (e.g., a SUMO tag, NUS A tag, SNUT tag, a Strep tag, or a monomeric mutant of the Ocr protein of bacteriophage T7). See, e.g., Esposito D and Chatterjee D K. Curr Opin Biotechnol.; 17(4):353-8 (2006). In some embodiments, a tag is cleavable, so that at least a portion of it can be removed, e.g., by a protease. In some embodiments, this is achieved by including a protease cleavage site in the tag, e.g., adjacent or linked to a functional portion of the tag. Exemplary proteases include, e.g., thrombin, TEV protease, Factor Xa, PreScission protease, etc. In some embodiments, a “self-cleaving” tag is used. See, e.g., PCT/US05/05763. In some embodiments, a tag comprises a fluorescent polypeptide (e.g., GFP or a derivative thereof such as enhanced GFP (EGFP)) or an enzyme that can act on a substrate to produce a detectable signal, e.g., a fluorescence or colorimetric signal. Luciferase (e.g., a firefly, Renilla, or Gaussia luciferase) is an example of such an enzyme. Examples of fluorescent proteins include GFP and derivatives thereof, proteins comprising chromophores that emit light of different colors such as red, yellow, and cyan fluorescent proteins, etc. A tag, e.g., a fluorescent protein, may be monomeric. In certain embodiments a fluorescent protein is e.g., Sirius, Azurite, EBFP2, TagBFP, mTurquoise, ECFP, Cerulean, TagCFP, mTFP1, mUkG1, mAG1, AcGFP1, TagGFP2, EGFP, mWasabi, EmGFP, TagYPF, EYFP, Topaz, SYFP2, Venus, Citrine, mKO, mKO2, mOrange, mOrange2, TagRFP, TagRFP-T, mStrawberry, mRuby, mCherry, mRaspberry, mKate2, mPlum, mNeptune, mTomato, T-Sapphire, mAmetrine, mKeima. See, e.g., Chalfie, M. and Kain, S R (eds.) Green fluorescent protein: properties, applications, and protocols (Methods of biochemical analysis, v. 47). Wiley-Interscience, Hoboken, N. J., 2006, and/or Chudakov, D M, et al., Physiol Rev. 90(3):1103-63, 2010 for discussion of GFP and numerous other fluorescent or luminescent proteins. In some embodiments a tag may comprise a domain that binds to and/or acts a sensor of a small molecule (e.g., a metabolite) or ion, e.g., calcium, chloride, or of intracellular voltage, pH, or other conditions. Any genetically encodable sensor may be used; a number of such sensors are known in the art. In some embodiments a FRET-based sensor may be used. In some embodiments different genes are modified to incorporate different tags, so that proteins encoded by the genes are distinguishably labeled. For example, between 2 and 20 distinct tags may be introduced. In some embodiments the tags have distinct emission and/or absorption spectra. In some embodiments a tag may absorb and/or emit light in the infrared or near-infrared region. It will be understood that any nucleic acid sequence encoding a tag may be codon-optimized for expression in a cell, zygote, embryo, or animal into which it is to be introduced.

In some embodiments it may be of interest to express fragments or domains of a protein, which may act in a dominant negative manner and may, for example, disrupt normal function or interaction of the protein.

In some embodiments a gene of interest encodes a protein the aggregation of which is associated with one or more diseases, which may be referred to as protein misfolding diseases. Examples include, e.g., alpha-synuclein (Parkinson's disease and related disorders), amyloid beta or tau (Alzheimer's disease), TDP-43 (frontotemporal dementia, ALS).

In some embodiments a gene of interest encodes a transcription factor, a transcriptional co-activator or co-repressor, an enzyme, a chaperone, a heat shock factor, a heat shock protein, a receptor, a secreted protein, a transmembrane protein, a histone (e.g., H1, H2A, H2B, H3, H4), a peripheral membrane protein, a soluble protein, a nuclear protein, a mitochondrial protein, a growth factor, a cytokine (e.g., an interleukin, e.g., any of IL-1-IL-33), an interferon (e.g., alpha, beta, or gamma), a chemokine (e.g., a CXC, CX3C, C (or XC), or CX3C chemokine) A chemokine may be CCL1-CCL28, CXCL1-CXCL17, XCL1 or XCL2, or CXC3L1). In some embodiments a gene encodes a colony-stimulating factor, a hormone (e.g., insulin, thyroid hormone, growth hormone, estrogen, progesterone, testosterone), an extracellular matrix protein (e.g., collagen, fibronectin), a motor protein (e.g., dynein, myosin), cell adhesion molecule, a major or minor histocompatibility (MHC) gene, a transporter, a channel (e.g., an ion channel), an immunoglobulin (Ig) superfamily (IgSF) gene (e.g., a gene encoding an antibody, T cell receptor, B cell receptor), tumor necrosis factor, an NF-kappaB protein, an integrin, a cadherin superfamily member (e.g., a cadherin), a selectin, a clotting factor, a complement factor, a plasminogen, plasminogen activating factor. Growth factors include, e.g., members of the vascular endothelial growth factor (VEGF, e.g., VEGF-A, VEGF-B, VEGF-C, VEGF-D), epidermal growth factor (EGF), insulin-like growth factor (IGF; IGF-1, IGF-2), fibroblast growth factor (FGF, e.g., FGF1-FGF22), platelet derived growth factor (PDGF), or nerve growth factor (NGF) families. It will be understood that the afore-mentioned protein families comprise multiple members. Any such member may be used in various embodiments. In some embodiments a growth factor promotes proliferation and/or differentiation of one or more hematopoietic cell types. For example, a growth factor may be CSF1 (macrophage colony-stimulating factor), CSF2 (granulocyte macrophage colony-stimulating factor, GM-CSF), or CSF3 (granulocyte colony-stimulating factors, G-CSF). In some embodiments a gene encodes erythropoietin (EPO). In some embodiments, a gene encodes a neurotrophic factor, i.e., a factor that promotes survival, development and/or function of neural lineage cells (which term as used herein includes neural progenitor cells, neurons, and glial cells, e.g., astrocytes, oligodendrocytes, microglia). For example, in some embodiments, the protein is a factor that promotes neurite outgrowth. In some embodiments, the protein is ciliary neurotrophic factor (CNTF) or brain-derived neurotrophic factor (BDNF).

In some embodiments a gene of interest encodes a polypeptide that is a subunit of any protein that is comprised of multiple subunits.

An enzyme may be any protein that catalyzes a reaction of a type that has been assigned an Enzyme Commission number (EC number) by the Nomenclature Committee of the International Union of Biochemistry and Molecular Biology (NC-IUBMB). Enzymes include, e.g., oxidoreductases, transferases, hydrolases, lyases, isomerases, ligases. Examples include, e.g., kinases (protein kinases, e.g., Ser/Thr kinase, Tyr kinase), lipid kinases (e.g., phosphatidylinositide 3-kinases (PI 3-kinases or PI3Ks)), phosphatases, acetyltransferases, methyltransferases, deacetylases, demethylases, lipases, cytochrome P450s, glucuronidases, recombinases (e.g., Rag-1, Rag-2). An enzyme may participate in the biosynthesis, modification, or degradation of nucleotides, nucleic acids, amino acids, proteins, neurotransmitters, xenobiotics (e.g., drugs) or other macromolecules.

The mammalian genome encodes at least about 500 different kinases. Kinases can be classified based on the nature of their typical substrates and include protein kinases (i.e., kinases that transfer phosphate to one or more protein(s)), lipid kinases (i.e., kinases that transfer a phosphate group to one or more lipid(s)), nucleotide kinases, etc. Protein kinases (PKs) are of particular interest in certain aspects of the invention. PKs are often referred to as serine/threonine kinases (S/TKs) or tyrosine kinases (TKs) based on their substrate preference. Serine/threonine kinases (EC 2.7.11.1) phosphorylate serine and/or threonine residues while TKs (EC 2.7.10.1 and EC 2.7.10.2) phosphorylate tyrosine residues. A number of “dual specificity” kinases (EC 2.7.12.1) that are capable of phosphorylating both serine/threonine and tyrosine residues are known. The human protein kinase family can be further divided based on sequence/structural similarity into the following groups: (1) AGC kinases—containing PKA, PKC and PKG; (2) CaM kinases—containing the calcium/calmodulin-dependent protein kinases; (3) CK1—containing the casein kinase 1 group; (4) CMGC—containing CDK, MAPK, GSK3 and CLK kinases; (5) STE—containing the homologs of yeast Sterile 7, Sterile 11, and Sterile 20 kinases; (6) TK—containing the tyrosine kinases; (7) TKL—containing the tyrosine-kinase like group of kinases. A further group referred to as “atypical protein kinases” contains proteins that lack sequence homology to the other groups but are known or predicted to have kinase activity, and in some instances are predicted to have a similar structural fold to typical kinases.

Receptors include, e.g., G protein coupled receptors, tyrosine kinase receptors, serine/threonine kinase receptors, Toll-like receptors, nuclear receptor, immune cell surface receptor. In some embodiments a receptor is a receptor for any of the hormones, cytokines, growth factors, or secreted proteins mentioned herein. Numerous G protein coupled receptors (GPCRs) are known in the art. See, e.g., Vroling B, GPCRDB: information system for G protein-coupled receptors. Nucleic Acids Res. 2011 January; 39 (Database issue):D309-19. Epub 2010 Nov. 2. The GPCRDB can be found online at http://www.gper.org/7tm/. G protein coupled receptors include, e.g., adrenergic, cannabinoid, purinergic receptors, neuropeptide receptors, olfactory receptors. Transcription factors (TFs) (sometimes called sequence-specific DNA-binding factors) bind to specific DNA sequences and (alone or in a complex with other proteins), regulate transcription, e.g., activating or repressing transcription. Exemplary TFs are listed, for example, in the TRANSFAC® database, Gene Ontology (http://www.geneonlology.org/) or DBD (www.transcriptionfactor.org) (Wilson, et al, DBD—taxonomically broad transcription factor predictions: new content and functionality Nucleic Acids Research 2008 doi:10.1093/nar/gkm964). TFs can be classified based on the structure of their DNA binding domains (DBD). For example in certain embodiments a TF is a helix-loop-helix, helix-turn-helix, winged helix, leucine zipper, bZIP, zinc finger, homeodomain, or beta-scaffold factor with minor groove contacts protein. Transcription factors include, e.g., p53, STAT3, PAS family transcription factors (e.g., HIF family: HIF1A, HIF2A, HIF3A), aryl hydrocarbon receptor.

In some embodiments it may be of interest to genetically modify multiple genes that function in the same biological pathway or process, e.g., signal transduction pathway, biosynthetic pathway, xenobiotic metabolizing pathway, anabolic or catabolic pathway, apoptosis, autophagy, endocytosis, exocytosis. In some embodiments an animal generated according to inventive methods is useful for studying drug metabolism. For example, it may be of interest to genetically modify multiple enzymes involved in xenobiotic metabolism (e.g., multiple P450s). In some embodiments an animal generated according to inventive methods is useful for studying the immune system and/or for generating animals that have a humanized immune system or that are immunocompromised and may serve as hosts for cells or tissues from other organisms of the same species or different species.

The foregoing written specification is considered to be sufficient to enable one skilled in the art to practice the invention. Various modifications of the invention in addition to those shown and described herein will become apparent to those skilled in the art from the foregoing description and fall within the scope of the appended claims. The advantages and objects of the invention are not necessarily encompassed by each embodiment of the invention. Those skilled in the art will recognize, or be able to ascertain using no more than routine experimentation, many equivalents to the specific embodiments described herein, which fall within the scope of the claims. The scope of the present invention is not to be limited by or to embodiments or examples described above.

Section headings used herein are not to be construed as limiting in any way. It is expressly contemplated that subject matter presented under any section heading may be applicable to any aspect or embodiment described herein.

Embodiments or aspects herein may be directed to any agent, composition, article, kit, and/or method described herein. It is contemplated that any one or more embodiments or aspects can be freely combined with any one or more other embodiments or aspects whenever appropriate. For example, any combination of two or more agents, compositions, articles, kits, and/or methods that are not mutually inconsistent, is provided.

Articles such as “a”, “an”, “the” and the like, may mean one or more than one unless indicated to the contrary or otherwise evident from the context.

The phrase “and/or” as used herein in the specification and in the claims, should be understood to mean “either or both” of the elements so conjoined. Multiple elements listed with “and/or” should be construed in the same fashion, i.e., “one or more” of the elements so conjoined. Other elements may optionally be present other than the elements specifically identified by the “and/or” clause. As used herein in the specification and in the claims, “or” should be understood to have the same meaning as “and/or” as defined above. For example, when used in a list of elements, “or” or “and/or” shall be interpreted as being inclusive, i.e., the inclusion of at least one, but optionally more than one, of list of elements, and, optionally, additional unlisted elements. Only terms clearly indicative to the contrary, such as “only one of” or “exactly one of” will refer to the inclusion of exactly one element of a number or list of elements. Thus claims that include “or” between one or more members of a group are considered satisfied if one, more than one, or all of the group members are present, employed in, or otherwise relevant to a given product or process unless indicated to the contrary. Embodiments are provided in which exactly one member of the group is present, employed in, or otherwise relevant to a given product or process. Embodiments are provided in which more than one, or all of the group members are present, employed in, or otherwise relevant to a given product or process. Any one or more claims may be amended to explicitly exclude any embodiment, aspect, feature, element, or characteristic, or any combination thereof. Any one or more claims may be amended to exclude any agent, composition, amount, dose, administration route, cell type, target, cellular marker, antigen, targeting moiety, or combination thereof.

Embodiments in which any one or more limitations, elements, clauses, descriptive terms, etc., of any claim (or relevant description from elsewhere in the specification) is introduced into another claim are provided. For example, a claim that is dependent on another claim may be modified to include one or more elements or limitations found in any other claim that is dependent on the same base claim. It is expressly contemplated that any amendment to a genus or generic claim may be applied to any species of the genus or any species claim that incorporates or depends on the generic claim.

Where a claim recites a composition, methods of using the composition as disclosed herein are provided, and methods of making the composition according to any of the methods of making disclosed herein are provided. Where a claim recites a method, a composition for performing the method is provided. Where elements are presented as lists or groups, each subgroup is also disclosed. It should also be understood that, in general, where embodiments or aspects is/are referred to herein as comprising particular element(s), feature(s), agent(s), substance(s), step(s), etc., (or combinations thereof), certain embodiments or aspects may consist of, or consist essentially of, such element(s), feature(s), agent(s), substance(s), step(s), etc. (or combinations thereof). It should also be understood that, unless clearly indicated to the contrary, in any methods claimed herein that include more than one step or act, the order of the steps or acts of the method is not necessarily limited to the order in which the steps or acts of the method are recited. Any method of treatment may comprise a step of providing a subject in need of such treatment, e.g., a subject having a disease for which such treatment is warranted. Any method of treatment may comprise a step of diagnosing a subject as being in need of such treatment, e.g., diagnosing a subject as having a disease for which such treatment is warranted.

Where ranges are given herein, embodiments in which the endpoints are included, embodiments in which both endpoints are excluded, and embodiments in which one endpoint is included and the other is excluded, are provided. It should be assumed that both endpoints are included unless indicated otherwise. Unless otherwise indicated or otherwise evident from the context and understanding of one of ordinary skill in the art, values that are expressed as ranges can assume any specific value or subrange within the stated ranges in various embodiments, to the tenth of the unit of the lower limit of the range, unless the context clearly dictates otherwise. “About” in reference to a numerical value generally refers to a range of values that fall within ±10%, in some embodiments ±5%, in some embodiments ±1%, in some embodiments ±0.5% of the value unless otherwise stated or otherwise evident from the context. In any embodiment in which a numerical value is prefaced by “about”, an embodiment in which the exact value is recited is provided. Where an embodiment in which a numerical value is not prefaced by “about” is provided, an embodiment in which the value is prefaced by “about” is also provided. Where a range is preceded by “about”, embodiments are provided in which “about” applies to the lower limit and to the upper limit of the range or to either the lower or the upper limit, unless the context clearly dictates otherwise. Where a phrase such as “at least”, “up to”, “no more than”, or similar phrases, precedes a series of numbers, it is to be understood that the phrase applies to each number in the list in various embodiments (it being understood that, depending on the context, 100% of a value, e.g., a value expressed as a percentage, may be an upper limit), unless the context clearly dictates otherwise. For example, “at least 1, 2, or 3” should be understood to mean “at least 1, at least 2, or at least 3” in various embodiments. It will also be understood that any and all reasonable lower limits and upper limits are expressly contemplated.

### EXEMPLIFICATION

### Example 1

### Experimental Procedures

Procedures for Generating sgRNAs Expressing Vector

Bicistronic expression vector expressing Cas9 and sgRNA (Cong et al., Science 339:819-823 (2013)) were digested with BbsI and treated with Antarctic Phosphatase, and the linearized vector was gel-purified. A pair of oligos (Table 6) for each targeting site was annealed, phosphorylated, and ligated to linearized vector.

Cell Culture and Transfection

V6.5 mESCs (on a 129/Sv×C57BL/6 F1 hybrid background) were cultured on gelatin-coated plates with standard mESC culture conditions. Cells were transfected with a plasmid expressing mammalian codon optimized Cas9 and sgRNA (single targeting), or three plasmids expressing Cas9 and sgRNAs targeting Tet1, Tet2, and Tet3 (triple targeting), or five PCR products each coding for sgRNA targeting Tet1, Tet2, Tet3, Sry, and Uty, along with a plasmid expressing PGK-puroR using FuGENE HD reagent (Promega), following manufacturer's instructions. 12 hours after transfection, mESC were re-plated at a low density on DR4 MEF feeder layers. Puromycin (2 μg/ml) was added one day after replating and taken off after 48 hours. After recovering for 4 to 6 days, individual colonies were picked and genotyped by RFLP and Southern blot analysis, and the leftover mES cells on plate were collected for Suveryor assay.

Suveryor Assay and RFLP Analysis for Genome Modification

Suveryor assay was performed as described by (Guschin et al., Methods Molec Biol, 649:247-256 (2010)). Genomic DNA from treated and control ES cells or targeted and control mice was extracted. Mouse genomic DNA samples were prepared from tail biopsies. PCR was performed using Tet1, 2, 3 specific primers (Table S3) under the following conditions: 95° C. for 5 min; 35×(95° C. for 30 s, 60° C. for 30 s, 68° C. for 40 s); 68° C. for 2 min; hold at 4° C. PCR products were then denatured, annealed, and treated with Suveryor nuclease (Transgenomic). DNA concentration of each band was measured on an ethidium bromide-stained 10% acrylamide Criterion TBE gel (BioRad) and quantified using Image J software. The same PCR products for Suveryor assay were used for RFLP analysis. 10 ul of Tet1, Tet2, or Tet3 PCR product was digested with SacI, EcoRV, or XhoI respectively. Digested DNA was separated on an ethidium bromide-stained agarose gel (2%). For sequencing, PCR products were cloned using the Original TA Cloning Kit (Invitrogen), and mutations were identified by Sanger sequencing.

Dot Blot

DNA was extracted from pre-plated mESCs following standard procedures. DNA was transferred to nylon membrane using BioRad slot blot vacuum manifold apparatus. Anti-5hmC (Active Motif 1:10000) was used to detect 5hmC following manufacturer's protocol.

Production of Cas9 mRNA and sgRNA

T7 promoter was added to Cas9 coding region by PCR amplification using primer Cas9 F and R (Table 6). T7-Cas9 PCR product was gel-purified and used as the template for in vitro transcription (IVT) using mMESSAGE mMACHINE T7 ULTRA kit (Life Technologies). T7 promoter was added to sgRNAs template by PCR amplification using primer Tet1 F and R, Tet2 F and R, Tet3 F and R (Table 6). The T7-sgRNA PCR product was gel-purified and used as the template for IVT using MEGAshortscript T7 kit (Life Technologies). Both the Cas9 mRNA and the sgRNAs were purified using MEGAclear kit (Life Technologies) and eluted in RNase-free water.

One Cell Embryo Injection

All animal procedures were performed according to NIH guidelines and approved by the Committee on Animal Care at MIT. B6D2F1 (C57BL/6×DBA2) female mice and ICR mouse strains were used as embryo donors and foster mothers, respectively. Super-ovulated female B6D2F1 mice (7-8 weeks old) were mated to B6D2F1 stud males, and fertilized embryos were collected from oviducts. Cas9 mRNAs (from 20 ng/μl to 200 ng/μl) and sgRNA (from 20 ng/μl to 50 ng/μl) was injected into the cytoplasm of fertilized eggs with well recognized pronuclei in M2 medium (Sigma). For oligos injection, Cas mRNA (100 ng/μl), sgRNA (50 ng/μl) and donor oligos (100 ng/μl) were mixed and injected into zygotes at the pronuclei stage. The injected zygotes were cultured in KSOM with amino acids at 37° C. under 5% CO₂ in air until blastocyst stage by 3.5 days. Thereafter, 15-25 blastocysts were transferred into uterus of pseudopregnant ICR females at 2.5 dpc.

Southern Blotting

Genomic DNA was separated on a 0.8% agarose gel after restriction digests with the appropriate enzymes, transferred to a nylon membrane (Amersham) and hybridized with 32P random primer (Stratagene)-labeled probes.

Prediction of Potential Off-Targets

Potential targets of CRISPR sgRNAs were found using the rules outline in Mali et al., Science, 339:823-826 (2013). For a 20 nt sgRNA sequence of nnnnn nnMMM MMMMM MMMMM (SEQ ID NO: 135), where M are the seed bases preceding the PAM sequence NGG, four search sequences (MMM MMMMM MMMMM AGG (SEQ ID NO: 136); MMM MMMMM MMMMM CGG (SEQ ID NO: 137); MMM MMMMM MMMMM GGG (SEQ ID NO: 138); MMM MMMMM MMMMM TGG (SEQ ID NO: 139)) were generated. Exact matches to these search sequences in the mouse genome (mm9) were found using bowtie and reported as potential targets of the CRISPR sgRNA.

Results

Simultaneous Targeting Up to Five Genes in ES Cells

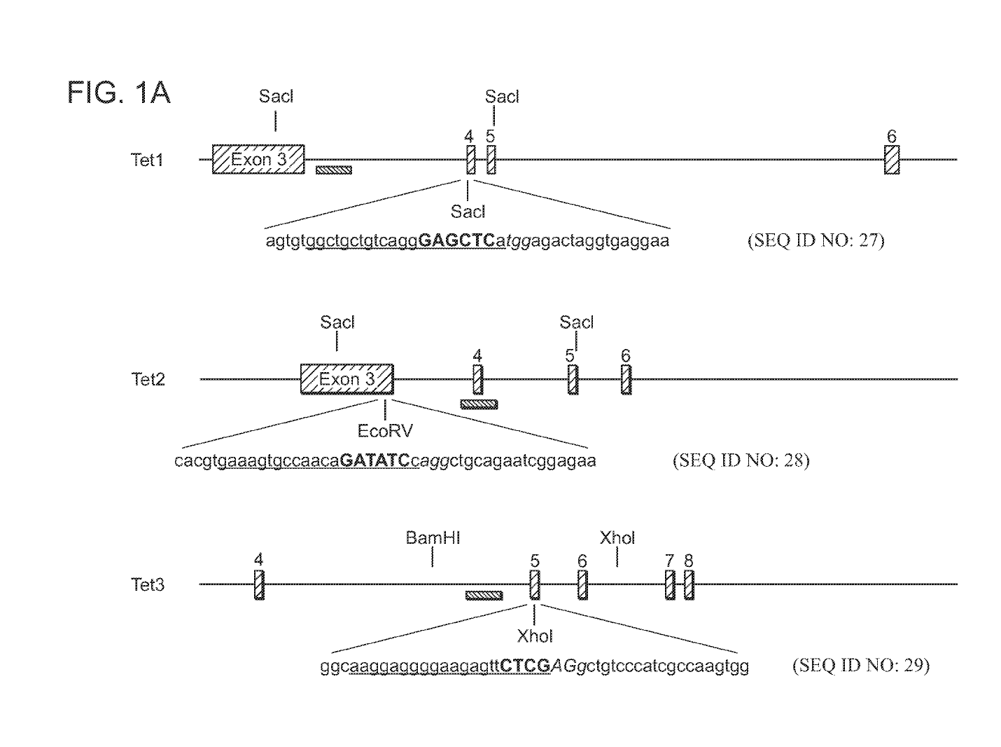

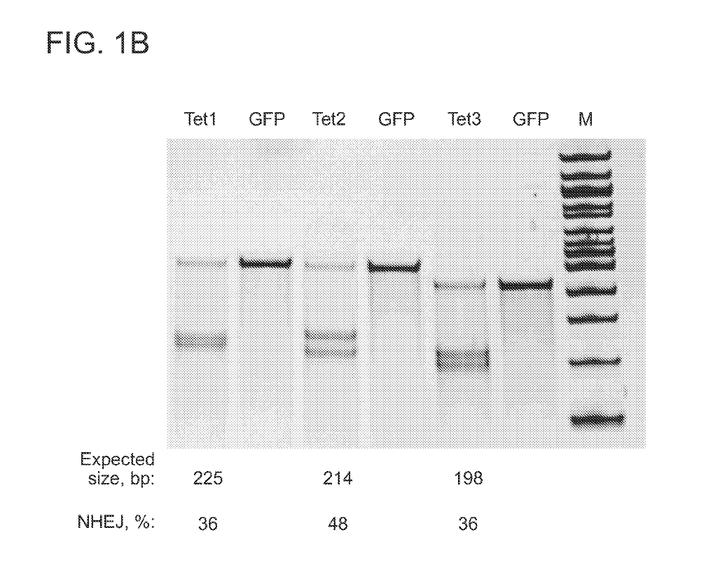

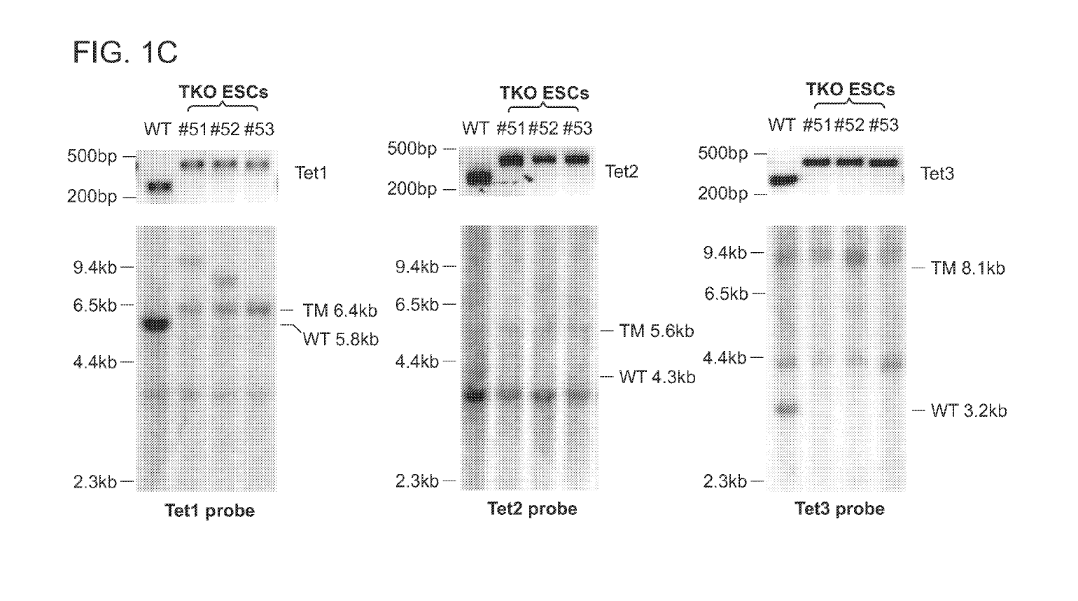

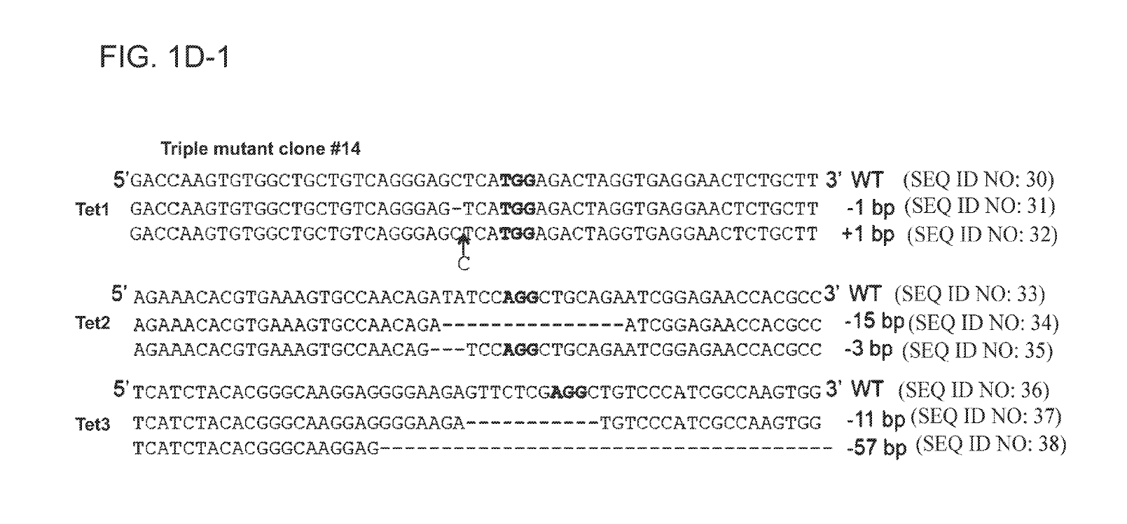

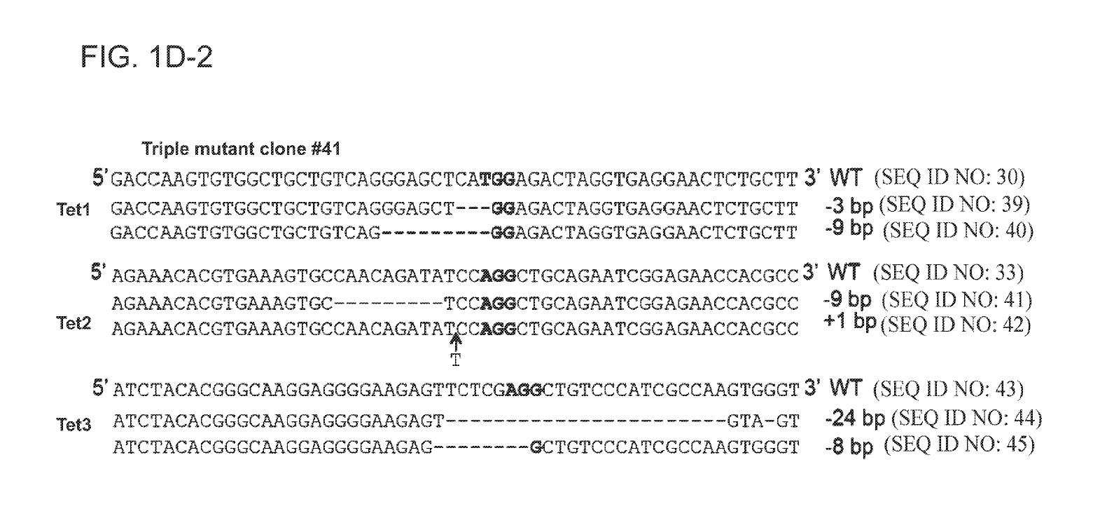

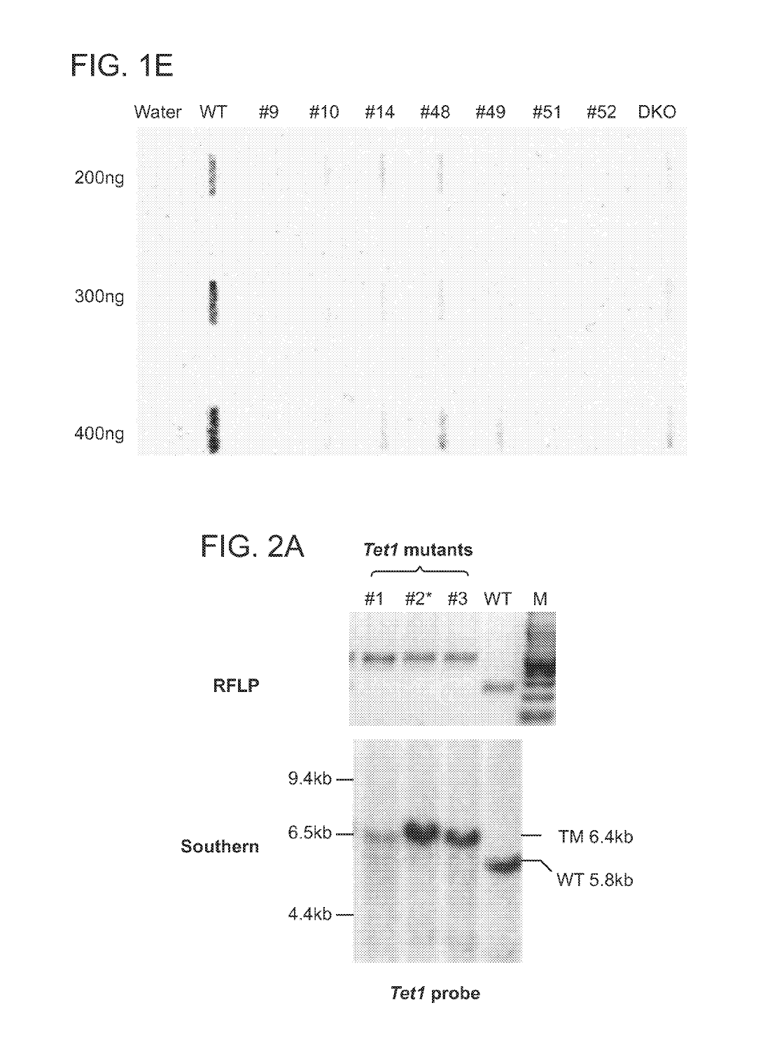

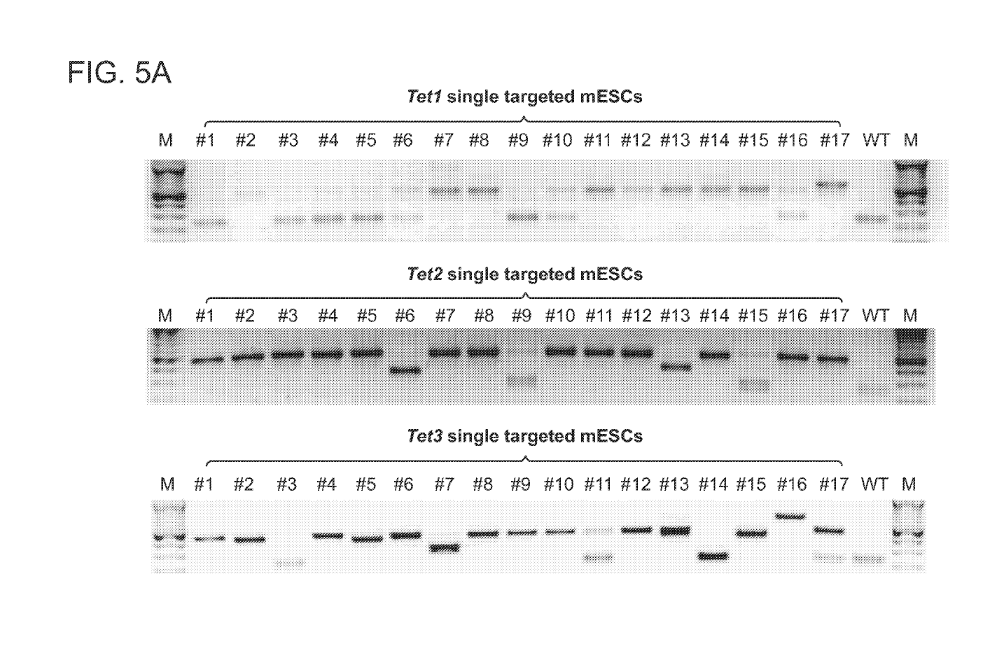

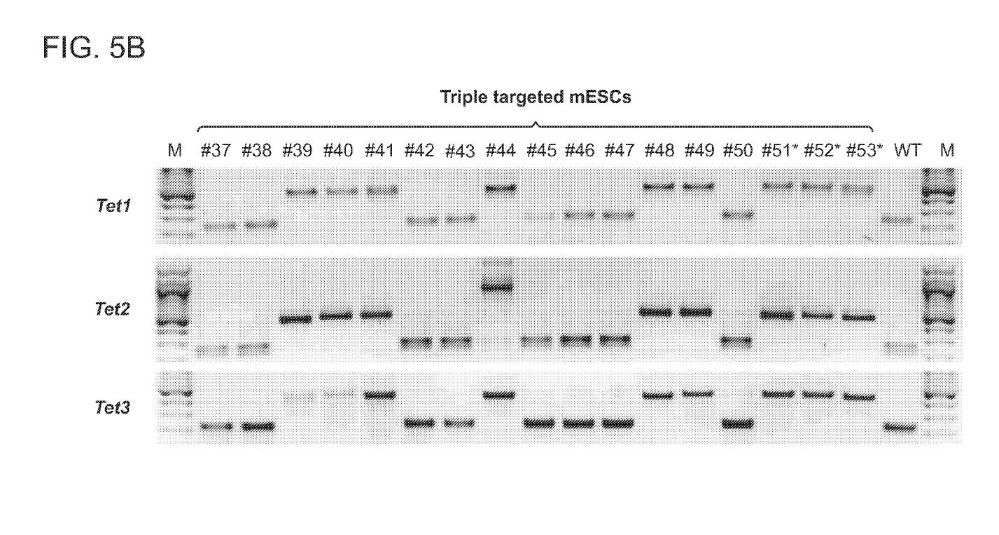

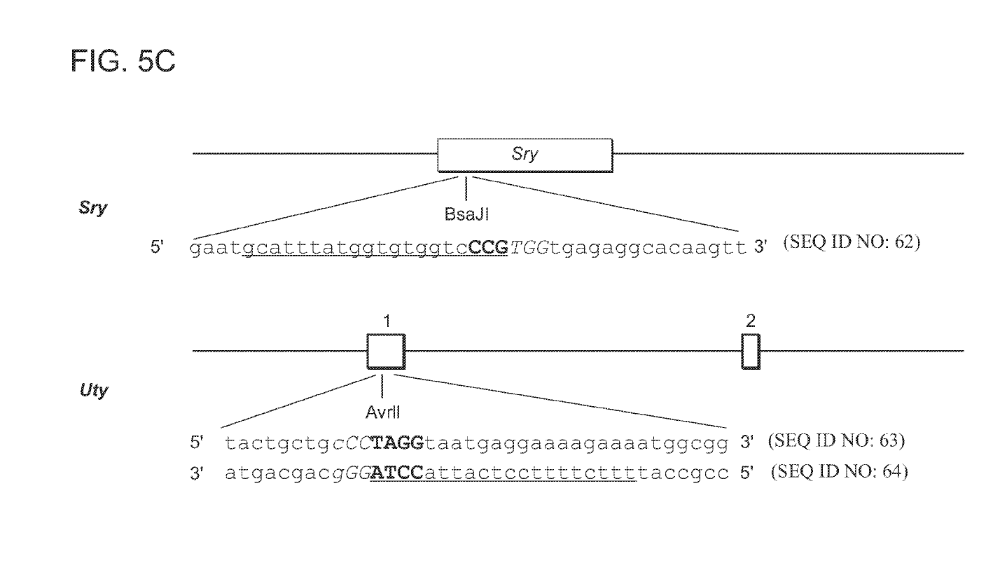

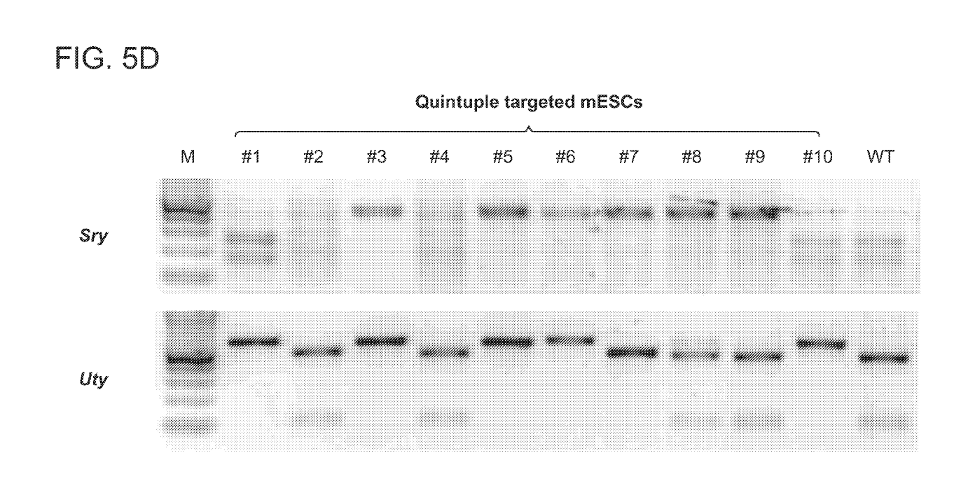

To test the possibility of targeting functionally redundant genes from the same gene family, sgRNAs targeting the Ten-eleven translocation (Tet) family members, Tet1, Tet2 and Tet3 were digested (FIG. 1A). Tet proteins (Tet1/2/3) convert 5-methylcytosine (5mC) to 5-hydroxymethylcytosine (5hmC) in various embryonic and adult tissues and mutant mice for each of these three genes have been produced by homologous recombination in ES cells (Dawlaty et al. Cell Stem Cell, 9:166-175 (2011); Gu et al., Nature, 477:606-610 (2011); Li et al., Blood, 118:4509-4518 (2011); Moran-Crusio et al., Cancer Cell, 20:11-24 (2011)). To test whether the CRISPR/Cas system could produce targeted cleavage in the mouse genome, plasmids expressing both the mammalian codon optimized Cas9 and a sgRNA targeting each gene (Cong et al., Science, 339:819-823(2013); Mali et al., Science, 339:823-826 (2013)) were transfected into mouse ES cells and determined the targeted cleavage efficiency by the Surveyor assay (Guschin et al., Methods Mol Biol, 649:247-256 (2010)). All three Cas9-sgRNA transfections produced cleavage at target loci with high efficiency of 36% at Tet1, 48% at Tet2, and 36% at Tet3 (FIG. 1B). Because each target locus contains a restriction enzyme recognition site (FIG. 1A), a ˜500 bp fragment around each target site was PCR amplified, and the PCR products were digested with the respective enzyme. A correctly targeted allele will lose the restriction site, which can be detected by failure to cleave upon enzyme treatment. Using this restriction fragment length polymorphism (RFLP) assay, 48 ES cell clones from each single targeting experiment were screened. Consistent with the Surveyor analysis, a high percentage of mESC clones were targeted, with a high probability of having both alleles mutated (FIG. 5A). The results summarized in Table 1 demonstrate that between about 65% and about 81% of the tested ES cell clones carried mutations in the Tet genes with up to abut 77% having mutations in both alleles.

The high efficiency of single gene modification prompted testing targeting of all three genes simultaneously. For this ES cells were co-transfected with the constructs expressing Cas9 and three sgRNAs targeting Tet1, 2 and 3. Of 96 clones screened using the RFLP assay, 20 clones were identified as having mutations in all six alleles of the three genes (FIG. 1C, 5B, Table 1). To exclude that a PCR bias could give false positive results, Southern blot analysis was performed and complete agreement with the RFLP results was confirmed (FIG. 1C). The PCR products of Tet1, Tet2, and Tet3 targeted regions were sub-cloned and sequenced to verify that all of eight tested clones carried biallelic mutations in all three genes with most clones displaying two mutant alleles for each gene with small insertions or deletions (indels) at the target site (FIG. 1D). To test whether these mutant alleles would abolish the function of Tet proteins, the 5hmC level of targeted clones were compared to wt mES cells. Previously, a depletion of 5hmC in Tet1/Tet2 double knockout mES cells derived using traditional gene targeting methods was reported (Dawlaty et al., Cell Stem Cell, 9:166-175 (2011)). As expected from loss of function alleles, a significant reduction of 5hmC levels in all clones carrying biallelic mutations in the three genes was found (FIG. 1E).

Recently efficient targeting of two Y-linked genes, Sry and Uty, using TALENs was demonstrated (Wang et al., in press). To further test the potential of multiplexed gene targeting by CRISPR/Cas system, sgRNAs targeting these two P-linked genes were designed (FIG. 5C). Short PCR products encoding sgRNAs targeting all five genes (Tet1, Tet2, Tet3, Sry, and Uty) were pooled and co-transfected with a Cas9 expressing plasmid and the PGK puroR cassette into ES cells. Of 96 clones that were screened using the RFLP assay, 10% carried mutations in all eight alleles of the five genes (FIG. 5D, Table 4), demonstrating the capacity of the CRISP/Cas9 system for highly efficient multiplexed gene targeting.

One Step Generation of Single Gene Mutant Mice by Zygote Injection

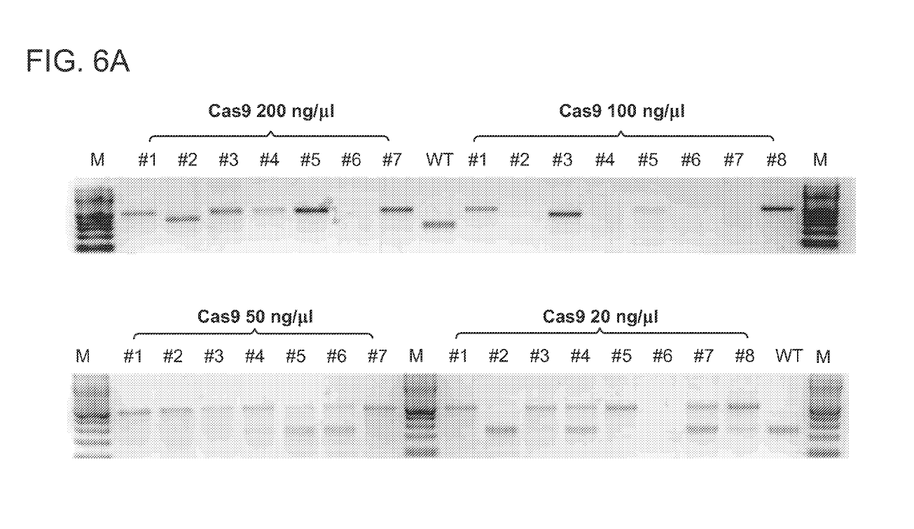

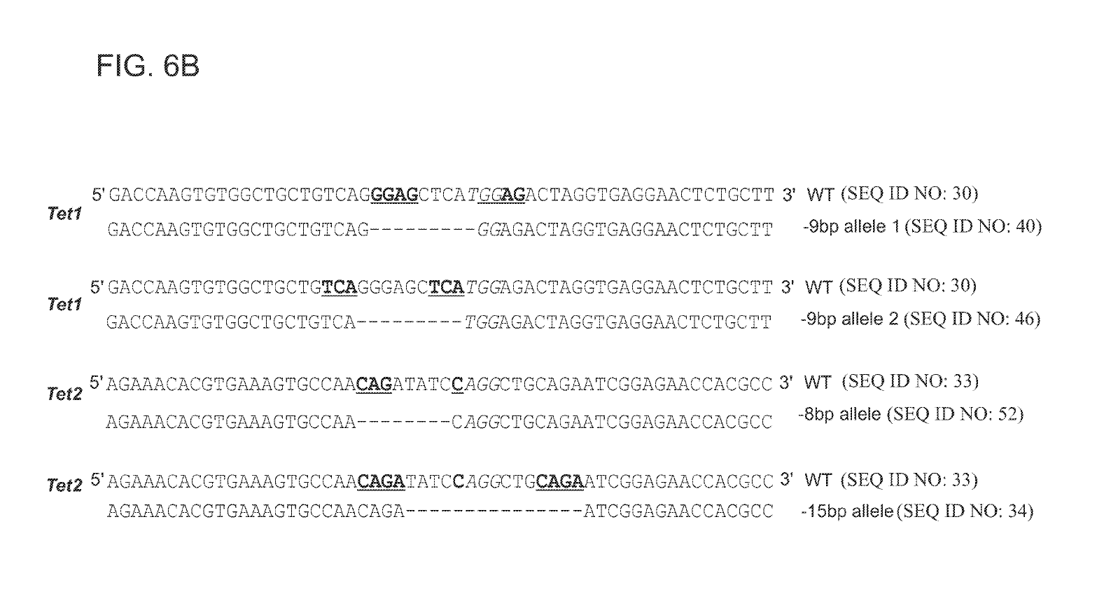

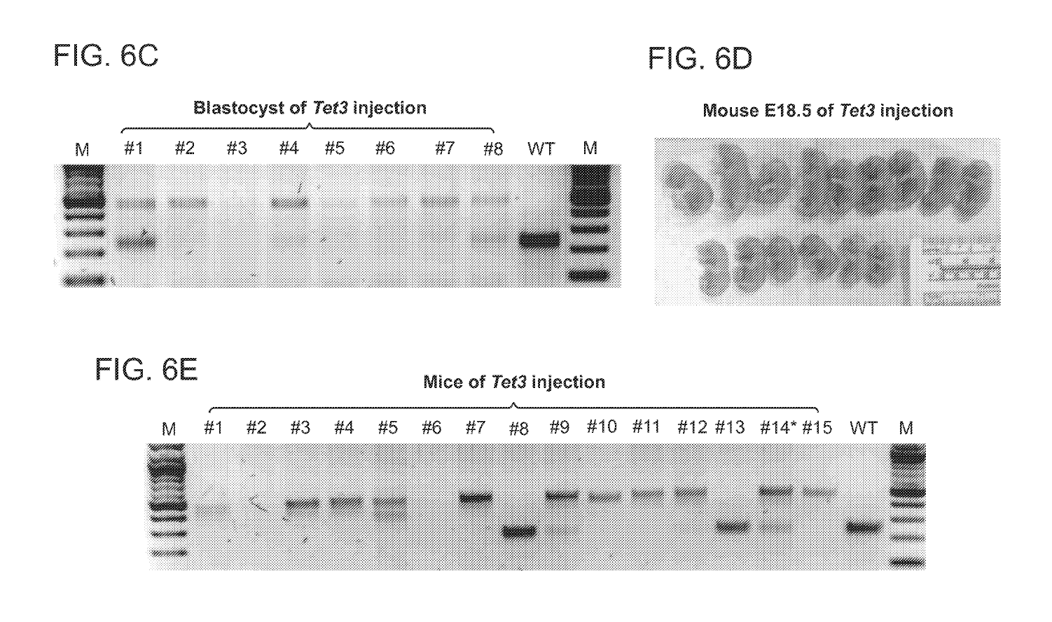

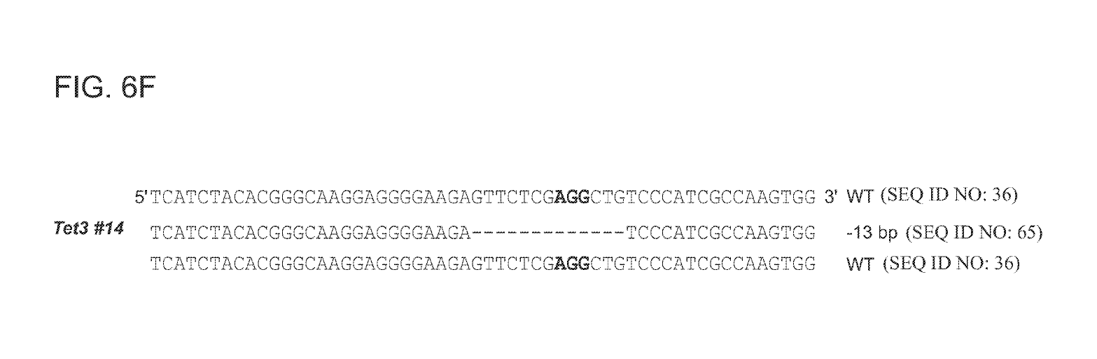

Whether mutant mice could be generated in vivo by direct embryo manipulation was tested. Capped polyadenylated Cas9 mRNA was produced by in vitro transcription and co-injected with sgRNAs. Initially, to determine the optimal concentration of Cas9 mRNA for targeting in vivo, varying amounts of Cas9-encoding mRNA were injected with Tet1 targeting sgRNA at constant concentration (20 ng/μl) into pronuclear (PN) stage one-cell mouse embryos and the frequency of altered alleles at the blastocyst stage was assessed using the RFLP assay. As expected, higher concentration of Cas9 mRNA led to more efficient gene disruption (FIG. 6A). Nevertheless, even embryos injected with the highest amount of Cas9 mRNA (200 ng/μl) showed normal blastocyst development, indicating low toxicity.

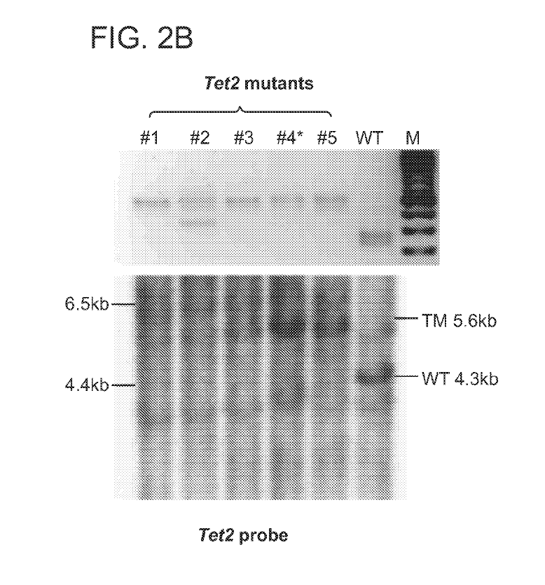

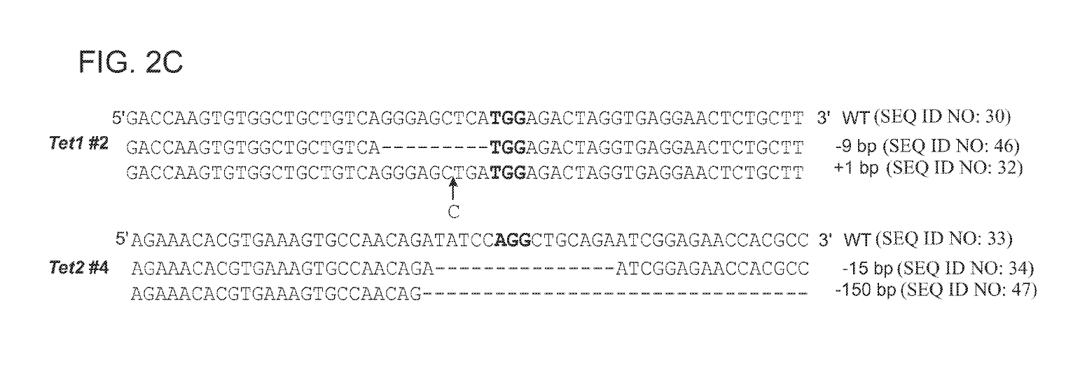

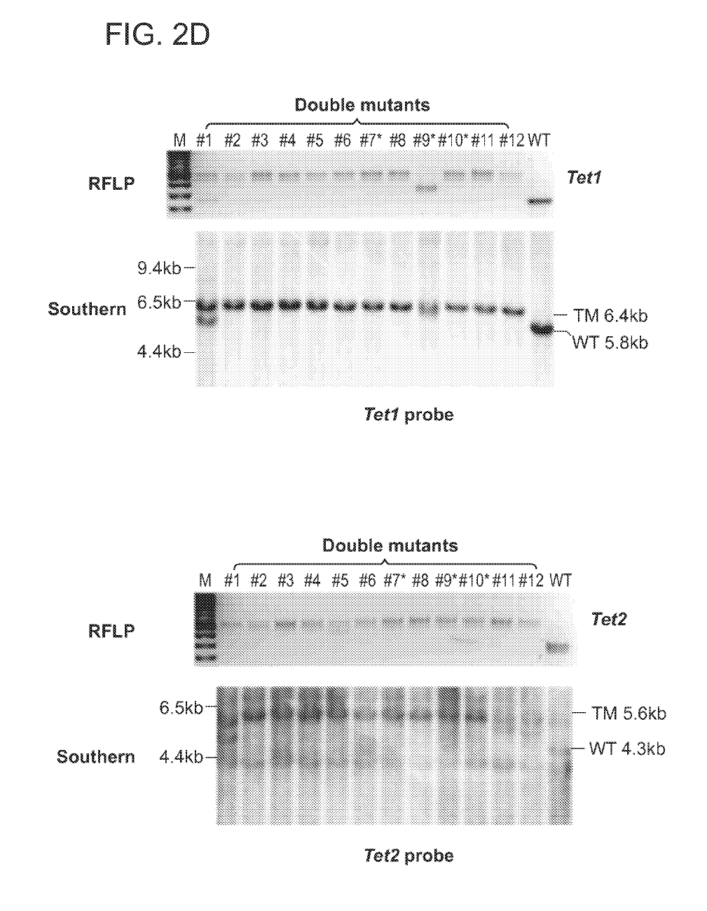

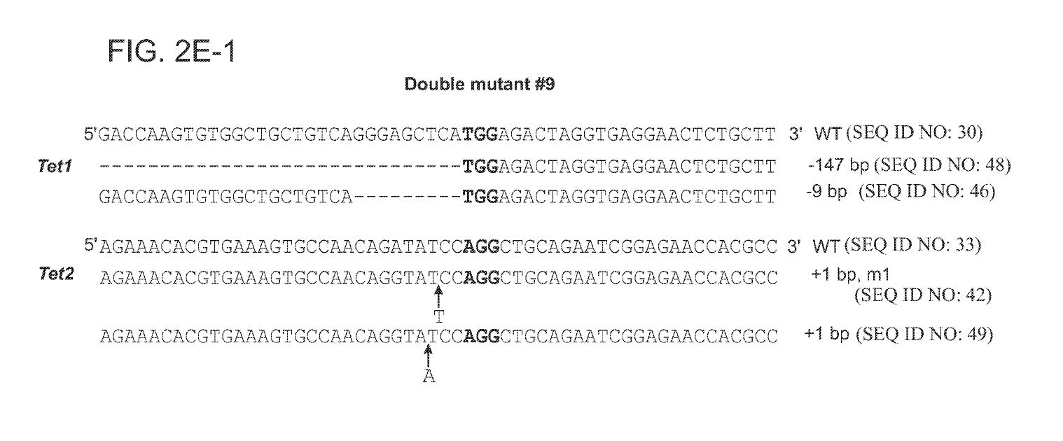

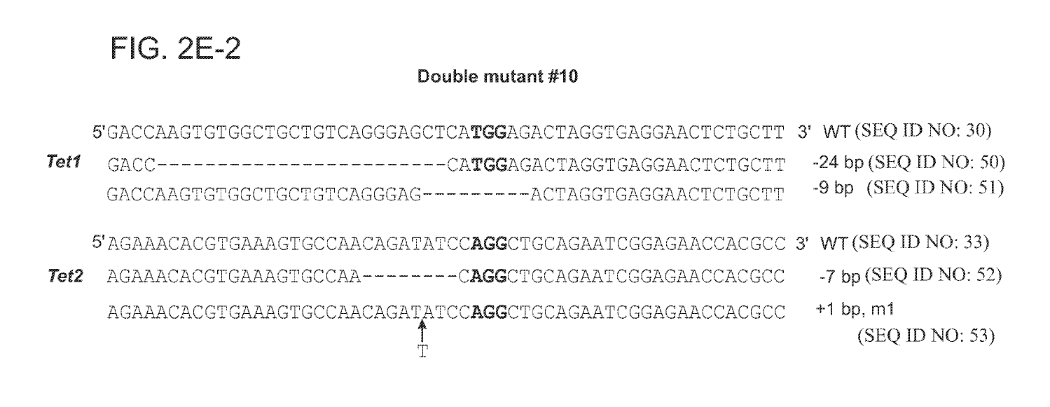

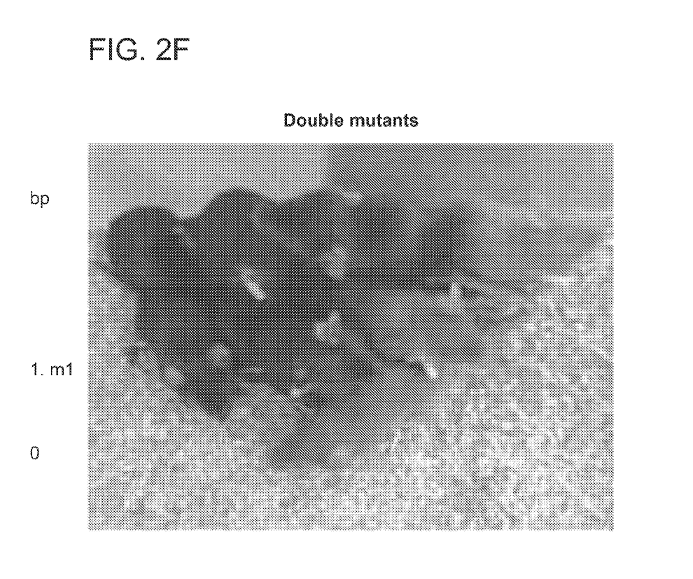

To investigate whether postnatal mice carrying targeted mutations could be generated, sgRNAs targeting Tet1 or Tet2 were co-injected with different concentrations of Cas9 mRNA. Blastocysts derived from the injected embryos were transplanted into foster mothers and newborn pups were obtained. As summarized in Table 2, about 10% of the transferred blastocysts developed to birth independent of the RNA concentrations used for injection indicating low fetal toxicity of the Cas9 mRNA and sgRNA. RFLP, Southern blot, and sequencing analysis demonstrated that between 50 and 90% of the postnatal mice carried biallelic mutations in either target gene (FIGS. 2A, 2B, 2C, Table 2).

Surprisingly, specific Δ9 Tet1 and specific Δ8 and Δ15 Tet2 mutant alleles were repeatedly recovered in independently derived mice. Preferential generation of these alleles is likely caused by a short sequence repeat flanking the DSB (see FIG. 6B) consistent with a previous report demonstrating that perfect microhomology sequences flanking the cleavage sites can generate microhomology-mediated precise deletions by end repair mechanism (MMEJ) (McVey and Lee, Trends Genet, 24:529-538(2008); Symington and Gautier, Annu Rev Genet, 11:636-646 (2011)) (FIG. 6B). A similar observation was also made when TALEN mRNA was injected into one cell rat embryos (Tesson et al., Nat Biotechnol, 29:695-696 (2011)).

Blastocysts were also derived from zygotes injected with Cas9 mRNA and Tet3 sgRNA. Genotyping of the blastocysts demonstrated that of eight embryos three were homozygous and three were heterozygous Tet3 mutants (two failed to amplify) (FIG. 6C). Some blastocysts were implanted into foster mothers and, upon C-section, multiple mice of smaller size (FIG. 6D), many of which died soon after delivery, were readily identified. Genotyping shown in FIG. 6E indicated that all pups with mutations in both Tet3 alleles died neonatally. Only two out of 15 mice survived that were either Tet3 heterozygous mutants or wt (FIG. 6F). These results are consistent with the lethal neonatal phenotype of Tet3 knockout mice generated using traditional methods (Gu et al, Nature, 477:606-610 (2011)), although which of the Tet3 mutations produced loss of function rather than hypomorphic alleles has not been established.

One Step Generation of Double Gene Mutant Mice by Zygote Injection

To test whether Tet1/Tet2 double mutant mice could be produced from single embryos, Tet1 and Tet2 sgRNAs were co-injected with 20 or 100 ng/μl Cas9 mRNA into zygotes. A total of 28 pups were born from 144 embryos transferred into foster mothers (21% live birth rate) that had been injected at the zygote stage with high concentrations of RNA (Cas9 mRNA at 100 ng/μl, sgRNAs at 50 ng/μl), consistent with low or no toxicity of the Cas9 mRNA and sgRNAs (Table 3). RFLP, Southern blot analysis and sequencing identified 22 mice carrying targeted mutations at all four alleles of the Tet1 and Tet2 genes (FIG. 2D, 2E) with the remaining mice carrying mutations in a subset of alleles (Table 3). Injection of zygotes with low concentration of RNA (Cas9 mRNA at 20 ng/μl, sgRNAs at 20 ng/μl) yielded 19 pups from 75 transferred embryos (about 25% live birth rate), which is a higher survival rate than from embryos injected with 100 ng/μl of Cas9 RNA. Nevertheless, more than about 50% of the pups were biallelic Tet1/Tet2 double mutants (Table 3). These results demonstrate that postnatal mice carrying biallelic mutations in two different genes can be generated within one month with high efficiency (FIG. 2F).

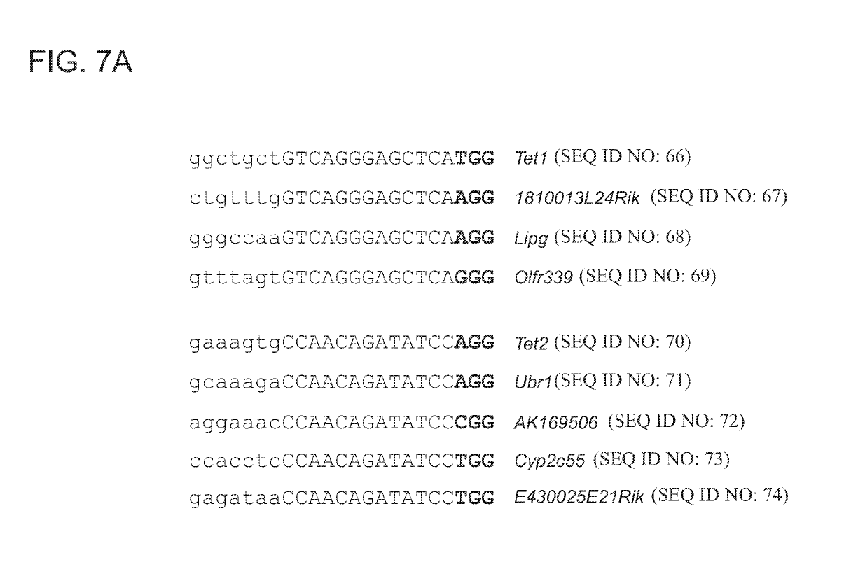

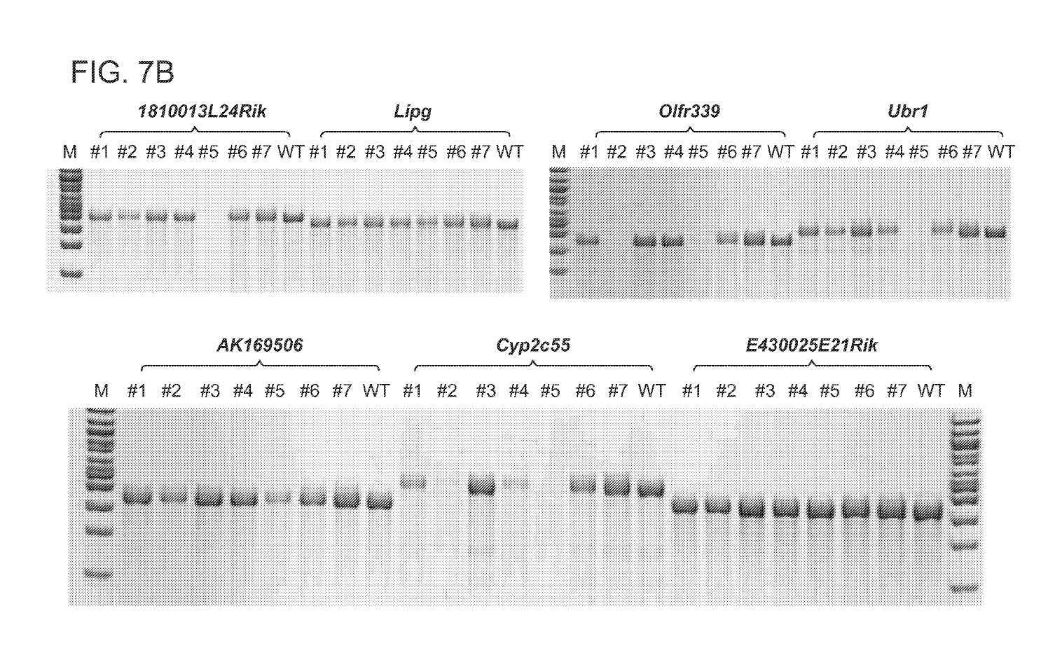

Although the high live birth rate and normal development of mutant mice indicate low toxicity of CRISPR/Cas9 system, the off-target effects in vivo were determined. Previous work in vitro, in bacteria, and in cultured human cells suggested that the protospacer-adjacent motif (PAM) sequence NGG and the 8-12 base “seed sequence” at the 3′ end of the sgRNA are most important for determining the DNA cleavage specificity (Cong et al., Science, 339:819-823(2013); Jiang et al., Nat Biotechnol, 31:233-239 (2013); Jinek et al., Science, 337:816-821 (2012)). Based on this rule, only three and four potential off targets exist in mouse genome for Tet1 and Tet2 sgRNA respectively (Table 5, Experimental procedures), with each of them perfectly matching the 12 bp seed sequence at the 3′ end and the NGG PAM sequence of the sgRNA (there is no potential off target site for Tet3 sgRNA using this prediction rule). From seven double mutant mice produced from injection with high RNA concentration ˜400 bp fragments from all seven potential off-target loci were PCR amplified and no cleavage was found in the Surveyor assay (FIGS. 7A-7B), indicating a high specificity of CRISPR/Cas system.

Multiplexed Precise HDR-Mediated Genome Editing In Vivo

The NHEJ-mediated gene mutations described above produced mutant alleles with different and unpredictable insertions and deletions of variable size. The possibility of precise homology directed repair (HDR)-mediated genome editing by co-injecting Cas9 mRNA, sgRNAs and single stranded DNA oligos into one-cell embryos was explored. For this an oligo targeting Tet1 so as to change two base pairs of a SacI restriction site and create instead an EcoRI site and a second oligo targeting Tet2 with two base pair changes that would convert an EcoRV site into an EcoRI site were designed (FIG. 3A). Blastocysts were derived from zygotes injected with Cas9 mRNA and sgRNAs and oligos targeting Tet1 or Tet2, respectively. DNA was isolated, amplified and digested with EcoRI to detect oligo mediated HDR events. Six out of nine Tet1 targeted embryos and nine out of 15 Tet2 targeted embryos incorporated an EcoRI site at the respective target locus, with several embryos having both alleles modified (FIG. 8A). When Cas9 mRNA, sgRNAs, and single stranded DNA oligos targeting both Tet1 and Tet2 were co-injected into zygotes, out of 14 embryos, four were identified that were targeted with the oligo at the Tet1 locus, seven that were targeted with the oligo at the Tet2 locus and one embryo (#2) that had one allele of each gene correctly modified (FIG. 8B). All four alleles of embryo #2 were sequenced, confirming that one allele of each gene contained the 2 bp changes directed by the oligo, while the other alleles were disrupted by NHEJ-mediated deletion and insertion (FIG. 8C).

Blastocysts with double oligo injections were implanted into foster mothers and a total of 10 pups were born from 48 embryos transferred (21% live birth rate). Upon RFLP analysis using EcoRI, seven mice containing EcoRI sites at the Tet1 locus and eight mice containing EcoRI sites at the Tet2 locus, with six mice containing EcoRI sites at both Tet1 and Tet2 loci were identified (FIG. 3B). RFLP analysis using SacI and EcoRV to Tet1 and Tet2 loci respectively was also applied showing that all alleles not targeted by oligos contained disruptions, which is in consistent with the high biallelic mutation rate by Cas9 mRNA and sgRNAs injection. These results were confirmed by sequencing demonstrating mutations in all four alleles of mouse #5 and #7 (FIG. 3C). The results herein demonstrate that mice with HR-mediated precise mutations in multiple genes can be generated in one step by CRISPR/Cas mediated genome editing.

TABLE 1
CRISPR/Cas mediated gene targeting in V6.5 mES cells
Plasmids encoding Cas9 and sgRNAs targeting Tet1, Tet2,
and Tet3 were transfected separately (single targeting)
or in a pool (triple targeting) into mES cells. The number
of total alleles mutated in each mES cell clone
is listed from 0 to 2 for single targeting experiment,
and 0 to 6 for triple targeting experiment. The number of
clones containing each specific number of mutated alleles is
shown in relation to the total number of clones
screened in each experiment.

| | Mutant alleles per clone/Total clones tested | | | | | | |
|---|---|---|---|---|---|---|---|
| Sg RNA | 6 | 5 | 4 | 3 | 2 | 1 | 0 |
| Tet1 | NA | NA | NA | NA | 27/48 | 4/48 | 17/48 |
| Tet2 | NA | NA | NA | NA | 37/48 | 2/48 | 9/48 |
| Tet3 | NA | NA | NA | NA | 32/48 | 3/48 | 13/48 |
| Tet1 + Tet2 + Tet3+ | 20/96 | 16/96 | 2/96 | 2/96 | 1/96 | 0/96 | 55/96 |

TABLE 2
CRISPR/Cas mediated single gene targeting in BDF2 mice.
Cas9 mRNA and sgRNAs targeting Tet1, Tet2, or Tet3 were injected
into fertilized eggs. The blastocysts derived from injected embryos were
transplanted into foster mothers and newborn pups were obtained and
genotyped. The number of total alleles mutated in each mouse is
listed from 0 to 2. The number of mice containing each specific
number of mutated alleles is shown in relation to the total number
of mice screened in each experiment.

| | Dose of | | | | | | |
|---|---|---|---|---|---|---|---|
| | Cas9/ | | | | Mutant alleles per | | |
| | gRNA | Blastocyst/ | Transferred | | mouse/Total mice | | |
| Sg | mRNA | Injected | embryos | Newborns | tested* | | |
| RNA | (ng/μl) | zygotes | (recipients) | (dead) | 2 | 1 | 0 |
| Tet1 | 200/20 | 38/50 | 19(1) | 2(0) | 2/2 | 0/2 | 0/2 |
| Tet1 | 100/20 | 50/60 | 25(1) | 3(0) | 2/3 | 0/3 | 1/3 |
| Tet1 | 50/20 | 40/50 | 40(2) | 8(3) | 4/7 | 2/7 | 1/7 |
| Tet1 | 100/50 | 167/198 | 60(3) | 12(2) | 9/11 | 1/11 | 1/11 |
| Tet2 | 100/50 | 176/203 | 108(5) | 22(3) | 19/20 | 0/20 | 1/20 |
| Tet3 | 100/50 | 85/112 | 64(4) | 15(13) | 9/13 | 2/13 | 2/13 |

*Some of the pups were cannibalized

TABLE 3
CRISPR/Cas mediated double gene targeting in BDF2 mice.
Cas9 mRNA and sgRNAs targeting Tet1 and Tet2 were co-injected into
fertilized eggs. The blastocysts derived from the injected embryos were transplanted
into foster mothers and newborn pups were obtained and genotyped. The number of
total alleles mutated in each mouse is listed from 0 to 4 for Tet1 and Tet2. The
number of mice containing each specific number of mutated alleles is shown in
relation to the number of total mice screened in each experiment.

| | Dose of | | | | | | | | |
|---|---|---|---|---|---|---|---|---|---|
| | Cas9/g | | | | | | | | |
| | RNA | Blastocyst/ | | | Mutant alleles per mouse/ | | | | |
| Sg | mRNA | Injected | Transferred | Newborns | Total mice tested* | | | | |
| RNA | (ng/μl) | zygotes | embryos | (dead) | 4 | 3 | 2 | 1 | 0 |
| Tet1 + | 100/50 | 194/229 | 144(7) | 31(8) | 22/28 | 4/28 | 1/28 | 1/28 | 0/28 |
| Tet2 | | | | | | | | | |
| Tet1 + | 20/20 | 92/109 | 75(5) | 19(3) | 11/19 | 1/19 | 2/19 | 3/19 | 2/19 |
| Tet2 | | | | | | | | | |

*Some of the pups were cannibaized

Table 4 Plasmids encoding Cas9 and five PCR products expressing sgRNAs targeting Tet1, Tet2, Tet3, Sry, and Uty were co-transfected into mES cells. The number of clones containing mutations in all six Tet alleles is listed in the Tet1, 2, 3 column; the number of clones containing mutations in all six Tet alleles and Sry allele is listed in the Tet1, 2, 3+Sry column; the number of clones containing mutations in all six Tet alleles and both Sry and Uty allele is listed in the Tet1, 2, 3+Sry+Uty column.

The increased efficiency of generating Tet1, 2, 3 triple targeted mES clones in this quintuple targeting experiment, compared to the triple targeting experiment (Table 1), is likely due to the use of short PCR products instead of plasmids that express sgRNAs. The much smaller size of pooled PCR products may ensure more efficient delivery into transfected cells. Table 4 is related to Table 1.

| Mutant | Tet | Tet 1, 2, | Tet 1, 2, 3 + |
|---|---|---|---|
| Genes | 1, 2, 3 | 3 + Sry | Sry + Uty |
| No. Mutant alleles | 6 and More | 7 | 8 |
| No. Mutant | 54/96 | 37/96 | 10/96 |
| clones/Total clones | | | |

TABLE 5
Potential off targets of Tet1 and Tete2 sgRNAs

| | | | | |
|---|---|---|---|---|
| | Coordinate | | | |
| MatchName | (mm9) | Strand | SEEDPAM | Gene |

Tet1

| | | | | |
|---|---|---|---|---|
| Tet1_1_ | chr10: | − | ggctgctGTCAG | Tet1 |
| TGG_3 | 62296293- | | GGAGCTCATGG | |
| | 62296308 | | (SEQ ID NO: | |
| | | | 140) | |
| | | | | 33 kb |
| Tet1_1_ | chr16: | + | ctgtttgGTCAG | 1810013L24Rik |
| AGG_1 | 8891779- | | GGAGCTCAAGG | |
| | 8891794 | | (SEQ ID NO: | |
| | | | 141) | |
| Tet1_1_ | chr18: | − | GggccaaGTCAG | 9.4 kb 5′ of |
| AGG_2 | 75130318- | | GGAGCTCAAGG | Lipg |
| | 75130333 | | (SEQ ID NO: | |
| | | | 142) | |
| Tet1_1_ | chr2: | + | gtttagtGTCAG | 9.8 kb 3′ of |
| GGG_4 | 36287584- | | GGAGCTCAGGG | Olfr339 |
| | 36287599 | | (SEQ ID NO: | |
| | | | 143) | |

Tet2

| | | | | |
|---|---|---|---|---|
| Tet2_1_ | chr3: | − | gaaagtgCCAAC | Tet2 |
| AGG_2 | 133148617- | | AGATATCCAGG | |
| | 133148632 | | (SEQ ID NO: | |
| | | | 144) | |
| Tet2_1_ | chr2: | + | gcaaagaCCAAC | Intron of |
| AGG_1 | 120696599- | | AGATATCCAGG | Ubr1 |
| | 120696614 | | (SEQ ID NO: | |
| | | | 145) | |
| Tet2_1_ | chr10: | + | aggaaacCCAAC | 2.4 kb 5′ of |
| CGG_3 | 95206326- | | AGATATCCCGG | AK169506 |
| | 95206341 | | (SEQ ID NO: | |
| | | | 146) | |
| Tet2_1_ | chr19: | + | ccacctcCCAAC | Intron of |
| TGG_4 | 39098539- | | AGATATCCTGG | Cyp2c55 |
| | 39098554 | | (SEQ ID NO: | |
| | | | 147) | |
| Tet2_1_ | chr15: | | gagataaCCAAC | Intron of |
| TGG_5 | 59188892- | | AGATATCCTGG | E430025E21Rik |
| | 59188907 | | (SEQ ID NO: | |
| | | | 148) | |

TABLE 6
Oligonucleotides used in this study. oligonucleotides used for
cloning sgRNA expression vector

| Gene | | |
|---|---|---|
| target | Direction | Sequence (5′ to 3′) |
| Tet1 | F | CACCGGCTGCTGTCAGGGAGCTCA (SEQ ID NO: 149) |
| | R | AAACTGAGCTCCCTGACAGCAGCC (SEQ ID NO: 150) |
| Tet2 | F | CACCGAAAGTGCCAACAGATATCC (SEQ ID NO: 151) |
| | R | AAACGGATATCTGTTGGCACTTTC (SEQ ID NO: 152) |
| Tet3 | F | CACCGAAGGAGGGGAAGAGTTCTCG (SEQ ID NO: 153) |
| | R | AAACCGAGAACTCTTCCCCTCCTTC (SEQ ID NO: 154) |
| Sry | F | CACCGCATTTATGGTGTGGTCCCG (SEQ ID NO: 155) |
| | R | AAACCGGGACCACACCATAAATGC (SEQ ID NO: 156) |
| Uty | F | CACCGTTTCTTTTCCTCATTACCTA (SEQ ID NO: 157) |
| | R | AAACTAGGTAATGAGGAAAAGAAAC (SEQ ID NO: 158) |

Oligonucleotides used forSuveryor assay and RFLP analysis

| Gene | | |
|---|---|---|
| target | Direction | Sequence (5′ to 3′) |
| Tet1 | F | TTGTTCTCTCCTCTGACTGC (SEQ ID NO: 159) |
| | R | TGATTGATCAAATAGGCCTGC (SEQ ID NO: 160) |
| Tet2 | F | CAGATGCTTAGGCCAATCAAG (SEQ ID NO: 161) |
| | R | AGAAGCAACACACATGAAGATG (SEQ ID NO: 162) |
| Tet3 | F | CCACCTCTGAGCGCAGAGTG (SEQ ID NO: 163) |
| | R | GATGAACACAGTTCCTGACAG (SEQ ID NO: 164) |
| Sry | F | GTCTGTCTTTGTCTGTCTGTC (SEQ ID NO: 165) |
| | R | GGGTATTTCTCTCTGTGTAGG (SEQ ID NO: 166) |
| Uty | F | GAGTTCTTCTTGCGTTCACC (SEQ ID NO: 167) |
| | R | AATGAGCACTTTCAGAGTAGG (SEQ ID NO: 168) |

Oligonucleotides used for making template for in vitro transcription

| Template | Direction | Sequence (5′ to 3′) |
|---|---|---|
| Cas9 | F | TAATACGACTCACTATAGGGAGAATGGACTATAAG |
| | | GACCACGAC (SEQ ID NO: 169) |
| | R | GCGAGCTCTAGGAATTCTTAC (SEQ ID NO: 170) |
| | | TTAATACGACTCACTATAGGCTGCTGTCAGGGAGC |
| Tet1 | F | TC (SEQ ID NO: 171) |
| sgRNA | R | AAAAGCACCGACTCGGTGCC (SEQ ID NO: 172) |
| Tet2 | F | TTAATACGACTCACTATAGGAAAGTGCCAACAGAT |
| sgRNA | | ATCC (SEQ ID NO: 173) |
| | R | AAAAGCACCGACTCGGTGCC (SEQ ID NO: 174) |
| Tet3 | F | TTAATACGACTCACTATAGGAAGGAGGGGAAGAG |
| sgRNA | | TTCTCG (SEQ ID NO: 175) |
| | R | AAAAGCACCGACTCGGTGCC (SEQ ID NO: 176) |

Oligonucleotides used for HDR-mediated repair through embryo injection

| Gene | |
|---|---|
| target | Sequence (5′ to 3′) |
| Tet1 | AaagaaaaaggcccatattatacacaccttggggcaggaccaagtgtggctgctgtcaggGAat |
| | TCatggagactaggtgaggaactctgcttcccgctaacccattcttcccggtgacctggctc |
| | (SEQ ID NO: 177) |
| Tet2 | TcactctgtgactataaggctctgactctcaagtcacagaaacacgtgaaagtgccaacaGAat |
| | TCcaggctgcagaatcggagaaccacgcccgagctgcagagcctcaagcaaccaaaagcaca |
| | (SEQ ID NO: 178) |

Discussion

The genetic manipulation of mice is a crucial approach for the study of development and disease. However, the generation of mice with specific mutations is labor intensive and involves gene targeting by homologous recombination in ES cells, the production of chimeric mice and, after germ line transmission of the targeted ES cells, the interbreeding of heterozygous mice to produce the homozygous experimental animals, a process that may take 6 to 12 months or longer (Capecchi, 2005). To produce mice carrying mutations in several genes requires time-consuming intercrossing of single mutant mice. Similarly, the generation of ES cells carrying homozygous mutations in several genes is usually achieved by sequential targeting, a process that is labor-intensive necessitating multiple consecutive cloning steps to target the genes and to delete the selectable markers.

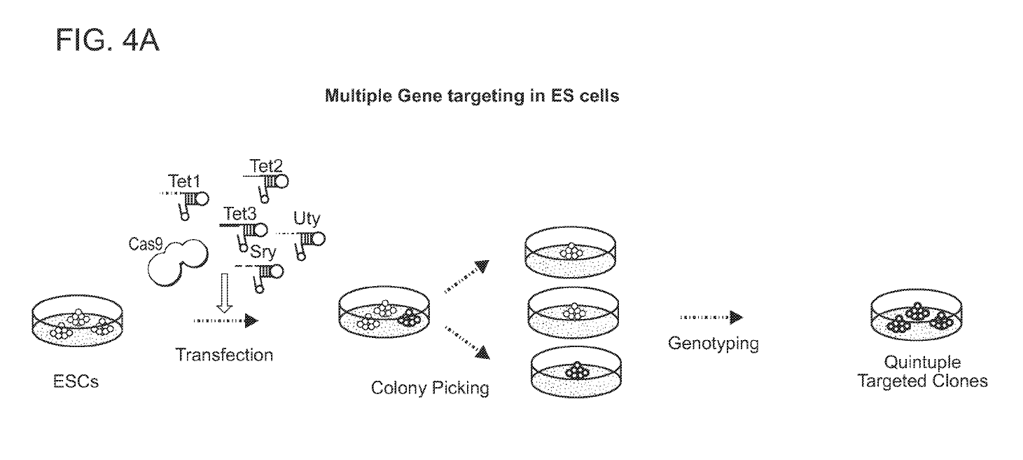

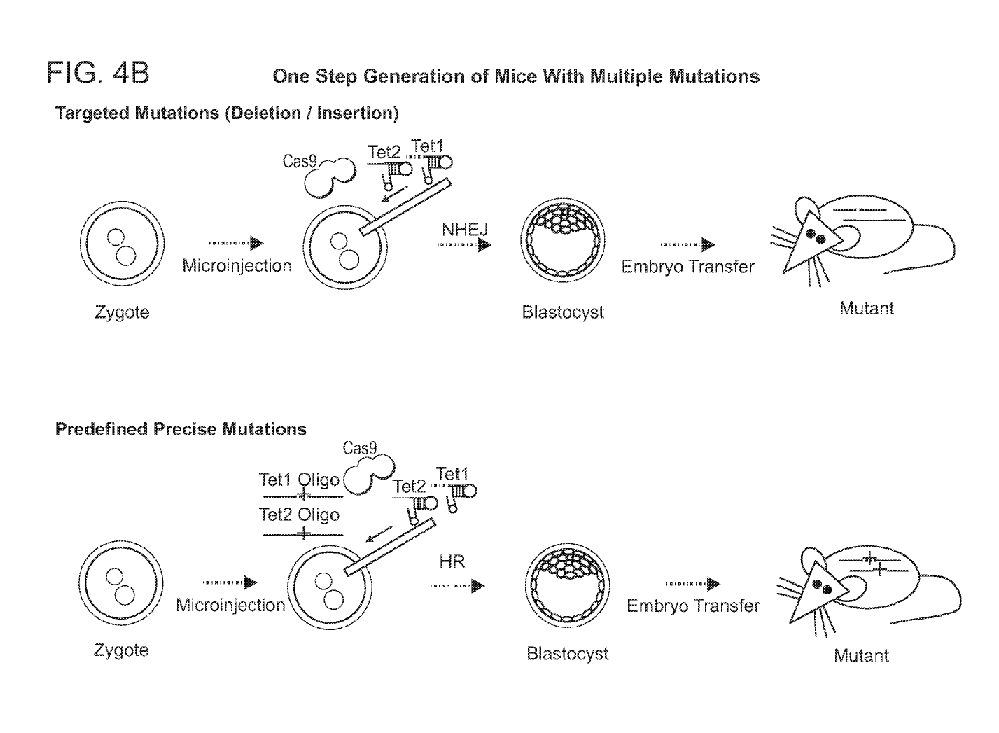

As summarized in FIGS. 4A-4B and described herein, three different approaches for the generation of mice carrying multiple genetic alterations have been established. Demonstrate d herein is that CRISPR/Cas-mediated genome editing in ES cells can generate the simultaneous mutations of several genes with high efficiency, a single-step approach allowing the production of cells with mutations in five different genes (FIG. 4A). Three Tet genes were chosen as targets because the respective mutant phenotypes have been well defined previously (Dawlaty et al, Cell Stem Cell, 9:166-175 (2011); Gu et al, Nature, 477:606-610 (2011)). Cells mutant for Tet1, 2 and 3 were depleted of 5hmC as would be expected for loss of function mutations of the genes (Dawlaty et al., Dev Cell, 24:310-323 (2013)). However, which of the Cas9-mediated gene mutations produced loss of function rather than hypomorphic alleles has not been established.

Also shown herein is that mouse embryos can be directly modified by injection of Cas9 mRNA and sgRNA into the fertilized egg resulting in the efficient production of mice carrying biallelic mutations in a given gene. More significantly, co-injection of Cas9 with Tet1 and Tet2 sgRNAs into zygotes produced mice that carried mutations in both genes (FIG. 4B, upper panel). It was found that up to about 95% of new-born mice were biallelic mutant in the targeted gene when single sgRNA was injected, and when co-injected with two different sgRNAs, up to about 80% carried biallelic mutations in both targeted genes. Thus, mice carrying multiple mutations can be generated within 4 weeks, which is a much shorter time frame than can be achieved by conventional consecutive targeting of genes in ES cells and avoids time-consuming intercrossing of single mutant mice.

The introduction of DSBs by CRISPR/Cas generates mutant alleles with varying deletions or insertions in contrast to designed precise mutations created by homologous recombination. The introduction of point mutations into human ES cells, cancer cell lines, and mouse by ZNF or TALEN along with DNA oligo has been demonstrated previously (Chen et al., Nat Methods, 8:753-755 (2011); Soldner et al., Cell, 146:318-331 (2011); Wefers et al., PNAS, USA, 110:3782-3787 (2013)). Demonstrated herein is that CRISPR/Cas mediated targeting is useful to generate mutant alleles with predetermined alterations, and co-injection of single stranded oligos can introduce designed point mutations into two target genes in one step, allowing for multiplexed gene editing in a strictly controlled manner (FIG. 4B, lower panel). This targeting system allows for the production of conditional alleles, or precise insertion of larger DNA fragments such as GFP markers so as to generate conditional knockout and reporter mice for specific genes.

It is likely that a much larger number of genomic loci than targeted in the present work can be modified simultaneously when pooled sgRNAs are introduced. The methods presented here provide for systematic genome engineering in mice, facilitating the investigation of entire signaling pathways, of synthetic lethal phenotypes or of genes that have redundant functions. A particularly interesting application is the possibility to produce mice carrying multiple alterations in candidate loci that have been identified in GWAS studies to play a role in the genesis of multigenic diseases. In summary, CRISPR/Cas mediated genome editing allows for the generation of ES cells and mice carrying multiple genetic alterations and facilitates the genetic dissection of development and complex diseases.

### Example 2

### RNA-Programmable DNA Binding Enzymes (CRISPRzymes)

Reported herein is the generation of an RNA-guided, programmable transactivator based on CRISPR/Cas system, CRISPRa, which provides a tool for modulation of a (one or more) nucleic acid sequence, e.g., gene activation, and serves as a proof of principle for CRISPR-based RNA-guided DNA binding enzymes (CRISPRzymes).

Results

dCas9ta Guided by sgRNA Targeting Tet Binding Site Activates TetO Promoter

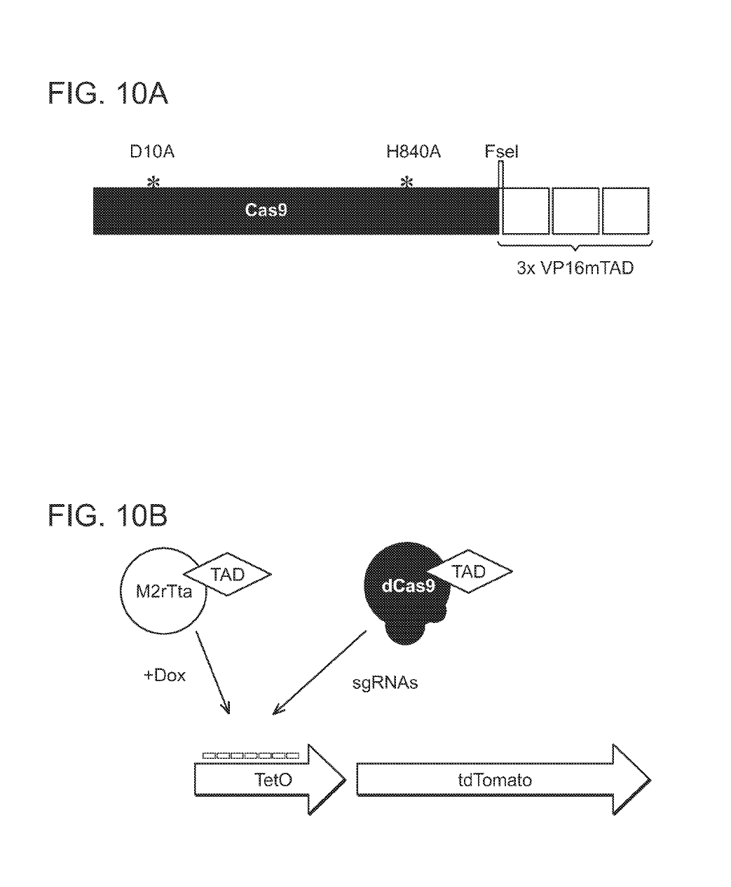

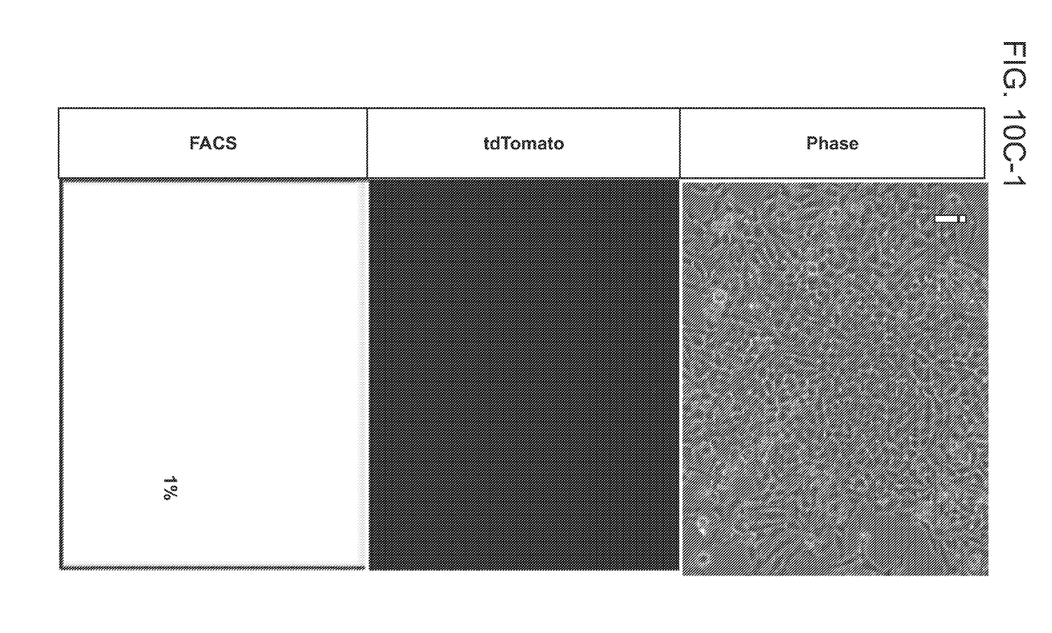

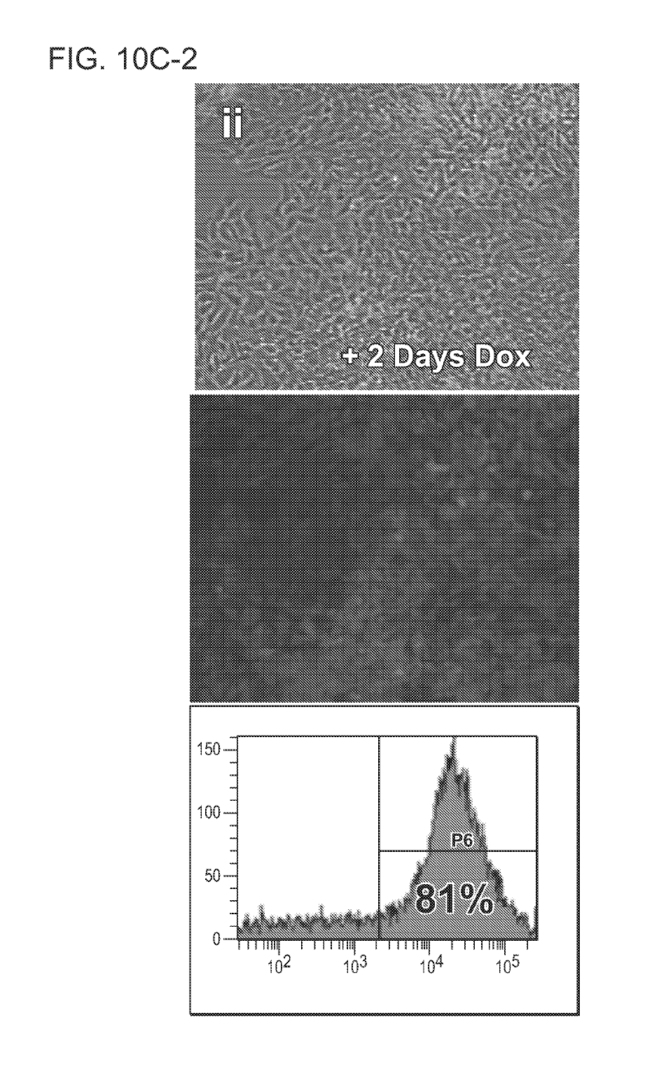

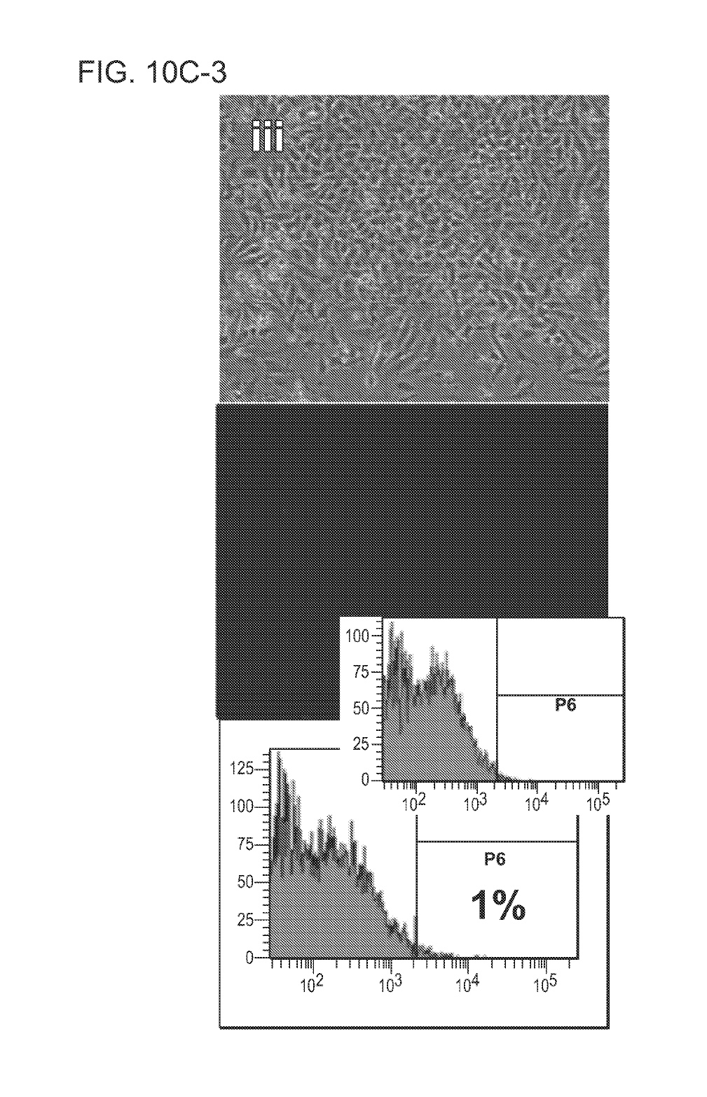

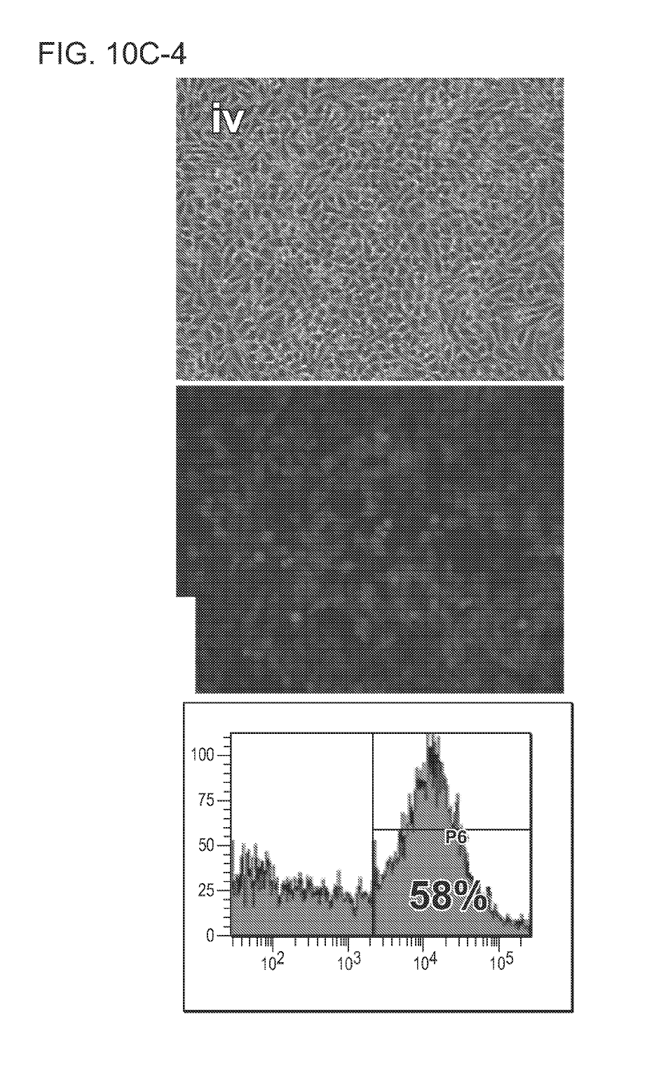

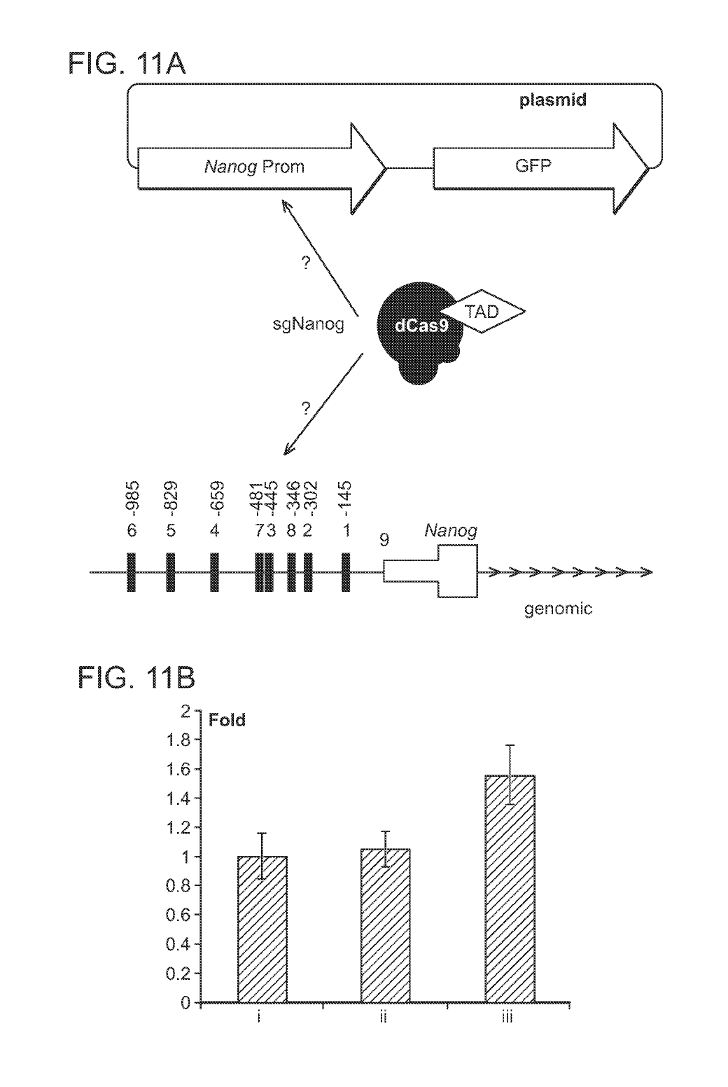

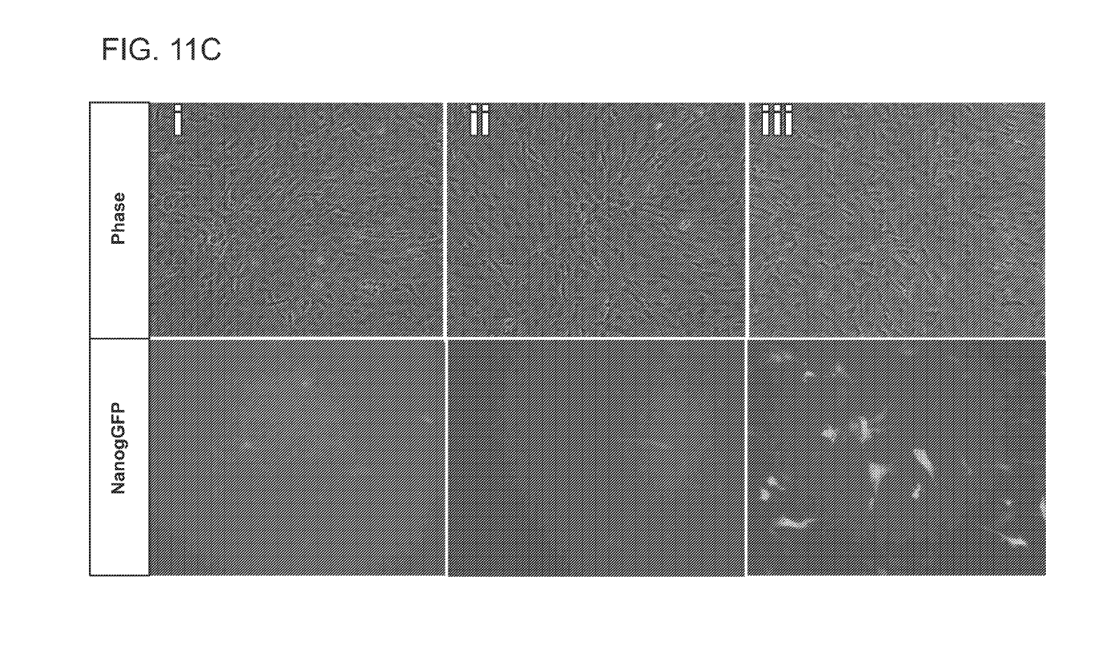

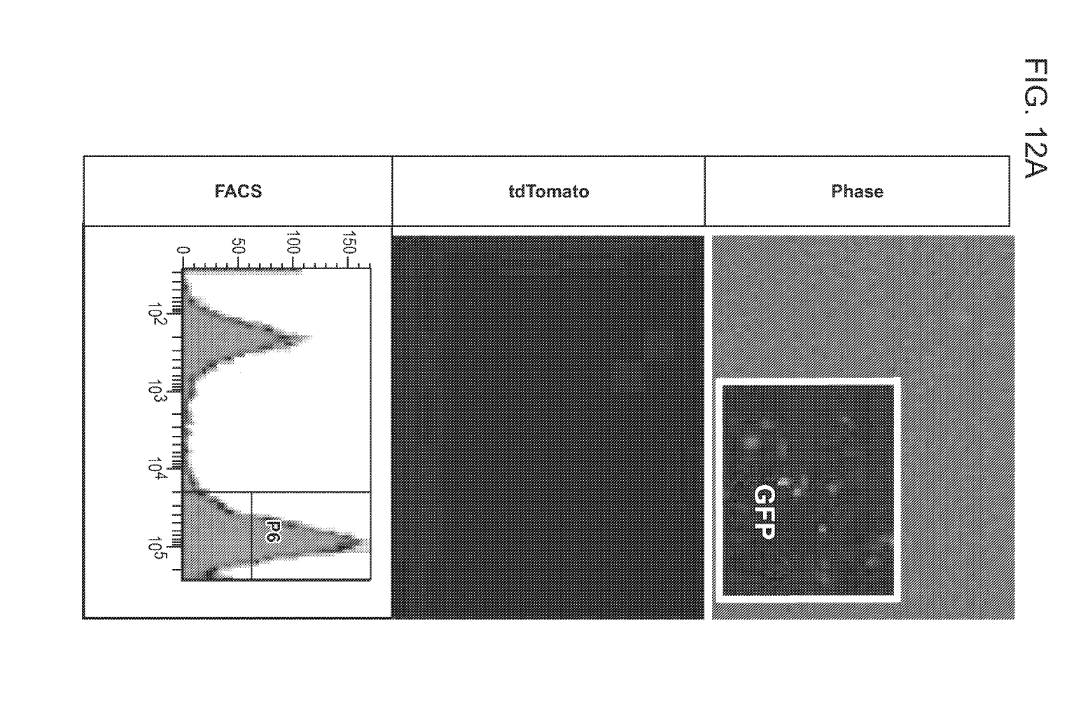

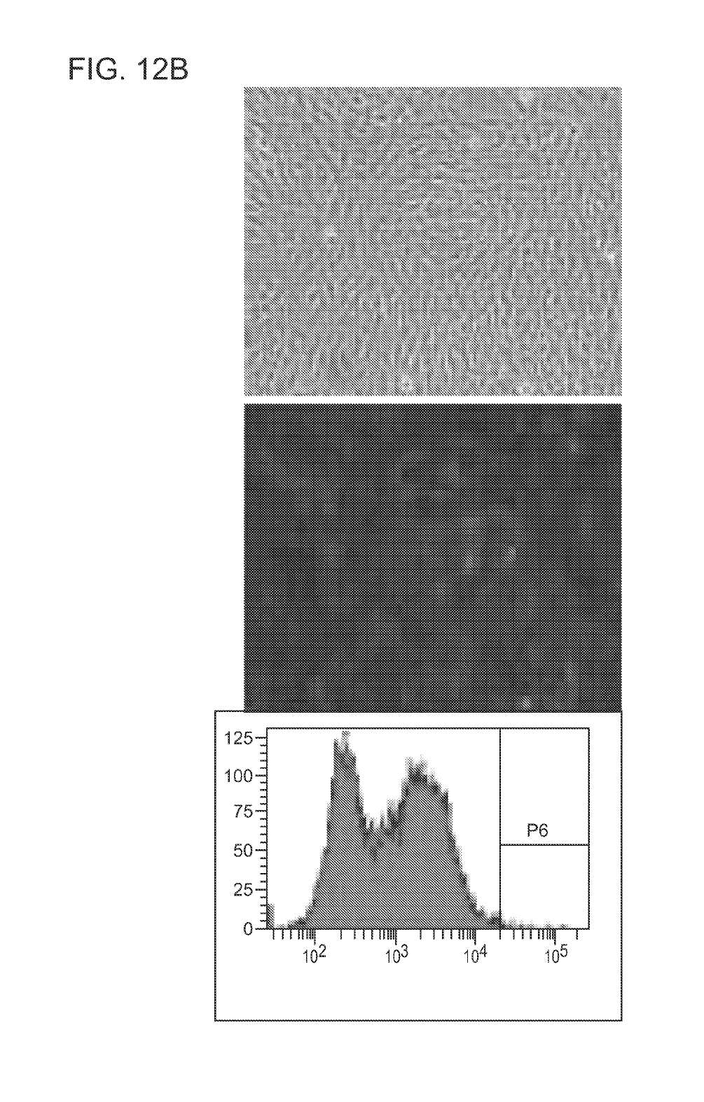

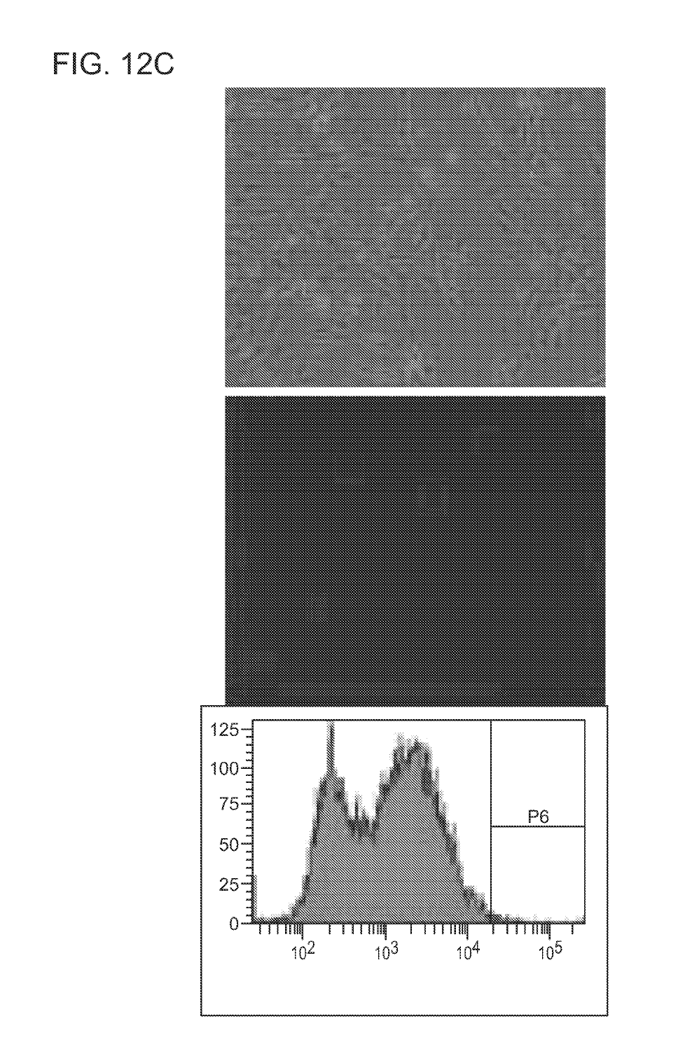

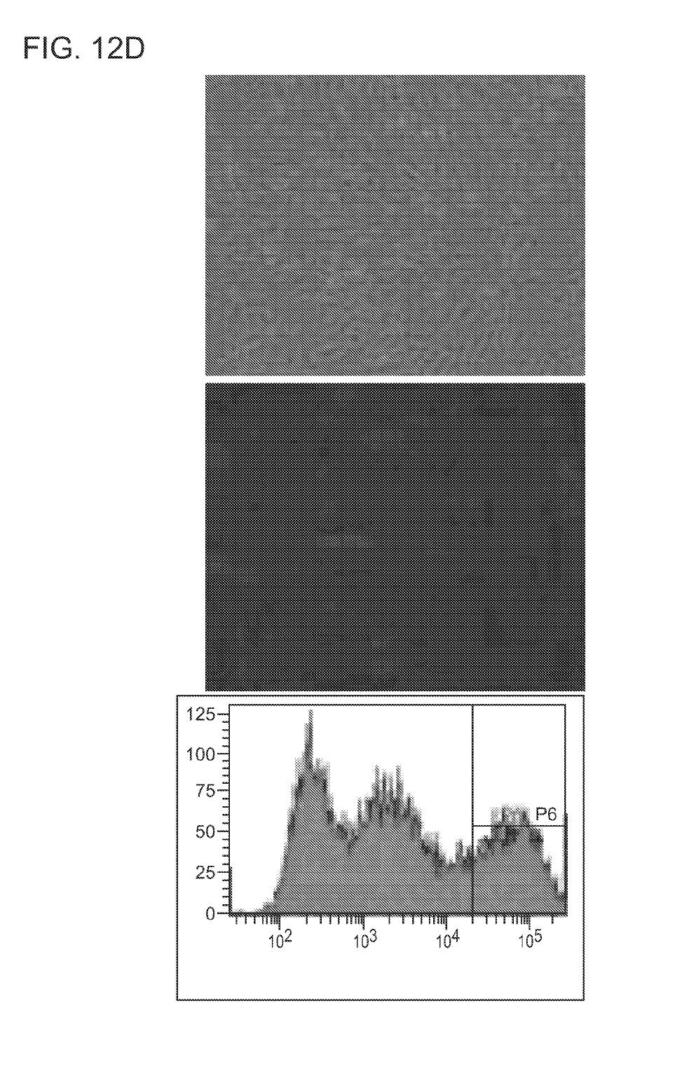

To build a CRISPR/Cas-based transcriptional activator, H840A of the human codon-optimized Cas9 nickase was mutated to generate nuclease-deficient dCas9 [PMID: 23452860] and a 3× minimal VP16 transcriptional activation domain (TAD) was fused to the C-terminal of the dCas9 protein (FIG. 10A) to generate dCas9ta. dCas9ta was first tested on a tdTomato reporter under the control of Tet-inducible promoter with seven copies of tet binding site upstream of a CMV minimal promoter (TetO::tdTomato) (FIG. 10B). HeLa and NIH3T3 cells with TetO::tdTomato transgene were generated by PiggyBac transposition [PMID: 17576687]. As a positive control for the reporter activity, these cells also constitutively expressed the rtTA-M2 transactivator that can induce tdTomato expression upon doxycycline treatment (FIG. 11C panel ii; FIG. 12B). Transfection of dCas9ta with sgRNA (sgTetO) complementary to tet binding site activated TetO::tdTomato reporter in the absence of doxycycline (FIG. 11C panel iv; FIG. 12D). Transfection of dCas9ta without sgRNA did not activate tdTomato expression, indicating the dCas9ta depends on sgRNA to bind to the target tet binding sites to activate tdTomato expression (FIG. 11C panel iii; FIG. 12C).

dCas9ta with sgRNA Targeting Nanog Promoter can Activate Both the NanogGFP Reporter and the Endogenous Nanog Expression in NIH3T3 Cells

To test whether dCas9ta can activate endogenous gene expression, dCas9ta chimeric expression construct was designed and cloned with 8 different sgRNAs targeting Nanog promoter (sgmNanog) and transfected in NIH3T3 cells. As a comparison, a NanogGFP plasmid [PMID: 18594521] containing 1.2 kb promoter of Nanog was co-transfected. Transfection of dCas9ta without sgRNA did not activate the exogenous NanogGFP reporter (FIG. 11C; panel ii) or the endogenous Nanog gene (FIG. 11B, column ii) while transfection of 8 constructs expressing dCas9ta and sgmNanog activated the NanogGFP reporter (FIG. 11C, panel iii) and endogenous Nanog expression (FIG. 11B, column iii).

dCas9 Fusion with P-TEFb Components Also Activate Gene Expression

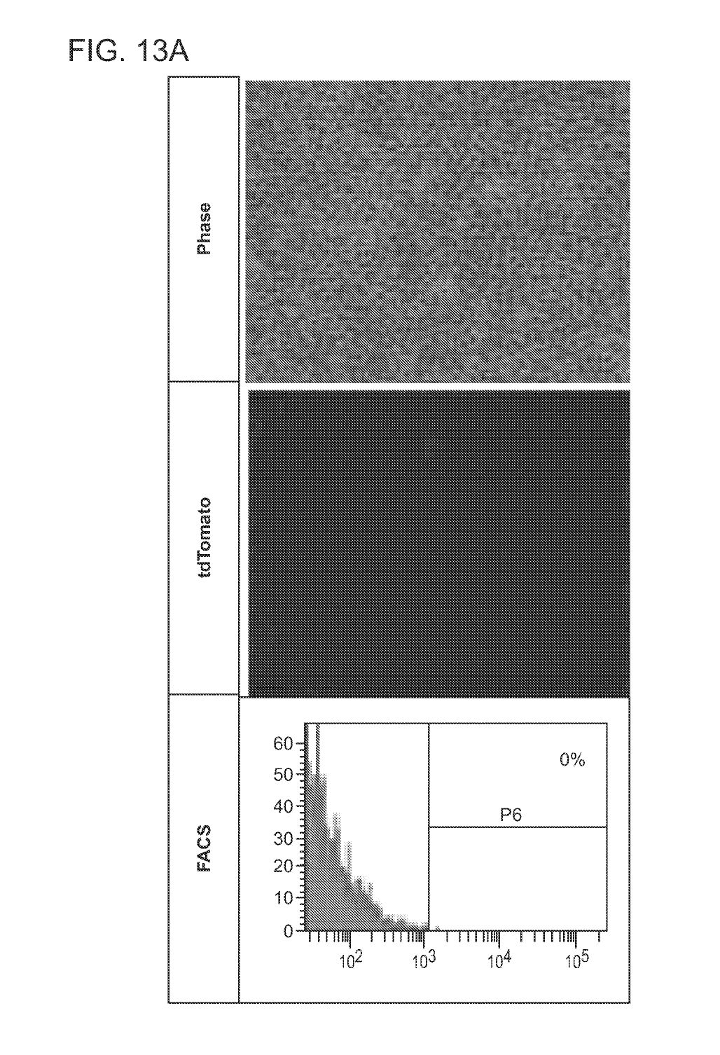

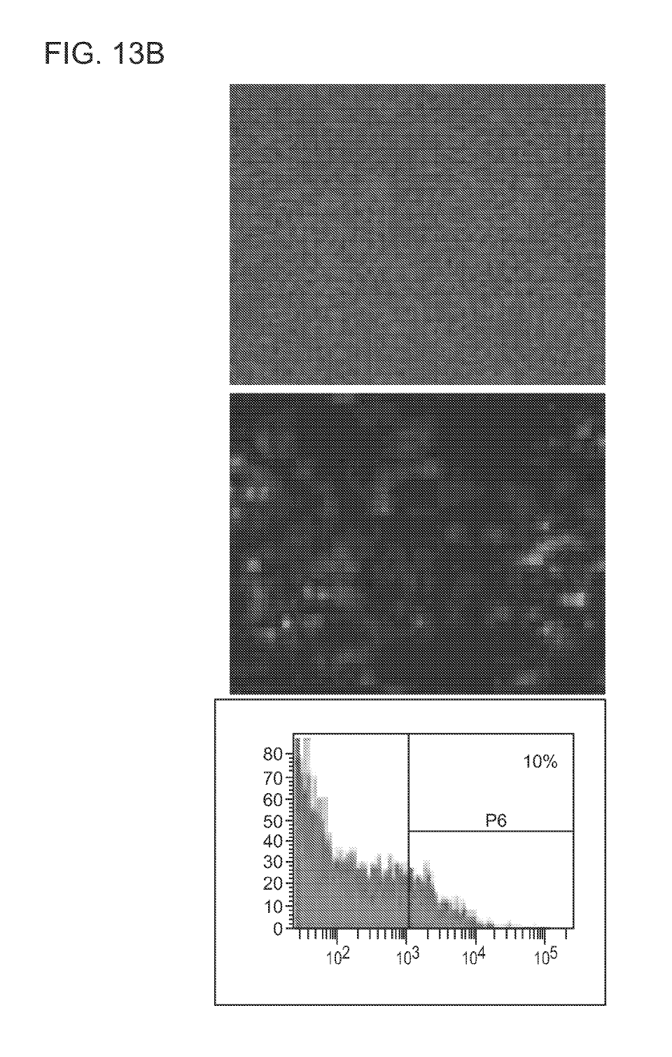

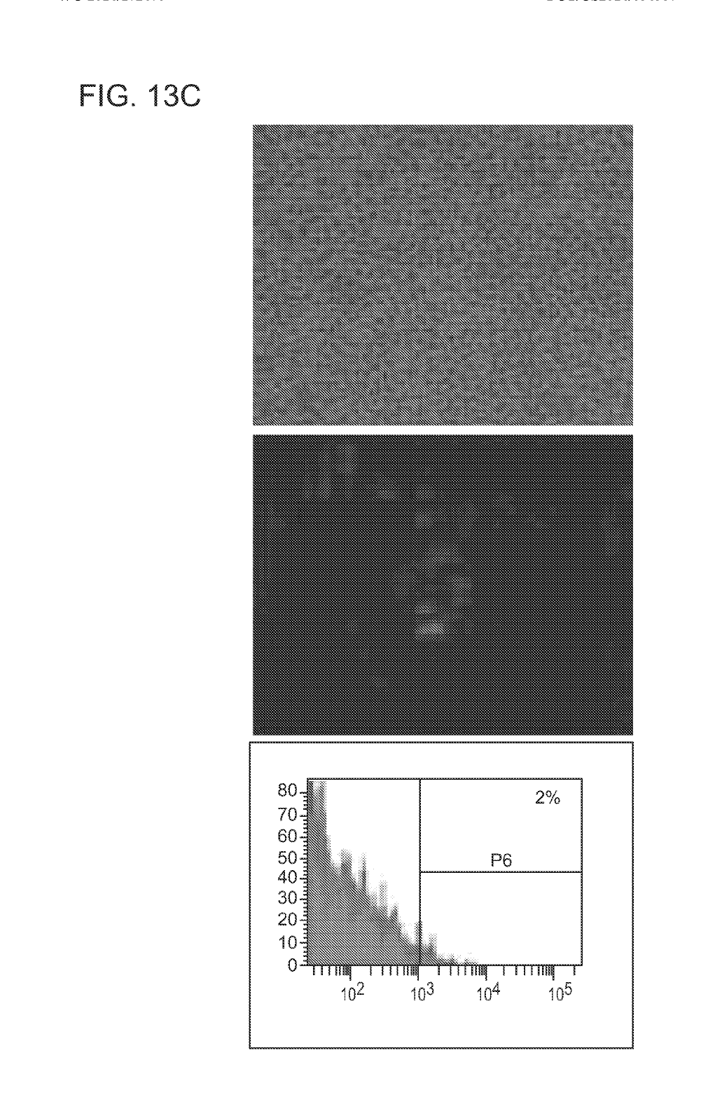

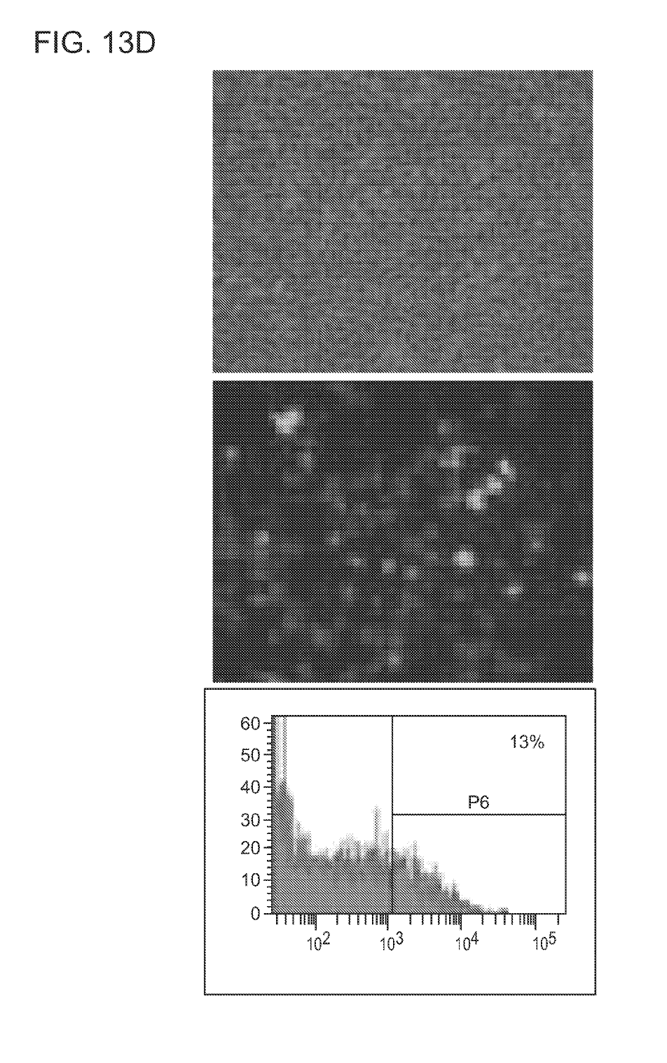

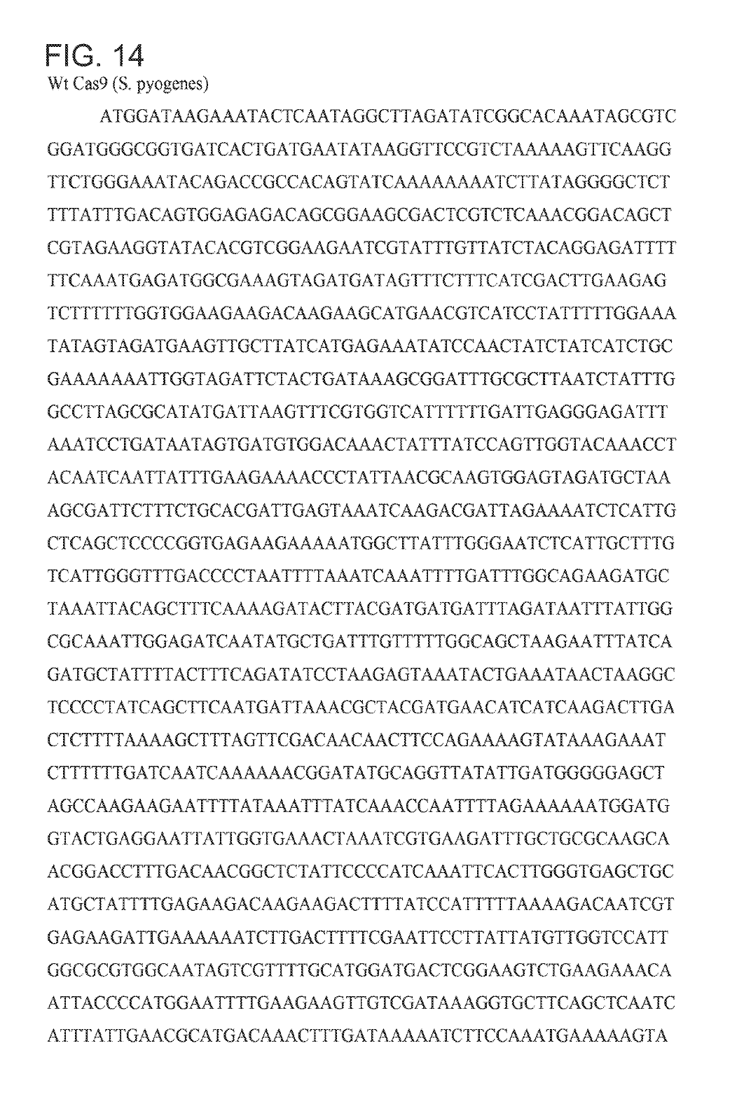

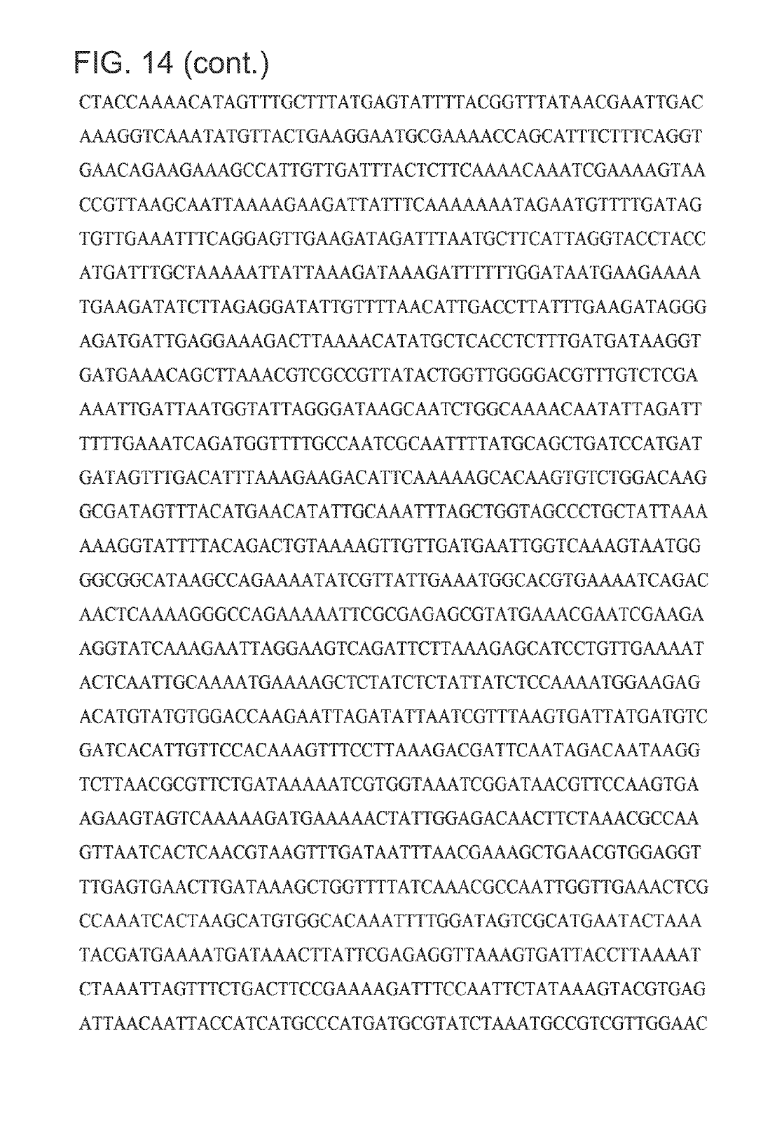

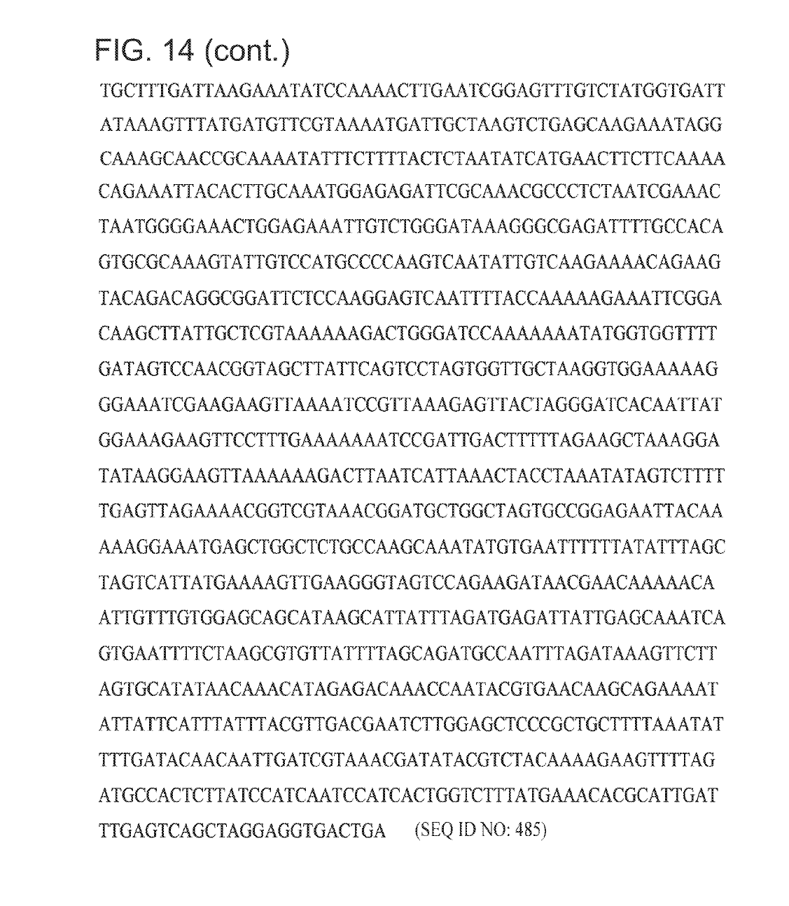

To test whether dCas9 can be used to bring other protein domains to DNA to regulate gene expression, dCas9 was fused to Cdk9 and CycT, two components of the P-TEFb complex involved in the transcriptional pause release [PMID: 22986266] and their transactivation was tested activity on the TetO::tdTomato with or without dCas9ta (FIGS. 13A-13D). Transfection of dCas9ta resulted in 10% of tdTomato positive cells. Transfection of both dCas9Cdk (pAC72) and dCas9CycT (pAC73) also activated tdTomato expression, though to a lesser extent (2%). Co-transfection of three plasmids, pAC5 (dCas9ta), pAC72 (dCas9Cdk9), pAC73 (dCas9CycT), with sgTetO resulted in 13% tdTomato positive cells. This additive effect indicates that co-transfection with or fusion of additional transcriptional activators or transactivation domains to dCas9ta likely further augment dCas9ta transactivation activity.

Materials and Methods

Cloning

A two-step fusion PCR was used to amplify Cas9 Nickase ORF without stop codon from the pX335 vector, incorporate H840A mutation, EcoRI-AgeI restriction site on the 5′ end as well as an FseI site on the 3′end (EcoRI-AgeI-dCas9-FseI fragment). The 3× minimal VP16 activation domain coding fragment (TAD) was excised from a vector (Addgene: 20342) containing NLSM2rtTA coding sequence by FseI and EcoRI digestion (FseI-TA-EcoRI fragment). The two fragments were ligated into pCR8/GW/TOPO (Invitrogen) vector digested by EcoRI to generate pAC1 which contains the dCas9ta gene. The dCas9ta coding sequence was subsequently excised from pAC1 and cloned into pX355 vector (Addgene: 42335) by AgeI-EcoRI digestion to replace dCas9 Nickase to create a chimeric vector pAC2 that expresses both the dCas9ta and the sgRNA. sgRNA spacers were cloned into the BbsI-digested pAC2 vector. For example, sgRNA targeting TetO (sgTet) was cloned by ligating phosphorylated and annealed oligos sgTet-F: caccGCTTTTCTCTATCACTGATA (SEQ ID NO: 179) and sgTet-R: aaacTATCAGTGATAGAGAAAAGC (SEQ ID NO: 180) onto BbsI-digested pAC2 vector to generate pAC5. To replace the 3× minimal activation domain (3×mTAD) in dCas9ta protein for other protein domains, FseI-EcoRI fragment from pAC5 or pAC1 was replaced by PCR amplicons of different domains or genes with FseI and EcoRI added on the primer sequences. dCas9 was cloned by PCR amplification of dCas9ta with reverse primer before the 3×TA domains and cloned into pCR8GWTOPO to create pAC84 and pAC5 to create pAC89. Non-chimeric versions of dCas9 fusions were generated by LR Clonase-medicated recombination to a pmax-DEST vector (pAC90).

| Domains/ | Forward | | |
|---|---|---|---|
| Genes | Primer | Reverse Primer | Plasmid |
| 3xVP16 | N/A (directly | N/A | pAC1, pAC2 |
| (TA) | excised from | | |
| | a plasmid) | | |
| mCdk9 | AAAAAAggccggc | AaaaaaGAATTCtc | pAC72 |
| | cATGGCCAAGCAG | aGAAGACACGTTCA | (dCas9Cdk9; |
| | TACGACTC (SEQ | AATTCCG (SEQ | U6-sgTetO) |
| | ID NO: 181) | ID NO: 182) | |
| mCycT | AAAAAAggccggc | AaaaaaGAATTCTC | pAC73 |
| | cATGGAGGGAGAG | ACTTAGGAAGAGGT | (dCas9CycT; |
| | AGGAAGAACAA | GGAAGTGGTGGA | U6-sgTetO) |
| | (SEQ ID NO: | (SEQ ID NO: | |
| | 183) | 184) | |

A Reporter Assay for dCas9ta Activity

A TetO::tdTomato (plasmid pAC3) transgene and a EF1a::NLSM2rtTA (plasmid pAC4) transgene were delivered into NIH3T3 (mouse) and HeLa (human) cells by PiggyBac transposition. sgRNAs were designed to target TetO binding site (sgTetO). pmaxGFP (Clontech) was used as a transfection control. Transfection was done using FuGene HD following manufacturer's instructions.

qRT Expression Analysis

Pellets were snap-frozen and stored at −80 C. RNA were prepared from the pellets by RNeasy kit (QIAGEN). cDNA were produced by Superscript III RT (Life Technology). qRT were done in triplicates using Gapdh as a control.

qRT primers:

| Gene | Forward primer | Reverese Primer |
|---|---|---|
| mGapdh | TGTGTCCGTCGTGGATCTGA | CCTGCTTCACCACCTTCTTGA |
| | (SEQ ID NO: 185) | (SEQ ID NO: 186) |
| mNanog | TTGCTTACAAGGGTCTGCTA | ACTGGTAGAAGAATCAGGGCT |
| | CT (SEQ ID NO: 187) | (SEQ ID NO: 188) |

sgRNA designs, DNA targets, oligos, and plasmids used to target different DNA. Last three bases are PAM (5′-NGG-3′) motif. Lowercase letters in the target sequences indicate changes made (first g) to allow efficient U6 transcription or for mutational analysis (other changes). Lowercase letters in the oligo sequences indicate overhang compatible to the BbsI-digested vectors. Target gene names with m prefix indicate mouse gene while those with h prefix indicates human genes.

| Name | Target | Target sequences | Forward oligo | Reverse oligo | Plasmid |
|---|---|---|---|---|---|
| sgTet | Tet | Gcttttctctatcact | CaccGCTTTTCTCTAT | AaacTATCAGTG | pAC5 |
| | binding | gataggg | CACTGATA | ATAGAGAAAAGC | |
| | site | (SEQ ID NO: 189) | (SEQ ID NO: 190) | (SEQ ID NO: 191) | |
| sgmNanog-1 | mNanog | GTAATGCAAAA | CaccGTAATGCAAAA | AaacTACAGCTT | pAC34 |
| | promoter | GAAGCTGTAAGG | GAAGCTGTA | CTTTTGCATTAC | |
| | | (SEQ ID NO: 192) | SEQ ID NO: (193) | (SEQ ID NO: 194) | |
| sgmNanog-2 | mNanog | GATCTCTAGTG | CaccGATCTCTAGTG | AaacGAAACTTC | pAC35 |
| | promoter | GGAAGTTTCAGG | GGAAGTTTC | CCACTAGAGATC | |
| | | (SEQ ID NO: 195) | (SEQ ID NO: 196) | (SEQ ID NO: 197) | |
| sgmNanog-3 | mNanog | GCTCTTCACAT | CaccGCTCTTCACAT | AaacGGTTTCCC | pAC36 |
| | promoter | TGGGAAACCTGG | TGGGAAACC | AATGTGAAGAGC | |
| | | (SEQ ID NO: 198) | (SEQ ID NO: 199) | (SEQ ID NO: 200) | |
| sgmNanog-4 | mNanog | GAGTGTTTAAA | CaccGAGTGTTTAAA | AaacCTACATTA | pAC37 |
| | promoter | TTAATGTAGAGG | TTAATGTAG | ATTTAAACACTC | |
| | | (SEQ ID NO: 201) | (SEQ ID NO: 202) | (SEQ ID NO: 203) | |
| sgmNanog-5 | mNanog | GAGTTTCACGT | CaccGAGTTTCACGT | AaacGTCTCGGG | pAC38 |
| | promoter | ACCCGAGACTGG | ACCCGAGAC | TACGTGAAACTC | |
| | | (SEQ ID NO: 204) | (SEQ ID NO: 205) | (SEQ ID NO: 206) | |
| sgmNanog-6 | mNanog | GCTTCTGTGTA | CaccGCTTCTGTGTA | AaacCTCTGCTT | pAC39 |
| | promoter | TAAGCAGAGAGG | TAAGCAGAG | ATACACAGAAGC | |
| | | (SEQ ID NO: 207) | (SEQ ID NO: 208) | (SEQ ID NO: 209) | |
| sgmNanog-7 | mNanog | GCGTTAAAAAG | CaccGCGTTAAAAAG | AaacAAAGTGCG | pAC47 |
| | promoter | CCGCACTTTTGG | CCGCACTTT | GCTTTTTAACGC | |
| | | (SEQ ID NO: 210) | (SEQ ID NO: 211) | (SEQ ID NO: 212) | |
| sgmNanog-8 | mNanog | GTCTGTAGAAA | CaccGTCTGTAGAAA | AaacCTTCCATT | pAC48 |
| | promoter | GAA | GAATGGAAG | CTTTCTACAGAC | |
| | | (SEQ ID NO: 213) | (SEQ ID NO: 214) | (SEQ ID NO: 215) | |

Genbank files

| Plasmid | |
|---|---|
| name | description |
| pAC1 | dCas9ta on pCR8GWTOPO |
| pAC2 | Dual expression construct expressing both dCas9ta |
| | and sgRNA from U6 promoter |
| pAC3 | TetO::tdTomato PiggyBac |
| pAC4 | EF1a::NLSM2rtTA PiggyBac |
| pAC5 | Dual expression construct expression both dCas9ta |
| | and sgTetO |
| pAC34 | Dual expression construct expression both dCas9ta |
| | and sgmNanog1 |
| pAC35 | Dual expression construct expression both dCas9ta |
| | and sgmNanog2 |
| pAC36 | Dual expression construct expression both dCas9ta |
| | and sgmNanog3 |
| pAC37 | Dual expression construct expression both dCas9ta |
| | and sgmNanog4 |
| pAC38 | Dual expression construct expression both dCas9ta |
| | and sgmNanog5 |
| pAC39 | Dual expression construct expression both dCas9ta |
| | and sgmNanog6 |
| pAC47 | Dual expression construct expression both dCas9ta |
| | and sgmNanog7 |
| pAC48 | Dual expression construct expression both dCas9ta |
| | and sgmNanog8 |
| pAC71 | Dual expression construct expression both dCas9Apobec1 |
| | and sgTetO |
| pAC72 | Dual expression construct expression both dCas9Cdk1 |
| | and sgTetO |
| pAC73 | Dual expression construct expression both dCas9CycT |
| | and sgTetO |
| pAC76 | Dual expression construct expression both dCas9Gadd45a |
| | and sgTetO |
| pAC78 | Dual expression construct expression both dCas9mCBPHAT |
| | and sgTetO |
| pAC80 | Dual expression construct expression both puroR and |
| | sgTetO (control) |
| pAC81 | dCas9ER on pCR8GWTOPO |
| pAC82 | dCas9p300HAT on pCR8GWTOPO |
| pAC83 | dCas9Tet1CD on pCR8GWTOPO |
| pAC84 | dCas9 on pCR8GWTOPO |
| pAC85 | dCas9EnR on pmax expression vector |
| pAC86 | dCas9p300HAT on pmax expression vector |
| pAC87 | dCas9Tet1CD on pmax expression vector |
| pAC88 | dCas9ta on pmax expression vector |
| pAC89 | Dual expression construct expression both dCas9 and sgTetO |

CRISPRzyme dCas9 Fusion Peptides

Sequences: dCas9TA peptide sequence is shown below. The underlined sequence indicates the 3×VP16 minimal transactivation domains.

dCas9TA peptide sequence (Underlined sequence indicate the 3x VP16
minimal Transactivation domains)
(SEQ ID NO: 1)
MYPYDVPDYASPKKKRKVEASDKKYSIGLAIGTNSVGWAVIT
DEYKVPSKKFKVLGNTDRHSIKKNLIGALLFDSGETAEATRLKRTARRRYTR
RKNRICYLQEIFSNEMAKVDDSFFHRLEESFLVEEDKKHERHPIFGNIVDEVA
YHEKYPTIYHLRKKLVDSTDKADLRLIYLALAHMIKFRGHFLIEGDLNPDNS
DVDKLFIQLVQTYNQLFEENPINASGVDAKAILSARLSKSRRLENLIAQLPGE
KKNGLFGNLIALSLGLTPNFKSNFDLAEDAKLQLSKDTYDDDLDNLLAQIG
DQYADLFLAAKNLSDAILLSDILRVNTEITKAPLSASMIKRYDEHHQDLTLL
KALVRQQLPEKYKEIFFDQSKNGYAGYIDGGASQEEFYKFIKPILEKMDGTE
ELLVKLNREDLLRKQRTFDNGSIPHQIHLGELHAILRRQEDFYPFLKDNREKI
EKILTFRIPYYVGPLARGNSRFAWMTRKSEETITPWNFEEVVDKGASAQSFIE
RMTNFDKNLPNEKVLPKHSLLYEYFTVYNELTKVKYVTEGMRKPAFLSGE
QKKAIVDLLFKTNRKVTVKQLKEDYFKKIECFDSVEISGVEDRFNASLGTYH
DLLKIIKDKDFLDNEENEDILEDIVLTLTLFEDREMIEERLKTYAHLFDDKVM
KQLKRRRYTGWGRLSRKLINGIRDKQSGKTILDFLKSDGFANRNFMQLIHD
DSLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHPVE
NTQLQNEKLYLYYLQNGRDMYVDQELDINRLSDYDVDAIVPQSFLKDDSID
NKVLTRSDKNRGKSDNVPSEEVVKKMKNYWRQLLNAKLITQRKFDNLTKA
ERGGLSELDKAGFIKRQLVETRQITKHVAQILDSRMNTKYDENDKLIREVKV
ITLKSKLVSDFRKDFQFYKVREINNYHHAHDAYLNAVVGTALIKKYPKLESE
FVYGDYKVYDVRKMIAKSEQEIGKATAKYFFYSNIMNFFKTEITLANGEIRK
RPLIETNGETGEIVWDKGRDFATVRKVLSMPQVNIVKKTEVQTGGFSKESIL
PKRNSDKLIARKKDWDPKKYGGFDSPTVAYSVLVVAKVEKGKSKKLKSVK
ELLGITIMERSSFEKNPIDFLEAKGYKEVKKDLIIKLPKYSLFELENGRKRML
ASAGELQKGNELALPSKYVNFLYLASHYEKLKGSPEDNEQKQLFVEQHKH
YLDEIIEQISEFSKRVILADANLDKVLSAYNKHRDKPIREQAENIIHLFTLTNL
GAPAAFKYFDTTIDRKRYTSTKEVLDATLIHQSITGLYETRIDLSQLGGDSPK
KKRKVEASGPADALDDFDLDMLPADALDDFDLDMLPADALDDFDLDMLPG-
dCas9Apobec 1
(SEQ ID NO: 2)
MYPYDVPDYASPKKKRKVEASDKKYSIGLAIGTNSVGWAVIT
DEYKVPSKKFKVLGNTDRHSIKKNLIGALLFDSGETAEATRLKRTARRRYTR
RKNRICYLQEIFSNEMAKVDDSFFHRLEESFLVEEDKKHERHPIFGNIVDEVA
YHEKYPTIYHLRKKLVDSTDKADLRLIYLALAHMIKFRGHFLIEGDLNPDNS
DVDKLFIQLVQTYNQLFEENPINASGVDAKAILSARLSKSRRLENLIAQLPGE
KKNGLFGNLIALSLGLTPNFKSNFDLAEDAKLQLSKDTYDDDLDNLLAQIG
DQYADLFLAAKNLSDAILLSDILRVNTEITKAPLSASMIKRYDEHHQDLTLL
KALVRQQLPEKYKEIFFDQSKNGYAGYIDGGASQEEFYKFIKPILEKMDGTE
ELLVKLNREDLLRKQRTFDNGSIPHQIHLGELHAILRRQEDFYPFLKDNREKI
EKILTFRIPYYVGPLARGNSRFAWMTRKSEETITPWNFEEVVDKGASAQSFIE
RMTNFDKNLPNEKVLPKHSLLYEYFTVYNELTKVKYVTEGMRKPAFLSGE
QKKAIVDLLFKTNRKVTVKQLKEDYFKKIECFDSVEISGVEDRFNASLGTYH
DLLKIIKDKDFLDNEENEDILEDIVLTLTLFEDREMIEERLKTYAHLFDDKVM
KQLKRRRYTGWGRLSRKLINGIRDKQSGKTILDFLKSDGFANRNFMQLIHD
DSLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHPVE
NTQLQNEKLYLYYLQNGRDMYVDQELDINRLSDYDVDAIVPQSFLKDDSID
NKVLTRSDKNRGKSDNVPSEEVVKKMKNYWRQLLNAKLITQRKFDNLTKA
ERGGLSELDKAGFIKRQLVETRQITKHVAQILDSRMNTKYDENDKLIREVKV
ITLKSKLVSDFRKDFQFYKVREINNYHHAHDAYLNAVVGTALIKKYPKLESE
FVYGDYKVYDVRKMIAKSEQEIGKATAKYFFYSNIMNFFKTEITLANGEIRK
RPLIETNGETGEIVWDKGRDFATVRKVLSMPQVNIVKKTEVQTGGFSKESIL
PKRNSDKLIARKKDWDPKKYGGFDSPTVAYSVLVVAKVEKGKSKKLKSVK
ELLGITIMERSSFEKNPIDFLEAKGYKEVKKDLIIKLPKYSLFELENGRKRML
ASAGELQKGNELALPSKYVNFLYLASHYEKLKGSPEDNEQKQLFVEQHKH
YLDEIIEQISEFSKRVILADANLDKVLSAYNKHRDKPIREQAENIIHLFTLTNL
GAPAAFKYFDTTIDRKRYTSTKEVLDATLIHQSITGLYETRIDLSQLGGDSPK
KKRKVEASGPAMSSETGPVAVDPTLRRRIEPHEFEVFFDPRELRKETCLLYEI
NWGGRHSVWRHTSQNTSNHVEVNFLEKFTTERYFRPNTRCSITWFLSWSPC
GECSRAITEFLSRHPYVTLFIYIARLYHHTDQRNRQGLRDLISSGVTIQIMTEQ
EYCYCWRNFVNYPPSNEAYWPRYPHLWVKLYVLELYCIILGLPPCLKILRR
KQPQLTFFTITLQTCHYQRIPPHLLWATGLK
dCas9Cdk9
(SEQ ID NO: 3)
MYPYDVPDYASPKKKRKVEASDKKYSIGLAIGTNSVGWAVIT
DEYKVPSKKFKVLGNTDRHSIKKNLIGALLFDSGETAEATRLKRTARRRYTR
RKNRICYLQEIFSNEMAKVDDSFFHRLEESFLVEEDKKHERHPIFGNIVDEVA
YHEKYPTIYHLRKKLVDSTDKADLRLIYLALAHMIKFRGHFLIEGDLNPDNS
DVDKLFIQLVQTYNQLFEENPINASGVDAKAILSARLSKSRRLENLIAQLPGE
KKNGLFGNLIALSLGLTPNFKSNFDLAEDAKLQLSKDTYDDDLDNLLAQIG
DQYADLFLAAKNLSDAILLSDILRVNTEITKAPLSASMIKRYDEHHQDLTLL
KALVRQQLPEKYKEIFFDQSKNGYAGYIDGGASQEEFYKFIKPILEKMDGTE
ELLVKLNREDLLRKQRTFDNGSIPHQIHLGELHAILRRQEDFYPFLKDNREKI
EKILTFRIPYYVGPLARGNSRFAWMTRKSEETITPWNFEEVVDKGASAQSFIE
RMTNFDKNLPNEKVLPKHSLLYEYFTVYNELTKVKYVTEGMRKPAFLSGE
QKKAIVDLLFKTNRKVTVKQLKEDYFKKIECFDSVEISGVEDRFNASLGTYH
DLLKIIKDKDFLDNEENEDILEDIVLTLTLFEDREMIEERLKTYAHLFDDKVM
KQLKRRRYTGWGRLSRKLINGIRDKQSGKTILDFLKSDGFANRNFMQLIHD
DSLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHPVE
NTQLQNEKLYLYYLQNGRDMYVDQELDINRLSDYDVDAIVPQSFLKDDSID
NKVLTRSDKNRGKSDNVPSEEVVKKMKNYWRQLLNAKLITQRKFDNLTKA
ERGGLSELDKAGFIKRQLVETRQITKHVAQILDSRMNTKYDENDKLIREVKV
ITLKSKLVSDFRKDFQFYKVREINNYHHAHDAYLNAVVGTALIKKYPKLESE
FVYGDYKVYDVRKMIAKSEQEIGKATAKYFFYSNIMNFFKTEITLANGEIRK
RPLIETNGETGEIVWDKGRDFATVRKVLSMPQVNIVKKTEVQTGGFSKESIL
PKRNSDKLIARKKDWDPKKYGGFDSPTVAYSVLVVAKVEKGKSKKLKSVK
ELLGITIMERSSFEKNPIDFLEAKGYKEVKKDLIIKLPKYSLFELENGRKRML
ASAGELQKGNELALPSKYVNFLYLASHYEKLKGSPEDNEQKQLFVEQHKH
YLDEIIEQISEFSKRVILADANLDKVLSAYNKHRDKPIREQAENIIHLFTLTNL
GAPAAFKYFDTTIDRKRYTSTKEVLDATLIHQSITGLYETRIDLSQLGGDSPK
KKRKVEASGPAMAKQYDSVECPFCDEVTKYEKLAKIGQGTFGEVFKAKHR
QTGQKVALKKVLMENEKEGFPITALREIKILQLLKHENVVNLIEICRTKASPY
NRCKGSIYLVFDFCEHDLAGLLSNVLVKFTLSEIKRVMQMLLNGLYYIHRN
KILHRDMKAANVLITRDGVLKLADFGLARAFSLAKNSQPNRYTNRVVTLW
YRPPELLLGERDYGPPIDLWGAGCIMAEMWTRSPIMQGNTEQHQLALISQL
CGSITPEVWPNVDKYELFEKLELVKGQKRKVKDRLKAYVRDPYALDLIDKL
LVLDPAQRIDSDDALNHDFFWSDPMPSDLKGMLSTHLTSMFEYLAPPRRKG
SQITQQSTNQSRNPATTNQTEFERVF
dCas9CycT
(SEQ ID NO: 4)
MYPYDVPDYASPKKKRKVEASDKKYSIGLAIGTNSVGWAVIT
DEYKVPSKKFKVLGNTDRHSIKKNLIGALLFDSGETAEATRLKRTARRRYTR
RKNRICYLQEIFSNEMAKVDDSFFHRLEESFLVEEDKKHERHPIFGNIVDEVA
YHEKYPTIYHLRKKLVDSTDKADLRLIYLALAHMIKFRGHFLIEGDLNPDNS
DVDKLFIQLVQTYNQLFEENPINASGVDAKAILSARLSKSRRLENLIAQLPGE
KKNGLFGNLIALSLGLTPNFKSNFDLAEDAKLQLSKDTYDDDLDNLLAQIG
DQYADLFLAAKNLSDAILLSDILRVNTEITKAPLSASMIKRYDEHHQDLTLL
KALVRQQLPEKYKEIFFDQSKNGYAGYIDGGASQEEFYKFIKPILEKMDGTE
ELLVKLNREDLLRKQRTFDNGSIPHQIHLGELHAILRRQEDFYPFLKDNREKI
EKILTFRIPYYVGPLARGNSRFAWMTRKSEETITPWNFEEVVDKGASAQSFIE
RMTNFDKNLPNEKVLPKHSLLYEYFTVYNELTKVKYVTEGMRKPAFLSGE
QKKAIVDLLFKTNRKVTVKQLKEDYFKKIECFDSVEISGVEDRFNASLGTYH
DLLKIIKDKDFLDNEENEDILEDIVLTLTLFEDREMIEERLKTYAHLFDDKVM
KQLKRRRYTGWGRLSRKLINGIRDKQSGKTILDFLKSDGFANRNFMQLIHD
DSLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHPVE
NTQLQNEKLYLYYLQNGRDMYVDQELDINRLSDYDVDAIVPQSFLKDDSID
NKVLTRSDKNRGKSDNVPSEEVVKKMKNYWRQLLNAKLITQRKFDNLTKA
ERGGLSELDKAGFIKRQLVETRQITKHVAQILDSRMNTKYDENDKLIREVKV
ITLKSKLVSDFRKDFQFYKVREINNYHHAHDAYLNAVVGTALIKKYPKLESE
FVYGDYKVYDVRKMIAKSEQEIGKATAKYFFYSNIMNFFKTEITLANGEIRK
RPLIETNGETGEIVWDKGRDFATVRKVLSMPQVNIVKKTEVQTGGFSKESIL
PKRNSDKLIARKKDWDPKKYGGFDSPTVAYSVLVVAKVEKGKSKKLKSVK
ELLGITIMERSSFEKNPIDFLEAKGYKEVKKDLIIKLPKYSLFELENGRKRML
ASAGELQKGNELALPSKYVNFLYLASHYEKLKGSPEDNEQKQLFVEQHKH
YLDEIIEQISEFSKRVILADANLDKVLSAYNKHRDKPIREQAENIIHLFTLTNL
GAPAAFKYFDTTIDRKRYTSTKEVLDATLIHQSITGLYETRIDLSQLGGDSPK
KKRKVEASGPAMEGERKNNNKRWYFTREQLENSPSRRFGVDSDKELSYRQ
QAANLLQDMGQRLNVSQLTINTAIVYMHRFYMIQSFTQFHRYSMAPAALFL
AAKVEEQPKKLEHVIKVAHTCLHPQESLPDTRSEAYLQQVQDLVILESIILQT
LGFELTIDHPHTHVVKCTQLVRASKDLAQTSYFMATNSLHLTTFSLQYTPPV
VACVCIHLACKWSNWEIPVSTDGKHWWEYVDATVTLELLDELTHEFLQILE
KTPSRLKRIRNWRAYQAAMKTKPDDRGADENTSEQTILNMISQTSSDTTIAG
LMSMSTASTSAVPSLPSSEESSSSLTSVDMLQGERWLSSQPPFKLEAAQGHR
TSESLALIGVDHSLQQDGSSAFGSQKQASKSVPSAKVSLKEYRAKHAEELAA
QKRQLENMEANVKSQYAYAAQNLLSHDSHSSVILKMPIESSENPERPFLDK
ADKSALKMRLPVASGDKAVSSKPEEIKMRIKVHSAGDKHNSIEDSVTKSRE
HKEKQRTHPSNHHHHHNHHSHRHSHLQLPAGPVSKRPSDPKHSSQTSTLAH
KTYSLSSTLSSSSSTRKRGPPEETGAAVFDHPAKIAKSTKSSLNFPFPPLPTMT
QLPGHSSDTSGLPFSQPSCKTRVPHMKLDKGPPGANGHNATQSIDYQDTVN
MLHSLLSAQGVQPTQAPAFEFVHSYGEYMNPRAGAISS
RSGTTDKPRPPPLPSEPPPPLPPLPK
dCas9EnR
(SEQ ID NO: 5)
MYPYDVPDYASPKKKRKVEASDKKYSIGLAIGTNSVGWAVIT
DEYKVPSKKFKVLGNTDRHSIKKNLIGALLFDSGETAEATRLKRTARRRYTR
RKNRICYLQEIFSNEMAKVDDSFFHRLEESFLVEEDKKHERHPIFGNIVDEVA
YHEKYPTIYHLRKKLVDSTDKADLRLIYLALAHMIKFRGHFLIEGDLNPDNS
DVDKLFIQLVQTYNQLFEENPINASGVDAKAILSARLSKSRRLENLIAQLPGE
KKNGLFGNLIALSLGLTPNFKSNFDLAEDAKLQLSKDTYDDDLDNLLAQIG
DQYADLFLAAKNLSDAILLSDILRVNTEITKAPLSASMIKRYDEHHQDLTLL
KALVRQQLPEKYKEIFFDQSKNGYAGYIDGGASQEEFYKFIKPILEKMDGTE
ELLVKLNREDLLRKQRTFDNGSIPHQIHLGELHAILRRQEDFYPFLKDNREKI
EKILTFRIPYYVGPLARGNSRFAWMTRKSEETITPWNFEEVVDKGASAQSFIE
RMTNFDKNLPNEKVLPKHSLLYEYFTVYNELTKVKYVTEGMRKPAFLSGE
QKKAIVDLLFKTNRKVTVKQLKEDYFKKIECFDSVEISGVEDRFNASLGTYH
DLLKIIKDKDFLDNEENEDILEDIVLTLTLFEDREMIEERLKTYAHLFDDKVM
KQLKRRRYTGWGRLSRKLINGIRDKQSGKTILDFLKSDGFANRNFMQLIHD
DSLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHPVE
NTQLQNEKLYLYYLQNGRDMYVDQELDINRLSDYDVDAIVPQSFLKDDSID
NKVLTRSDKNRGKSDNVPSEEVVKKMKNYWRQLLNAKLITQRKFDNLTKA
ERGGLSELDKAGFIKRQLVETRQITKHVAQILDSRMNTKYDENDKLIREVKV
ITLKSKLVSDFRKDFQFYKVREINNYHHAHDAYLNAVVGTALIKKYPKLESE
FVYGDYKVYDVRKMIAKSEQEIGKATAKYFFYSNIMNFFKTEITLANGEIRK
RPLIETNGETGEIVWDKGRDFATVRKVLSMPQVNIVKKTEVQTGGFSKESIL
PKRNSDKLIARKKDWDPKKYGGFDSPTVAYSVLVVAKVEKGKKKLKSVK
ELLGITIMERSSFEKNPIDFLEAKGYKEVKKDLIIKLPKYSLFELENGRKRML
ASAGELQKGNELALPSKYVNFLYLASHYEKLKGSPEDNEQKQLFVEQHKH
YLDEIIEQISEFSKRVILADANLDKVLSAYNKHRDKPIREQAENIIHLFTLTNL
GAPAAFKYFDTTIDRKRYTSTKEVLDATLIHQSITGLYETRIDLSQLGGDSPK
KKRKVEASGPALEDRCSPQSAPSPITLQMQHLHHQQQQQQQQQQQMQHLH
QLQQLQQLHQQQLAAGVFHHPAMAFDAAAAAAAAAAAAAAHAHAAALQ
QRLSGSGSPASCSTPASSTPLTIKEEESDSVIGDMSFHNQTHTTNEEEEAEED
DDIDVDVDDTSAGGRLPPPAHQQQSTAKPSLAFSISNILSDRFGDVQKPGKSI
ENQASIFRPFEANRSQTATPSAFTRVDLLEFSRQQQAAAAAATAAMMLERA
NFLNCFNPAAYPRIHEEIVQSRLRRSAANAVIPPPMSSKMSDANPEKSALGS
MQPKLEQKLISEEDLN
dCas9Gadd45a
(SEQ ID NO: 6)
MYPYDVPDYASPKKKRKVEASDKKYSIGLAIGTNSVGWAVIT
DEYKVPSKKFKVLGNTDRHSIKKNLIGALLFDSGETAEATRLKRTARRRYTR
RKNRICYLQEIFSNEMAKVDDSFFHRLEESFLVEEDKKHERHPIFGNIVDEVA
YHEKYPTIYHLRKKLVDSTDKADLRLIYLALAHMIKFRGHFLIEGDLNPDNS
DVDKLFIQLVQTYNQLFEENPINASGVDAKAILSARLSKSRRLENLIAQLPGE
KKNGLFGNLIALSLGLTPNFKSNFDLAEDAKLQLSKDTYDDDLDNLLAQIG
DQYADLFLAAKNLSDAILLSDILRVNTEITKAPLSASMIKRYDEHHQDLTLL
KALVRQQLPEKYKEIFFDQSKNGYAGYIDGGASQEEFYKFIKPILEKMDGTE
ELLVKLNREDLLRKQRTFDNGSIPHQIHLGELHAILRRQEDFYPFLKDNREKI
EKILTFRIPYYVGPLARGNSRFAWMTRKSEETITPWNFEEVVDKGASAQSFIE
RMTNFDKNLPNEKVLPKHSLLYEYFTVYNELTKVKYVTEGMRKPAFLSGE
QKKAIVDLLFKTNRKVTVKQLKEDYFKKIECFDSVEISGVEDRFNASLGTYH
DLLKIIKDKDFLDNEENEDILEDIVLTLTLFEDREMIEERLKTYAHLFDDKVM
KQLKRRRYTGWGRLSRKLINGIRDKQSGKTILDFLKSDGFANRNFMQLIHD
DSLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHPVE
NTQLQNEKLYLYYLQNGRDMYVDQELDINRLSDYDVDAIVPQSFLKDDSID
NKVLTRSDKNRGKSDNVPSEEVVKKMKNYWRQLLNAKLITQRKFDNLTKA
ERGGLSELDKAGFIKRQLVETRQITKHVAQILDSRMNTKYDENDKLIREVKV
ITLKSKLVSDFRKDFQFYKVREINNYHHAHDAYLNAVVGTALIKKYPKLESE
FVYGDYKVYDVRKMIAKSEQEIGKATAKYFFYSNIMNFFKTEITLANGEIRK
RPLIETNGETGEIVWDKGRDFATVRKVLSMPQVNIVKKTEVQTGGFSKESIL
PKRNSDKLIARKKDWDPKKYGGFDSPTVAYSVLVVAKVEKGKSKKLKSVK
ELLGITIMERSSFEKNPIDFLEAKGYKEVKKDLIIKLPKYSLFELENGRKRML
ASAGELQKGNELALPSKYVNFLYLASHYEKLKGSPEDNEQKQLFVEQHKH
YLDEIIEQISEFSKRVILADANLDKVLSAYNKHRDKPIREQAENIIHLFTLTNL
GAPAAFKYFDTTIDRKRYTSTKEVLDATLIHQSITGLYETRIDLSQLGGDSPK
KKRKVEASGPAMTLEEFSAAEQKTERMDTVGDALEEVLSKARSQRTITVGV
YEAAKLLNVDPDNVVLCLLAADEDDDRDVALQIHFTLIRAFCCENDINILRV
SNPGRLAELLLLENDAGPAESGGAAQTPDLHCVLVTNPHSSQWKDPALSQL
ICFCRESRYMDQWVPVINLPER
dCas9p300HAT
(SEQ ID NO: 7)
MYPYDVPDYASPKKKRKVEASDKKYSIGLAIGTNSVGWAVIT
DEYKVPSKKFKVLGNTDRHSIKKNLIGALLFDSGETAEATRLKRTARRRYTR
RKNRICYLQEIFSNEMAKVDDSFFHRLEESFLVEEDKKHERHPIFGNIVDEVA
YHEKYPTIYHLRKKLVDSTDKADLRLIYLALAHMIKFRGHFLIEGDLNPDNS
DVDKLFIQLVQTYNQLFEENPINASGVDAKAILSARLSKSRRLENLIAQLPGE
KKNGLFGNLIALSLGLTPNFKSNFDLAEDAKLQLSKDTYDDDLDNLLAQIG
DQYADLFLAAKNLSDAILLSDILRVNTEITKAPLSASMIKRYDEHHQDLTLL
KALVRQQLPEKYKEIFFDQSKNGYAGYIDGGASQEEFYKFIKPILEKMDGTE
ELLVKLNREDLLRKQRTFDNGSIPHQIHLGELHAILRRQEDFYPFLKDNREKI
EKILTFRIPYYVGPLARGNSRFAWMTRKSEETITPWNFEEVVDKGASAQSFIE
RMTNFDKNLPNEKVLPKHSLLYEYFTVYNELTKVKYVTEGMRKPAFLSGE
QKKAIVDLLFKTNRKVTVKQLKEDYFKKIECFDSVEISGVEDRFNASLGTYH
DLLKIIKDKDFLDNEENEDILEDIVLTLTLFEDREMIEERLKTYAHLFDDKVM
KQLKRRRYTGWGRLSRKLINGIRDKQSGKTILDFLKSDGFANRNFMQLIHD
DSLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHPVE
NTQLQNEKLYLYYLQNGRDMYVDQELDINRLSDYDVDAIVPQSFLKDDSID
NKVLTRSDKNRGKSDNVPSEEVVKKMKNYWRQLLNAKLITQRKFDNLTKA
ERGGLSELDKAGFIKRQLVETRQITKHVAQILDSRMNTKYDENDKLIREVKV
ITLKSKLVSDFRKDFQFYKVREINNYHHAHDAYLNAVVGTALIKKYPKLESE
FVYGDYKVYDVRKMIAKSEQEIGKATAKYFFYSNIMNFFKTEITLANGEIRK
RPLIETNGETGEIVWDKGRDFATVRKVLSMPQVNIVKKTEVQTGGFSKESIL
PKRNSDKLIARKKDWDPKKYGGFDSPTVAYSVLVVAKVEKGKSKKLKSVK
ELLGITIMERSSFEKNPIDFLEAKGYKEVKKDLIIKLPKYSLFELENGRKRML
ASAGELQKGNELALPSKYVNFLYLASHYEKLKGSPEDNEQKQLFVEQHKH
YLDEIIEQISEFSKRVILADANLDKVLSAYNKHRDKPIREQAENIIHLFTLTNL
GAPAAFKYFDTTIDRKRYTSTKEVLDATLIHQSITGLYETRIDLSQLGGDSPK
KKRKVEASGPAYRQDPESLPFRQPVDPQLLGIPDYFDIVKSPMDLSTIKRKLD
TGQYQEPWQYIDDIWLMFNNAWLYNRKTSRVYKYCSKLSEVFEQEIDPVM
QSLGYCCGRKLEFSPQTLCCYGKQLCTIPRDATYYSYQNRYHFCEKCFNEIQ
GESVSLGDDPSQPQTTINKEQFSKRKNDTLDPELFVECTECGRKMHQICVLH
HEIIWPSGFVCDGCLKKTARTRKENKLSAKRLPSTRLGTFLENRVNDFLRRQ
NHPESGEVTVRVVHASDKTVEVKPGMKARFVDSGEMAESFPYRTKALFAF
EEIDGVDLCFFGMHVQEYGSDCPPPNQRRVYISYLDSVHFFRPKCLRTAVYH
EILIGYLEYVKKLGYTTGHIWACPPSEGDDYIFHCHPPDQKIPKPKRLQEWY
KKMLDKAVSERIVHDYKDILKQATEDRLTSAKELPYFEGDFWPNVLEESIKE
LEQEEEERKREENTSNESTDVTKGDSKNAKKKNNKKTSKNKSSLSRGNKKK
PGMPNVSNDLSQKLYATMEKHKEVFFVIRLIACPAPNSLPPIVDPDPLIPCDL
MDGRDAFLTLARDKHLEFSSLRRAQWSTMCMLVELHTQSQDRFVYTCNEC
KHHVETRWHCTVCEDYDLCITCYNTKNHDHKMEK
dCas9mTet1CD
(SEQ ID NO: 8)
MYPYDVPDYASPKKKRKVEASDKKYSIGLAIGTNSVGWAVIT
DEYKVPSKKFKVLGNTDRHSIKKNLIGALLFDSGETAEATRLKRTARRRYTR
RKNRICYLQEIFSNEMAKVDDSFFHRLEESFLVEEDKKHERHPIFGNIVDEVA
YHEKYPTIYHLRKKLVDSTDKADLRLIYLALAHMIKFRGHFLIEGDLNPDNS
DVDKLFIQLVQTYNQLFEENPINASGVDAKAILSARLSKSRRLENLIAQLPGE
KKNGLFGNLIALSLGLTPNFKSNFDLAEDAKLQLSKDTYDDDLDNLLAQIG
DQYADLFLAAKNLSDAILLSDILRVNTEITKAPLSASMIKRYDEHHQDLTLL
KALVRQQLPEKYKEIFFDQSKNGYAGYIDGGASQEEFYKFIKPILEKMDGTE
ELLVKLNREDLLRKQRTFDNGSIPHQIHLGELHAILRRQEDFYPFLKDNREKI
EKILTFRIPYYVGPLARGNSRFAWMTRKSEETITPWNFEEVVDKGASAQSFIE
RMTNFDKNLPNEKVLPKHSLLYEYFTVYNELTKVKYVTEGMRKPAFLSGE
QKKAIVDLLFKTNRKVTVKQLKEDYFKKIECFDSVEISGVEDRFNASLGTYH
DLLKIIKDKDFLDNEENEDILEDIVLTLTLFEDREMIEERLKTYAHLFDDKVM
KQLKRRRYTGWGRLSRKLINGIRDKQSGKTILDFLKSDGFANRNFMQLIHD
DSLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHPVE
NTQLQNEKLYLYYLQNGRDMYVDQELDINRLSDYDVDAIVPQSFLKDDSID
NKVLTRSDKNRGKSDNVPSEEVVKKMKNYWRQLLNAKLITQRKFDNLTKA
ERGGLSELDKAGFIKRQLVETRQITKHVAQILDSRMNTKYDENDKLIREVKV
ITLKSKLVSDFRKDFQFYKVREINNYHHAHDAYLNAVVGTALIKKYPKLESE
FVYGDYKVYDVRKMIAKSEQEIGKATAKYFFYSNIMNFFKTEITLANGEIRK
RPLIETNGETGEIVWDKGRDFATVRKVLSMPQVNIVKKTEVQTGGFSKESIL
PKRNSDKLIARKKDWDPKKYGGFDSPTVAYSVLVVAKVEKGKSKKLKSVK
ELLGITIMERSSFEKNPIDFLEAKGYKEVKKDLIIKLPKYSLFELENGRKRML
ASAGELQKGNELALPSKYVNFLYLASHYEKLKGSPEDNEQKQLFVEQHKH
YLDEIIEQISEFSKRVILADANLDKVLSAYNKHRDKPIREQAENIIHLFTLTNL
GAPAAFKYFDTTIDRKRYTSTKEVLDATLIHQSITGLYETRIDLSQLGGDSPK
KKRKVEASGPAEAAPCDCDGGTQKEKGPYYTHLGAGPSVAAVRELMETRF
GQKGKAIRIEKIVFTGKEGKSSQGCPVAKWVIRRSGPEEKLICLVRERVDHH
CSTAVIVVLILLWEGIPRLMADRLYKELTENLRSYSGHPTDRRCTLNKKRTC
TCQGIDPKTCGASFSFGCSWSMYFNGCKFGRSENPRKFRLAPNYPLHNYYK
RITGMSSEGSDVKTGWIIPDRKTLISREEKQLEKNLQELATVLAPLYKQMAP
VAYQNQVEYEEVAGDCRLGNEEGRPFSGVTCCMDFCAHSHKDIHNMHNGS
TVVCTLIRADGRDTNCPEDEQLHVLPLYRLADTDEFGSVEGMKAKIKSGAIQ
VNGPTRKRRLRFTEPVPRCGKRAKMKQNHNKSGSHNTKSFSSASSTSHLVK
DESTDFCPLQASSAETSTCTYSKTASGGFAETSSILHCTMPSGAHSGANAAA
GECTGTVQPAEVAAHPHQSLPTADSPVHAEPLTSPSEQLTSNQSNQQLPLLS
NSQKLASCQVEDERHPEADEPQHPEDDNLPQLDEFWSDSEEIYADPSFGGV
AIAPIHGSVLIECARKELHATTSLRSPKRGVPFRVSLVFYQHKSLNKPNHGFD
INKIKCKCKKVTKKKPADRECPDVSPEANLSHQIPSRVASTLTRDNVVTVSP
YSLTHVAGPYNRWV
dCas9hACIDA
(SEQ ID NO: 9)
MYPYDVPDYASPKKKRKVEASDKKYSIGLAIGTNSVGWAVITDE
YKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYH
EKYPTIYHLRKKLVDSTDKADLRLIYLALAHMIKFRGHFLIEGDLNPDNSDV
DKLFIQLVQTYNQLFEENPINASGVDAKAILSARLSKSRRLENLIAQLPGEKK
NGLFGNLIALSLGLTPNFKSNFDLAEDAKLQLSKDTYDDDLDNLLAQIGDQ
YADLFLAAKNLSDAILLSDILRVNTEITKAPLSASMIKRYDEHHQDLTLLKAL
VRQQLPEKYKEIFFDQSKNGYAGYIDGGASQEEFYKFIKPILEKMDGTEELL
VKLNREDLLRKQRTFDNGSIPHQIHLGELHAILRRQEDFYPFLKDNREKIEKI
LTFRIPYYVGPLARGNSRFAWMTRKSEETITPWNFEEVVDKGASAQSFIERM
TNFDKNLPNEKVLPKHSLLYEYFTVYNELTKVKYVTEGMRKPAFLSGEQKK
AIVDLLFKTNRKVTVKQLKEDYFKKIECFDSVEISGVEDRFNASLGTYHDLL
KIIKDKDFLDNEENEDILEDIVLTLTLFEDREMIEERLKTYAHLFDDKVMKQL
KRRRYTGWGRLSRKLINGIRDKQSGKTILDFLKSDGFANRNFMQLIHDDSLT
FKEDIQKAQVSGQGDSLHEHIANLAGSPAIKKGILQTVKVVDELVKVMGRH
KPENIVIEMARENQTTQKGQKNSRERMKRIEEGIKELGSQILKEHPVENTQL
QNEKLYLYYLQNGRDMYVDQELDINRLSDYDVDAIVPQSFLKDDSIDNKVL
TRSDKNRGKSDNVPSEEVVKKMKNYWRQLLNAKLITQRKFDNLTKAERGG
LSELDKAGFIKRQLVETRQITKHVAQILDSRMNTKYDENDKLIREVKVITLK
SKLVSDFRKDFQFYKVREINNYHHAHDAYLNAVVGTALIKKYPKLESEFVY
GDYKVYDVRKMIAKSEQEIGKATAKYFFYSNIMNFFKTEITLANGEIRKRPLI
ETNGETGEIVWDKGRDFATVRKVLSMPQVNIVKKTEVQTGGFSKESILPKR
NSDKLIARKKDWDPKKYGGFDSPTVAYSVLVVAKVEKGKSKKLKSVKELL
GITIMERSSFEKNPIDFLEAKGYKEVKKDLIIKLPKYSLFELENGRKRMLASA
GELQKGNELALPSKYVNFLYLASHYEKLKGSPEDNEQKQLFVEQHKHYLDE
IIEQISEFSKRVILADANLDKVLSAYNKHRDKPIREQAENIIHLFTLTNLGAPA
AFKYFDTTIDRKRYTSTKEVLDATLIHQSITGLYETRIDLSQLGGDSPKKKRK
VEASGPAMDSLLMNRRKFLYQFKNVRWAKGRRETYLCYVVKRRDSATSFS
LDFGYLRNKNGCHVELLFLRYISDWDLDPGRCYRVTWFTSWSPCYDCARH
VADFLRGNPNLSLRIFTARLYFCEDRKAEPEGLRRLHRAGVQIAIMTFKDYF
YCWNTFVENHERTFKAWEGLHENSVRLSRQLRRILLPLYEVDDLRDAFRTLGL
dCas9hDMNT1
(SEQ ID NO: 10)
MYPYDVPDYASPKKKRKVEASDKKYSIGLAIGTNSVGWAVIT
DEYKVPSKKFKVLGNTDRHSIKKNLIGALLFDSGETAEATRLKRTARRRYTR
RKNRICYLQEIFSNEMAKVDDSFFHRLEESFLVEEDKKHERHPIFGNIVDEVA
YHEKYPTIYHLRKKLVDSTDKADLRLIYLALAHMIKFRGHFLIEGDLNPDNS
DVDKLFIQLVQTYNQLFEENPINASGVDAKAILSARLSKSRRLENLIAQLPGE
KKNGLFGNLIALSLGLTPNFKSNFDLAEDAKLQLSKDTYDDDLDNLLAQIG
DQYADLFLAAKNLSDAILLSDILRVNTEITKAPLSASMIKRYDEHHQDLTLL
KALVRQQLPEKYKEIFFDQSKNGYAGYIDGGASQEEFYKFIKPILEKMDGTE
ELLVKLNREDLLRKQRTFDNGSIPHQIHLGELHAILRRQEDFYPFLKDNREKI
EKILTFRIPYYVGPLARGNSRFAWMTRKSEETITPWNFEEVVDKGASAQSFIE
RMTNFDKNLPNEKVLPKHSLLYEYFTVYNELTKVKYVTEGMRKPAFLSGE
QKKAIVDLLFKTNRKVTVKQLKEDYFKKIECFDSVEISGVEDRFNASLGTYH
DLLKIIKDKDFLDNEENEDILEDIVLTLTLFEDREMIEERLKTYAHLFDDKVM
KQLKRRRYTGWGRLSRKLINGIRDKQSGKTILDFLKSDGFANRNFMQLIHD
DSLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHPVE
NTQLQNEKLYLYYLQNGRDMYVDQELDINRLSDYDVDAIVPQSFLKDDSID
NKVLTRSDKNRGKSDNVPSEEVVKKMKNYWRQLLNAKLITQRKFDNLTKA
ERGGLSELDKAGFIKRQLVETRQITKHVAQILDSRMNTKYDENDKLIREVKV
ITLKSKLVSDFRKDFQFYKVREINNYHHAHDAYLNAVVGTALIKKYPKLESE
FVYGDYKVYDVRKMIAKSEQEIGKATAKYFFYSNIMNFFKTEITLANGEIRK
RPLIETNGETGEIVWDKGRDFATVRKVLSMPQVNIVKKTEVQTGGFSKESIL
PKRNSDKLIARKKDWDPKKYGGFDSPTVAYSVLVVAKVEKGKSKKLKSVK
ELLGITIMERSSFEKNPIDFLEAKGYKEVKKDLIIKLPKYSLFELENGRKRML
ASAGELQKGNELALPSKYVNFLYLASHYEKLKGSPEDNEQKQLFVEQHKH
YLDEIIEQISEFSKRVILADANLDKVLSAYNKHRDKPIREQAENIIHLFTLTNL
GAPAAFKYFDTTIDRKRYTSTKEVLDATLIHQSITGLYETRIDLSQLGGDSPK
KKRKVEASGPAMPARTAPARVPTLAVPAISLPDDVRRRLKDLERDSLTEKE
CVKEKLNLLHEFLQTEIKNQLCDLETKLRKEELSEEGYLAKVKSLLNKDLSL
ENGAHAYNREVNGRLENGNQARSEARRVGMADANSPPKPLSKPRTPRRSK
SDGEAKRSRDPPASASQVTGIRAEPSPSPRITRKSTRQTTITSHFAKGPAKRKP
QEESERAKSDESIKEEDKDQDEKRRRVTSRERVARPLPAEEPERAKSGTRTE
KEEERDEKEEKRLRSQTKEPTPKQKLKEEPDREARAGVQADEDEDGDEKDE
KKHRSQPKDLAAKRRPEEKEPEKVNPQISDEKDEDEKEEKRRKTTPKEPTEK
KMARAKTVMNSKTHPPKCIQCGQYLDDPDLKYGQHPPDAVDEPQMLTNEK
LSIFDANESGFESYEALPQHKLTCFSVYCKHGHLCPIDTGLIEKNIELFFSGSA
KPIYDDDPSLEGGVNGKNLGPINEWWITGFDGGEKALIGFSTSFAEYILMDPS
PEYAPIFGLMQEKIYISKIVVEFLQSNSDSTYEDLINKIETTVPPSGLNLNRFTE
DSLLRHAQFVVEQVESYDEAGDSDEQPIFLTPCMRDLIKLAGVTLGQRRAQ
ARRQTIRHSTREKDRGPTKATTTKLVYQIFDTFFAEQIEKDDREDKENAFKR
RRCGVCEVCQQPECGKCKACKDMVKFGGSGRSKQACQERRCPNMAMKEA
DDDEEVDDNIPEMPSPKKMHQGKKKKQNKNRISWVGEAVKTDGKKSYYK
KVCIDAETLEVGDCVSVIPDDSSKPLYLARVTALWEDSSNGQMFHAHWFCA
GTDTVLGATSDPLELFLVDECEDMQLSYIHSKVKVIYKAPSENWAMEGGM
DPESLLEGDDGKTYFYQLWYDQDYARFESPPKTQPTEDNKFKFCVSCARLA
EMRQKEIPRVLEQLEDLDSRVLYYSATKNGILYRVGDGVYLPPEAFTFNIKL
SSPVKRPRKEPVDEDLYPEHYRKYSDYIKGSNLDAPEPYRIGRIKEIFCPKKS
NGRPNETDIKIRVNKFYRPENTHKSTPASYHADINLLYWSDEEAVVDFKAV
QGRCTVEYGEDLPECVQVYSMGGPNRFYFLEAYNAKSKSFEDPPNHARSPG
NKGKGKGKGKGKPKSQACEPSEPEIEIKLPKLRTLDVFSGCGGLSEGFHQAG
ISDTLWAIEMWDPAAQAFRLNNPGSTVFTEDCNILLKLVMAGETTNSRGQR
LPQKGDVEMLCGGPPCQGFSGMNRFNSRTYSKFKNSLVVSFLSYCDYYRPR
FFLLENVRNFVSFKRSMVLKLTLRCLVRMGYQCTFGVLQAGQYGVAQTRR
RAIILAAAPGEKLPLFPEPLHVFAPRACQLSVVVDDKKFVSNITRLSSGPFRTI
TVRDTMSDLPEVRNGASALEISYNGEPQSWFQRQLRGAQYQPILRDHICKD
MSALVAARMRHIPLAPGSDWRDLPNIEVRLSDGTMARKLRYTHHDRKNGR
SSSGALRGVCSCVEAGKACDPAARQFNTLIPWCLPHTGNRHNHWAGLYGR
LEWDGFFSTTVTNPEPMGKQGRVLHPEQHRVVSVRECARSQGFPDTYRLFG
NILDKHRQVGNAVPPPLAKAIGLEIKLCMLAKARESASAKIKEEEAAKDID
dCas9hDNMT3a
(SEQ ID NO: 11)
MYPYDVPDYASPKKKRKVEASDKKYSIGLAIGTNSVGWAVIT
DEYKVPSKKFKVLGNTDRHSIKKNLIGALLFDSGETAEATRLKRTARRRYTR
RKNRICYLQEIFSNEMAKVDDSFFHRLEESFLVEEDKKHERHPIFGNIVDEVA
YHEKYPTIYHLRKKLVDSTDKADLRLIYLALAHMIKFRGHFLIEGDLNPDNS
DVDKLFIQLVQTYNQLFEENPINASGVDAKAILSARLSKSRRLENLIAQLPGE
KKNGLFGNLIALSLGLTPNFKSNFDLAEDAKLQLSKDTYDDDLDNLLAQIG
DQYADLFLAAKNLSDAILLSDILRVNTEITKAPLSASMIKRYDEHHQDLTLL
KALVRQQLPEKYKEIFFDQSKNGYAGYIDGGASQEEFYKFIKPILEKMDGTE
ELLVKLNREDLLRKQRTFDNGSIPHQIHLGELHAILRRQEDFYPFLKDNREKI
EKILTFRIPYYVGPLARGNSRFAWMTRKSEETITPWNFEEVVDKGASAQSFIE
RMTNFDKNLPNEKVLPKHSLLYEYFTVYNELTKVKYVTEGMRKPAFLSGE
QKKAIVDLLFKTNRKVTVKQLKEDYFKKIECFDSVEISGVEDRFNASLGTYH
DLLKIIKDKDFLDNEENEDILEDIVLTLTLFEDREMIEERLKTYAHLFDDKVM
KQLKRRRYTGWGRLSRKLINGIRDKQSGKTILDFLKSDGFANRNFMQLIHD
DSLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHPVE
NTQLQNEKLYLYYLQNGRDMYVDQELDINRLSDYDVDAIVPQSFLKDDSID
NKVLTRSDKNRGKSDNVPSEEVVKKMKNYWRQLLNAKLITQRKFDNLTKA
ERGGLSELDKAGFIKRQLVETRQITKHVAQILDSRMNTKYDENDKLIREVKV
ITLKSKLVSDFRKDFQFYKVREINNYHHAHDAYLNAVVGTALIKKYPKLESE
FVYGDYKVYDVRKMIAKSEQEIGKATAKYFFYSNIMNFFKTEITLANGEIRK
RPLIETNGETGEIVWDKGRDFATVRKVLSMPQVNIVKKTEVQTGGFSKESIL
PKRNSDKLIARKKDWDPKKYGGFDSPTVAYSVLVVAKVEKGKSKKLKSVK
ELLGITIMERSSFEKNPIDFLEAKGYKEVKKDLIIKLPKYSLFELENGRKRML
ASAGELQKGNELALPSKYVNFLYLASHYEKLKGSPEDNEQKQLFVEQHKH
YLDEIIEQISEFSKRVILADANLDKVLSAYNKHRDKPIREQAENIIHLFTLTNL
GAPAAFKYFDTTIDRKRYTSTKEVLDATLIHQSITGLYETRIDLSQLGGDSPK
KKRKVEASGPAMPAMPSSGPGDTSSSAAEREEDRKDGEEQEEPRGKEERQE
PSTTARKVGRPGRKRKHPPVESGDTPKDPAVISKSPSMAQDSGASELLPNGD
LEKRSEPQPEEGSPAGGQKGGAPAEGEGAAETLPEASRAVENGCCTPKEGR
GAPAEAGKEQKETNIESMKMEGSRGRLRGGLGWESSLRQRPMPRLTFQAG
DPYYISKRKRDEWLARWKREAEKKAKVIAGMNAVEENQGPGESQKVEEAS
PPAVQQPTDPASPTVATTPEPVGSDAGDKNATKAGDDEPEYEDGRGFGIGE
LVWGKLRGFSWWPGRIVSWWMTGRSRAAEGTRWVMWFGDGKFSVVCVE
KLMPLSSFCSAFHQATYNKQPMYRKAIYEVLQVASSRAGKLFPVCHDSDES
DTAKAVEVQNKPMIEWALGGFQPSGPKGLEPPEEEKNPYKEVYTDMWVEP
EAAAYAPPPPAKKPRKSTAEKPKVKEIIDERTRERLVYEVRQKCRNIEDICIS
CGSLNVTLEHPLFVGGMCQNCKNCFLECAYQYDDDGYQSYCTICCGGREV
LMCGNNNCCRCFCVECVDLLVGPGAAQAAIKEDPWNCYMCGHKGTYGLL
RRREDWPSRLQMFFANNHDQEFDPPKVYPPVPAEKRKPIRVLSLFDGIATGL
LVLKDLGIQVDRYIASEVCEDSITVGMVRHQGKIMYVGDVRSVTQKHIQEW
GPFDLVIGGSPCNDLSIVNPARKGLYEGTGRLFFEFYRLLHDARPKEGDDRP
FFWLFENVVAMGVSDKRDISRFLESNPVMIDAKEVSAAHRARYFWGNLPG
MNRPLASTVNDKLELQECLEHGRIAKFSKVRTITTRSNSIKQGKDQHFPVFM
NEKEDILWCTEMERVFGFPVHYTDVSNMSRLARQRLLGRSWSVPVIRHLFA
PLKEYFACVID
dCas9hDNMT3b
(SEQ ID NO: 12)
MYPYDVPDYASPKKKRKVEASDKKYSIGLAIGTNSVGWAVIT
DEYKVPSKKFKVLGNTDRHSIKKNLIGALLFDSGETAEATRLKRTARRRYTR
RKNRICYLQEIFSNEMAKVDDSFFHRLEESFLVEEDKKHERHPIFGNIVDEVA
YHEKYPTIYHLRKKLVDSTDKADLRLIYLALAHMIKFRGHFLIEGDLNPDNS
DVDKLFIQLVQTYNQLFEENPINASGVDAKAILSARLSKSRRLENLIAQLPGE
KKNGLFGNLIALSLGLTPNFKSNFDLAEDAKLQLSKDTYDDDLDNLLAQIG
DQYADLFLAAKNLSDAILLSDILRVNTEITKAPLSASMIKRYDEHHQDLTLL
KALVRQQLPEKYKEIFFDQSKNGYAGYIDGGASQEEFYKFIKPILEKMDGTE
ELLVKLNREDLLRKQRTFDNGSIPHQIHLGELHAILRRQEDFYPFLKDNREKI
EKILTFRIPYYVGPLARGNSRFAWMTRKSEETITPWNFEEVVDKGASAQSFIE
RMTNFDKNLPNEKVLPKHSLLYEYFTVYNELTKVKYVTEGMRKPAFLSGE
QKKAIVDLLFKTNRKVTVKQLKEDYFKKIECFDSVEISGVEDRFNASLGTYH
DLLKIIKDKDFLDNEENEDILEDIVLTLTLFEDREMIEERLKTYAHLFDDKVM
KQLKRRRYTGWGRLSRKLINGIRDKQSGKTILDFLKSDGFANRNFMQLIHD
DSLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHPVE
NTQLQNEKLYLYYLQNGRDMYVDQELDINRLSDYDVDAIVPQSFLKDDSID
NKVLTRSDKNRGKSDNVPSEEVVKKMKNYWRQLLNAKLITQRKFDNLTKA
ERGGLSELDKAGFIKRQLVETRQITKHVAQILDSRMNTKYDENDKLIREVKV
ITLKSKLVSDFRKDFQFYKVREINNYHHAHDAYLNAVVGTALIKKYPKLESE
FVYGDYKVYDVRKMIAKSEQEIGKATAKYFFYSNIMNFFKTEITLANGEIRK
RPLIETNGETGEIVWDKGRDFATVRKVLSMPQVNIVKKTEVQTGGFSKESIL
PKRNSDKLIARKKDWDPKKYGGFDSPTVAYSVLVVAKVEKGKSKKLKSVK
ELLGITIMERSSFEKNPIDFLEAKGYKEVKKDLIIKLPKYSLFELENGRKRML
ASAGELQKGNELALPSKYVNFLYLASHYEKLKGSPEDNEQKQLFVEQHKH
YLDEIIEQISEFSKRVILADANLDKVLSAYNKHRDKPIREQAENIIHLFTLTNL
GAPAAFKYFDTTIDRKRYTSTKEVLDATLIHQSITGLYETRIDLSQLGGDSPK
KKRKVEASGPAMKGDTRHLNGEEDAGGREDSILVNGACSDQSSDSPPILEAI
RTPEIRGRRSSSRLSKREVSSLLSYTQDLTGDGDGEDGDGSDTPVMPKLFRE
TRTRSESPAVRTRNNNSVSSRERHRPSPRSTRGRQGRNHVDESPVEFPATRSL
RRRATASAGTPWPSPPSSYLTIDLTDDTEDTHGTPQSSSTPYARLAQDSQQG
GMESPQVEADSGDGDSSEYQDGKEFGIGDLVWGKIKGFSWWPAMVVSWK
ATSKRQAMSGMRWVQWFGDGKFSEVSADKLVALGLFSQHFNLATFNKLV
SYRKAMYHALEKARVRAGKTFPSSPGDSLEDQLKPMLEWAHGGFKPTGIE
GLKPNNTQPENKTRRRTADDSATSDYCPAPKRLKTNCYNNGKDRGDEDQS
REQMASDVANNKSSLEDGCLSCGRKNPVSFHPLFEGGLCQTCRDRFLELFY
MYDDDGYQSYCTVCCEGRELLLCSNTSCCRCFCVECLEVLVGTGTAAEAK
LQEPWSCYMCLPQRCHGVLRRRKDWNVRLQAFFTSDTGLEYEAPKLYPAIP
AARRRPIRVLSLFDGIATGYLVLKELGIVGKYVASEVCEESIAVGTVKHEGNI
KYVNDVRNITKKNIEEWGPFDLVIGGSPCDLSNVNPARKGLYEGTGRLFFEF
YHLLNYSRPKEGDDRPFFWMFENVVAMKVGDKRDISRFLECNPVMIDAIKV
SAAHRARYFWGNLPGMNRIFGFPVHYTDVSNMGRGARQKLLGRSWSVPVI
RHLFAPLKDYFACEID
dCas9hG9a
(SEQ ID NO: 13)
MYPYDVPDYASPKKKRKVEASDKKYSIGLAIGTNSVGWAVIT
DEYKVPSKKFKVLGNTDRHSIKKNLIGALLFDSGETAEATRLKRTARRRYTR
RKNRICYLQEIFSNEMAKVDDSFFHRLEESFLVEEDKKHERHPIFGNIVDEVA
YHEKYPTIYHLRKKLVDSTDKADLRLIYLALAHMIKFRGHFLIEGDLNPDNS
DVDKLFIQLVQTYNQLFEENPINASGVDAKAILSARLSKSRRLENLIAQLPGE
KKNGLFGNLIALSLGLTPNFKSNFDLAEDAKLQLSKDTYDDDLDNLLAQIG
DQYADLFLAAKNLSDAILLSDILRVNTEITKAPLSASMIKRYDEHHQDLTLL
KALVRQQLPEKYKEIFFDQSKNGYAGYIDGGASQEEFYKFIKPILEKMDGTE
ELLVKLNREDLLRKQRTFDNGSIPHQIHLGELHAILRRQEDFYPFLKDNREKI
EKILTFRIPYYVGPLARGNSRFAWMTRKSEETITPWNFEEVVDKGASAQSFIE
RMTNFDKNLPNEKVLPKHSLLEYFTVYNELTKVKYVTEGMRKPAFLSGEQ
KKAIVDLLFKTNRKVTVKQLKEDYFKKIECFDSVEISGVEDRFNASLGTYHD
LLKIIKDKDFLDNEENEDILEDIVLTLTLFEDREMIEERLKTYAHLFDDKVMK
QLKRRRYTGWGRLSRKLINGIRDKQSGKTILDFLKSDGFANRNFMQLIHDDS
LTFKEDIQKAQVSGQGDSLHEHIANLAGSPAIKKGILQTVKVVDELVKVMG
RHKPENIVIEMARENQTTQKGQKNSRERMKRIEEGIKELGSQILKEHPVENT
QLQNEKLYLYYLQNGRDMYVDQELDINRLSDYDVDAIVPQSFLKDDSIDNK
VLTRSDKNRGKSDNVPSEEVVKKMKNYWRQLLNAKLITQRKFDNLTKAER
GGLSELDKAGFIKRQLVETRQITKHVAQILDSRMNTKYDENDKLIREVKVIT
LKSKLVSDFRKDFQFYKVREINNYHHAHDAYLNAVVGTALKKYPKLESEFV
YGDYKVYDVRKMIAKSEQEIGKATAKYFFYSNIMNFFKTEITLANGEIRKRP
LIETNGETGEIVWDKGRDFATVRKVLSMPQVNIVKKTEVQTGGFSKESILPK
RNSDKLIARKKDWDPKKYGGFDSPTVAYSVLVVAKVEKGKSKKLKSVKEL
LGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYL
DEIIEQISEFSKRVILADANLDKVLSAYNKHRDKPIREQAENIIHLFTLTNLGA
PAAFKYFDTTIDRKRYTSTKEVLDATLIHQSITGLYETRIDLSQLGGDSPKKK
RKVEASGPAMAAAAGAAAAAAAEGEAPAEMGALLLEKETRGATERVHGS
LGDTPRSEETLPKATPDSLEPAGPSSPASVTVTVGDEGADTPVGATPLIGDES
ENLEGDGDLRGGRILLGHATKSFPSSPSKGGSCPSRAKMSMTGAGKSPPSVQ
SLAMRLLSMPGAQGAAAAGSEPPPATTSPEGQPKVHRARKTMSKPGNGQPP
VPEKRPPEIQHFRMSDDVHSLGKVTSDLAKRRKLNSGGGLSEELGSARRSGE
VTLTKGDPGSLEEWETVVGDDFSLYYDSYSVDERVDSDSKSEVEALTEQLS
EEEEEEEEEEEEEEEEEEEEEEEEDEESGNQSDRSGSSGRRKAKKKWRKDSP
WVKPSRKRRKREPPRAKEPRGVSNDTSSLETERGFEELPLCSCRMEAPKIDRI
SERAGHKCMATESVDGELSGCNAAILKRETMRPSSRVALMVLCETHRARM
VKHHCCPGCGYFCTAGTFLECHPDFRVAHRFHKACVSQLNGMVFCPHCGE
DASEAQEVTIPRGDGVTPPAGTAAPAPPPLSQDVPGRADTSQPSARMRGHG
EPRRPPCDPLADTIDSSGPSLTLPNGGCLSAVGLPLGPGREALEKALVIQESE
RRKKLRFHPRQLYLSVKQGELQKVILMLLDNLDPNFQSDQQSKRTPLHAAA
QKGSVEICHVLLQAGANINAVDKQQRTPLMEAVNNHLEVARYMVQRGGC
VYSKEEDGSTCLHHAAKIGNLEMVSLLLSTGQVDVNAQDSGGWTPIIWAAE
HKHIEVIRMLLTRGADVTLTDNEENICLHWASFTGSAAIAEVLLNARCDLHA
VNYHGDTPLHIAARESYHDCVLLFLSRGANPELRNKEGDTAWDLTPERSDV
WFALQLNRKLRLGVGNRAIRTEKIICRDVARGYENVPIPCVNGVDGEPCPED
YKYISENCETSTMNIDRNITHLQHCTCVDDCSSSNCLCGQLSIRCWYDKDGR
LLQEFNKIEPPLIFECNQACSCWRNCKNRVVQSGIKVRLQLYRTAKMGWGV
RALQTIPQGTFICEYVGELISDAEADVREDDSYLFDLDNKDGEVYCIDARYY
GNISRFINHLCDPNIIPVRVFMLHQDLRFPRIAFFSSRDIRTGEELGFDYGDRF
WDIKSKYFTCQCGSEKCKHSAEAIALEQSRLARLDPHPELLPELGSLPPVNTD
dCas9VP64
(SEQ ID NO: 14)
MYPYDVPDYASPKKKRKVEASDKKYSIGLAIGTNSVGWAVIT
DEYKVPSKKFKVLGNTDRHSIKKNLIGALLFDSGETAEATRLKRTARRRYTR
RKNRICYLQEIFSNEMAKVDDSFFHRLEESFLVEEDKKHERHPIFGNIVDEVA
YHEKYPTIYHLRKKLVDSTDKADLRLIYLALAHMIKFRGHFLIEGDLNPDNS
DVDKLFIQLVQTYNQLFEENPINASGVDAKAILSARLSKSRRLENLIAQLPGE
KKNGLFGNLIALSLGLTPNFKSNFDLAEDAKLQLSKDTYDDDLDNLLAQIG
DQYADLFLAAKNLSDAILLSDILRVNTEITKAPLSASMIKRYDEHHQDLTLL
KALVRQQLPEKYKEIFFDQSKNGYAGYIDGGASQEEFYKFIKPILEKMDGTE
ELLVKLNREDLLRKQRTFDNGSIPHQIHLGELHAILRRQEDFYPFLKDNREKI
EKILTFRIPYYVGPLARGNSRFAWMTRKSEETITPWNFEEVVDKGASAQSFIE
RMTNFDKNLPNEKVLPKHSLLEYFTVYNELTKVKYVTEGMRKPAFLSGEQ
KKAIVDLLFKTNRKVTVKQLKEDYFKKIECFDSVEISGVEDRFNASLGTYHD
LLKIIKDKDFLDNEENEDILEDIVLTLTLFEDREMIEERLKTYAHLFDDKVMK
QLKRRRYTGWGRLSRKLINGIRDKQSGKTILDFLKSDGFANRNFMQLIHDDS
LTFKEDIQKAQVSGQGDSLHEHIANLAGSPAIKKGILQTVKVVDELVKVMG
RHKPENIVIEMARENQTTQKGQKNSRERMKRIEEGIKELGSQILKEHPVENT
QLQNEKLYLYYLQNGRDMYVDQELDINRLSDYDVAIVPQSFLKDDSIDNKV
LTRSDKNRGKSDNVPSEEVVKKMKNYWRQLLNAKLITQRKFDNLTKAERG
GLSELDKAGFIKRQLVETRQITKHVAQILDSRMNTKYDENDKLIREVKVITL
KSKLVSDFRKDFQFYKVREINNYHHAHDAYLNAVVGTALIKKYPKLESEFV
YGDYKVYDVRKMIAKSEQEIGKATAKYFFYSNIMNFFKTEITLANGEIRKRP
LIETNGETGEIVWDKGRDFATVRKVLSMPQVNIVKKTEVQTGGFSKESILPK
RNSDKLIARKKDWDPKKYGGFDSPTVAYSVLVVAKVEKGKSKKLKSVKEL
LGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYL
DEIIEQISEFSKRVILADANLDKVLSAYNKHRDKPIREQAENIIHLFTLTNLGA
PAAFKYFDTTIDRKRYTSTKEVLDATLIHQSITGLYETRIDLSQLGGDSPKKK
RKVEASGPAGSGRADALDDFDLDMLGSDALDDFDLDMLGSDALDDFDLD
MLGSDALDDFDLDMLYID
dCas9VP96
(SEQ ID NO: 15)
MYPYDVPDYASPKKKRKVEASDKKYSIGLAIGTNSVGWAVIT
DEYKVPSKKFKVLGNTDRHSIKKNLIGALLFDSGETAEATRLKRTARRRYTR
RKNRICYLQEIFSNEMAKVDDSFFHRLEESFLVEEDKKHERHPIFGNIVDEVA
YHEKYPTIYHLRKKLVDSTDKADLRLIYLALAHMIKFRGHFLIEGDLNPDNS
DVDKLFIQLVQTYNQLFEENPINASGVDAKAILSARLSKSRRLENLIAQLPGE
KKNGLFGNLIALSLGLTPNFKSNFDLAEDAKLQLSKDTYDDDLDNLLAQIG
DQYADLFLAAKNLSDAILLSDILRVNTEITKAPLSASMIKRYDEHHQDLTLL
KALVRQQLPEKYKEIFFDQSKNGYAGYIDGGASQEEFYKFIKPILEKMDGTE
ELLVKLNREDLLRKQRTFDNGSIPHQIHLGELHAILRRQEDFYPFLKDNREKI
EKILTFRIPYYVGPLARGNSRFAWMTRKSEETITPWNFEEVVDKGASAQSFIE
RMTNFDKNLPNEKVLPKHSLLYEYFTVYNELTKVKYVTEGMRKPAFLSGE
QKKAIVDLLFKTNRKVTVKQLKEDYFKKIECFDSVEISGVEDRFNASLGTYH
DLLKIIKDKDFLDNEENEDILEDIVLTLTLFEDREMIEERLKTYAHLFDDKVM
KQLKRRRYTGWGRLSRKLINGIRDKQSGKTILDFLKSDGFANRNFMQLIHD
DSLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHPVE
NTQLQNEKLYLYYLQNGRDMYVDQELDINRLSDYDVDAIVPQSFLKDDSID
NKVLTRSDKNRGKSDNVPSEEVVKKMKNYWRQLLNAKLITQRKFDNLTKA
ERGGLSELDKAGFIKRQLVETRQITKHVAQILDSRMNTKYDENDKLIREVKV
ITLKSKLVSDFRKDFQFYKVREINNYHHAHDAYLNAVVGTALIKKYPKLESE
FVYGDYKVYDVRKMIAKSEQEIGKATAKYFFYSNIMNFFKTEITLANGEIRK
RPLIETNGETGEIVWDKGRDFATVRKVLSMPQVNIVKKTEVQTGGFSKESIL
PKRNSDKLIARKKDWDPKKYGGFDSPTVAYSVLVVAKVEKGKSKKLKSVK
ELLGITIMERSSFEKNPIDFLEAKGYKEVKKDLIIKLPKYSLFELENGRKRML
ASAGELQKGNELALPSKYVNFLYLASHYEKLKGSPEDNEQKQLFVEQHKH
YLDEIIEQISEFSKRVILADANLDKVLSAYNKHRDKPIREQAENIIHLFTLTNL
GAPAAFKYFDTTIDRKRYTSTKEVLDATLIHQSITGLYETRIDLSQLGGDSPK
KKRKVEASGPAGSGRADALDDFDLDMLGSDALDDFDLDMLGSDALDDFDL
DMLGSDALDDFDLDMLGSDALDDFDLDMLGSDALDDFDLDMLYID
dCas9VP160
(SEQ ID NO: 16)
MYPYDVPDYASPKKKRKVEASDKKYSIGLAIGTNSVGWAVIT
DEYKVPSKKFKVLGNTDRHSIKKNLIGALLFDSGETAEATRLKRTARRRYTR
RKNRICYLQEIFSNEMAKVDDSFFHRLEESFLVEEDKKHERHPIFGNIVDEVA
YHEKYPTIYHLRKKLVDSTDKADLRLIYLALAHMIKFRGHFLIEGDLNPDNS
DVDKLFIQLVQTYNQLFEENPINASGVDAKAILSARLSKSRRLENLIAQLPGE
KKNGLFGNLIALSLGLTPNFKSNFDLAEDAKLQLSKDTYDDDLDNLLAQIG
DQYADLFLAAKNLSDAILLSDILRVNTEITKAPLSASMIKRYDEHHQDLTLL
KALVRQQLPEKYKEIFFDQSKNGYAGYIDGGASQEEFYKFIKPILEKMDGTE
ELLVKLNREDLLRKQRTFDNGSIPHQIHLGELHAILRRQEDFYPFLKDNREKI
EKILTFRIPYYVGPLARGNSRFAWMTRKSEETITPWNFEEVVDKGASAQSFIE
RMTNFDKNLPNEKVLPKHSLLYEYFTVYNELTKVKYVTEGMRKPAFLSGE
QKKAIVDLLFKTNRKVTVKQLKEDYFKKIECFDSVEISGVEDRFNASLGTYH
DLLKIIKDKDFLDNEENEDILEDIVLTLTLFEDREMIEERLKTYAHLFDDKVM
KQLKRRRYTGWGRLSRKLINGIRDKQSGKTILDFLKSDGFANRNFMQLIHD
DSLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHPVE
NTQLQNEKLYLYYLQNGRDMYVDQELDINRLSDYDVDAIVPQSFLKDDSID
NKVLTRSDKNRGKSDNVPSEEVVKKMKNYWRQLLNAKLITQRKFDNLTKA
ERGGLSELDKAGFIKRQLVETRQITKHVAQILDSRMNTKYDENDKLIREVKV
ITLKSKLVSDFRKDFQFYKVREINNYHHAHDAYLNAVVGTALIKKYPKLESE
FVYGDYKVYDVRKMIAKSEQEIGKATAKYFFYSNIMNFFKTEITLANGEIRK
RPLIETNGETGEIVWDKGRDFATVRKVLSMPQVNIVKKTEVQTGGFSKESIL
PKRNSDKLIARKKDWDPKKYGGFDSPTVAYSVLVVAKVEKGKSKKLKSVK
ELLGITIMERSSFEKNPIDFLEAKGYKEVKKDLIIKLPKYSLFELENGRKRML
ASAGELQKGNELALPSKYVNFLYLASHYEKLKGSPEDNEQKQLFVEQHKH
YLDEIIEQISEFSKRVILADANLDKVLSAYNKHRDKPIREQAENIIHLFTLTNL
GAPAAFKYFDTTIDRKRYTSTKEVLDATLIHQSITGLYETRIDLSQLGGDSPK
KKRKVEASGPAGSGRADALDDFDLDMLGSDALDDFDLDMLGSDALDDFDL
DMLGSDALDDFDLDMLGSDALDDFDLDMLGSDALDDFDLDMLGSDALDD
FDLDMLGDALDDFDLDMLGSDALDDFDLDMLGSDALDDFDLDMLYID
dCas9hINI1
(SEQ ID NO: 17)
MYPYDVPDYASPKKKRKVEASDKKYSIGLAIGTNSVGWAVIT
DEYKVPSKKFKVLGNTDRHSIKKNLIGALLFDSGETAEATRLKRTARRRYTR
RKNRICYLQEIFSNEMAKVDDSFFHRLEESFLVEEDKKHERHPIFGNIVDEVA
YHEKYPTIYHLRKKLVDSTDKADLRLIYLALAHMIKFRGHFLIEGDLNPDNS
DVDKLFIQLVQTYNQLFEENPINASGVDAKAILSARLSKSRRLENLIAQLPGE
KKNGLFGNLIALSLGLTPNFKSNFDLAEDAKLQLSKDTYDDDLDNLLAQIG
DQYADLFLAAKNLSDAILLSDILRVNTEITKAPLSASMIKRYDEHHQDLTLL
KALVRQQLPEKYKEIFFDQSKNGYAGYIDGGASQEEFYKFIKPILEKMDGTE
ELLVKLNREDLLRKQRTFDNGSIPHQIHLGELHAILRRQEDFYPFLKDNREKI
EKILTFRIPYYVGPLARGNSRFAWMTRKSEETITPWNFEEVVDKGASAQSFIE
RMTNFDKNLPNEKVLPKHSLLYEYFTVYNELTKVKYVTEGMRKPAFLSGE
QKKAIVDLLFKTNRKVTVKQLKEDYFKKIECFDSVEISGVEDRFNASLGTYH
DLLKIIKDKDFLDNEENEDILEDIVLTLTLFEDREMIEERLKTYAHLFDDKVM
KQLKRRRYTGWGRLSRKLINGIRDKQSGKTILDFLKSDGFANRNFMQLIHD
DSLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHPVE
NTQLQNEKLYLYYLQNGRDMYVDQELDINRLSDYDVDAIVPQSFLKDDSID
NKVLTRSDKNRGKSDNVPSEEVVKKMKNYWRQLLNAKLITQRKFDNLTKA
ERGGLSELDKAGFIKRQLVETRQITKHVAQILDSRMNTKYDENDKLIREVKV
ITLKSKLVSDFRKDFQFYKVREINNYHHAHDAYLNAVVGTALIKKYPKLESE
FVYGDYKVYDVRKMIAKSEQEIGKATAKYFFYSNIMNFFKTEITLANGEIRK
RPLIETNGETGEIVWDKGRDFATVRKVLSMPQVNIVKKTEVQTGGFSKESIL
PKRNSDKLIARKKDWDPKKYGGFDSPTVAYSVLVVAKVEKGKSKKLKSVK
ELLGITIMERSSFEKNPIDFLEAKGYKEVKKDLIIKLPKYSLFELENGRKRML
ASAGELQKGNELALPSKYVNFLYLASHYEKLKGSPEDNEQKQLFVEQHKH
YLDEIIEQISEFSKRVILADANLDKVLSAYNKHRDKPIREQAENIIHLFTLTNL
GAPAAFKYFDTTIDRKRYTSTKEVLDATLIHQSITGLYETRIDLSQLGGDSPK
KKRKVEASGPAMMMMALSKTFGQKPVKFQLEDDGEFYMIGSEVGNYLRM
FRGSLYKRYPSLWRRLATVEERKKIVASSHGKKTKPNTKDHGYTTLATSVT
LLKASEVEEILDGNDEKYKAVSISTEPPTYLREQKAKRNSQWVPTLPNSSHH
LDAVPCSTTINRNRMGRDKKRTFPLCFDDHDPAVIHENASQPEVLVPIRLDM
EIDGQKLRDAFTWNMNEKLMTPEMFSEILCDDLDLNPLTFVPAIASAIRQQIE
SYPTDSILEDQSDQRVIIKLNIHVGNISLVDQFEWDMSEKENSPEKFALKLCS
ELGLGGEFVTTIAYSIRGQLSWHQKTYAFSENPLPTVEIAIRNTGDADQWCP
LLETLTDAEMEKKIRDQDRNTRRMRRLANTAPAW
dCas9hMBD4
(SEQ ID NO: 18)
MYPYDVPDYASPKKKRKVEASDKKYSIGLAIGTNSVGWAVIT
DEYKVPSKKFKVLGNTDRHSIKKNLIGALLFDSGETAEATRLKRTARRRYTR
RKNRICYLQEIFSNEMAKVDDSFFHRLEESFLVEEDKKHERHPIFGNIVDEVA
YHEKYPTIYHLRKKLVDSTDKADLRLIYLALAHMIKFRGHFLIEGDLNPDNS
DVDKLFIQLVQTYNQLFEENPINASGVDAKAILSARLSKSRRLENLIAQLPGE
KKNGLFGNLIALSLGLTPNFKSNFDLAEDAKLQLSKDTYDDDLDNLLAQIG
DQYADLFLAAKNLSDAILLSDILRVNTEITKAPLSASMIKRYDEHHQDLTLL
KALVRQQLPEKYKEIFFDQSKNGYAGYIDGGASQEEFYKFIKPILEKMDGTE
ELLVKLNREDLLRKQRTFDNGSIPHQIHLGELHAILRRQEDFYPFLKDNREKI
EKILTFRIPYYVGPLARGNSRFAWMTRKSEETITPWNFEEVVDKGASAQSFIE
RMTNFDKNLPNEKVLPKHSLLYEYFTVYNELTKVKYVTEGMRKPAFLSGE
QKKAIVDLLFKTNRKVTVKQLKEDYFKKIECFDSVEISGVEDRFNASLGTYH
DLLKIIKDKDFLDNEENEDILEDIVLTLTLFEDREMIEERLKTYAHLFDDKVM
KQLKRRRYTGWGRLSRKLINGIRDKQSGKTILDFLKSDGFANRNFMQLIHD
DSLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHPVE
NTQLQNEKLYLYYLQNGRDMYVDQELDINRLSDYDVDAIVPQSFLKDDSID
NKVLTRSDKNRGKSDNVPSEEVVKKMKNYWRQLLNAKLITQRKFDNLTKA
ERGGLSELDKAGFIKRQLVETRQITKHVAQILDSRMNTKYDENDKLIREVKV
ITLKSKLVSDFRKDFQFYKVREINNYHHAHDAYLNAVVGTALIKKYPKLESE
FVYGDYKVYDVRKMIAKSEQEIGKATAKYFFYSNIMNFFKTEITLANGEIRK
RPLIETNGETGEIVWDKGRDFATVRKVLSMPQVNIVKKTEVQTGGFSKESIL
PKRNSDKLIARKKDWDPKKYGGFDSPTVAYSVLVVAKVEKGKSKKLKSVK
ELLGITIMERSSFEKNPIDFLEAKGYKEVKKDLIIKLPKYSLFELENGRKRML
ASAGELQKGNELALPSKYVNFLYLASHYEKLKGSPEDNEQKQLFVEQHKH
YLDEIIEQISEFSKRVILADANLDKVLSAYNKHRDKPIREQAENIIHLFTLTNL
GAPAAFKYFDTTIDRKRYTSTKEVLDATLIHQSITGLYETRIDLSQLGGDSPK
KKRKVEASGPAMGTTGLESLSLGDRGAAPTVTSSERLVPDPPNDLRKEDVA
MELERVGEDEEQMMIKRSSECNPLLQEPIASAQFGATAGTECRKSVPCGWE
RVVKQRLFGKTAGRFDVYFISPQGLKFRSKSSLANYLHKNGETSLKPEDFDF
TVLSKRGIKSRYKDCSMAALTSHLQNQSNNSNWNLRTRSKCKKDVFMPPSS
SSELQESRGLSNFTSTHLLLKEDEGVDDVNFRKVRKPKGKVTILKGIPIKKTK
KGCRKSCSGFVQSDSKRESVCNKADAESEPVAQKSQLDRTVCISDAGACGE
TLSVTSEENSLVKKKERSLSSGSNFCSEQKTSGIINKFCSAKDSEHNEKYEDT
FLESEEIGTKVEVVERKEHLHTDILKRGSEMDNNCSPTRKDFTGEKIFQEDTI
PRTQIERRKTSLYFSSKYNKEALSPPRRKAFKKWTPPRSPFNLVQETLFHDP
WKLLIATIFLNRTSGKMAIPVLWKFLEKYPSAEVARTADWRDVSELLKPLG
LYDLRAKTIVKFSDEYLTKQWKYPIELHGIGKYGNDSYRIFCVNEWKQVHP
EDHKLNKYHDWLWENHEKLSLS
dCas9hTDG
(SEQ ID NO: 19)
MYPYDVPDYASPKKKRKVEASDKKYSIGLAIGTNSVGWAVIT
DEYKVPSKKFKVLGNTDRHSIKKNLIGALLFDSGETAEATRLKRTARRRYTR
RKNRICYLQEIFSNEMAKVDDSFFHRLEESFLVEEDKKHERHPIFGNIVDEVA
YHEKYPTIYHLRKKLVDSTDKADLRLIYLALAHMIKFRGHFLIEGDLNPDNS
DVDKLFIQLVQTYNQLFEENPINASGVDAKAILSARLSKSRRLENLIAQLPGE
KKNGLFGNLIALSLGLTPNFKSNFDLAEDAKLQLSKDTYDDDLDNLLAQIG
DQYADLFLAAKNLSDAILLSDILRVNTEITKAPLSASMIKRYDEHHQDLTLL
KALVRQQLPEKYKEIFFDQSKNGYAGYIDGGASQEEFYKFIKPILEKMDGTE
ELLVKLNREDLLRKQRTFDNGSIPHQIHLGELHAILRRQEDFYPFLKDNREKI
EKILTFRIPYYVGPLARGNSRFAWMTRKSEETITPWNFEEVVDKGASAQSFIE
RMTNFDKNLPNEKVLPKHSLLYEYFTVYNELTKVKYVTEGMRKPAFLSGE
QKKAIVDLLFKTNRKVTVKQLKEDYFKKIECFDSVEISGVEDRFNASLGTYH
DLLKIIKDKDFLDNEENEDILEDIVLTLTLFEDREMIEERLKTYAHLFDDKVM
KQLKRRRYTGWGRLSRKLINGIRDKQSGKTILDFLKSDGFANRNFMQLIHD
DSLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHPVE
NTQLQNEKLYLYYLQNGRDMYVDQELDINRLSDYDVDAIVPQSFLKDDSID
NKVLTRSDKNRGKSDNVPSEEVVKKMKNYWRQLLNAKLITQRKFDNLTKA
ERGGLSELDKAGFIKRQLVETRQITKHVAQILDSRMNTKYDENDKLIREVKV
ITLKSKLVSDFRKDFQFYKVREINNYHHAHDAYLNAVVGTALIKKYPKLESE
FVYGDYKVYDVRKMIAKSEQEIGKATAKYFFYSNIMNFFKTEITLANGEIRK
RPLIETNGETGEIVWDKGRDFATVRKVLSMPQVNIVKKTEVQTGGFSKESIL
PKRNSDKLIARKKDWDPKKYGGFDSPTVAYSVLVVAKVEKGKSKKLKSVK
ELLGITIMERSSFEKNPIDFLEAKGYKEVKKDLIIKLPKYSLFELENGRKRML
ASAGELQKGNELALPSKYVNFLYLASHYEKLKGSPEDNEQKQLFVEQHKH
YLDEIIEQISEFSKRVILADANLDKVLSAYNKHRDKPIREQAENIIHLFTLTNL
GAPAAFKYFDTTIDRKRYTSTKEVLDATLIHQSITGLYETRIDLSQLGGDSPK
KKRKVEASGPAMEAENAGSYSLQQAQAFYTFPFQQLMAEAPNMAVVNEQ
QMPEEVPAPAPAQEPVQEAPKGRKRKPRTTEPKQPVEPKKPVESKKSGKSA
KSKEKQEKITDTFKVKRKVDRFNGVSEAELLTKTLPDILTFNLDIVIIGINPGL
MAAYKGHHYPGPGNHFWKCLFMSGLSEVQLNHMDDHTLPGKYG GFTNM
VERTTPGSKDLSSKEFREGGRILVQKLQKYQPRIAVFNGKCIYEIFSKEVFGV
KVKNLEFGLQPHKIPDTETLCYVMPSSSARCAQFPRAQDKVHYYIKLKDLR
DQLKGIERNMDVQEVQYTFDLQLAQEDAKKMAVKEEKYDPGYEAAYGGA
YGENPCSSEPCGFSSNGLIESVELRGESAFSGIPNGQWMTQSFTDQIPSFSNH
CGTQEQEEESHA
dCas9hTET1CD
(SEQ ID NO: 20)
MYPYDVPDYASPKKKRKVEASDKKYSIGLAIGTNSVGWAVIT
DEYKVPSKKFKVLGNTDRHSIKKNLIGALLFDSGETAEATRLKRTARRRYTR
RKNRICYLQEIFSNEMAKVDDSFFHRLEESFLVEEDKKHERHPIFGNIVDEVA
YHEKYPTIYHLRKKLVDSTDKADLRLIYLALAHMIKFRGHFLIEGDLNPDNS
DVDKLFIQLVQTYNQLFEENPINASGVDAKAILSARLSKSRRLENLIAQLPGE
KKNGLFGNLIALSLGLTPNFKSNFDLAEDAKLQLSKDTYDDDLDNLLAQIG
DQYADLFLAAKNLSDAILLSDILRVNTEITKAPLSASMIKRYDEHHQDLTLL
KALVRQQLPEKYKEIFFDQSKNGYAGYIDGGASQEEFYKFIKPILEKMDGTE
ELLVKLNREDLLRKQRTFDNGSIPHQIHLGELHAILRRQEDFYPFLKDNREKI
EKILTFRIPYYVGPLARGNSRFAWMTRKSEETITPWNFEEVVDKGASAQSFIE
RMTNFDKNLPNEKVLPKHSLLYEYFTVYNELTKVKYVTEGMRKPAFLSGE
QKKAIVDLLFKTNRKVTVKQLKEDYFKKIECFDSVEISGVEDRFNASLGTYH
DLLKIIKDKDFLDNEENEDILEDIVLTLTLFEDREMIEERLKTYAHLFDDKVM
KQLKRRRYTGWGRLSRKLINGIRDKQSGKTILDFLKSDGFANRNFMQLIHD
DSLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHPVE
NTQLQNEKLYLYYLQNGRDMYVDQELDINRLSDYDVDAIVPQSFLKDDSID
NKVLTRSDKNRGKSDNVPSEEVVKKMKNYWRQLLNAKLITQRKFDNLTKA
ERGGLSELDKAGFIKRQLVETRQITKHVAQILDSRMNTKYDENDKLIREVKV
ITLKSKLVSDFRKDFQFYKVREINNYHHAHDAYLNAVVGTALIKKYPKLESE
FVYGDYKVYDVRKMIAKSEQEIGKATAKYFFYSNIMNFFKTEITLANGEIRK
RPLIETNGETGEIVWDKGRDFATVRKVLSMPQVNIVKKTEVQTGGFSKESIL
PKRNSDKLIARKKDWDPKKYGGFDSPTVAYSVLVVAKVEKGKSKKLKSVK
ELLGITIMERSSFEKNPIDFLEAKGYKEVKKDLIIKLPKYSLFELENGRKRML
ASAGELQKGNELALPSKYVNFLYLASHYEKLKGSPEDNEQKQLFVEQHKH
YLDEIIEQISEFSKRVILADANLDKVLSAYNKHRDKPIREQAENIIHLFTLTNL
GAPAAFKYFDTTIDRKRYTSTKEVLDATLIHQSITGLYETRIDLSQLGGDSPK
KKRKVEASGPAELPTCSCLDRVIQKDKGPYYTHLGAGPSVAAVREIMENRY
GQKGNAIRIEIVVYTGKEGKSSHGCPIAKWVLRRSSDEEKVLCLVRQRTGHH
CPTAVMVVLIMVWDGIPLPMADRLYTELTENLKSYNGHPTDRRCTLNENRT
CTCQGIDPETCGASFSFGCSWSMYFNGCKFGRSPSPRRFRIDPSSPLHEKNLE
DNLQSLATRLAPIYKQYAPVAYQNQVEYENVARECRLGSKEGRPFSGVTAC
LDFCAHPHRDIHNMNNGSTVVCTLTREDNRSLGVIPQDEQLHVLPLYKLSD
TDEFGSKEGMEAKIKSGAIEVLAPRRKKRTCFTQPVPRSGKKRAAMMTEVL
AHKIRAVEKKPIPRIKRKNNSTTTNNSKPSSLPTLGSNTETVQPEVKSETEPHF
ILKSSDNTKTYSLMPSAPHPVKEASPGFSWSPKTASATPAPLKNDATASCGF
SERSSTPHCTMPSGRLSGANAAAADGPGISQLGEVAPLPTLSAPVMEPLINSE
PSTGVTEPLTPHQPNHQPSFLTSPQDLASSPMEEDEQHSEADEPPSDEPLSDD
PLSPAEEKLPHIDEYWSDSEHIFLDANIGGVAIAPAHGSVLIECARRELHATTP
VEHPNRNHPTRLSLVFYQHKNLNKPQHGFELNKIKFEAKEAKNKKMKASE
QKDQAANEGPEQSSEVNELNQIPSHKALTLTHDNVVTVSPYALTHVAGPYN
HWVID
dCas9hPRMT1
(SEQ ID NO: 21)
MYPYDVPDYASPKKKRKVEASDKKYSIGLAIGTNSVGWAVITDE
YKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYH
EKYPTIYHLRKKLVDSTDKADLRLIYLALAHMIKFRGHFLIEGDLNPDNSDV
DKLFIQLVQTYNQLFEENPINASGVDAKAILSARLSKSRRLENLIAQLPGEKK
NGLFGNLIALSLGLTPNFKSNFDLAEDAKLQLSKDTYDDDLDNLLAQIGDQ
YADLFLAAKNLSDAILLSDILRVNTEITKAPLSASMIKRYDEHHQDLTLLKAL
VRQQLPEKYKEIFFDQSKNGYAGYIDGGASQEEFYKFIKPILEKMDGTEELL
VKLNREDLLRKQRTFDNGSIPHQIHLGELHAILRRQEDFYPFLKDNREKIEKI
LTFRIPYYVGPLARGNSRFAWMTRKSEETITPWNFEEVVDKGASAQSFIERM
TNFDKNLPNEKVLPKHSLLYEYFTVYNELTKVKYVTEGMRKPAFLSGEQKK
AIVDLLFKTNRKVTVKQLKEDYFKKIECFDSVEISGVEDRFNASLGTYHDLL
KIIKDKDFLDNEENEDILEDIVLTLTLFEDREMIEERLKTYAHLFDDKVMKQL
KRRRYTGWGRLSRKLINGIRDKQSGKTILDFLKSDGFANRNFMQLIHDDSLT
FKEDIQKAQVSGQGDSLHEHIANLAGSPAIKKGILQTVKVVDELVKVMGRH
KPENIVIEMARENQTTQKGQKNSRERMKRIEEGIKELGSQILKEHPVENTQL
QNEKLYLYYLQNGRDMYVDQELDINRLSDYDVDAIVPQSFLKDDSIDNKVL
TRSDKNRGKSDNVPSEEVVKKMKNYWRQLLNAKLITQRKFDNLTKAERGG
LSELDKAGFIKRQLVETRQITKHVAQILDSRMNTKYDENDKLIREVKVITLK
SKLVSDFRKDFQFYKVREINNYHHAHDAYLNAVVGTALIKKYPKLESEFVY
GDYKVYDVRKMIAKSEQEIGKATAKYFFYSNIMNFFKTEITLANGEIRKRPLI
ETNGETGEIVWDKGRDFATVRKVLSMPQVNIVKKTEVQTGGFSKESILPKR
NSDKLIARKKDWDPKKYGGFDSPTVAYSVLVVAKVEKGKSKKLKSVKELL
GITIMERSSFEKNPIDFLEAKGYKEVKKDLIIKLPKYSLFELENGRKRMLASA
GELQKGNELALPSKYVNFLYLASHYEKLKGSPEDNEQKQLFVEQHKHYLDE
IIEQISEFSKRVILADANLDKVLSAYNKHRDKPIREQAENIIHLFTLTNLGAPA
AFKYFDTTIDRKRYTSTKEVLDATLIHQSITGLYETRIDLSQLGGDSPKKKRK
VEASGPAMAAAEAANCIMENFVATLANGMSLQPPLEEVSCGQAESSEKPNA
EDMTSKDYYFDSYAHFGIHEEMLKDEVRTLTYRNSMFHNRHLFKDKVVLD
VGSGTGILCMFAAKAGARKVIGIECSSISDYAVKIVKANKLDHVVTIIKGKV
EEVELPVEKVDIIISEWMGYCLFYESMLNTVLYARDKWLAPDGLIFPDRATL
YVTAIEDRQYKDYKIHWWENVYGFDMSCIKDVAIKEPLVDVVDPKQLVTN
ACLIKEVDIYTVKVEDLTFTSPFCLQVKRNDYVHALVAYFNIEFTRCHKRTG
FSTSPESPYTHWKQTVFYMEDYLTVKTGEEIFGTIGMRPNAKNNRDLDFTID
LDFKGQLCELSCSTDYRMRID
dCas9hSET7
(SEQ ID NO: 22)
MYPYDVPDYASPKKKRKVEASDKKYSIGLAIGTNSVGWAVITDE
YKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYH
EKYPTIYHLRKKLVDSTDKADLRLIYLALAHMIKFRGHFLIEGDLNPDNSDV
DKLFIQLVQTYNQLFEENPINASGVDAKAILSARLSKSRRLENLIAQLPGEKK
NGLFGNLIALSLGLTPNFKSNFDLAEDAKLQLSKDTYDDDLDNLLAQIGDQ
YADLFLAAKNLSDAILLSDILRVNTEITKAPLSASMIKRYDEHHQDLTLLKAL
VRQQLPEKYKEIFFDQSKNGYAGYIDGGASQEEFYKFIKPILEKMDGTEELL
VKLNREDLLRKQRTFDNGSIPHQIHLGELHAILRRQEDFYPFLKDNREKIEKI
LTFRIPYYVGPLARGNSRFAWMTRKSEETITPWNFEEVVDKGASAQSFIERM
TNFDKNLPNEKVLPKHSLLYEYFTVYNELTKVKYVTEGMRKPAFLSGEQKK
AIVDLLFKTNRKVTVKQLKEDYFKKIECFDSVEISGVEDRFNASLGTYHDLL
KIIKDKDFLDNEENEDILEDIVLTLTLFEDREMIEERLKTYAHLFDDKVMKQL
KRRRYTGWGRLSRKLINGIRDKQSGKTILDFLKSDGFANRNFMQLIHDDSLT
FKEDIQKAQVSGQGDSLHEHIANLAGSPAIKKGILQTVKVVDELVKVMGRH
KPENIVIEMARENQTTQKGQKNSRERMKRIEEGIKELGSQILKEHPVENTQL
QNEKLYLYYLQNGRDMYVDQELDINRLSDYDVDAIVPQSFLKDDSIDNKVL
TRSDKNRGKSDNVPSEEVVKKMKNYWRQLLNAKLITQRKFDNLTKAERGG
LSELDKAGFIKRQLVETRQITKHVAQILDSRMNTKYDENDKLIREVKVITLK
SKLVSDFRKDFQFYKVREINNYHHAHDAYLNAVVGTALIKKYPKLESEFVY
GDYKVYDVRKMIAKSEQEIGKATAKYFFYSNIMNFFKTEITLANGEIRKRPLI
ETNGETGEIVWDKGRDFATVRKVLSMPQVNIVKKTEVQTGGFSKESILPKR
NSDKLIARKKDWDPKKYGGFDSPTVAYSVLVVAKVEKGKSKKLKSVKELL
GITIMERSSFEKNPIDFLEAKGYKEVKKDLIIKLPKYSLFELENGRKRMLASA
GELQKGNELALPSKYVNFLYLASHYEKLKGSPEDNEQKQLFVEQHKHYLDE
IIEQISEFSKRVILADANLDKVLSAYNKHRDKPIREQAENIIHLFTLTNLGAPA
AFKYFDTTIDRKRYTSTKEVLDATLIHQSITGLYETRIDLSQLGGDSPKKKRK
VEASGPAMDSDDEMVEEAVEGHLDDDGLPHGFCTVTYSSTDRFEGNFVHG
EKNGRGKFFFFDGSTLEGYYVDDALQGQGVYTYEDGGVLQGTYVDGELNG
PAQEYDTDGRLIFKGQYKDNIRHGVCWIYYPDGGSLVGEVNEDGEMTGEKI
AYVYPDERTALYGKFIDGEMIEGKLATLMSTEEGRPHFELMPGNSVYHFDK
STSSCISTNALLPDPYESERVYVAESLISSAGEGLFSKVAVGPNTVMSFYNGV
RITHQEVDSRDWALNGNTLSLDEETVIDVPEPYNHVSKYCASLGHKANHSF
TPNCIYDMFVHPRFGPIKCIRTLRAVEADEELTVAYGYDHSPPGKSGPEAPE
WYQVELKAFQATQQKID
dCas9SID4x
(SEQ ID NO: 23)
MYPYDVPDYASPKKKRKVEASDKKYSIGLAIGTNSVGWAVITDE
YKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYH
EKYPTIYHLRKKLVDSTDKADLRLIYLALAHMIKFRGHFLIEGDLNPDNSDV
DKLFIQLVQTYNQLFEENPINASGVDAKAILSARLSKSRRLENLIAQLPGEKK
NGLFGNLIALSLGLTPNFKSNFDLAEDAKLQLSKDTYDDDLDNLLAQIGDQ
YADLFLAAKNLSDAILLSDILRVNTEITKAPLSASMIKRYDEHHQDLTLLKAL
VRQQLPEKYKEIFFDQSKNGYAGYIDGGASQEEFYKFIKPILEKMDGTEELL
VKLNREDLLRKQRTFDNGSIPHQIHLGELHAILRRQEDFYPFLKDNREKIEKI
LTFRIPYYVGPLARGNSRFAWMTRKSEETITPWNFEEVVDKGASAQSFIERM
TNFDKNLPNEKVLPKHSLLYEYFTVYNELTKVKYVTEGMRKPAFLSGEQKK
AIVDLLFKTNRKVTVKQLKEDYFKKIECFDSVEISGVEDRFNASLGTYHDLL
KIIKDKDFLDNEENEDILEDIVLTLTLFEDREMIEERLKTYAHLFDDKVMKQL
KRRRYTGWGRLSRKLINGIRDKQSGKTILDFLKSDGFANRNFMQLIHDDSLT
FKEDIQKAQVSGQGDSLHEHIANLAGSPAIKKGILQTVKVVDELVKVMGRH
KPENIVIEMARENQTTQKGQKNSRERMKRIEEGIKELGSQILKEHPVENTQL
QNEKLYLYYLQNGRDMYVDQELDINRLSDYDVDAIVPQSFLKDDSIDNKVL
TRSDKNRGKSDNVPSEEVVKKMKNYWRQLLNAKLITQRKFDNLTKAERGG
LSELDKAGFIKRQLVETRQITKHVAQILDSRMNTKYDENDKLIREVKVITLK
SKLVSDFRKDFQFYKVREINNYHHAHDAYLNAVVGTALIKKYPKLESEFVY
GDYKVYDVRKMIAKSEQEIGKATAKYFFYSNIMNFFKTEITLANGEIRKRPLI
ETNGETGEIVWDKGRDFATVRKVLSMPQVNIVKKTEVQTGGFSKESILPKR
NSDKLIARKKDWDPKKYGGFDSPTVAYSVLVVAKVEKGKSKKLKSVKELL
GITIMERSSFEKNPIDFLEAKGYKEVKKDLIIKLPKYSLFELENGRKRMLASA
GELQKGNELALPSKYVNFLYLASHYEKLKGSPEDNEQKQLFVEQHKHYLDE
IIEQISEFSKRVILADANLDKVLSAYNKHRDKPIREQAENIIHLFTLTNLGAPA
AFKYFDTTIDRKRYTSTKEVLDATLIHQSITGLYETRIDLSQLGGDSPKKKRK
VEASGPAASPKKKRKVEASGSGMNIQMLLEAADYLERREREAEHGYASML
PGSGMNIQMLLEAADYLERREREAEHGYASMLPGSGMNIQMLLEAADYLE
RREREAEHGYASMLPGSGMNIQMLLEAADYLERREREAEHGYASMLPSRS
RIDID
dCas9hsSssIM
(SEQ ID NO: 24)
MYPYDVPDYASPKKKRKVEASDKKYSIGLAIGTNSVGWAVITDE
YKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYH
EKYPTIYHLRKKLVDSTDKADLRLIYLALAHMIKFRGHFLIEGDLNPDNSDV
DKLFIQLVQTYNQLFEENPINASGVDAKAILSARLSKSRRLENLIAQLPGEKK
NGLFGNLIALSLGLTPNFKSNFDLAEDAKLQLSKDTYDDDLDNLLAQIGDQ
YADLFLAAKNLSDAILLSDILRVNTEITKAPLSASMIKRYDEHHQDLTLLKAL
VRQQLPEKYKEIFFDQSKNGYAGYIDGGASQEEFYKFIKPILEKMDGTEELL
VKLNREDLLRKQRTFDNGSIPHQIHLGELHAILRRQEDFYPFLKDNREKIEKI
LTFRIPYYVGPLARGNSRFAWMTRKSEETITPWNFEEVVDKGASAQSFIERM
TNFDKNLPNEKVLPKHSLLYEYFTVYNELTKVKYVTEGMRKPAFLSGEQKK
AIVDLLFKTNRKVTVKQLKEDYFKKIECFDSVEISGVEDRFNASLGTYHDLL
KIIKDKDFLDNEENEDILEDIVLTLTLFEDREMIEERLKTYAHLFDDKVMKQL
KRRRYTGWGRLSRKLINGIRDKQSGKTILDFLKSDGFANRNFMQLIHDDSLT
FKEDIQKAQVSGQGDSLHEHIANLAGSPAIKKGILQTVKVVDELVKVMGRH
KPENIVIEMARENQTTQKGQKNSRERMKRIEEGIKELGSQILKEHPVENTQL
QNEKLYLYYLQNGRDMYVDQELDINRLSDYDVDAIVPQSFLKDDSIDNKVL
TRSDKNRGKSDNVPSEEVVKKMKNYWRQLLNAKLITQRKFDNLTKAERGG
LSELDKAGFIKRQLVETRQITKHVAQILDSRMNTKYDENDKLIREVKVITLK
SKLVSDFRKDFQFYKVREINNYHHAHDAYLNAVVGTALIKKYPKLESEFVY
GDYKVYDVRKMIAKSEQEIGKATAKYFFYSNIMNFFKTEITLANGEIRKRPLI
ETNGETGEIVWDKGRDFATVRKVLSMPQVNIVKKTEVQTGGFSKESILPKR
NSDKLIARKKDWDPKKYGGFDSPTVAYSVLVVAKVEKGKSKKLKSVKELL
GITIMERSSFEKNPIDFLEAKGYKEVKKDLIIKLPKYSLFELENGRKRMLASA
GELQKGNELALPSKYVNFLYLASHYEKLKGSPEDNEQKQLFVEQHKHYLDE
IIEQISEFSKRVILADANLDKVLSAYNKHRDKPIREQAENIIHLFTLTNLGAPA
AFKYFDTTIDRKRYTSTKEVLDATLIHQSITGLYETRIDLSQLGGDSPKKKRK
VEASGPAMSKVENKTKKLRVFEAFAGIGAQRKALEKVRKDEYEIVGLAEW
YVPAIVMYQAIHNNFHTKLEYKSVSREEMIDYLENKTLSWNSKNPVSNGY
WKRKKDDELKIIYNAIKLSEKEGNIFDIRDLYKRTLKNIDLLTYSFPCQDLSQ
QGIQKGMKRGSGTRSGLLWEIERALDSTEKNDLPKYLLMENVGALLHKKN
EEELNQWKQKLESLGYQNSIEVLNAADFGSSQARRRVFMISTLNEFVELPKG
DKKPKSIKKVLNKIVSEKDILNNLLKYNLTEFKKTKSNINKASLIGYSKFNSE
GYVYDPEFTGPTLTASGANSRIKIKDGSNIRKMNSDETFLYMGFDSQDGKRV
NEIEFLTENQKIFVAGNSISVEVLEAIIDKIGG
dCas9hsSssIMA (split SssIM fragment A fusion)
(SEQ ID NO: 25)
MYPYDVPDYASPKKKRKVEASDKKYSIGLAIGTNSVGWAVITDE
YKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYH
EKYPTIYHLRKKLVDSTDKADLRLIYLALAHMIKFRGHFLIE GDLNPDNSDV
DKLFIQLVQTYNQLFEENPINASGVDAKAILSARLSKSRRLENLIAQLPGEKK
NGLFGNLIALSLGLTPNFKSNFDLAEDAKLQLSKDTYDDDLDNLLAQIGDQ
YADLFLAAKNLSDAILLSDILRVNTEITKAPLSASMIKRYDEHHQDLTLLKAL
VRQQLPEKYKEIFFDQSKNGYAGYIDGGASQEEFYKFIKPILEKMDGTEELL
VKLNREDLLRKQRTFDNGSIPHQIHLGELHAILRRQEDFYPFLKDNREKIEKI
LTFRIPYYVGPLARGNSRFAWMTRKSEETITPWNFEEVVDKGASAQSFIERM
TNFDKNLPNEKVLPKHSLLYEYFTVYNELTKVKYVTEGMRKPAFLSGEQKK
AIVDLLFKTNRKVTVKQLKEDYFKKIECFDSVEISGVEDRFNASLGTYHDLL
KIIKDKDFLDNEENEDILEDIVLTLTLFEDREMIEERLKTYAHLFDDKVMKQL
KRRRYTGWGRLSRKLINGIRDKQSGKTILDFLKSDGFANRNFMQLIHDDSLT
FKEDIQKAQVSGQGDSLHEHIANLAGSPAIKKGILQTVKVVDELVKVMGRH
KPENIVIEMARENQTTQKGQKNSRERMKRIEEGIKELGSQILKEHPVENTQL
QNEKLYLYYLQNGRDMYVDQELDINRLSDYDVDAIVPQSFLKDDSIDNKVL
TRSDKNRGKSDNVPSEEVVKKMKNYWRQLLNAKLITQRKFDNLTKAERGG
LSELDKAGFIKRQLVETRQITKHVAQILDSRMNTKYDENDKLIREVKVITLK
SKLVSDFRKDFQFYKVREINNYHHAHDAYLNAVVGTALIKKYPKLESEFVY
GDYKVYDVRKMIAKSEQEIGKATAKYFFYSNIMNFFKTEITLANGEIRKRPLI
ETNGETGEIVWDKGRDFATVRKVLSMPQVNIVKKTEVQTGGFSKESILPKR
NSDKLIARKKDWDPKKYGGFDSPTVAYSVLVVAKVEKGKSKKLKSVKELL
GITIMERSSFEKNPIDFLEAKGYKEVKKDLIIKLPKYSLFELENGRKRMLASA
GELQKGNELALPSKYVNFLYLASHYEKLKGSPEDNEQKQLFVEQHKHYLDE
IIEQISEFSKRVILADANLDKVLSAYNKHRDKPIREQAENIIHLFTLTNLGAPA
AFKYFDTTIDRKRYTSTKEVLDATLIHQSITGLYETRIDLSQLGGDSPKKKRK
VEASGPAMSKVENKTKKLRVFEAFAGIGAQRKALEKVRKDEYEIVGLAEW
YVPAIVMYQAIHNNFHTKLEYKSVSREEMIDYLENKTLSWNSKNPVSNGY
WKRKKDDELKIIYNAIKLSEKEGNIFDIRDLYKRTLKNIDLLTYSFPCQDLSQ
QGIQKGMKRGSGTRSGLLWEIERALDSTEKNDLPKYLLMENVGALLHKKN
EEELNQWKQKLESLGYQNSIEVLNAADFGSSQARRRVFMISTLNEFVELPKG
DKKPKSIKKVLNKIVSEKDILNNLLKYNLTEFKKTKSNINKASLIGYSKFNSE
GYV
dCas9hsSssIMB (split SssIM fragment B fusion)
(SEQ ID NO: 26)
MYPYDVPDYASPKKKRKVEASDKKYSIGLAIGTNSVGWAVITDE
YKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYH
EKYPTIYHLRKKLVDSTDKADLRLIYLALAHMIKFRGHFLIEGDLNPDNSDV
DKLFIQLVQTYNQLFEENPINASGVDAKAILSARLSKSRRLENLIAQLPGEKK
NGLFGNLIALSLGLTPNFKSNFDLAEDAKLQLSKDTYDDDLDNLLAQIGDQ
YADLFLAAKNLSDAILLSDILRVNTEITKAPLSASMIKRYDEHHQDLTLLKAL
VRQQLPEKYKEIFFDQSKNGYAGYIDGGASQEEFYKFIKPILEKMDGTEELL
VKLNREDLLRKQRTFDNGSIPHQIHLGELHAILRRQEDFYPFLKDNREKIEKI
LTFRIPYYVGPLARGNSRFAWMTRKSEETITPWNFEEVVDKGASAQSFIERM
TNFDKNLPNEKVLPKHSLLYEYFTVYNELTKVKYVTEGMRKPAFLSGEQKK
AIVDLLFKTNRKVTVKQLKEDYFKKIECFDSVEISGVEDRFNASLGTYHDLL
KIIKDKDFLDNEENEDILEDIVLTLTLFEDREMIEERLKTYAHLFDDKVMKQL
KRRRYTGWGRLSRKLINGIRDKQSGKTILDFLKSDGFANRNFMQLIHDDSLT
FKEDIQKAQVSGQGDSLHEHIANLAGSPAIKKGILQTVKVVDELVKVMGRH
KPENIVIEMARENQTTQKGQKNSRERMKRIEEGIKELGSQILKEHPVENTQL
QNEKLYLYYLQNGRDMYVDQELDINRLSDYDVDAIVPQSFLKDDSIDNKVL
TRSDKNRGKSDNVPSEEVVKKMKNYWRQLLNAKLITQRKFDNLTKAERGG
LSELDKAGFIKRQLVETRQITKHVAQILDSRMNTKYDENDKLIREVKVITLK
SKLVSDFRKDFQFYKVREINNYHHAHDAYLNAVVGTALIKKYPKLESEFVY
GDYKVYDVRKMIAKSEQEIGKATAKYFFYSNIMNFFKTEITLANGEIRKRPLI
ETNGETGEIVWDKGRDFATVRKVLSMPQVNIVKKTEVQTGGFSKESILPKR
NSDKLIARKKDWDPKKYGGFDSPTVAYSVLVVAKVEKGKSKKLKSVKELL
GITIMERSSFEKNPIDFLEAKGYKEVKKDLIIKLPKYSLFELENGRKRMLASA
GELQKGNELALPSKYVNFLYLASHYEKLKGSPEDNEQKQLFVEQHKHYLDE
IIEQISEFSKRVILADANLDKVLSAYNKHRDKPIREQAENIIHLFTLTNLGAPA
AFKYFDTTIDRKRYTSTKEVLDATLIHQSITGLYETRIDLSQLGGDSPKKKRK
VEASGPAEFVELPKGDKKPKSIKKVLNKIVSEKDILNNLLKYNLTEFKKTKS
NINKASLIGYSKFNSEGYVYDPEFTGPTLTASGANSRIKIKDGSNIRKMNSDE
TFLYMGFDSQDGKRVNEIEFLTENQKIFVAGNSISVEVLEAIIDKIGG

### Example 3

### One Step Generation of Mice Carrying Reporter and Conditional Allele by CRISPR/Cas Mediated Genome Editing

Here, reporter and conditional mutant mice were created by co-injection of zygotes with Cas9 mRNA, different guide RNAs (sgRNAs) as well as DNA vectors of different sizes. Using this one step procedure, mice carrying a tag or a fluorescent reporter construct in the Nanog, the Sox2 and the Oct4 gene as well as Mecp2 conditional mutant mice were generated. In addition, using sgRNAs targeting two separate sites in the Mecp2 gene, mice harboring the predicted deletions of about 700 bps were produced. Finally, potential off-targets of four sgRNAs in gene-modified mice and ESC lines were analyzed and off-target mutations were identified in only rare instances indicating high specificity of genome editing by the CRISPR/Cas system.

Experimental Procedures

Production of Cas9 mRNA and sgRNA

Bicistronic expression vector px330 expressing Cas9 and sgRNA (Cong, L., et al. Science 339, 819-823 (2013)) was digested with BbsI and treated with Antarctic Phosphatase, and the linearized vector was gel-purified. A pair of oligos (Table 11) for each targeting site was annealed, phosphorylated, and ligated to the linearized vector.

T7 promoter was added to Cas9 coding region by PCR amplification using primer Cas9 F and R (Table 11). T7-Cas9 PCR product was gel-purified and used as the template for in vitro transcription (IVT) using mMESSAGE mMACHINE T7 ULTRA kit (Life Technologies). T7 promoter was added to sgRNAs template by PCR amplification using primer listed in Table 11. The T7-sgRNA PCR product was gel-purified and used as the template for IVT using MEGAshortscript T7 kit (Life Technologies). Both the Cas9 mRNA and the sgRNAs were purified using MEGAclear kit (Life Technologies) and eluted in RNase-free water.

Single Stranded and Double Stranded DNA Donors

All single stranded oligos were ordered as Ultramer DNA oligos from Integrated DNA Technologies. Nanog-2A-mCherry vector was modified from previously published targeting vector Nanog-2A-mCherry-PGK-Neo (Faddah et al., 2013). Nanog-2A-mCherry-PGK-Neo was digested with PacI and AscI to drop out the PGK-Neo cassette, the 9.7 kb fragment was gel purified and blunt-ended using T4 DNA polymerase (New England Biolabs), then self-ligated using T4 DNA ligase (New England Biolabs). Oct4-IRES-eGFP-PGK-Neo vector is previously published (Lengner et al., 2007).

Suveryor Assay and RFLP Analysis for Genome Modification

Suveryor assay was performed as described (Guschin, D. Y., et al., Methods Mol Biol 649, 247-256 (2010)). Genomic DNA from targeted and control mice or embryos was extracted and PCR was performed using gene specific primers (Table 11) under the following conditions: 95° C. for 5 min; 35×(95° C. for 30 s, 60° C. for 30 s, 68° C. for 40 s); 68° C. for 2 min; hold at 4° C. PCR products were then denatured, annealed, and treated with Suveryor nuclease (Transgenomic). DNA concentration of each band was measured on an ethidium bromide-stained 10% acrylamide Criterion TBE gel (BioRad) and quantified using Image J software. For RFLP analysis, 10 μl of Tet1, Tet2, Mecp2-R1, R2 PCR product was digested with EcoRI, 10 μl of Mecp2-L1, L2 PCR product was digested with NheI. Digested DNA was separated on an ethidium bromide-stained agarose gel (2%). For sequencing, PCR products were cloned using the Original TA Cloning Kit (Invitrogen), and mutations were identified by Sanger sequencing.

One Cell Embryo Injection

All animal procedures were performed according to NIH guidelines and approved by the Committee on Animal Care at MIT. B6D2F1 (C57BL/6×DBA2) female mice and ICR mouse strains were used as embryo donors and foster mothers, respectively. Super-ovulated female B6D2F1 mice (7-8 weeks old) were mated to B6D2F1 stud males, and fertilized embryos were collected from oviducts. Cas mRNA (100 ng/μl), sgRNA (50 ng/μl) and donor oligos (100 ng/μl) were mixed and injected into the cytoplasm of fertilized eggs with well-recognized pronuclei in M2 medium (Sigma). The injected zygotes were cultured in KSOM with amino acids at 37° C. under 5% CO₂ in air until blastocyst stage by 3.5 days. Thereafter, 15-25 blastocysts were transferred into uterus of pseudopregnant ICR females at 2.5 dpc.

Southern Blotting

Genomic DNA was separated on a 0.8% agarose gel after restriction digests with the appropriate enzymes, transferred to a nylon membrane (Amersham) and hybridized with ³²P random primer (Stratagene)-labeled probes. Between hybridizations, blots were stripped and checked for complete removal of radioactivity before rehybridization with a different probe.

In Vivo Cre Recombination

A 20-μl reaction containing 1 μg of genomic DNA and 10 units of recombinant Cre recombinase (New England Biolabs) in 1× buffer was incubated at 37° C. for one hour. For all targets, 1 μl of the Cre reaction mix was used as template for PCR reactions with gene-specific primers. For each target, primers DF and DR were used for detecting the deletion products, and primers CF and CR were used to detect the circle product. All products were sequenced.

Immunostaining and Western Blot Analysis

For immunostaining, cells in 24-well were fixed in PBS supplemented with 4% paraformaldehyde for 15 min at room temperature (RT). The cells were then permeabilized using 0.2% Triton X-100 in PBS for 15 min at RT. The cells were blocked for 30 min in 1% BSA in PBS. Primary antibody against V5 (ab9137, abcam) was diluted in the same blocking buffer and incubated with the samples overnight at 4° C. The cells were treated with a fluorescently coupled secondary antibody and then incubated for 1 hr at RT. The nuclei were stained with Hoechst 33342 (Sigma) for 5 min at RT.

For western blot, Cell pellets were lysed on ice in Laemmli buffer (62.5 mM Tris-HCl, pH 6.8, 2% sodium dodecyl sulfate, 5% b-mercaptoethanol, 10% glycerol and 0.01% bromophenol blue) for 30 min in presence of protease inhibitors (Roche Diagnostics), boiled for 5-7 min at 100° C., and subjected to western blot analysis. Primary antibodies: V5 (1:1,000, ab9137, abcam), beta-actin (1:2,000). Blots were probed with anti-goat, or anti-rabbit IgG-HRP secondary antibody (1:10,000) and visualized using ECL detection kit (GE Healthcare).

ESC Derivation and Differentiation

Morulas or blastocysts were selected to generate ES cell lines. The zona pellucida was removed using acid Tyrode solution. Each embryo was transferred into one well of a 96-well plate seeded with ICR embryonic fibroblast feeders in ESC medium supplemented with 20% knockout serum replacement, 1,500 U/ml leukemia inhibitory factor (LIF), 3 M CHIR99021, and 1 M PD0325901. After 4-5 days in culture, the colonies were trypsinized and transferred to a 96-well plate with a fresh feeder layer in fresh medium. Clonal expansion of the ESCs proceeded from 48-well plates to 6-well plates with feeder cells and then to 6-well plates for routine culture.

For ESC differentiation, cells were harvested by trypsinization and transferred to bacterial culture dishes in the ES medium without or LIF. After 3 days, aggregated cells were plated onto gelatin-coated tissue culture dishes and incubated for another 3 days.

Prediction of Potential Off-Targets

Potential off-targets were predicted by searching the mouse genome (mm9) for matches to the 20-nt sgRNA sequence allowing for up to 4 mismatches (Nanog) or 3 mismatches (Sox2, Mecp2-L2 and Mecp2-R1) followed by NGG PAM sequence. Matches were ranked first by ascending number of mismatches, then by ascending distance from the PAM sequence.

Results

Targeted Insertion of Short DNA Fragments

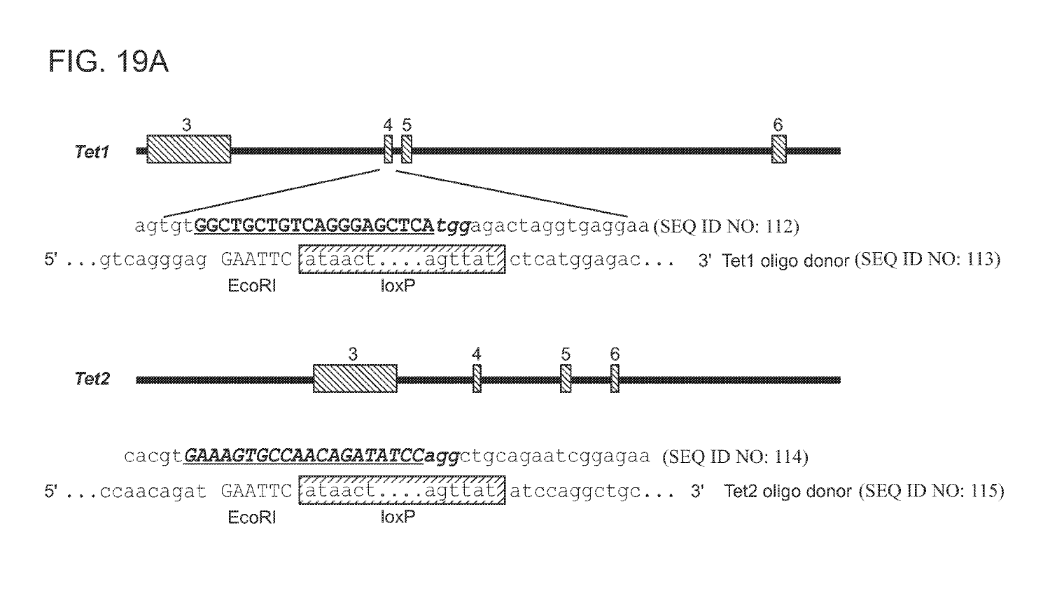

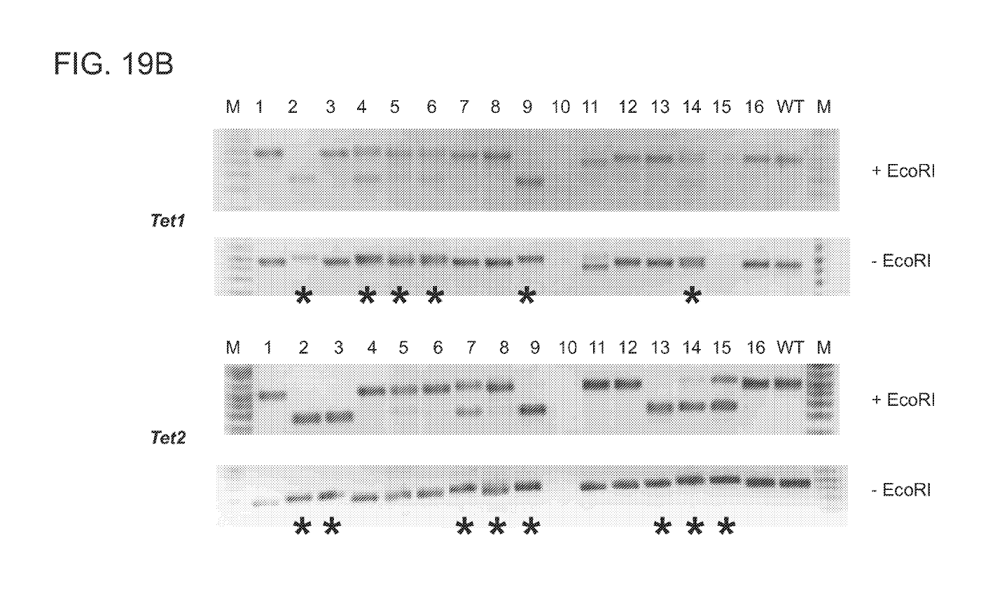

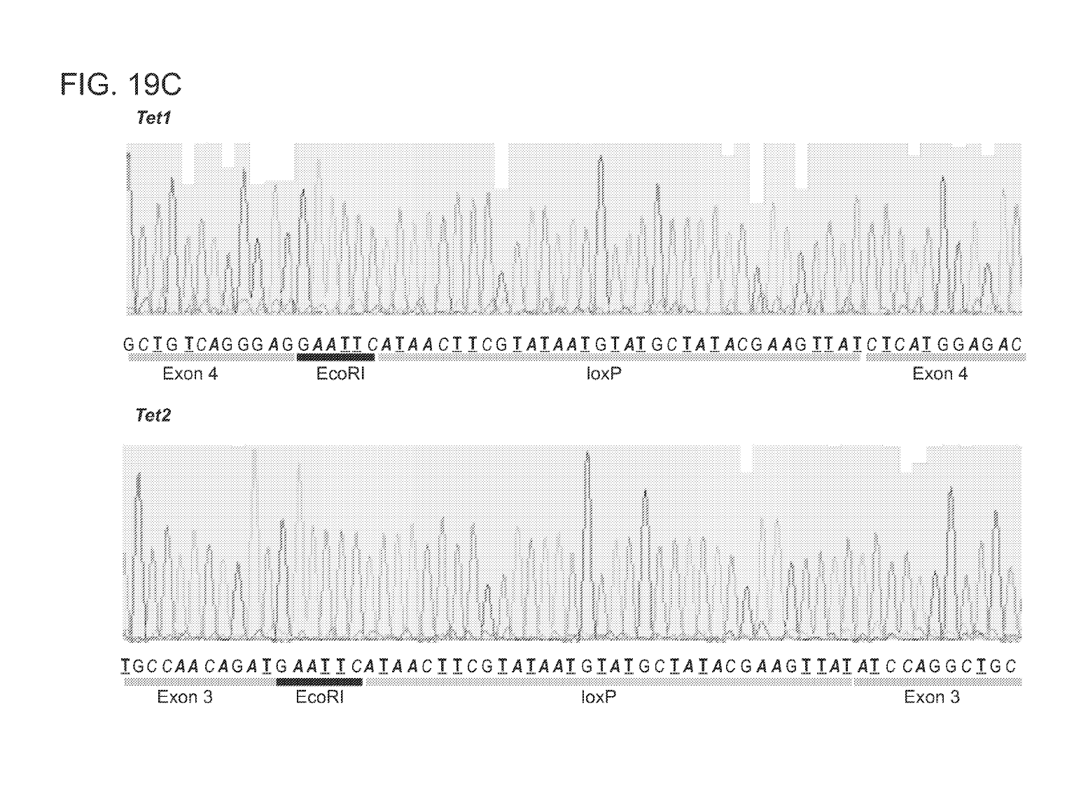

As described herein, precise introduction of base pair mutations into the Tet1 and Tet2 genes was done through homology directed repair (HDR)-mediated genome editing following co-injection of single stranded mutant DNA oligos, sgRNAs and Cas9 mRNA (Wang, H., et al., Cell 153, 910-918 (2013)). To test whether a larger DNA construct could be inserted at the same DSBs at Tet1 exon 4 and Tet2 exon 3, oligos were designed containing the 34 bp loxP site and a 6 bp EcoRI site flanked by 60 bps sequences adjoining the DSBs (FIG. 19A). Cas9 mRNA was co-injected with sgRNAs and single stranded DNA oligos targeting both Tet1 and Tet2 into zygotes. The restriction fragment length polymorphism (RFLP) assay shown in FIG. 19B identified six out of 15 tested embryos carrying the loxP site at the Tet1 locus, eight carrying the loxP site at the Tet2 locus and three had at least one allele of each gene correctly modified. The correct integration of loxP sites was confirmed by sequencing PCR products of sample 2, 9 and 14 (FIG. 19C) These results demonstrate that HDR-mediated repair can introduce targeted integration of 40 bp DNA elements efficiently through CRISPR/Cas mediated genome editing (summarized in Table 7).

Mice with Reporters in the Endogenous Nanog, Sox2 and Oct4 Genes

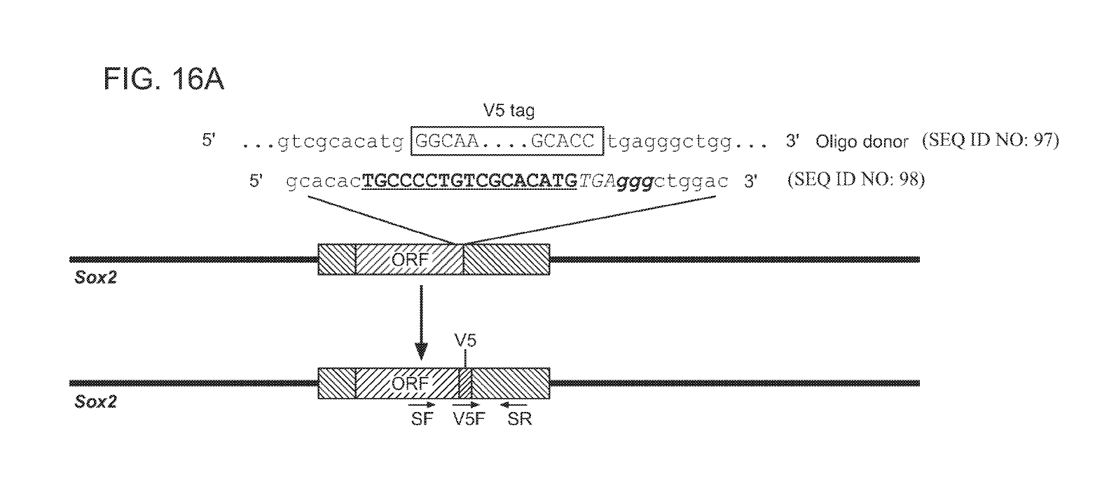

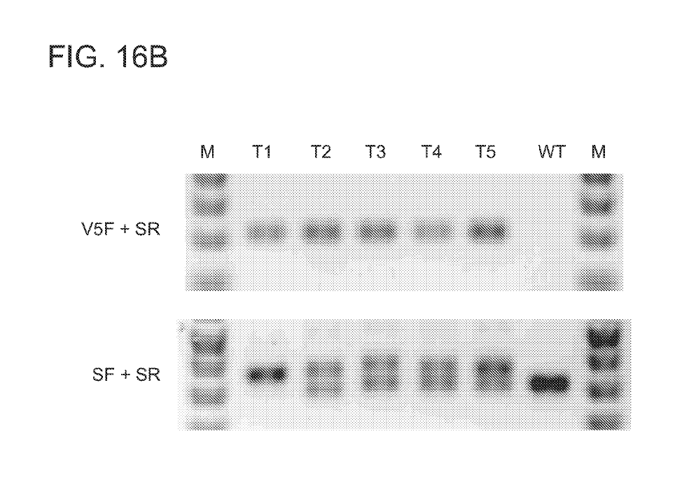

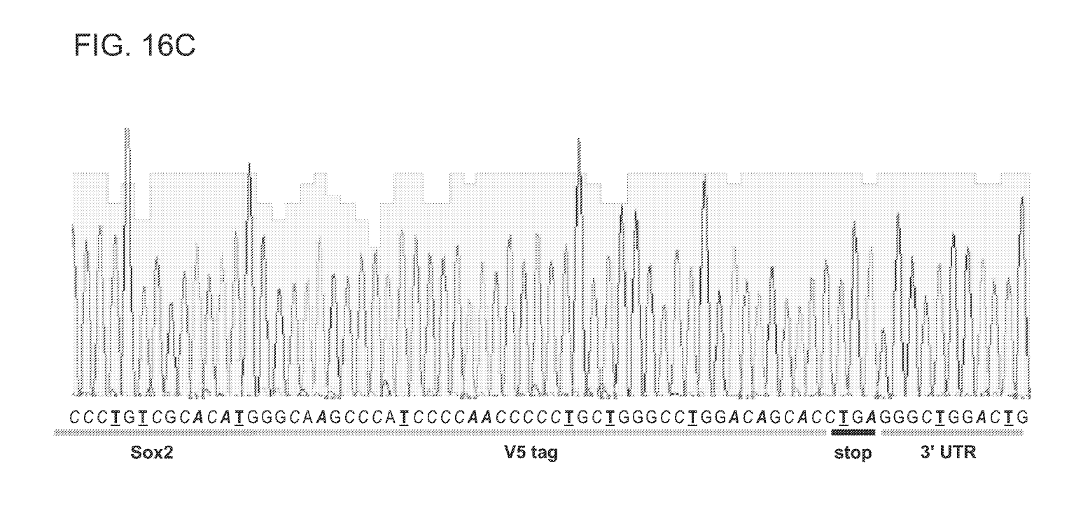

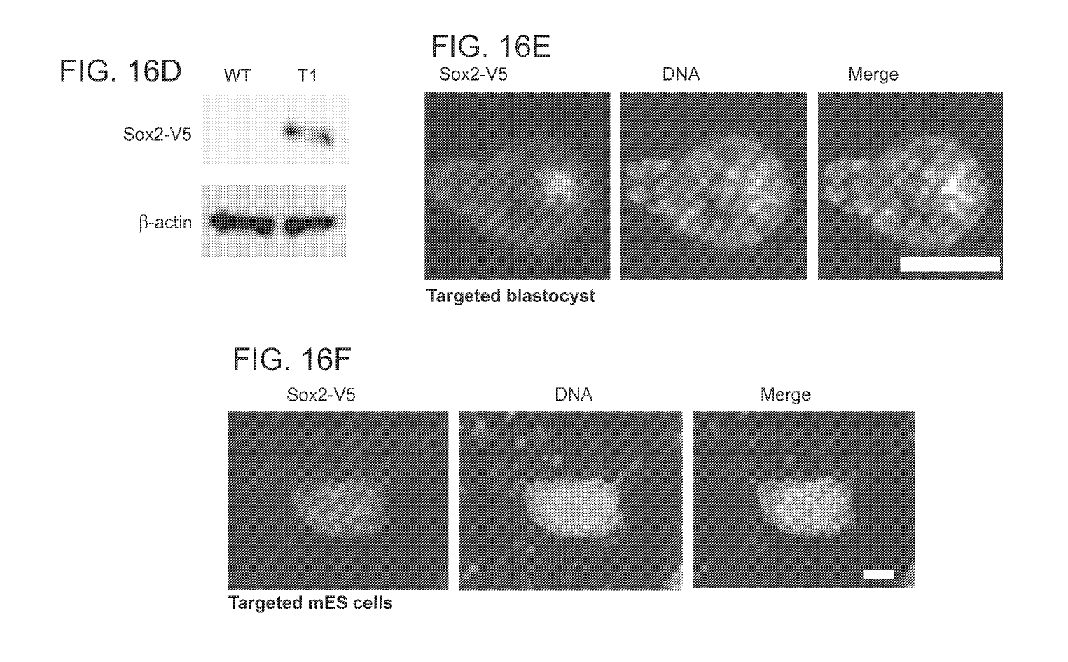

Since the study of many genes and their protein products are limited by the availability of high quality antibodies, the potential of fusing a short epitope tag to an endogenous gene was explored. sgRNA was designed to target the stop codon of Sox2 and a corresponding oligo to fuse the 42 bp V5 tag into the last codon (FIG. 16A). After injection of the sgRNA, Cas9 mRNA and the oligo into zygotes, in vitro differentiated blastocysts were explanted into culture to derive ES cells. PCR genotyping and sequencing identified seven out of 16 ES cell lines carrying a correctly targeted insert (FIGS. 16B and 16C). Western blot analysis revealed a protein band at the predicted size using V5 antibody in targeted ES cells but not in the control cells (FIG. 16D). As expected from a correctly targeted and functional allele, Sox2 expression was seen in targeted blastocysts and ES cells using V5 antibody (FIGS. 16E and 16F). 12 of 35 E 13.5 (Embryonic Day 13) embryos and live born mice derived from injected zygotes carried the V5 tag correctly targeted into the Sox2 gene as indicated by PCR genotyping and sequencing (Table 7).

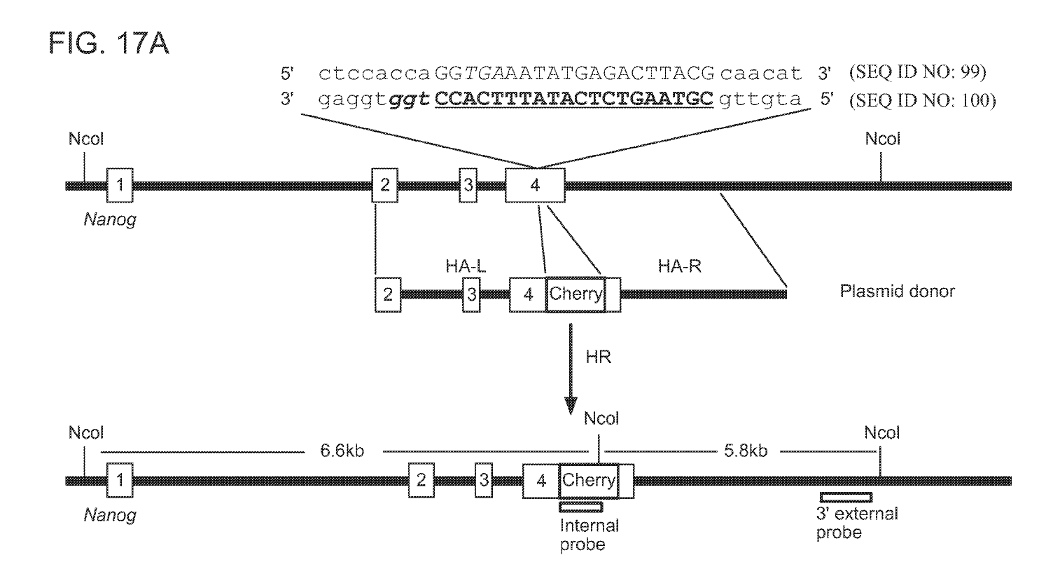

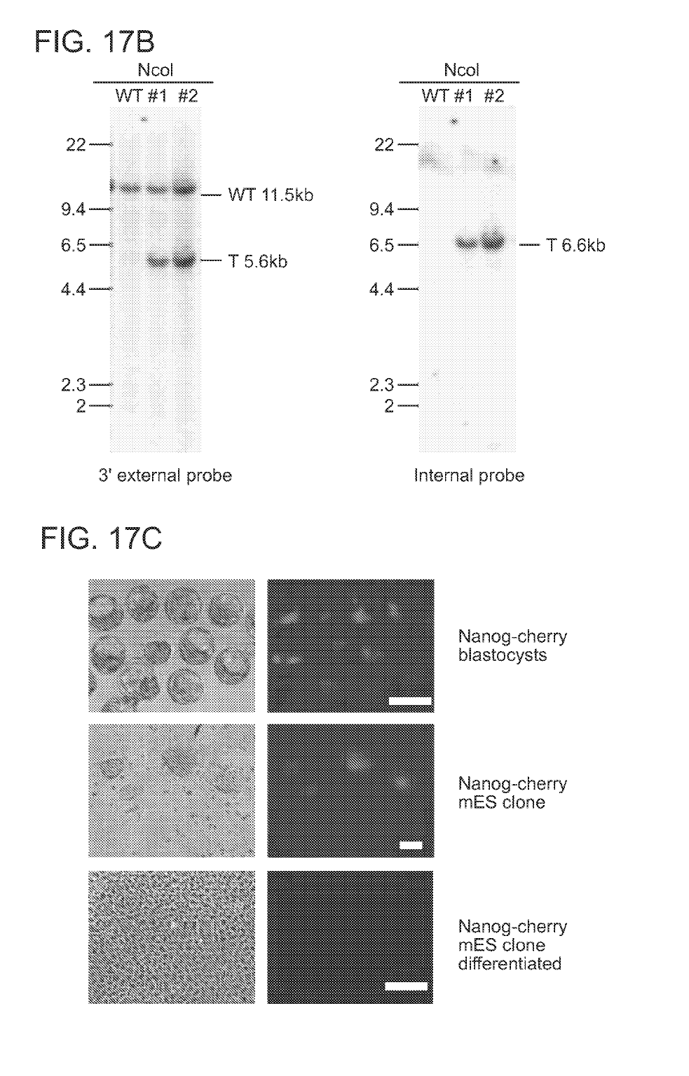

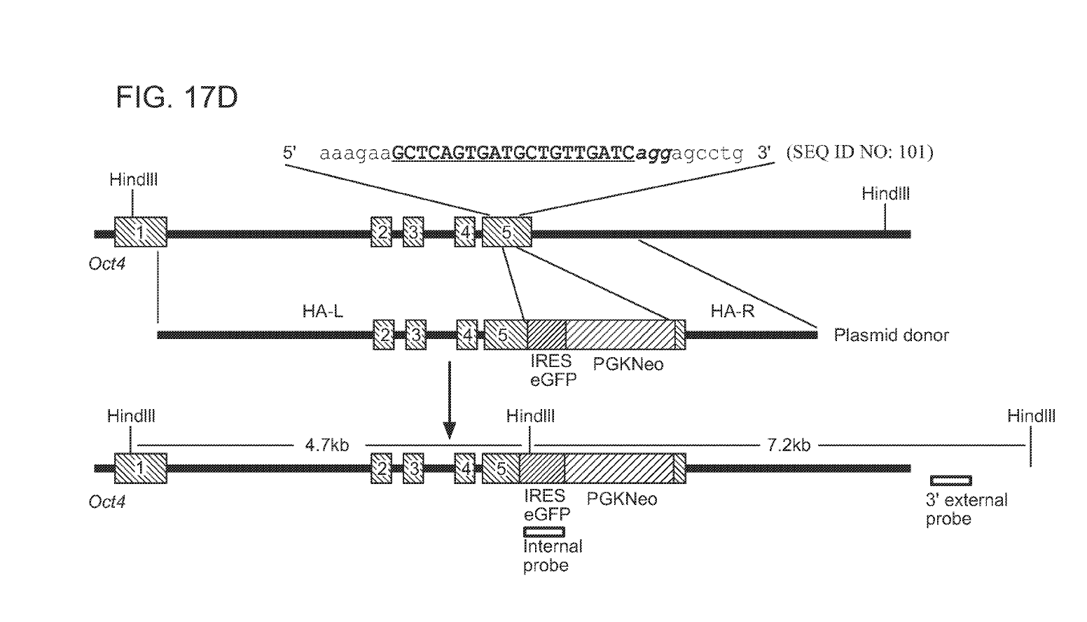

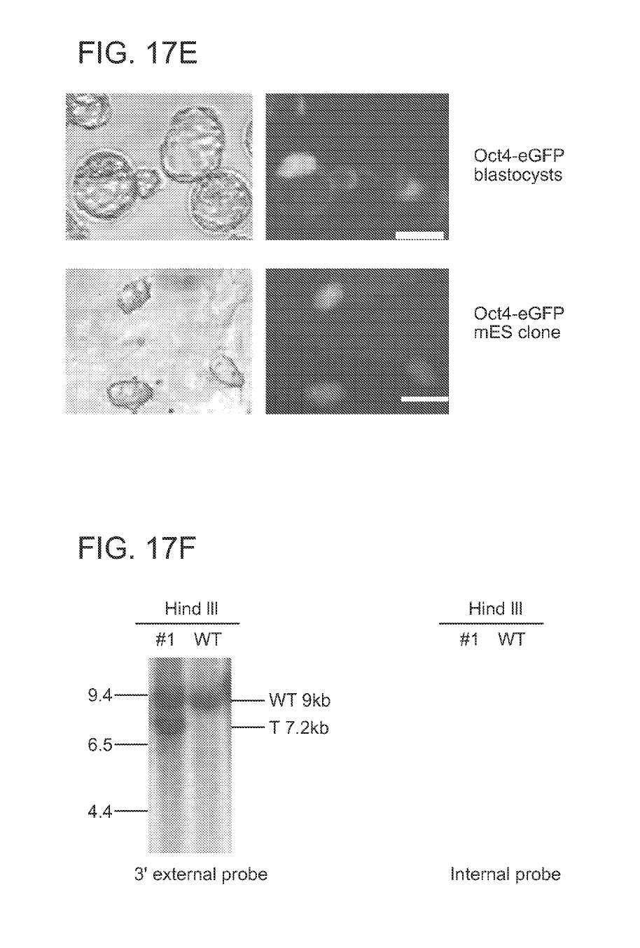

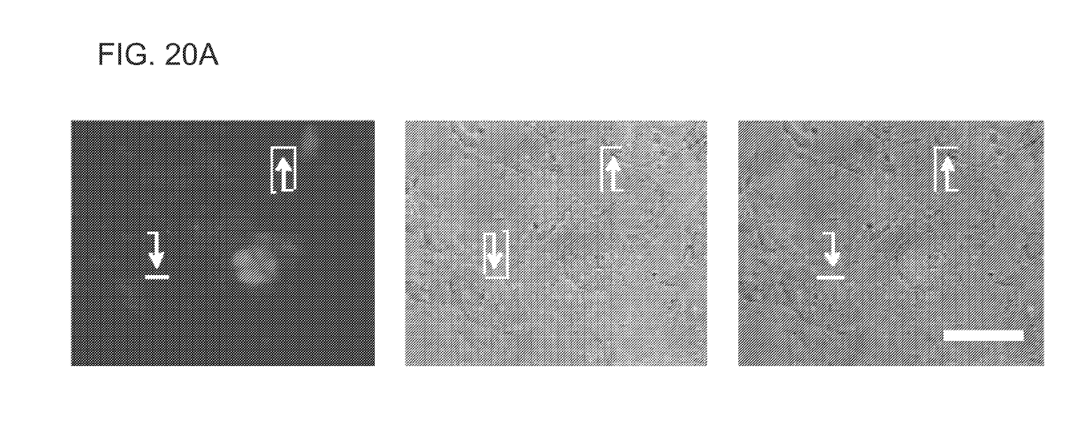

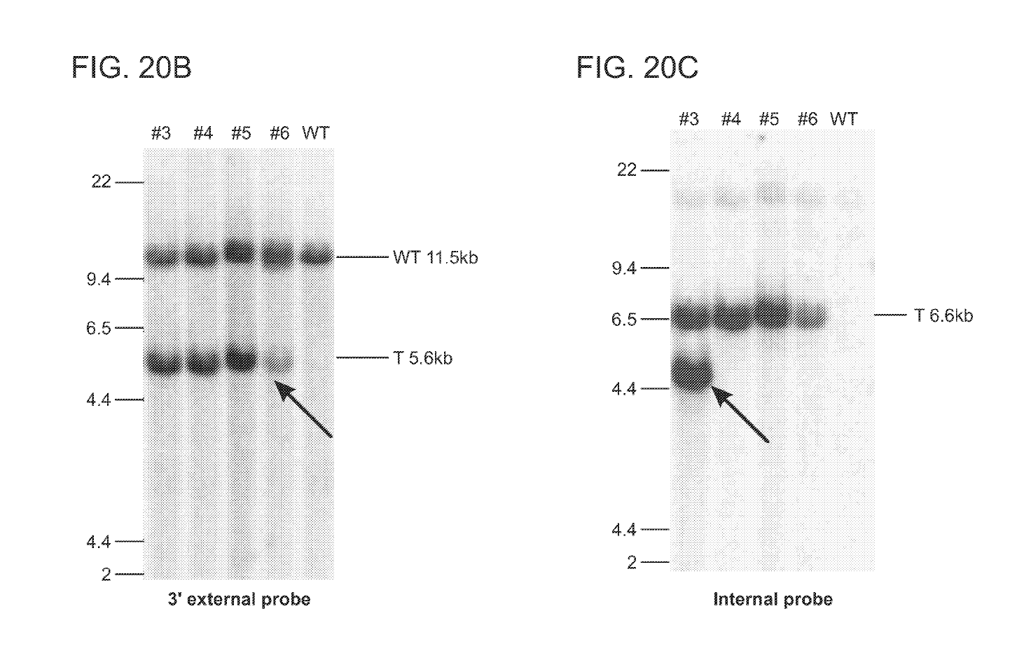

To assess whether a marker transgene could be inserted into an endogenous locus, Cas9 mRNA, sgRNA and a double stranded donor vector which was designed to fuse a p2A-mCherry reporter with the last codon of the Nanog gene were co-injected (FIG. 17A). A circular donor vector was used to minimize random integrations. To assess toxicity and to optimize the concentration of donor DNA, different amounts of Nanog-2A-mCherry vector were microinjected. Injection with high a concentration of donor DNA (500 ng/μl) yielded mCherry-positive embryos with high efficiency with most blastocysts being retarded, whereas injection with a lower donor DNA concentration (10 ng/μl) yielded mostly healthy blastocysts most of which were mCherry-negative. When 200 ng/μl donor DNA was used, 75% of the injected zygotes developed to blastocysts, 9% of which were mCherry-positive (FIG. 17C, Table 9). mCherry was mainly expressed in the inner cell mass (ICM) consistent with targeted integration of the mCherry transgene into the Nanog gene. Six ES cell lines were derived from mCherry positive blastocysts, four of which uniformly expressed mCherry with the signal disappearing upon cellular differentiation (FIG. 17C). The other two lines showed variegated mCherry expression with some colonies being mCherry positive and others negative (FIG. 20A) consistent with mosaic donor embryos, which would be expected if transgene insertion occurred at a later than the zygote stage as has been previously observed with ZNF and TALEN-mediated targeting (Cui, X., et al., Nat Biotechnol 29, 64-67 (2011); Wefers, B., et al., Proc Natl Acad Sci USA 110, 3782-3787 (2013); Brown, A. J., et al. Nat Methods (2013)). Correct transgene integration in ES cell lines was confirmed by Southern blot analysis (FIG. 17B). Postnatal mice from injected zygotes were also generated. Southern blot analysis (FIGS. 20B and 20C) revealed that eight out of 86 E13.5 embryos and live born mice carried the mCherry transgene in the Nanog locus. One targeted mouse was mosaic, since the intensity of targeted allele was lower than the wild type allele (FIG. 20B, #6). Two of the mice carried an additional randomly integrated transgene (FIG. 20C, #3). As summarized in Tables 7 and 9, the efficiency of targeted insertion of the transgene was about 10% in blastocysts and postnatal mice derived from injected zygotes.

Finally, sgRNA targeting the Oct4 3′ UTR was designed, which was co-injected with a published donor vector designed to integrate the 3 kb transgene cassette (IRES-eGFP-loxP-Neo-loxP; FIG. 17D) at the 3′ end of the Oct4 gene (Lengner, C. J., et al. Cell Stem Cell 1, 403-415 (2007)). Blastocysts were derived from injected zygotes, inspected for GFP expression and explanted to derive ES cells. About 20% of the blastocysts displayed uniform GFP expression in the inner cell mass (ICM) region. Three of nice derived ES cell lines expressed GFP (FIG. 17E), including one showed mosaic expression (Table 10). Three out of ten live born mice contained the targeted allele (Table 7). Correct targeting in mice and ES cell lines was confirmed by Southern blot analysis (FIG. 17F). Conventionally, transgenic mice are generated by pronuclear instead of cytoplasmic injection of DNA. To optimize the generation of CRISPR/Cas9 targeted embryos, different concentrations of RNA and of the Nanog-mCherry and the Oct4-GFP DNA vectors were compared as well as three different delivery modes: (i) simultaneous injection of all constructs into the cytoplasm, (ii) simultaneous injection of the RNA and the DNA into the pronucleus and (iii) injection of Cas9/sgRNA into the cytoplasm followed 2 hours later by pronuclear injection of the DNA vector. Table 9 shows that simultaneous injection of all constructs into the cytoplasm at a concentration of 100 ng/μl Cas9 RNA, 50 ng/μl of sgRNA and 200 ng/μl of vector DNA was optimal, resulting in 8% to 18% of targeted blastocysts. Similarly, the simultaneous injection of 5 ng/μl Cas9 RNA, 2.5 ng/μl of sgRNA and 10 ng/μl of DNA vector yielded between 10% and 18% targeted blastocysts. In contrast, the two step procedure with Cas9 and sgRNA simultaneous injected into the cytoplasm followed 2 hours later by pronuclear injection of different concentrations of DNA vector yielded no or at most 3% positive blastocysts. Simultaneous injection of RNA and DNA into the cytoplasm or nucleus is an efficient procedure to achieve targeted insertion.

Conditional Mecp2 Mutant Mice

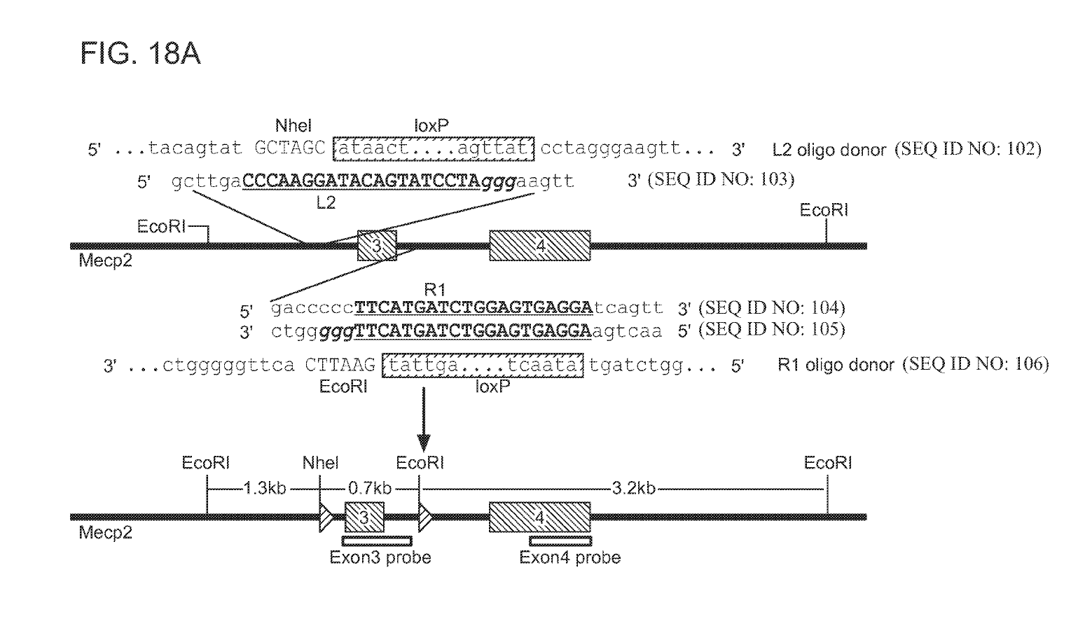

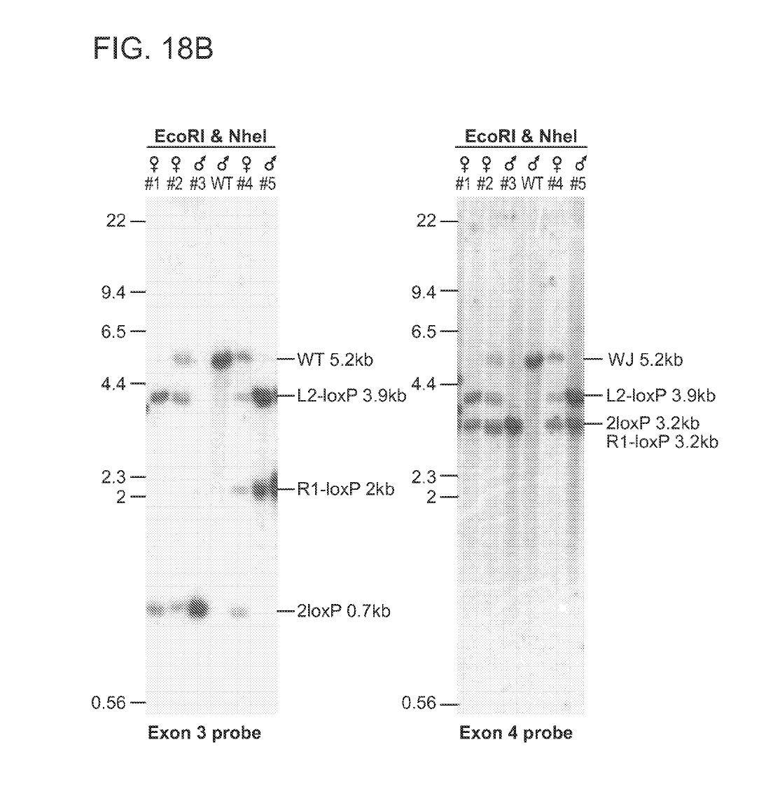

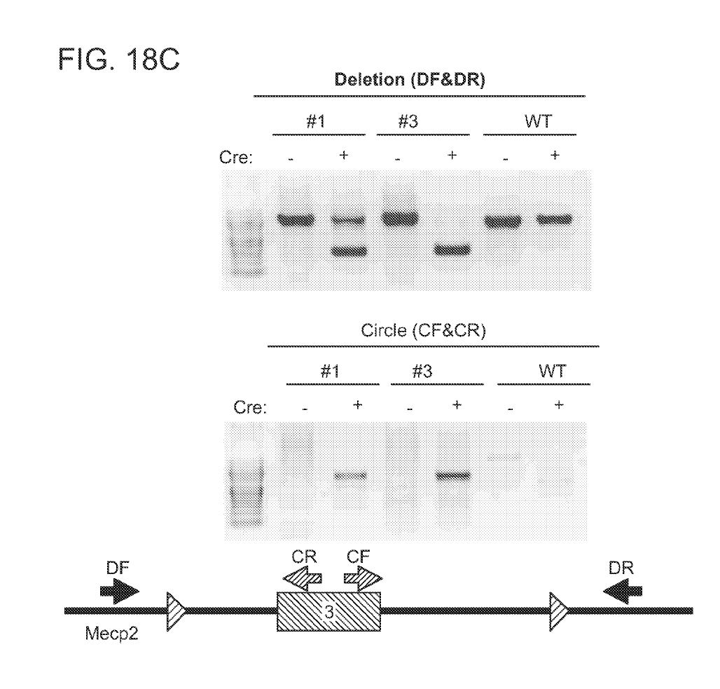

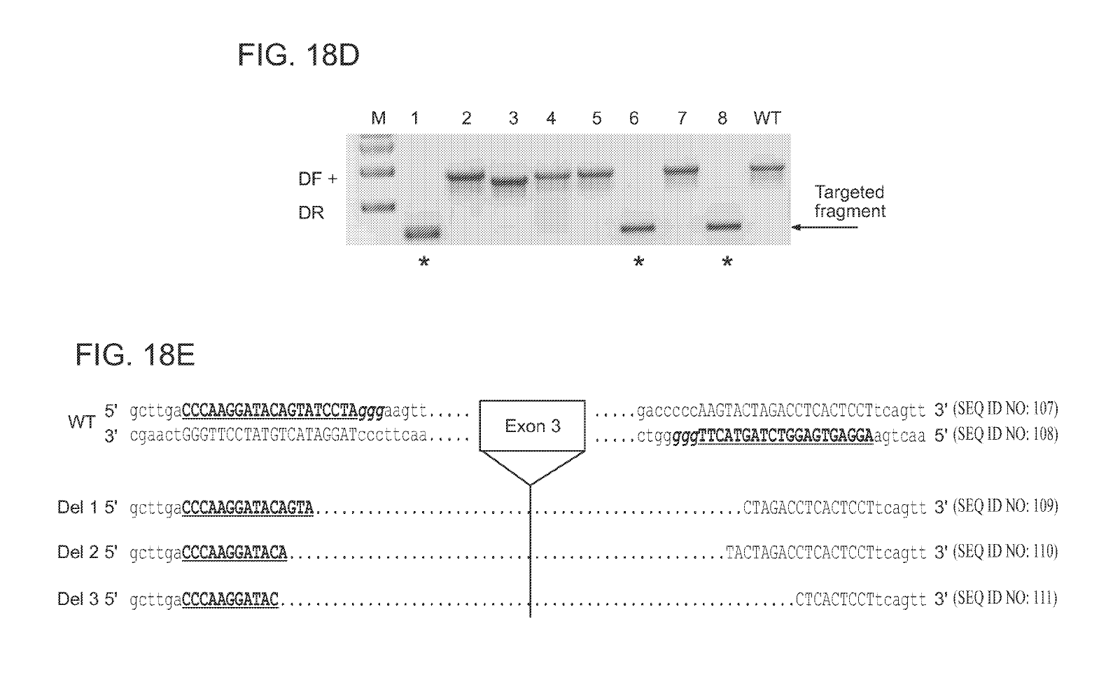

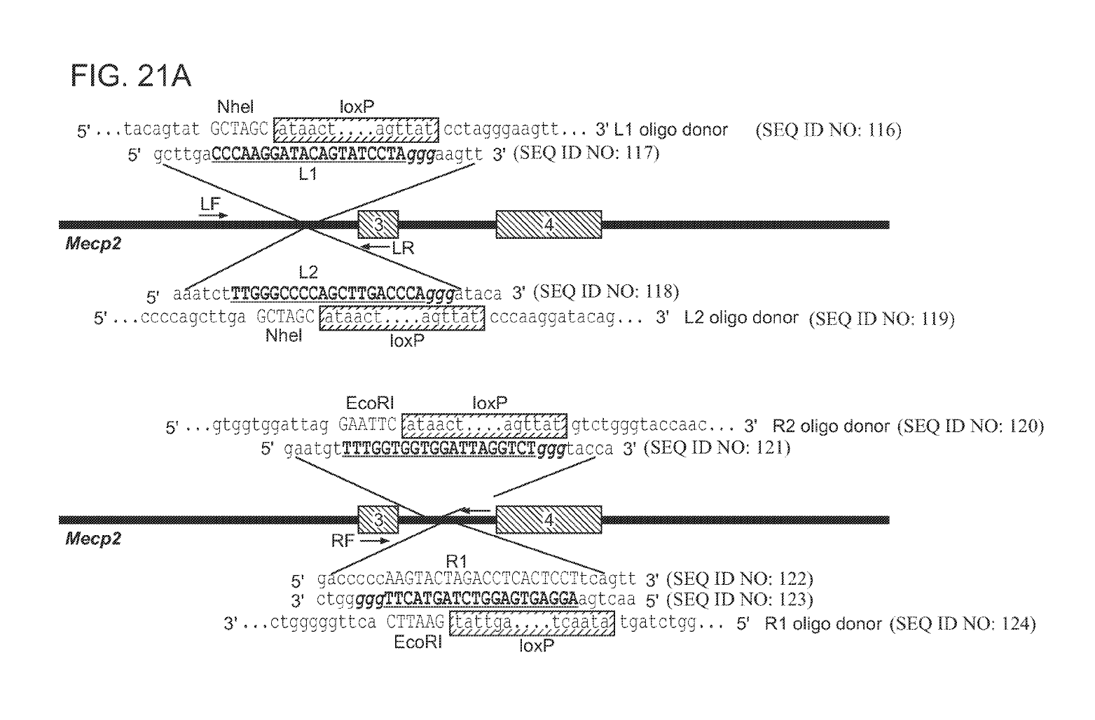

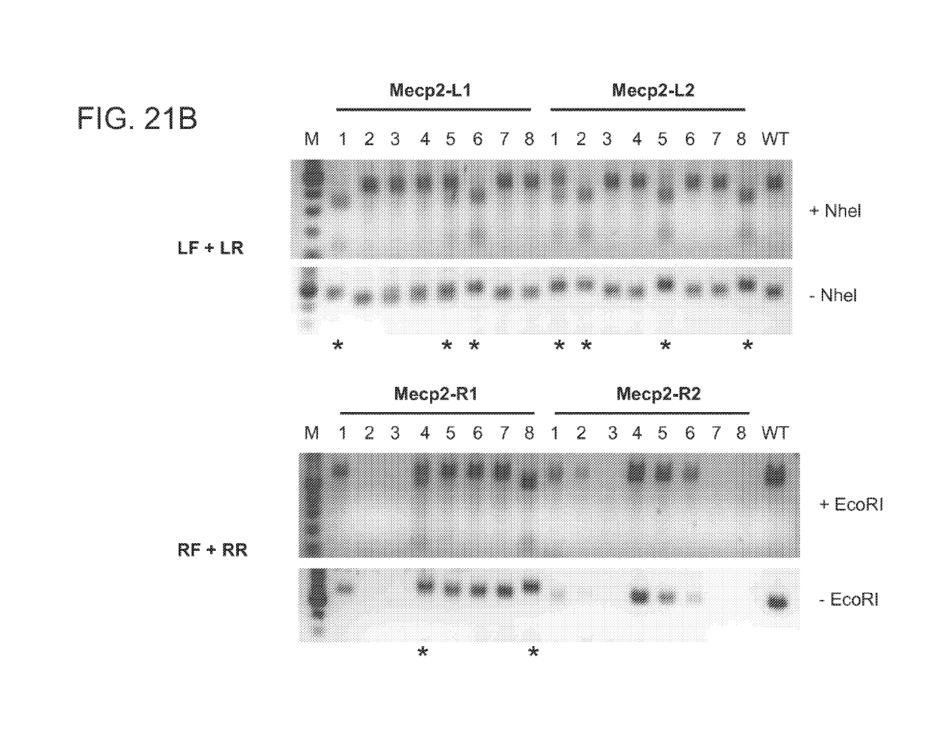

Whether conditional mutant mice can be generated in one step by insertion of two loxP sites into the same allele of the Mecp2 gene was also investigated herein. To derive conditional mutant mice similar to those previously described using traditional homologous recombination methods in ES cells (Chen, R. Z., et al., Nat Genet 27, 327-331 2001)), two sgRNAs targeting Mecp2 intron 2 (L1, L2), and two sgRNAs targeting intron 3 (R1, R2) as well as the corresponding loxP site oligos with 60 bp homology to sequences surrounding each sgRNA mediated DSB were designed (FIG. 21A) To facilitate detection of correct insertions, the oligos targeting intron 2 were engineered to contain an NheI restriction site and the oligos targeting intron 3 to contain an EcoRI site in addition to the LoxP sequences (FIG. 18A, 21A). To determine the efficiency of single loxP site integration at the Mecp2 locus, Cas9 mRNA was injected and each single sgRNA and corresponding oligo into zygotes, which were cultured to the blastocyst stage and genotyped by the RFLP assay. As shown in FIG. 21B, the L2 and R1 sgRNAs were more efficient in integrating the oligos with 4 out of 8 embryos carrying the L2 oligo and 2 out of 6 embryos the carrying R1 oligo (there was no amplification in DNA from 2 embryos). Therefore, L2 and R1 sgRNAs and the corresponding oligos were chosen for the generation of a floxed allele (FIG. 21A).

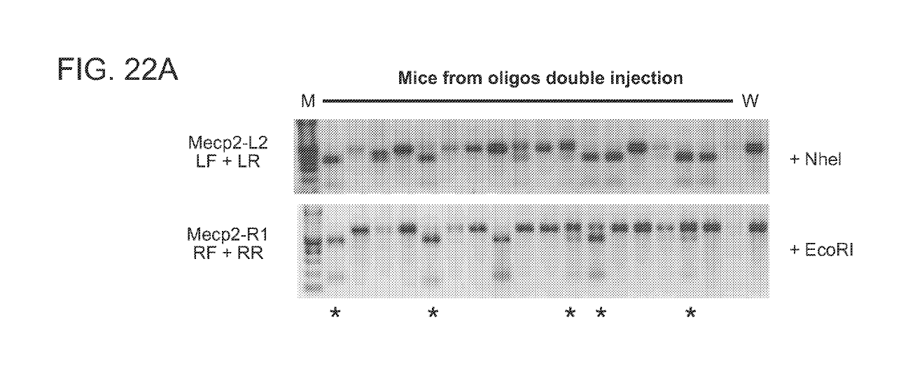

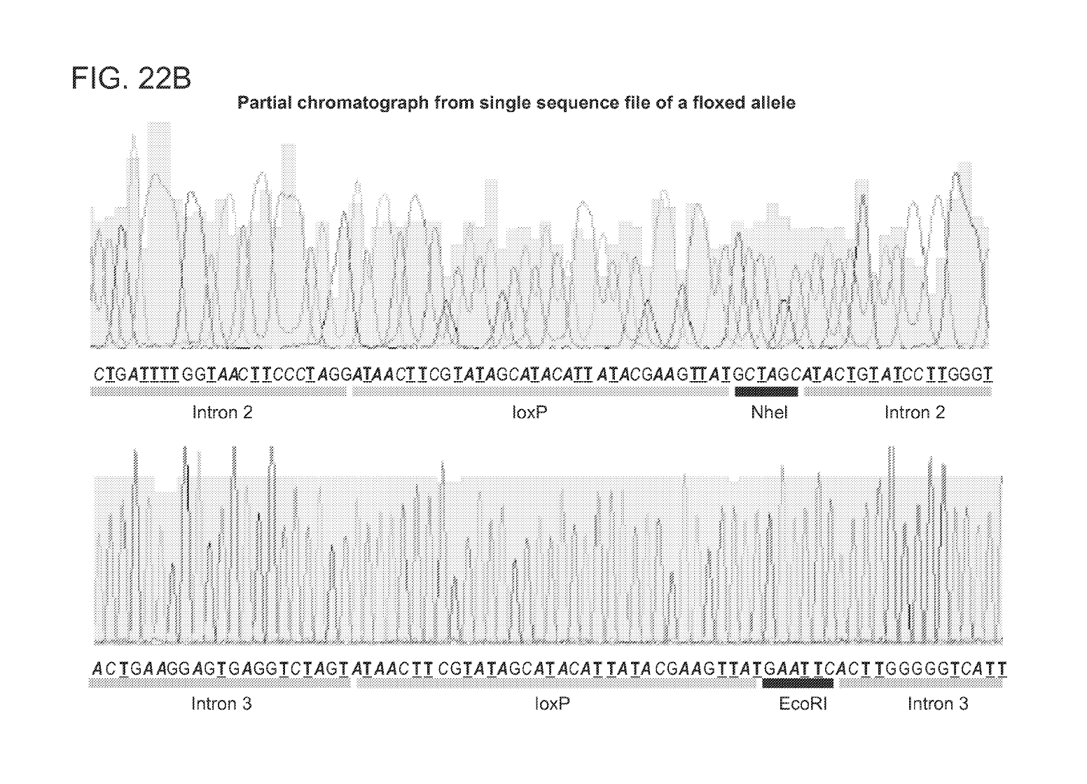

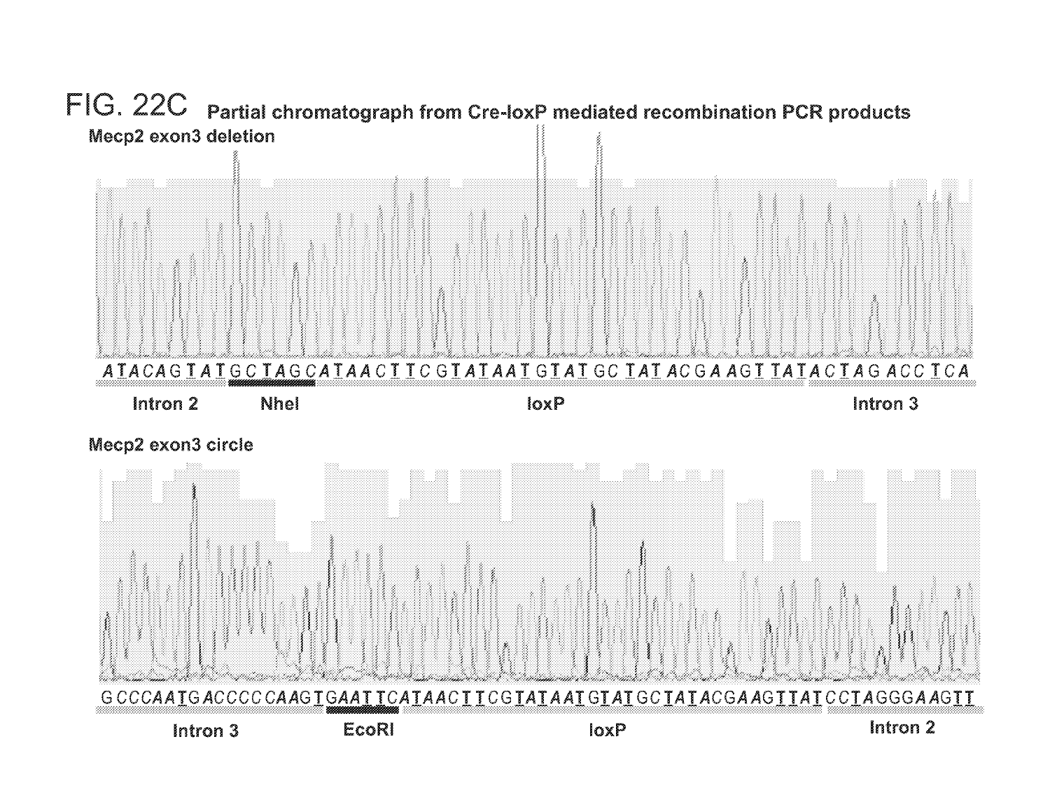

A total of 98 E 13.5 (Embryonic Day) embryos or mice were generated from zygotes injected with Cas9 mRNA, sgRNAs, and DNA oligos targeting the L2 and R1 sites. Genomic DNA was digested with both NheI and EcoRI, and analyzed by Southern blot using exon 3 and 4 probes (FIGS. 18A and 18B). The L2 and R1 oligos contained, in addition to the loxP site, different restriction sites (NheI or EcoRI). Thus, single loxP site integration at L2 or R1 will produce either a 3.9 kb or a 2 kb band, respectively, when hybridized with the exon3 probe (FIGS. 18A and 18B). It was found herein that about 50% of the embryos or mice carried a loxP site at the L2 site and about 25% at the R1 site. Importantly, integration of both loxP sites on the same DNA molecule, generating a floxed allele, produces a 700 bp band as detected by exon 3 probe hybridization (FIGS. 18A and 18B). RFLP analysis, sequencing (FIGS. 22A and 22B) and Southern blot analysis (FIG. 18B) showed that 16 out of the 98 mice tested contained two loxP sites flanking exon 3 on the same allele. Table 8 summarizes the frequency of all alleles and shows that the overall insertion frequency of an L2 or R1 insertion was slightly higher in females (32/38) than in males (38/60) consistent with the higher copy number of the X-linked Mecp2 gene in females. To confirm that the floxed allele was functional, genomic DNA was used for in vitro Cre-mediated recombination. Upon Cre treatment, both the deletion and circular products were detected by PCR in targeted mice, but not in DNA from wild type mice (FIG. 18C). The PCR products were sequenced and confirmed the precise Cre-loxP mediated recombination (FIG. 22C).

ome pups, herein, carried large deletions but no LoxP insertions, raising the possibility that two cleavage events may generate defined deletions. To confirm this, Cas9 mRNA, Mecp2-L2 and R1 sgRNAs were coinjected but without oligos. PCR genotyping and sequencing (FIGS. 18D and 18E) revealed that eight out of 23 mice carried deletions of about 700 bp spanning the L2 and R1 sites removing exon 3. This was confirmed by Southern analysis (not shown). Because DNA breaks are repaired through the non-homologous end joining (NHEJ) pathway, the ends of the breaks are different in different deletion alleles (FIG. 18E).

Mosaicism

As mentioned above, some animals were mosaic for the targeted insertion. The frequency of mosaicism in Mecp2 targeted mice by Southern blot analysis was characterized. Since Mecp2 is an X-linked gene, in males more than one allele and in females more than two different alleles suggest mosaicism, which would be expected if integration occurred later than the zygote stage. For example, as shown in FIG. 18B, female mouse #2 contained three different alleles (one WT allele, one floxed allele, and one L2-loxP allele), and female mouse #4 contained four different alleles (one WT allele, one floxed allele, one L2-loxP allele, and one R1-loxP allele). Male mouse #5 contained two different alleles, with each allele carrying a single loxP site (FIG. 18B). Eight mosaics out of 16 mice were identified containing a Mecp2 floxed allele. The frequency of mosaicism among 49 embryos and mice containing loxP site was about 40% (Table 10). Since Southern blot analysis cannot detect small in-del mutations caused by NHEJ repair, it is possible that this underestimates the overall mosaicism frequency.

Off-Target Analysis

Recent studies identified a high level of off-target cleavage in human cell lines using the CRISPR/Cas system, with Cas9 targeting specificity being shown to tolerate small numbers of mismatches between sgRNA and target DNA in a sequence and position dependent manner (Fu, Y., et al., Nat Biotechnol. (2013); Hsu, P. D., et al., Nat Biotechnol (2013)). Potential off-target (OT) mutations in mice and ES cell lines were characterized derived from zygotes injected with Cas9 and sgRNAs targeting the Sox2, the Nanog and the Mecp2 gene. All genomic loci containing up to three base pair mismatches were identified compared to the 20 bp sgRNA coding sequence (Table 11). All 13 potential OT sites of Sox2 sgRNA was amplified in six mice and four ES cell lines carrying the Sox2-V5 allele and was tested for potential off target mutations using the Surveyor assay. No mutation was detected in any locus. When nine Nanog sgRNA potential OT sites were tested in five correctly targeted mice and four targeted ES cell lines, mutations were found in seven samples at OT1 (Table 11). Since Nanog OT1 has only one base pair difference at the very 5′ end of the sgRNA, it may be not surprising to find such a high frequency of mutations at this locus. In contrast, no off-target mutation was seen in any other Nanog OTs, which contain three or four base pair difference. Finally, four potential off-targets sites for Mecp2 L2, and ten sites for Mecp2 R1 were analyzed in ten mice carrying a Mecp2 floxed allele. Only one off-target mutation was identified in one mouse at the Mecp2 R1 OT2 (Table 11). In summary, all potential off-target sites differing up to three or four base pairs in 29 mice or ES lines were tested and identified mutations in only one off-target site for Nanog (7/9 samples) and Mecp2 (1/10 samples). Thus, the off-target mutation rate is substantially lower than was observed in previous studies using cultured human cancer cell lines (Fu, Y., et al., Nat Biotechnol. (2013); Hsu, P. D., et al., Nat Biotechnol (2013)).

Discussion

In this study, CRISPR/Cas technology can be used for efficient one-step insertions of a short epitope or longer fluorescent tags into precise genomic locations, which will facilitate the generation of mice carrying reporters in endogenous genes. Mice and/or embryos carrying reporter constructs in the Sox2, the Nanog and the Oct4 gene were derived from zygotes injected with Cas9 mRNA, sgRNAs and DNA oligo or vectors encoding a tag or a fluorescent marker. Also, microinjection of two Mecp2 specific sgRNAs, Cas9 mRNA and two different oligos encoding LoxP sites into fertilized eggs allowed the one-step generation of conditional mutant mice. In addition the introduction of two spaced sgRNAs targeting the Mecp2 gene produced mice carrying defined deletions of about 700 bp. Though all RNA and DNA constructs were injected into the cytoplasm or nucleus of zygotes, the gene modification events could happen at the one cell stage or later. Indeed, Southern analyses revealed mosaicism in 13% to 40% of the targeted mice and ES cell lines indicating that the insertion of the transgenes had occurred after the zygote stage (Table 10).

Previous experiments (Wang, H., et al., Cell 153, 910-918 (2013)) demonstrated herein is an efficiency of CRISPR/Cas sgRNA mediated cleavage that was high enough to allow for the one-step production of engineered mice up to 90% of which carried homozygous mutations in two genes (4 mutant alleles). The results reported here show that the sgRNA mediated DSBs occur at a significantly higher frequency than insertion of exogenous DNA sequences. Therefore, the allele not carrying the insert will likely be mutated as a consequence of NHEJ-based gene disruption. Thus, the reporter allele would need to be segregated away from the mutant allele in order to produce mice carrying a reporter as well as a wt allele.

Two recent studies reported a high off-target mutation rate in CRISPR/Cas9 transfected human cell lines (Fu, Y., et al., Nat Biotechnol. (2013); Hsu, P. D., et al., Nat Biotechnol (2013)). The off-target rate for four different sgRNAs was analyzed and identified the cleavage of Nanog OT1 in 7 out of 9 samples and of Mecp2 R1 OT2 in 1/10 mice tested. Nanog OT1 has only one base pair difference from the targeting sequence at the extreme 5′ end (position 20, numbered 1-20 in the 3′ to 5′ direction of gRNA target site), while Mecp2 R1 OT2 has one base pair mismatch at position 20, and one mismatch at position 7. No mutations were detected in 34 potential OTs of Sox2, Nanog, Oct4 or Mecp2 containing 2, 3, or 4 bp mismatches in a total of 29 mice and ES cell lines tested. This result is consistent with the previous findings that Cas9 can catalyze DNA cleavage in the presence of single-base mismatches in the PAM-distal region (Cong et al., 2013; Hsu et al., 2013; Jiang et al., 2013; Jinek et al., 2012). Consistent with the observation that three or more interspaced mismatches dramatically reduce Cas9 cleavage (Hsu et al., 2013), there were no observed off-target mutations at loci containing 3 bp mismatches.

There are several possibilities to explain the significant difference in off-target cleavage rate seen in animals derived from manipulated zygotes and the results reported for CRISPR/Cas treated human cell lines (Fu, Y., et al., Nat Biotechnol. (2013); Hsu, P. D., et al., Nat Biotechnol (2013)). The off-target mutagenesis was analyzed based on the analysis of a “clonal genome” in animals derived from a single manipulated zygote, in contrast to the two previous reports that analyzed heterogeneous cell populations. The surveyor assay, based upon extensive PCR amplification, may identify any mutation, even very rare alleles that may be present in the heterogeneous population. The transformed human cell lines may have different DNA damage responses resulting in a different mutagenesis rate than the normal one cell embryo. In the experiments described herein, CRISPR/Cas was injecting as short-lived RNA in contrast to Fu et al. and Hsu et al. who used DNA plasmid transfection, which may express the Cas9/sgRNA for longer time periods leading to more extensive cleavage. Thus, this data suggests high specificity of the CRISPR/Cas9 system for gene editing in early embryos aimed at generating gene-modified mice. Nevertheless, characterization of off-target mutagenesis of CRISPR/Cas system using whole genome sequencing would be highly informative and may allow designing sgRNAs with higher specificity.

In summary, CRISPR/Cas mediated genome editing represents an efficient and simple method of generating sophisticated genetic modifications in mice such as conditional alleles and endogenous reporters in one step. The principles described in this study could be directly adapted to other mammalian species, which provides sophisticated genome engineering in many species where ES cells are not available.

TABLE 7
Mice with reporters in the endogenous genes
Cas9 mRNA, sgRNAs targeting Tet1, Tet2, Sox2, Nanog, or Oct4,
and single stranded DNA oligos or double stranded donor vectors
were injected into fertilized eggs. Targeted blastocysts were identified
by RFLP or fluorescence of reporters. The blastocysts derived from
injected embryos were derived ES cell lines or transplanted into
foster mothers and E13.5 embryos and postnatal mice
were obtained and genotyped.

| | | | | | | Knock-in |
|---|---|---|---|---|---|---|
| | Blasto- | | | Target- | Trans- | pre- and |
| | cysts/ | Targeted | | ed | ferred | postnatal |
| | Injected | blastocysts/ | | ESCs/ | embryos | mice/ |
| Donor | zygotes | Total | | Total | (recipients) | Total |
| Tet1-loxP + | 65/89 | Tex1- | 6/15 | N/A | N/A | N/A |
| Tet2-loxP | | loxP | | | | |
| | | Tex2- | 6/15 | | | |
| | | loxP | | | | |
| | | Both | 3/15 | | | |

| | | | | | | |
|---|---|---|---|---|---|---|
| Sox2-V5 | 414/498 | N/A | 7/16 | 200 | (10) | 12/35 |
| Nanog- | 936/1262 | 86/936 | ND^{a} | 415 | (21) | 8/86 |
| mCherry | | | | | | |
| Oct4-GFP | 254/345 | 47/254 | 3/9 | 100 | (4) | 3/10 |

^{a}Only mCherry positive blastocysts were selected to generate ES cell lines.
ND, not determined;
N/A, not applicable.

TABLE 8
Conditional Mecp2 mutant mice
Cas9 mRNA, sgRNAs targeting Mecp2-L2 and Mecp2-R1, and single stranded DNA
oligos were injected into fertilized eggs. The blastocysts derived from the injected embryos
were transplanted into foster mothers and pre- and postnatal mice were genotyped.

| | | | | (Pre and post) Mice with loxP/Total | | | | |
|---|---|---|---|---|---|---|---|---|
| | Blastocyst/ | Transferred | | | | | Two loxP | Two loxP |
| | Injected | embryos | | | | | in two | in one |
| Donor | zygotes | (recipients) | Sex | Total^{a} | L2^{b} | R1^{c} | alleles | alleles |
| Mecp2-L2 + | 367/451 | 360(18) | Male | 28/60 | 26/60 | 12/60 | 2^{d}/60 | 8/60 |
| Mecpe-R1 | | | Female | 21/38 | 19/38 | 13/38 | 3/38 | 8/38 |
| | | | Total | 49/98 | 45/98 | 25/98 | 5/98 | 16/98 |

^{a}Total mice containing loxP site integration in the genome.
^{b}Mice containing loxP site integrated at L2 site.
^{c}Mice containing loxP site integrated at R1 site.
^{d}These male mice were mosaic.

TABLE 9
Efficiency of generation of reporter embryos
by cytoplasm and pronuclear injection

| | Dose of | Dose of | | |
|---|---|---|---|---|
| | Cas9/ | Donor | | Targeted |
| | sgRNA | vector | Injected | Blastocysts/ |
| Donor | (ng/μl) | (ng/μl) | zygotes | Total |
| One-step injection | | | | |
| Nanog-mCherry | 100/50 (Cyto) | 500 (Cyto) | 186 | 1/81 |
| Nanog-mCherry | 100/50 (Cyto) | 200 (Cyto) | 1262 | 86/936 |
| Nanog-mCherry | 100/50 (Cyto) | 50 (Cyto) | 402 | 7/308 |
| Nanog-mCherry | 100/50 (Cyto) | 10 (Cyto) | 333 | 1/278 |
| Oct4-GFP | 100/50 (Cyto) | 200 (Cyto) | 345 | 47/254 |
| Nanog-mCherry | 5/2.5 (Nuc) | 10 (Nuc) | 98 | 7/75 |
| Oct4-GFP | 5/2.5 (Nuc) | 10 (Nuc) | 105 | 13/72 |
| Two-step injection | | | | |
| Nanog-mCherry | 100/50 (Cyto) | 50 (Nuc) | 45 | 0/0 |
| Nanog-mCherry | 100/50 (Cyto) | 10 (Nuc) | 91 | 1/34 |
| Nanog-mCherry | 100/50 (Cyto) | 2 (Nuc) | 85 | 1/68 |

Cas9 mRNA, sgRNAs targeting Nanog, or Oct4, and double stranded donor vectors were injected into cytoplasm or pronuclei of zygotes. In one-step injection, the RNA and the DNA were simultaneously injected into the cytoplasm or pronucleus. In two-step injection, Cas9/sgRNA were injected into the cytoplasm followed 2 hours later by pronuclear injection of the DNA vector. Targeted blastocysts were identified by fluorescence of reporters. Cyto, cytoplasm; Nuc, nucleus.

TABLE 10
Mosaicism in targeted mice

| Donor | Mosaic/Total targeted | |
|---|---|---|
| Nanog-cherry | Mice | 1/8 |
| | ESCs | 2/6 |
| Oct4-EGFP | Mice | 0/3 |
| | ESCs | 1/3 |
| Mecp2-L2 + | Male | 11/28 |
| Mecpe-R1 | Female | 9/21 |
| | Total | 20/49* |

Targeted mice or ESCs were identified by RFLP, Southern bolt or Sequencing. The frequency of mosaicism in targeted mice was determined by fluorescent reporter or Southern blot analysis.
*These 49 mice contain at least one loxP integration.

TABLE 11
Off-target analysis

| | | Indel mutation | |
|---|---|---|---|
| | | frequency | |
| Site name | Sequence | (Mutant/Total) | Coordinate |
| Target_Sox2_Stop | TGCCCCTGTCGCACATGTGAGGG | / | chr3: 34550278-34550300 |
| | (SEQ ID NO: 216) | | |
| OT1_Sox2_Stop | TaCCCtTGTtGCACATGTGAAGG | 0/10 | chr4: 126636377-126636399 |
| | (SEQ ID NO: 217) | | |
| OT2_Sox2_Stop | TtCCCaTGTaGCACATGTGAGGG | 0/10 | chr14: 58830941-58830963 |
| | (SEQ ID NO: 218) | | |
| OT3_Sox2_Stop | TcCCCCTGTCaCACATGTGgTGG | 0/10 | chr1: 136641174-136641196 |
| | (SEQ ID NO: 219) | | |
| OT4_Sox2_Stop | TGCaCCTGTgGCACATGTGgGGG | 0/10 | chr9: 69081892-69081914 |
| | (SEQ ID NO: 220) | | |
| OT5_Sox2_Stop | TGCCaCaGTtGCACATGTGAGGG | 0/10 | chr1: 130633965-130633987 |
| | (SEQ ID NO: 221) | | |
| OT6_Sox2_Stop | TGCCaCTGTtGCAaATGTGAGGG | 0/10 | chr18: 61611640-61611662 |
| | (SEQ ID NO: 222) | | |
| OT7_Sox2_Stop | TGCCtCTGTCaCAgATGTGACGG | 0/10 | chr5: 136841014-136841036 |
| | (SEQ ID NO: 223) | | |
| OT8_Sox2_Stop | TGCCtCTGTCtCACATGTGcTGG | 0/10 | chr4: 141162434-141162456 |
| | (SEQ ID NO: 224) | | |
| OT9_Sox2_Stop | TGCCtCTGTCGCtCATGgGATGG | 0/10 | chr9: 48224429-48224451 |
| | (SEQ ID NO: 225) | | |
| OT10_Sox2_Stop | TGCCCaTGTCcCACATGgGATGG | 0/10 | chr7: 72596616-72596638 |
| | (SEQ ID NO: 226) | | |
| OT11_Sox2_Stop | TGCCCCTcTgtCACATGTGAAGG | 0/10 | chr18: 56473819-56473841 |
| | (SEQ ID NO: 227) | | |
| OT12_Sox2_Stop | TGCCCCTGTCatACATGTGgAGG | 0/10 | chr6: 98776389-98776411 |
| | (SEQ ID NO: 228) | | |
| OT13_Sox2_Stop | TGCCCCTGTCtCcCATGTGcTGG | 0/10 | chr4: 148868089-148868111 |
| | (SEQ ID NO: 229) | | |
| Target_Nanog_Stop | CGTAAGTCTCATATTTCACCTGG | / | chr6: 122663559-122663581 |
| | (SEQ ID NO: 230) | | |
| OT1_Nanog_Stop | tGTAAGTCTCATATTTCACCTGG | 7/9^{b} | chrX: 87128718-87128740 |
| | (SEQ ID NO: 231) | | |
| OT2_Nanog_Stop | CccAAGTCTCATtTTTCACCAGG | 0/9 | chr14: 21598653-21598675 |
| | (SEQ ID NO: 232) | | |
| OT3_Nanog_Stop | GaTAAGgaTCATATTTCACCCGG | 0/9 | chrX: 88301349-88301371 |
| | (SEQ ID NO: 233) | | |
| OT4_Nanog_Stop | TGcAAtTtTCATATTTCACCTGG | 0/9 | chr12: 71841888-71841910 |
| | (SEQ ID NO: 234) | | |
| OT5_Nanog_Stop | GGTcAtcCTCATATTTCACCAGG | 0/9 | chr11: 13346951-13346973 |
| | (SEQ ID NO: 235) | | |
| OT6_Nanog_Stop | TGTtAtTCaCATATTTCACCTGG | 0/9 | chr6:13888078-13888100 |
| | (SEQ ID NO: 236) | | |
| OT7_Nanog_Stop | TGTgAGTagCATATTTCACCTGG | 0/9 | chr 1 8:41037112-41037134 |
| | (SEQ ID NO: 237) | | |
| OT8_Nanog_Stop | TGTAAaTaaCATATTTCACCCGG | 0/9 | chrX: 70168441-70168463 |
| | (SEQ ID NO: 238) | | |
| OT9_Nanog_Stop | CaTAgagCTCATATTTCACCAGG | 0/9 | chr4: 80388067-80388089 |
| | (SEQ ID NO: 239) | | |
| Target_Mecp2_L2 | CCCAAGGATACAGTATCCTAGGG | / | chrX: 71282802-71282824 |
| | (SEQ ID NO: 240) | | |
| OT1_Mecp2_L2 | CCCAAGGATgCttTATCCTAAGG | 0/10 | chr8: 121441299-121441321 |
| | (SEQ ID NO: 241) | | |
| OT2_Mecp2_L2 | CCCAtGGATAgAGTAgCCTAAGG | 0/10 | chr15: 55526927-55526949 |
| | (SEQ ID NO: 242) | | |
| OT3_Mecp2_L2 | CCCAAGaATACAGTgTgCTAAGG | 0/10 | chr4: 83371755-83371777 |
| | (SEQ ID NO: 243) | | |
| OT4_Mecp2_L2 | CCCAAGGAcACAGgATCCcAAGG | 0/10 | chr17: 27887352-27887374 |
| | (SEQ ID NO: 244) | | |
| Target_Mecp2_R1 | AGGAGTGAGGTCTAGTACTTGGG | / | chrX: 71282103-71282125 |
| | (SEQ ID NO: 245) | | |
| OT1_Mecp2_R1 | AGGgGTGAGtTCTAGTACTTCGG | 0/10 | chr13: 48912459-48912481 |
| | (SEQ ID NO: 246) | | |
| OT2_Mecp2_R1 | TGGAGTGAGGTCTtGTACTTGGG | 1/10^{b} | chr12: 15404584-15404606 |
| | (SEQ ID NO: 247) | | |
| OT3_Mecp2_R1 | AGGAGTctGGgCTAGTACTTGGG | 0/10 | chr6: 115474148-115474170 |
| | (SEQ ID NO: 248) | | |

Mismatches from the on-target sequence are lower-case, boldface and underlined.
Indel mutation frequencies in targeted mice or ESCs were calculated by Suveryor assay.
Coordinate in which sites were located are shown.
OT, off-target;
/, not tested.
^{a}Nanog OT1 and 2 contain 3 bp mismatches; OT3 to 9 contain 4 bp mismatches lying in PAM distal region.
^{b}PCR products were cloned and sequenced to confirm off-target mutations.

TABLE 12
Oligonucleotides used in this study.
Oligonucleotides used for cloning sgRNA expression vector

| Gene target | Direction | Sequence (5′ to 3′) |
|---|---|---|
| Tet1 | F | Caccggctgctgtcagggagctca (SEQ ID NO: 249) |
| | R | Aaactgagctccctgacagcagcc (SEQ ID NO: 250) |
| Tet2 | F | caccgaaagtgccaacagatatcc (SEQ ID NO: 251) |
| | R | aaacggatatctgttggcactttc (SEQ ID NO: 252) |
| Sox2 | F | caccgtgcccctgtcgcacatgtga (SEQ ID NO: 253) |
| | R | aaactcacatgtgcgacaggggcac (SEQ ID NO: 254) |
| Nanog | F | caccgcgtaagtctcatatttcacc (SEQ ID NO: 255) |
| | R | aaacggtgaaatatgagacttacgc (SEQ ID NO: 256) |
| Oct4 | F | caccgctcagtgatgctgttgatc (SEQ ID NO: 257) |
| | R | aaacgatcaacagcatcactgagc (SEQ ID NO: 258) |
| Mecp2 L1 | F | caccgttgggccccagcttgaccca (SEQ ID NO: 259) |
| | R | aaactgggtcaagctggggcccaac (SEQ ID NO: 260) |
| Mecp2 L2 | F | caccgcccaaggatacagtatccta (SEQ ID NO: 261) |
| | R | aaactaggatactgtatccttgggc (SEQ ID NO: 262) |
| Mecp2 R1 | F | caccgaggagtgaggtctagtactt (SEQ ID NO: 263) |
| | R | aaacaagtactagacctcactcctc (SEQ ID NO: 264) |
| Mecp2 R2 | F | caccgtttggtggtggattaggtct (SEQ ID NO: 265) |
| | R | aaacagacctaatccaccaccaaac (SEQ ID NO: 266) |

Oligonucleotides used for RFLP analysis and PCR genotyping.

| Gene target | Direction | Sequence (5′ to 3′) |
|---|---|---|
| Tet1 | F | ttgttctctcctctgactgc (SEQ ID NO: 267) |
| | R | tgattgatcaaataggcctgc (SEQ ID NO: 268) |
| Tet2 | F | cagatgcttaggccaatcaag (SEQ ID NO: 269) |
| | R | agaagcaacacacatgaagatg (SEQ ID NO: 270) |
| Sax2 | SF | acatgatcagcatgtacctcc (SEQ ID NO: 271) |
| | SR | taatttggatgggattggtgg (SEQ ID NO: 272) |
| V5 | V5F | acatgggcaagcccatcc (SEQ ID NO: 273) |
| Mecp2 | LF | aatgtgccactttaacagcac (SEQ ID NO: 274) |
| L1, L2 | LR | ttctgatgtttctgctttgcc (SEQ ID NO: 275) |
| Mecp2 | RF | aagcatgagccactacaacc (SEQ ID NO: 276) |
| R1, R2 | RR | cttgctcagaagccaaaacag (SEQ ID NO: 277) |

Oligonucleotides used for making template for in vitro transcription

| Template | Direction | Sequence (5′ to 3′) |
|---|---|---|
| Cas9 | F | Taatacgactcactatagggagaatggactataaggacca |
| | | cgac (SEQ ID NO: 278) |
| | R | gcgagctctaggaattcttac (SEQ ID NO: 279) |
| Tet1 | F | Ttaatacgactcactataggctgctgtcagggagctc |
| sgRNA | | (SEQ ID NO: 280) |
| | R | aaaagcaccgactcggtgcc (SEQ ID NO: 281) |
| Tet2 | F | Ttaatacgactcactataggaaagtgccaacagatatcc |
| sgRNA | | (SEQ ID NO: 282) |
| | R | aaaagcaccgactcggtgcc (SEQ ID NO: 283) |
| Sox2 | F | Ttaatacgactcactatagtgcccctgtcgcacatgtga |
| sgRNA | | (SEQ ID NO: 284) |
| | R | aaaagcaccgactcggtgcc (SEQ ID NO: 285) |
| Nanog | F | Ttaatacgactcactatagcgtaagtctcatatttcacc |
| sgRNA | | (SEQ ID NO: 286) |
| | R | aaaagcaccgactcggtgcc (SEQ ID NO: 287) |
| Oct4 | F | Ttaatacgactcactataggctcagtgatgctgttgatc |
| sgRNA | | (SEQ ID NO: 288) |
| | R | aaaagcaccgactcggtgcc (SEQ ID NO: 289) |
| Mecp2-L1 | F | Ttaatacgactcactatagttgggccccagcttgaccca |
| sgRNA | | (SEQ ID NO: 290) |
| | R | aaaagcaccgactcggtgcc (SEQ ID NO: 291) |
| Mecp2-L2 | F | Ttaatacgactcactatagcccaaggatacagtatccta |
| sgRNA | | (SEQ ID NO: 292) |
| | R | aaaagcaccgactcggtgcc (SEQ ID NO: 293) |
| Mecp2-R1 | F | Ttaatacgactcactatagaggagtgaggtctagtactt |
| sgRNA | | (SEQ ID NO: 294) |
| | R | aaaagcaccgactcggtgcc (SEQ ID NO: 295) |
| Mecp2-R2 | F | Ttaatacgactcactatagtttggtggtggattaggtct |
| sgRNA | | (SEQ ID NO: 296) |
| | R | aaaagcaccgactcggtgcc (SEQ ID NO: 297) |

Oligonucleotides used for HDR-mediated repair through embryo
injection

| Gene | |
|---|---|
| target | Sequence (5′ to 3′) |
| Tet1-loxP | Gaaaaaggcccatattatacacaccttggggcaggaccaagtgtggctgctgtcaggga |
| | gGAATTCataacttcgtataatgtatgctatacgaagttatctcatggagactaggtga |
| | ggaactctgcttcccgctaacccattcttcccggtgacctgg (SEQ ID NO: 298) |
| Tet2-loxP | Ctctgtgactataaggctctgactctcaagtcacagaaacacgtgaaagtgccaacaga |
| | tGAATTCataacttcgtataatgtatgctatacgaagttatatccaggctgcagaatcg |
| | gagaaccacgcccgagctgcagagcctcaagcaaccaaaagc (SEQ ID NO: 299) |
| Sox2-v5 | Taccagagcggcccggtgcccggcacggccattaacggcacactgcccctgtcgcacat |
| | gGGCAAGCCCATCCCCAACCCCCTGCTGGGCCTGGACAGCACCtgagggctggactgcg |
| | aactggagaaggggagagattttcaaagagatacaagggaattg (SEQ ID NO: 300) |
| Mecp2-L1-loxP | Tgtttgaccaatatcaccagcaacctaaagctgttaagaaatctttgggccccagcttg |
| | aGCTAGCataacttcgtataatgtatgctatacgaagttatcccaaggatacagtatcc |
| | tagggaagttaccaaaatcagagatagtatgcagcagccagg (SEQ ID NO: 301) |
| Mecp2-L2-loxP | Ccagcaacctaaagctgttaagaaatctttgggccccagcttgacccaaggatacagta |
| | tGCTAGCataacttcgtataatgtatgctatacgaagttatcctagggaagttaccaaa |
| | atcagagatagtatgcagcagccaggggtctcatgtgtggca (SEQ ID NO: 302) |
| Mecp2-R1-loxP | Ccactcctctgtactccctggcttttccacaatccttaaactgaaggagtgaggtctag |
| | tataacttcgtatagcatacattatacgaagttatGAATTCacttgggggtcattgggc |
| | tagactgaatatctttggttggtacccagacctaatccacca (SEQ ID NO: 303) |
| Mecp2-R2-loxP | Ccaaaaaggctggacaccatgccttggttaaaatggaggaatgttttggtggtggatta |
| | gGAATTCataacttcgtataatgtatgctatacgaagttatgtctgggtaccaaccaaa |
| | gatattcagtctagcccaatgacccccaagtactagacctca (SEQ ID NO: 304) |

Oligonucleotides used for off-targeted analysis

| Gene target | Direction | Sequence (5′ to 3′) |
|---|---|---|
| OT1_Sox2_Stop | F | Atgacatgacctaagtaaaccc (SEQ ID NO: 305) |
| | R | Ctccactctgtactaggcac (SEQ ID NO: 306) |
| OT2_Sox2_Stop | F | tgatggtttttggtgactgcc (SEQ ID NO: 307) |
| | R | gacagatcatagatagaaaattg (SEQ ID NO: 308) |
| OT3_Sox2_Stop | F | aaactgaggcacagagtctg (SEQ ID NO: 309) |
| | R | gtgacgaagccactttgacc (SEQ ID NO: 310) |
| OT4_Sox2_Stop | F | caccttaggttcatggcattc (SEQ ID NO: 311) |
| | R | gatggatcagtgattaagagc (SEQ ID NO: 312) |
| OT5_Sox2_Stop | F | accatgatggactgtaccatc (SEQ ID NO: 313) |
| | R | catggacgtcattactagatg (SEQ ID NO: 314) |
| OT6_Sox2_Stop | F | ttcctcgaagatgaaatgatt (SEQ ID NO: 315) |
| | R | cagtgtgcagactctgagag (SEQ ID NO: 316) |
| OT7_Sox2_Stop | F | atgtgccacacaaggcaggc (SEQ ID NO: 317) |
| | R | gcaaaacctctgaaagttgac (SEQ ID NO: 318) |
| OT8_Sox2_Stop | F | ttcctgtcctggcttccttc (SEQ ID NO: 319) |
| | R | gcactagttgtcacgtgatg (SEQ ID NO: 320) |
| OT9_Sox2_Stop | F | gactcagatttccaagccatg (SEQ ID NO: 321) |
| | R | acatctctgagctctaagcc (SEQ ID NO: 322) |
| OT10_Sox2_Stop | F | tgccatgtgctgtgttcacc (SEQ ID NO: 323) |
| | R | ttgatatttaagacagggtctc (SEQ ID NO: 324) |
| OT11_Sox2_Stop | F | gtaaggaatgtaagaactcttg (SEQ ID NO: 325) |
| | R | aattctcaactgaggaatactg (SEQ ID NO: 326) |
| OT12_Sox2_Stop | F | tctcagacagaaacgctgtg (SEQ ID NO: 327) |
| | R | gacttgatatgccaggatgag (SEQ ID NO: 328) |
| OT13_Sox2_Stop | F | agctgacagaagacgatgag (SEQ ID NO: 329) |
| | R | taaacccaagcaaaggtcatg (SEQ ID NO: 330) |
| OT1_Nanog_Stop | F | gctggtgagatggctcagtg (SEQ ID NO: 331) |
| | R | gtcttaacctgcttatagcaac (SEQ ID NO: 332) |
| OT2_Nanog_Stop | F | agatcccattacggatggttg (SEQ ID NO: 333) |
| | R | ggacactcaccaatgtttgg (SEQ ID NO: 334) |
| OT3_Nanog_Stop | F | tagattatctagtgtgttccac (SEQ ID NO: 335) |
| | R | agtttcagtgctcagagcac (SEQ ID NO: 336) |
| OT4_Nanog_Stop | F | gacactttctaagtgggcttg (SEQ ID NO: 337) |
| | R | gttaagggacagtgaatatcc (SEQ ID NO: 338) |
| OTS_Nanog_Stop | F | tcccatctaccctctgactg (SEQ ID NO: 339) |
| | R | gcctgaagaaaagaaggtcc (SEQ ID NO: 340) |
| OT6_Nanog_Stop | F | tctgaggtgagcaaagcatg (SEQ ID NO: 341) |
| | R | aatccaccatgtcttccgtg (SEQ ID NO: 342) |
| OT7_Nanog_Stop | F | caattttctcagtgaggtagg (SEQ ID NO: 343) |
| | R | cttgttcagtgcattgctgc (SEQ ID NO: 344) |
| OT8_Nanog_Stop | F | tctcttcagaaaagagtaggc (SEQ ID NO: 345) |
| | R | gttggcaactgcactctgtg (SEQ ID NO: 346) |
| OT9_Nanog_Stop | F | agctcatgcatgctgagctg (SEQ ID NO: 347) |
| | R | aacttcaagtggaactgcttg (SEQ ID NO: 348) |
| OT1_Mecp2 Left2 | F | cacacacacactgaataaaatg (SEQ ID NO: 349) |
| | R | aagctggctttgagcaggac (SEQ ID NO: 350) |
| OT2_Mecp2 Left2 | F | tagtcacttatgtttactcctc (SEQ ID NO: 351) |
| | R | gtgatgccagcagttggcag (SEQ ID NO: 352) |
| OT3_Mecp2 Left2 | F | tcactttccctcagtactcc (SEQ ID NO: 353) |
| | R | caagtatcattctctgaacaag (SEQ ID NO: 354) |
| OT4_Mecp2 Left2 | F | gaactttgagacagggtctc (SEQ ID NO: 355) |
| | R | gacagagcagcttggccttc (SEQ ID NO: 356) |
| OT1_Mecp2_Right1 | F | gcagcaccagtggaatattac (SEQ ID NO: 357) |
| | R | gcctattgatgaatctgccc (SEQ ID NO: 358) |
| OT2_Mecp2_Right1 | F | acagatgcagccactcacag (SEQ ID NO: 359) |
| | R | gtccaagtcacttctcccac (SEQ ID NO: 360) |
| OT3_Mecp2_Right1 | F | tccgacaatggtttatgtctg (SEQ ID NO: 361) |
| | R | agatactagcagtgcagctg (SEQ ID NO: 362) |
| OT4_Mecp2_Right1 | F | gttcctgctggttttgtttcg (SEQ ID NO: 363) |
| | R | tagaccaatctacaaccacag (SEQ ID NO: 364) |
| OT5_Mecp2_Right1 | F | tgctgtgaaactcaggcatg (SEQ ID NO: 365) |
| | R | cttctaagacaagccagaaag (SEQ ID NO: 366) |
| OT6_Mecp2_Right1 | F | cggcataaacctcccattag (SEQ ID NO: 367) |
| | R | ctctgtgcttgtaaggcaaac (SEQ ID NO: 368) |
| OT7_Mecp2_Right1 | F | gccagacaataattcccaag (SEQ ID NO: 369) |
| | R | ctgatattgctactgctaacc (SEQ ID NO: 370) |
| OT8_Mecp2_Right1 | F | ccattgtgaaagtgggatgc (SEQ ID NO: 371) |
| | R | ggctgctctcgtaaacaaaac (SEQ ID NO: 372) |
| OT9_Mecp2_Right1 | F | gtcactctcatgtgcaggtg (SEQ ID NO: 373) |
| | R | ctagcacttgggaagcaaatg (SEQ ID NO: 374) |
| OT10_Mecp2_Right1 | F | ctaatcacacttctacaagctg (SEQ ID NO: 375) |
| | R | agagaggctccaattgttag (SEQ ID NO: 376) |

### Example 4

### Multiplexed Activation of Endogenous Genes by CRISPR-On, a RNA-Guided Transcriptional Activator System

As described in Example 2, a two-component transcriptional activator consisting of a nuclease-dead Cas9 (dCas9) protein fused with a transcriptional activation domain and single guide RNAs (sgRNAs) with complementary sequence to gene promoters. It is demonstrated that CRISPR-on can efficiently activate exogenous reporter genes in both human and mouse cells in a tunable manner. In addition, robust reporter gene activation in vivo can be achieved by injecting the system components into mouse zygotes. Furthermore, CRISPR-on can activate the endogenous IL1RN, SOX2, and OCT4 genes. The most efficient gene activation was achieved by clusters of 3 to 4 sgRNAs binding to the proximal promoters suggesting their synergistic action in gene induction. Significantly, when sgRNAs targeting multiple genes were simultaneously introduced into cells, robust multiplexed endogenous gene activation was achieved. Genome-wide expression profiling demonstrated high specificity of the system.

Materials and Methods

Cloning

A two-step fusion PCR was performed to amplify Cas9 Nickase ORF without stop codon from the pX335 vector (Addgene: 42335), incorporate H840A mutation, EcoRI-AgeI restriction site on the 5′ end as well as an FseI site on the 3′end (EcoRI-AgeI-dCas9-FseI fragment). The 3× minimal VP16 activation domain coding fragment (VP48) was excised from a vector (Addgene: 20342) containing NLSM2rtTA coding sequence by FseI and EcoRI digestion (FseI-TA-EcoRI fragment). The two fragments were ligated into pCR8/GW/TOPO (Invitrogen) vector digested by EcoRI to generate a gateway compatible dCas9VP48 coding plasmid. The dCas9VP48 coding sequence was subsequently excised and cloned into pX355 vector (Addgene: 42335) by AgeI-EcoRI digestion to replace dCas9 Nickase to create a chimeric vector that expresses both the dCas9VP48 and the sgRNA (dCas9VP48-U6-sgRNA-chimeric). sgRNA spacers were cloned into the BbsI-digested vector by annealing oligos as previously described (Cong et al., Science; 339 (6121):819-823(2013)). For construction of dCas9VP160 (SEQ ID NO:16), a gBlocks gene fragment containing coding sequence for 10 tandem repeats of VP16 domains separated by Glycine-Serine (GS) linker was ordered from Integrated DNA Technology (IDT) and amplified by PCR primers containing FseI and EcoRI sites to replace VP48 fragment in pCR8-dCas9VP48 to generate pCR8-dCas9VP160. A pmax-DEST gateway destination vector was constructed by replacing GFP coding sequence in pmaxGFP (Clontech) by a gateway destination cassette (Invitrogen). The pCR8-dCas9VP160 vector was then recombined with pmax-DEST via LR Clonase-medicated to create pmax-dCas9VP160 expression plasmid. For the endogenous gene experiments, sgRNAs were cloned by oligo clonding method mentioned above to a PBneo-sgRNA expression vector.

Culturing and Transfection of HeLa, HEK293T and NIH3T3

HeLa, HEK293T and NIH3T3 cells were cultured in DMEM with 10% inactivated FBS, 1% Penn/Strep, 1% Glutamine, 1% non-essential amino acids. Transfection was done using Fugene HD (Promega) using a 2:6 ratio (A total DNA amount of 2 μg and 6 μl of Fugene HD reagent) in 6-well plates. For TetO::tdTomato experiments, 2 μg of the chimeric vector was used. For endogenous gene activation experiments, the U6 promoter—sgRNA—terminator sequence was amplified from the PBneo-sgRNA plasmids, purified by PCR purification kit (QIAGEN), and transfected as linear DNA (1 μg Total sgRNA expressing DNA) with 1 μg of pmax-dCas9VP160 plasmid. Where there are multiple sgRNAs for multiple genes, the amount per sgRNA was evenly divided among genes first, then among the sgRNAs targeting each gene.

Transgene Activation Experiment in Mouse Embryonic Stem Cells (mESC)

Mouse ESCs from mice carrying a Dox-inducible Musashi-1 (MSI1) allele in the Col1A1 locus (Kharas et al., Nature medicine; 16 (8):903-908 (2010)) were transfected with dCas9VP48 using Xfect mESC transfection reagent (Clontech) or were cultured in mouse ES medium with 2 μg/ml Doxycycline. 48 hours later, Protein lysates were prepared on ice from cell pellets in SDS-Tris lysis buffer (10% SDS, 10% Glycerol, 0.1M DTT, 0.12 g/ml Urea) supplemented with protease and phosphatase inhibitor tables (1 tablet/10 ml, Roche) and analyzed by western blot. Blots were probed with primary rabbit anti-MSI1 (Cell Signaling Technologies, #2154), mouse anti-Alpha-Tubulin (SIGMA) antibodies. Secondary HRP-conjugated anti-rabbit/anti-mouse IgG were used and visualized with ECL (GE Healthcare).

One Cell Embryo Injection

All animal procedures were performed according to NIH guidelines and approved by the Committee on Animal Care at MIT. B6D2F1 (C57BL/6×DBA2) female mice and ICR mouse strains were used as embryo donors and foster mothers, respectively. Super-ovulated female B6D2F1 mice (7-8 weeks old) were mated to B6D2F1 stud males, and fertilized embryos were collected from oviducts. Cas9VP48 plasmid (200 ng/μl), Nanog::EGFP construct (200 ng/μl), and sgRNAs (50 ng/μl for each) were mixed and injected into the cytoplasm of fertilized eggs with well-recognized pronuclei in M2 medium (Sigma). Injected oocytes were cultured in KSOM medium for 96 h to examine their development in vitro. Images of resulting embryos were acquired with an inverted microscope under the same exposure parameters.

Bioinformatics Analysis of Gene Expression and CRISPR Off-Target Analysis

Affymetrix U133A 2.0 array was used for microarray gene expression analysis. Gene expression values were processed and normalized using affy package for R {Gautier, 2004 #27}.

qRT-PCR Expression Analysis

Total RNA was isolated using the Rneasy Kit (QIAGEN) and reversed transcribed using the Superscript III First Strand Synthesis kit (Invitrogen). Quantitative RT-PCR analysis was performed in triplicate using the ABI 7900 HT system with FAST SYBR Green Master Mix (Applied Biosystems). Gene expression was normalized to GAPDH. Error bars represent the standard deviation (SD) of the mean of triplicate reactions. Primer sequences are included in Table 13.

TABLE 13
qRT-PCR primers.

| Gene | Forward primer | Reverse primer |
|---|---|---|
| SOX2 | CACCTACAGCATGTCCTACT | GGTTTTCTCCATGCTGTTTC |
| | CG | TT |
| | (SEQ ID NO: 377) | (SEQ ID NO: 378) |
| IL1RN | GACCCTCTGGGAGAAAATCC | GTCCTTGCAAGTATCCAGCA |
| | (SEQ ID NO: 379) | (SEQ ID NO: 380) |
| OCT4 | GCTCGAGAAGGATGTGGTCC | CGTTGTGCATAGTCGCTGCT |
| | (SEQ ID NO: 381) | (SEQ ID NO: 382) |
| GAPDH | CGAGATCCCTCCAAAATCAA | ATCCACAGTCTTCTGGGTGG |
| | (SEQ ID NO: 383) | (SEQ ID NO: 384) |

TABLE 14
sgRNA designs, DNA targets, oligos for cloning.

| Name | Target | Target_sequences | Forward oligo | Reverse oligo |
|---|---|---|---|---|
| sgTetO | Tet binding | gCTTTTCTCTATC | caccGCTTTTCTCTAT | aaacTATCAGTGAT |
| | site | ACTGATAGGG | CACTGATA | AGAGAAAAGC |
| | | (SEQ ID NO: 385) | (SEQ ID NO: 386) | (SEQ ID NO: 387) |
| sgTetOMut | Mutant | gCTTTTCTtTAtCAt | caccGCTTTTCTTTAT | aaacTACCAATGAT |
| | version of | TGgTAGGG | CATTGGTA | AAAGAAAAGC |
| | sgTetO | (SEQ ID NO: 388) | (SEQ ID NO: 389) | (SEQ ID NO: 390) |
| sgNanog-1 | mouse Nanog | GTAATGCAAAAG | caccGTAATGCAAAA | aaacTACAGCTTCT |
| | | AAGCTGTAAGG | GAAGCTGTA | TTTGCATTAC |
| | | (SEQ ID NO: 391) | (SEQ ID NO: 392) | (SEQ ID NO: 393) |
| sgNanog-2 | mouse Nanog | GATCTCTAGTGG | caccGATCTCTAGTG | aaacGAAACTTCCC |
| | | GAAGTTTCAGG | GGAAGTTTC | ACTAGAGATC |
| | | (SEQ ID NO: 394) | (SEQ ID NO: 395) | (SEQ ID NO: 396) |
| sgNanog-3 | mouse Nanog | GTCTGTAGAAAG | caccGTCTGTAGAAA | aaacCTTCCATTCT |
| | | AATGGAAGAGG | GAATGGAAG | TTCTACAGAC |
| | | (SEQ ID NO: 397) | (SEQ ID NO: 398) | (SEQ ID NO: 399) |
| sgNanog-4 | mouse Nanog | GCTCTTCACATTG | caccGCTCTTCACATT | aaacGGTTTCCCAA |
| | | GGAAACCTGG | GGGAAACC | TGTGAAGAGC |
| | | (SEQ ID NO: 400) | (SEQ ID NO: 401) | (SEQ ID NO: 402) |
| sgNanog-5 | mouse Nanog | GCGTTAAAAAGC | caccGCGTTAAAAAG | aaacAAAGTGCGGC |
| | | CGCACTTTTGG | CCGCACTTT | TTTTTAACGC |
| | | (SEQ ID NO: 403) | (SEQ ID NO: 404) | (SEQ ID NO: 405) |
| sgNanog-6 | mouse Nanog | GAGTGTTTAAATT | caccGAGTGTTTAAA | aaacCTACATTAAT |
| | | AATGTAGAGG | TTAATGTAG | TTAAACACTC |
| | | (SEQ ID NO: 406) | (SEQ ID NO: 407) | (SEQ ID NO: 408) |
| sgNanog-7 | mouse Nanog | GAGTTTCACGTAC | caccGAGTTTCACGT | aaacGTCTCGGGTA |
| | | CCGAGACTGG | ACCCGAGAC | CGTGAAACTC |
| | | (SEQ ID NO: 409) | (SEQ ID NO: 410) | (SEQ ID NO: 411) |
| sgIL1RN-1 | IL1RN | GCACCTCAGAGA | caccGCACCTCAGAG | aaacCCTTGTACTC |
| | | GTACAAGGAGG | AGTACAAGG | TCTGAGGTGC |
| | | (SEQ ID NO: 412) | (SEQ ID NO: 413) | (SEQ ID NO: 414) |
| sgIL1RN-2 | IL1RN | gGGCTGACTTGAT | caccGGGCTGACTTG | aaacGCTTGGCATC |
| | | GCCAAGCAGG | ATGCCAAGC | AAGTCAGCCC |
| | | (SEQ ID NO: 415) | (SEQ ID NO: 416) | (SEQ ID NO: 417) |
| sgIL1RN-3 | IL1RN | gGTTTCCAGGAGG | caccGGTTTCCAGGA | aaacGAGTCACCCT |
| | | GTGACTCAGG | GGGTGACTC | CCTGGAAACC |
| | | (SEQ ID NO: 418) | (SEQ ID NO: 419) | (SEQ ID NO: 420) |
| sgIL1RN-4 | IL1RN | gGGTTCTTATCTG | caccGGGTTCTTATCT | aaacTCTTACGCAG |
| | | CGTAAGATGG | GCGTAAGA | ATAAGAACCC |
| | | (SEQ ID NO: 421) | (SEQ ID NO: 422) | (SEQ ID NO: 423) |
| sgIL1RN-5 | IL1RN | gATTGGGAACAA | caccGATTGGGAACA | aaacTGTCTGGCTT |
| | | GCCAGACAAGG | AGCCAGACA | GTTCCCAATC |
| | | (SEQ ID NO: 424) | (SEQ ID NO: 425) | (SEQ ID NO: 426) |
| sgIL1RN-6 | IL1RN | GATATGCTTTTGA | caccGATATGCTTTTG | aaacAGGTCCCTCA |
| | | GGGACCTAGG | AGGGACCT | AAAGCATATC |
| | | (SEQ ID NO: 427) | (SEQ ID NO: 428) | (SEQ ID NO: 429) |
| sgSOX2-1 | SOX2 | GGGGAGAGGAGG | caccGGGGAGAGGAG | aaacCCTCCCCTCC |
| | | AGGGGAGGCGG | GAGGGGAGG | TCCTCTCCCC |
| | | (SEQ ID NO: 430) | (SEQ ID NO: 431) | (SEQ ID NO: 432) |
| sgSOX2-2 | SOX2 | GAGAGAGGCAAA | caccGAGAGAGGCAA | aaacGATTCCAGTT |
| | | CTGGAATCAGG | ACTGGAATC | TGCCTCTCTC |
| | | (SEQ ID NO: 433) | (SEQ ID NO: 434) | (SEQ ID NO: 435) |
| sgSOX2-3 | SOX2 | gCATGTGACGGG | caccGCATGTGACGG | aaacTGACAGCCCC |
| | | GGCTGTCAGGG | GGGCTGTCA | CGTCACATGC |
| | | (SEQ ID NO: 436) | (SEQ ID NO: 437) | (SEQ ID NO: 438) |
| sgSOX2-4 | SOX2 | GCTGCCGGGTTTT | caccGCTGCCGGGTT | aaacTTCATGCAAA |
| | | GCATGAAAGG | TTGCATGAA | ACCCGGCAGC |
| | | (SEQ ID NO: 439) | (SEQ ID NO: 440) | (SEQ ID NO: 441) |
| sgSOX2-5 | SOX2 | GCCGGCCGCGCG | caccGCCGGCCGCGC | aaacGCCTCCCCCG |
| | | GGGGAGGCCGG | GGGGGAGGC | CGCGGCCGGC |
| | | (SEQ ID NO: 442) | (SEQ ID NO: 443) | (SEQ ID NO: 444) |
| sgSOX2-6 | SOX2 | GGCAGGCGAGGA | caccGGCAGGCGAGG | aaacCCTCCCCCTC |
| | | GGGGGAGGAGG | AGGGGGAGG | CTCGCCTGCC |
| | | (SEQ ID NO: 445) | (SEQ ID NO: 446) | (SEQ ID NO: 447) |
| sgSOX2-7 | SOX2 | GTATCCCCTCTCG | caccGTATCCCCTCTC | aaacGTTGCTGCGA |
| | | CAGCAACAGG | GCAGCAAC | GAGGGGATAC |
| | | (SEQ ID NO: 448) | (SEQ ID NO: 449) | (SEQ ID NO: 450) |
| sgSOX2-8 | SOX2 | GCAGGGTACTTA | caccGCAGGGTACTT | aaacTCCTCATTTA |
| | | AATGAGGATGG | AAATGAGGA | AGTACCCTGC |
| | | (SEQ ID NO: 451) | (SEQ ID NO: 452) | (SEQ ID NO: 453) |
| sgSOX2-9 | SOX2 | GCAGCTAAGGTG | caccGCAGCTAAGGT | aaacCACCCCCGCA |
| | | CGGGGGTGGGG | GCGGGGGTG | CCTTAGCTGC |
| | | (SEQ ID NO: 454) | (SEQ ID NO: 455) | (SEQ ID NO: 456) |
| sgS0X2-10 | SOX2 | GGCTGTCCAACTC | caccGGCTGTCCAAC | aaacGAAATACGA |
| | | GTATTTCTGG | TCGTATTTC | GTTGGACAGCC |
| | | (SEQ ID NO: 457) | (SEQ ID NO: 458) | (SEQ ID NO: 459) |
| sgOCT4-1 | OCT4 | GAAGGAAGGCGC | caccGAAGGAAGGCG | aaacGGCTTGGGGC |
| | | CCCAAGCCGGG | CCCCAAGCC | GCCTTCCTTC |
| | | (SEQ ID NO: 460) | (SEQ ID NO: 461) | (SEQ ID NO: 462) |
| sgOCT4-2 | OCT4 | GGTGAAATGAGG | caccGGTGAAATGAG | AaacTCGCAAGCC |
| | | GCTTGCGAAGG | GGCTTGCGA | CTCATTTCACC |
| | | (SEQ ID NO: 463) | (SEQ ID NO: 464) | (SEQ ID NO: 465) |
| sgOCT4-3 | OCT4 | GGCCCCGCCCCCT | caccGGCCCCGCCCC | aaacCCCATCCAGG |
| | | GGATGGGTGG | CTGGATGGG | GGGCGGGGCC |
| | | (SEQ ID NO: 466) | (SEQ ID NO: 467) | (SEQ ID NO: 468) |
| sgOCT4-4 | OCT4 | GGGGGGAGAAAC | caccGGGGGGAGAAA | aaacTCGCCTCAGT |
| | | TGAGGCGAAGG | CTGAGGCGA | TTCTCCCCCC |
| | | (SEQ ID NO: 469) | (SEQ ID NO: 470) | (SEQ ID NO: 471) |
| sgOCT4-5 | OCT4 | GGTGGTGGCAAT | caccGGTGGTGGCAA | aaacCAGACACCAT |
| | | GGTGTCTGTGG | TGGTGTCTG | TGCCACCACC |
| | | (SEQ ID NO: 472) | (SEQ ID NO: 473) | (SEQ ID NO: 474) |
| sgOCT4-6 | OCT4 | GACACAACTGGC | caccGACACAACTGG | aaacGGAGGGGCG |
| | | GCCCCTCCAGG | CGCCCCTCC | CCAGTTGTGTC |
| | | (SEQ ID NO: 475) | (SEQ ID NO: 476) | (SEQ ID NO: 477) |

Last three bases are PAM (5′-NGG-3′) motif.
Lowercase italic letters in the target sequences indicate mismatch (first g to allow efficient U6 transcription) or as mutant control (other changes; as in sgTetO-mut).
Lowercase letters in the oligo sequences indicate overhang compatible to the BbsI-digested vectors.

Results

Fusion of Nuclease-Deficient Cas9 to Transactivation Domain Generated an RNA-Programmable Transcription Factor

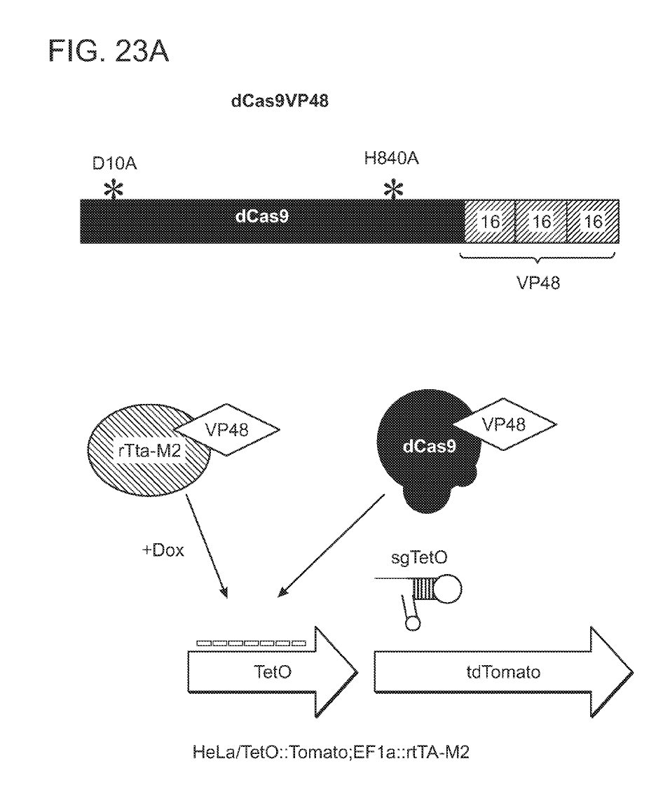

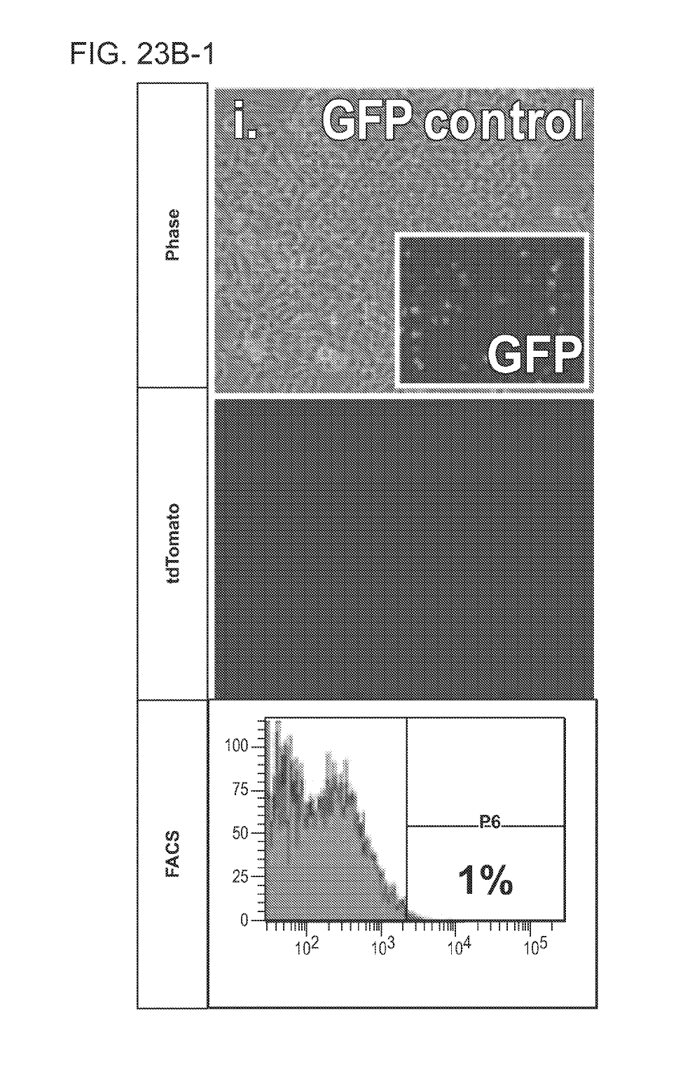

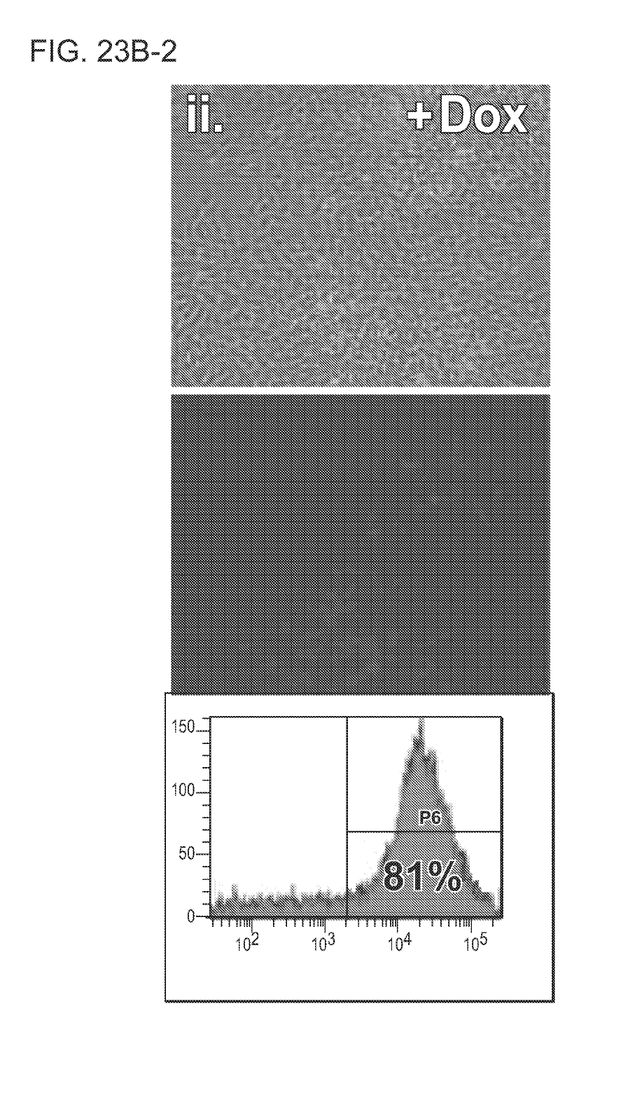

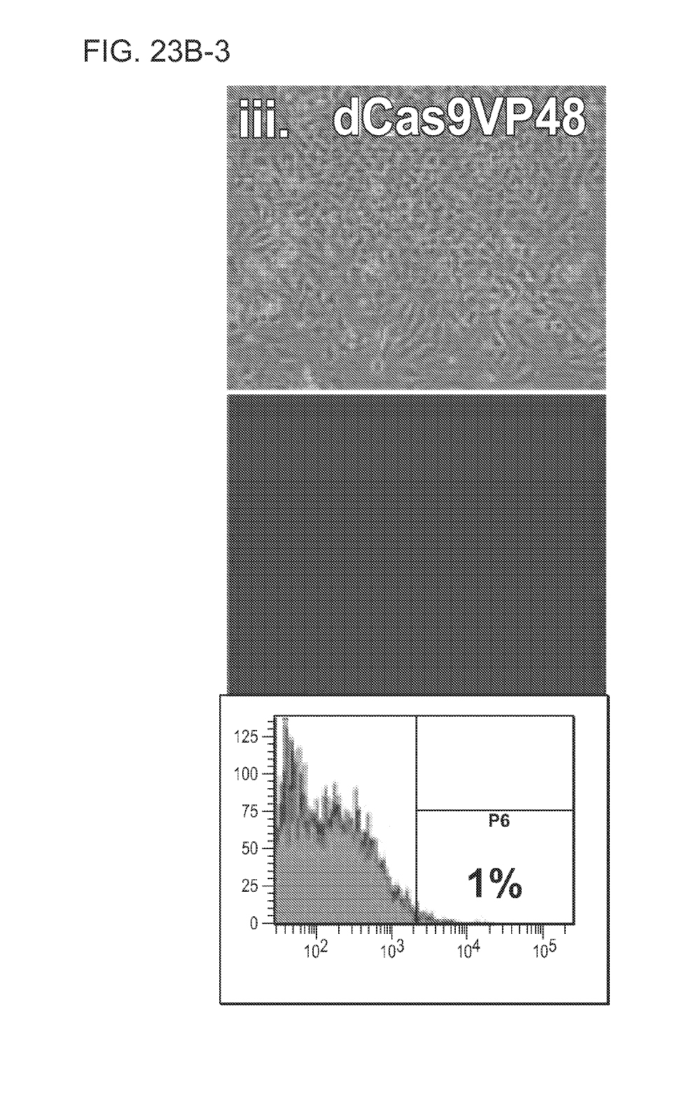

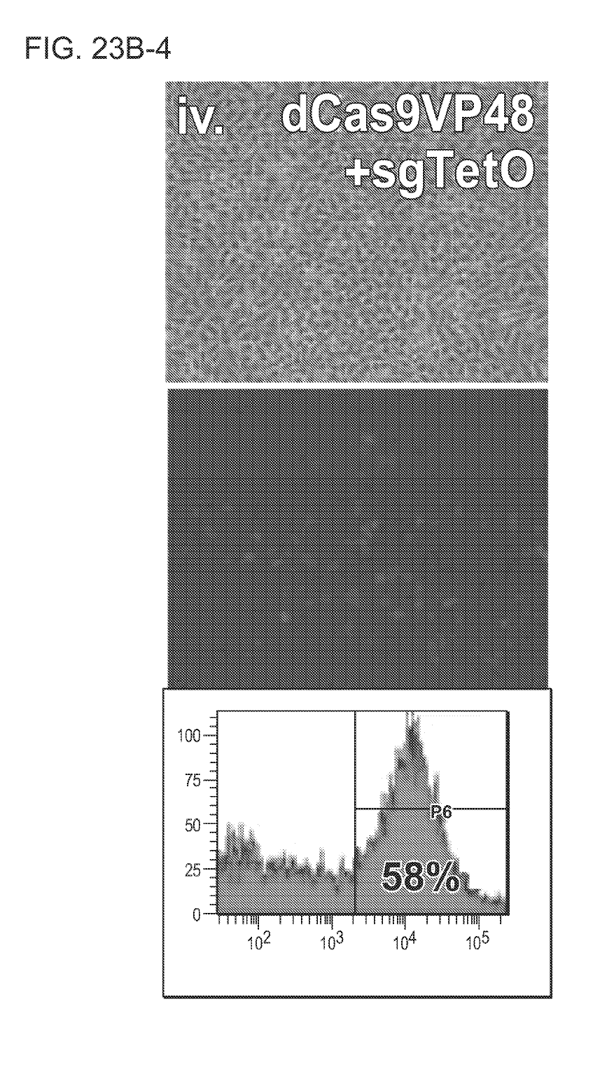

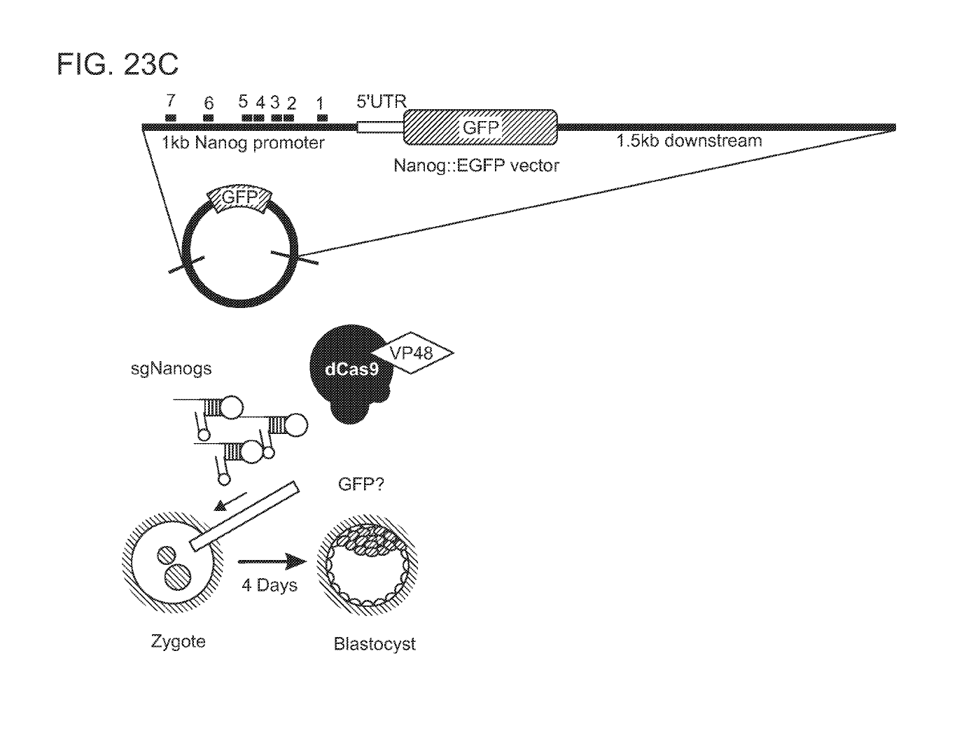

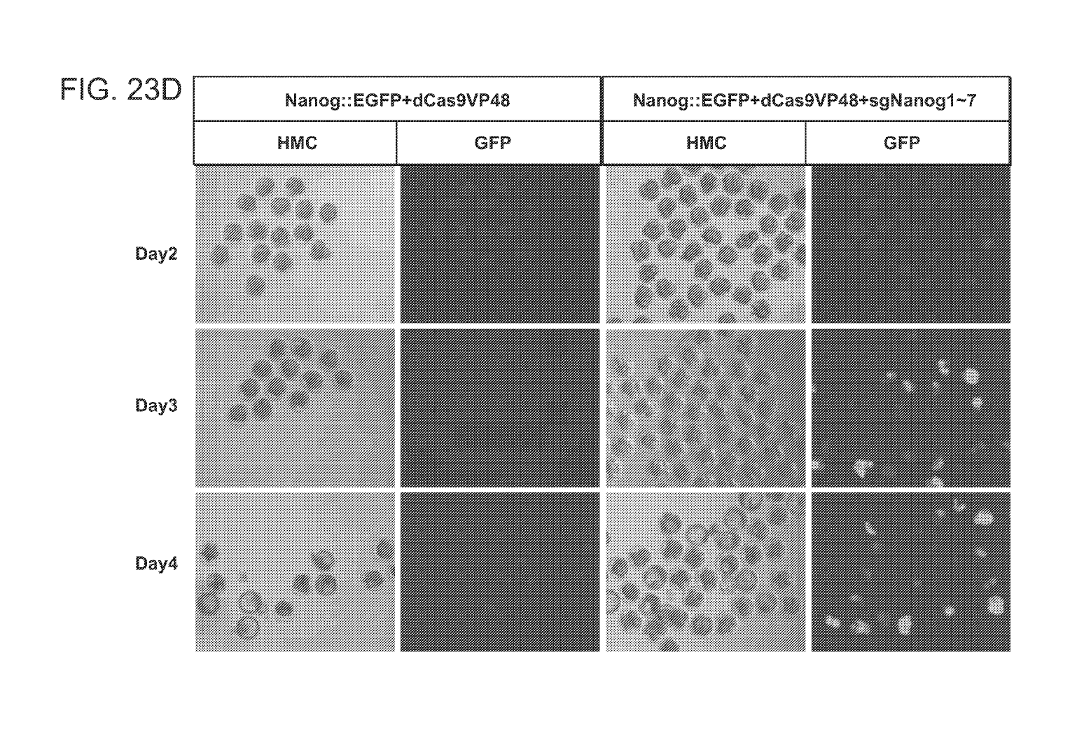

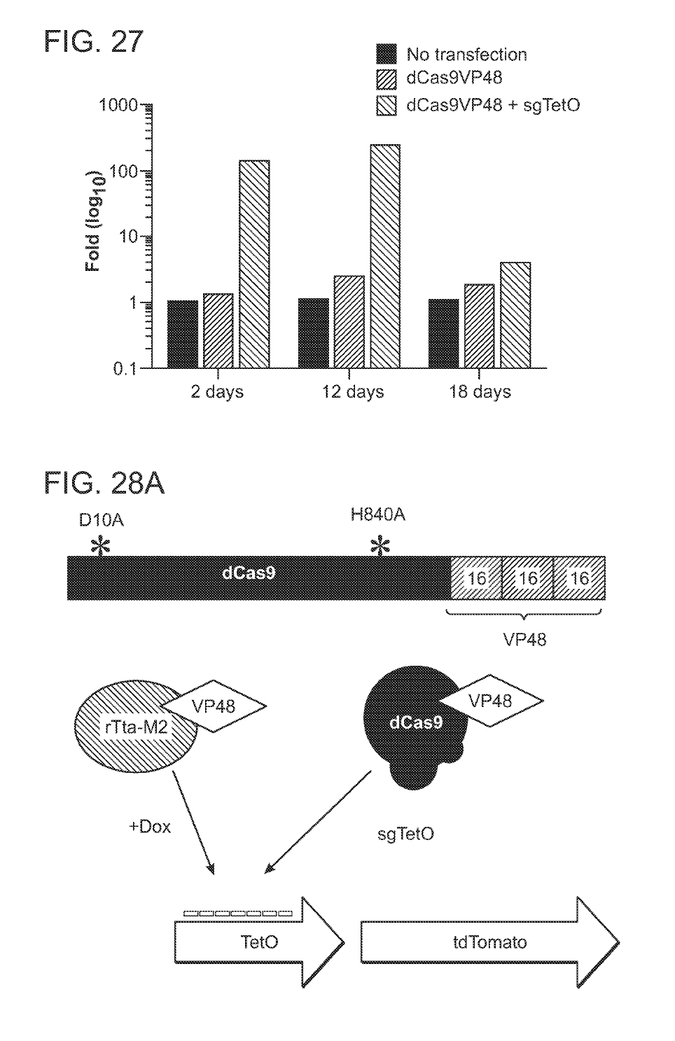

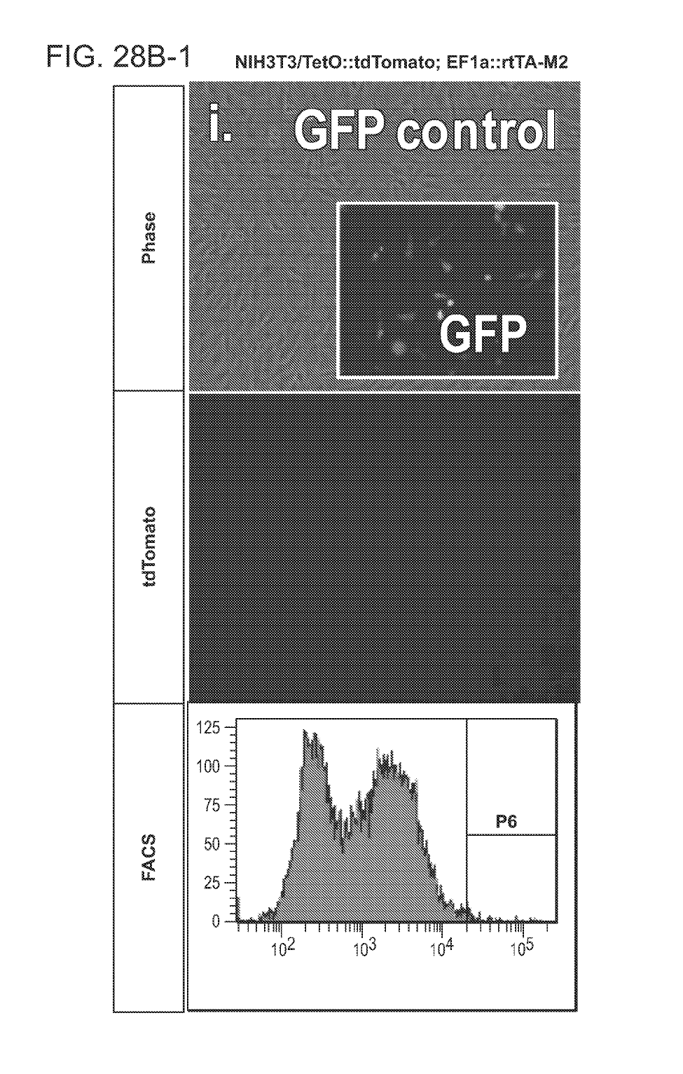

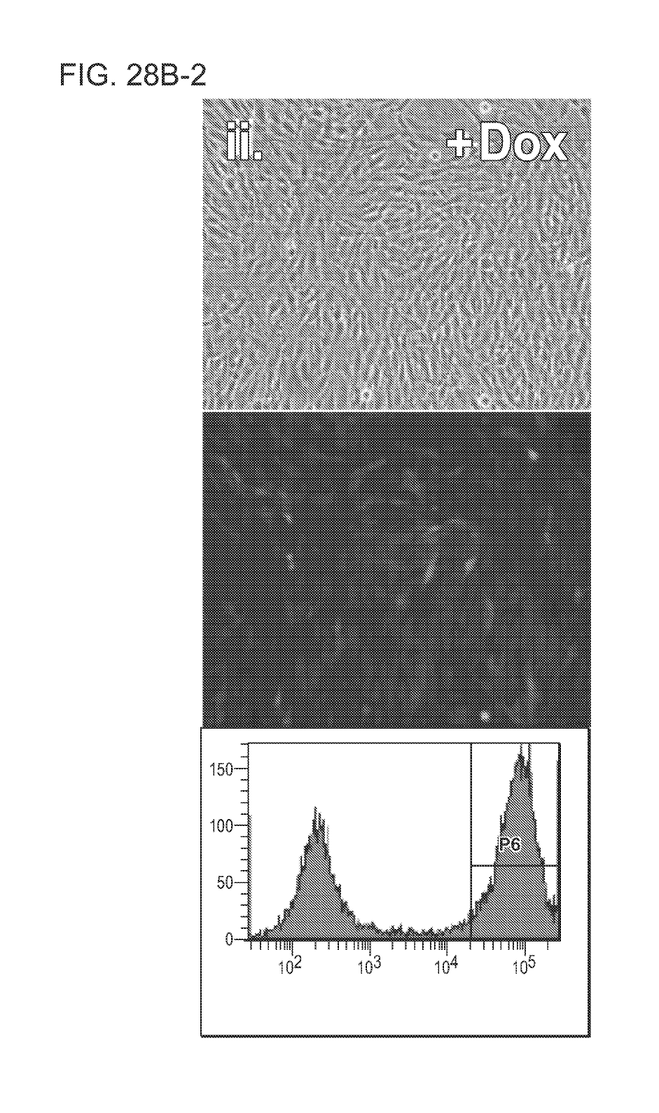

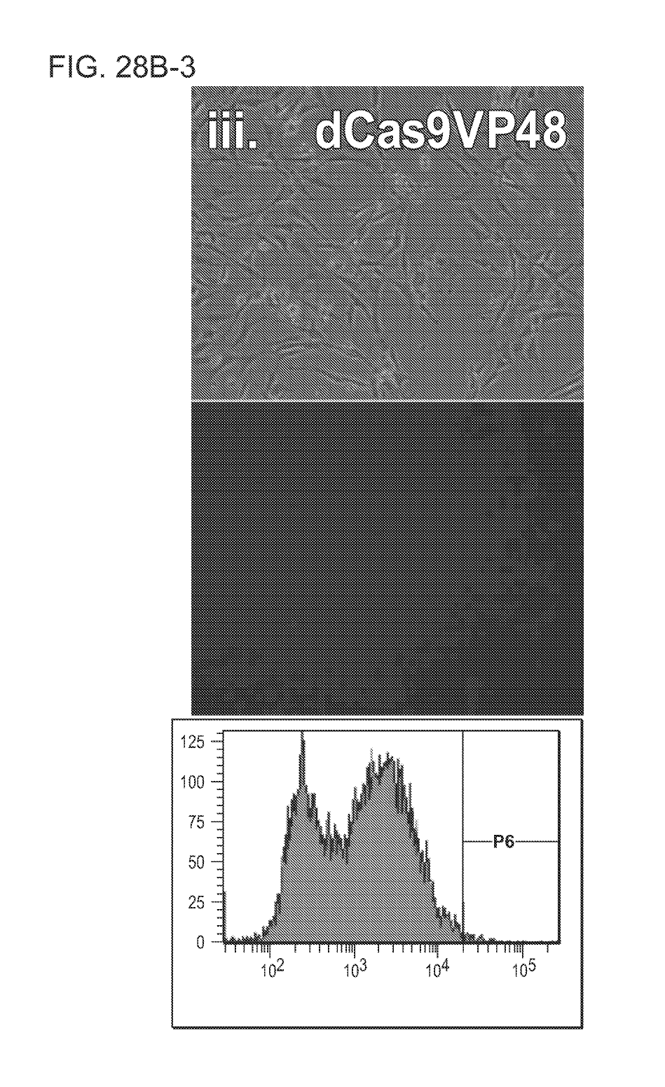

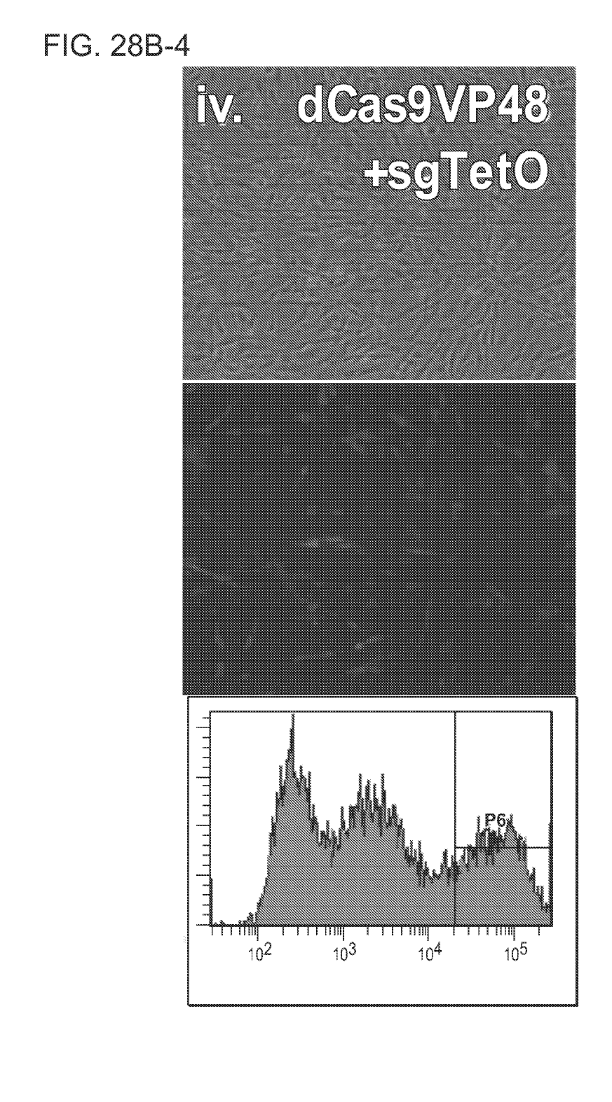

To generate a CRISPR/Cas-based transcription activator, the H840A mutation was introduced in the human codon-optimized Cas9 (D10A) nickase (Cong et al., Science; 339 (6121):819-823(2013)) to create a nuclease-deficient dCas9 (H840A; D10A) and fused a 3× minimal VP16 transcriptional activation domain (VP48) to its C-terminus (FIG. 23A). We first tested dCas9VP48 in human HeLa cells carrying integrated tdTomato reporter transgene under the control of a Tetracycline-inducible promoter composed of seven copies of rtTA binding sites and a CMV minimal promoter (TetO::tdTomato). As a positive control, these cells constitutively express the rtTA transactivator that induced tdTomato expression upon doxycycline treatment (FIG. 23B panel ii). Transient transfection of dCas9VP48 with sgRNA complementary to rtTA binding site (sgTetO) activated the TetO::tdTomato reporter in the absence of doxycycline at almost the same efficiency as the positive control (FIG. 23B panel iv). Transfection of dCas9VP48 without sgRNA did not activate tdTomato expression. (FIG. 23B panel iii). Activation of a TetO::tdTomato reporter lasted for one week but became weak afterwards (FIG. 27). Similarly, co-expression of dCas9VP48 with sgTetO activated tdTomato transgene in mouse NIH3T3 cells carrying an integrated TetO::tdTomato reporter (FIG. 28B, panel iv), while expression of dCas9VP48 alone did not activate tdTomato expression (FIG. 28B, panel iii). These results indicate that CRISPR-on activates a transgene reporter robustly in human and mouse cells to a similar level as rtTA in the presence of doxycycline and that the binding of dCas9VP48 to the tetO promoter is strictly dependent on sgTetO. The higher fraction of fluorescent HeLa cells as compared to that in NIH3T3 cells is likely due to higher transfection efficiency.

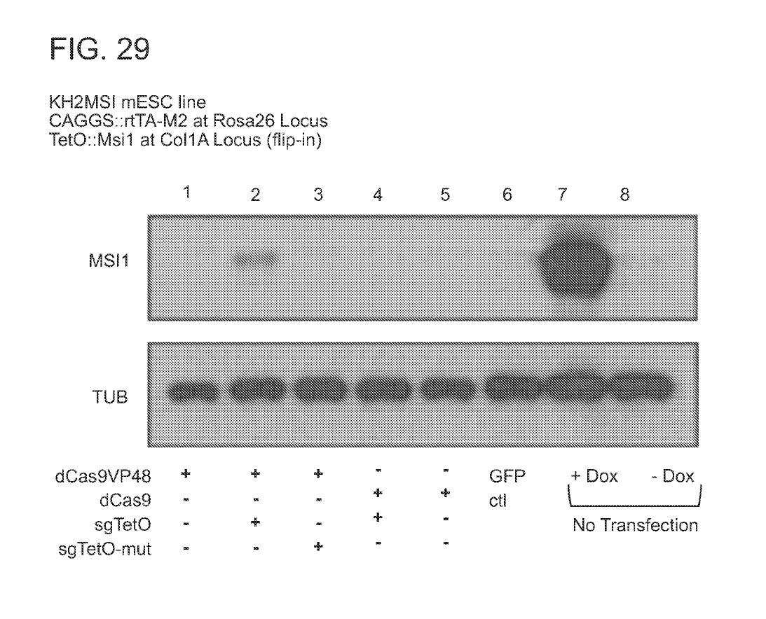

CRISPR-on was tested whether it could activate a single-copy transgene in embryonic stem cells (ESC). For this dCas9VP48 was co-transfected with sgTetO into ESC cells carrying a Tet-inducible Musashil (MSI1) transgene at the Col1a locus and the rtTA-M2 in the Rosa26 locus (Kharas et al., Nature medicine; 16 (8):903-908 (2010)) (FIG. 29). Transient transfection of dCas9VP48 alone did not activate MSI1 expression (FIG. 29, Lane 1) while co-transfection of dCas9VP48 with sgTetO or addition of doxycycline (positive control) activated MSI1 expression (FIG. 29, Lanes 2 and 7). Neither expression of dCas9VP48 with a mutant TetO sgRNA (sgTetO-mut) carrying mismatches to the TetO binding sites (FIG. 29, Lane 3) nor expression of sgTetO with dCas9 lacking an activation domain activated MSI1 expression (FIG. 29, Lane 4).

To further characterize the system, HEK293T/TetO::tdTomato cells were transfected with dCas9 activator and a serial titration of sgRNAs (FIG. 30). A near-linear relationship was observed between the amount of sgTetO transfected with the mean fluorescence by FACS (FIG. 30B), indicating that the level of gene activation could be controlled precisely using CRISPR-on.

To test whether CRISPR-on can activate genes in vivo, dCas9VP48 plasmid, seven different sgRNAs (sgNanog-1-7) targeting the mouse Nanog promoter and a Nanog::EGFP construct containing 1 kb promoter and 5′ UTR of Nanog were co-injected into mouse zygotes (FIGS. 23C and 23D). Two days after injection, a GFP signal was detected in 4-cell embryos by fluorescence microscopy and higher GFP expression was observed in morula and blastocyst on day 3 and day 4, whereas no GFP signal was observed in control embryos without the sgRNAs. This indicates that dCas9VP48/sgNanogs can specifically activate the GFP transgene in mouse embryos.

Activation of Endogenous Genes

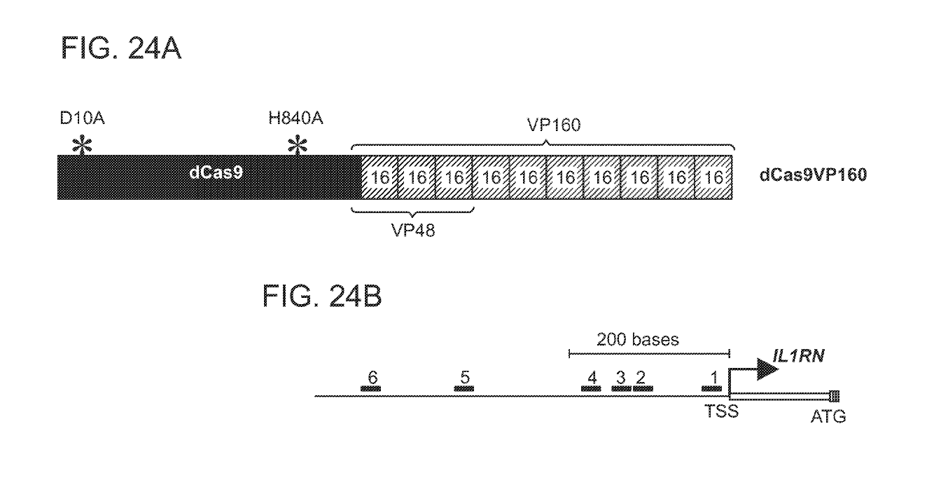

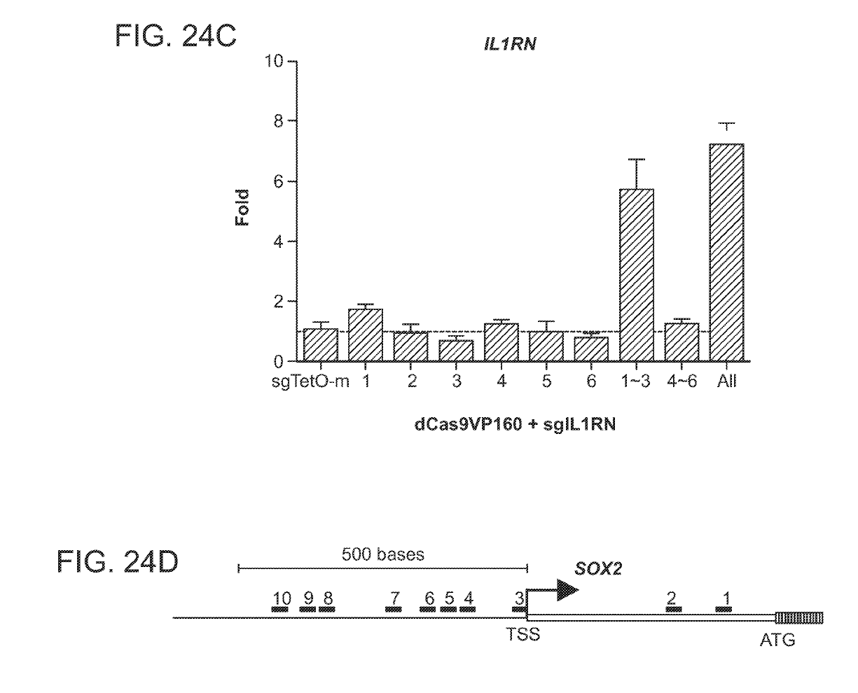

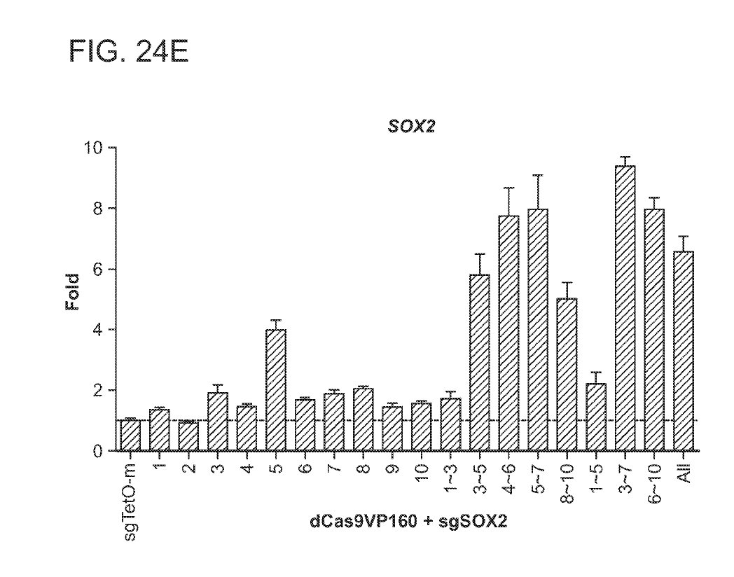

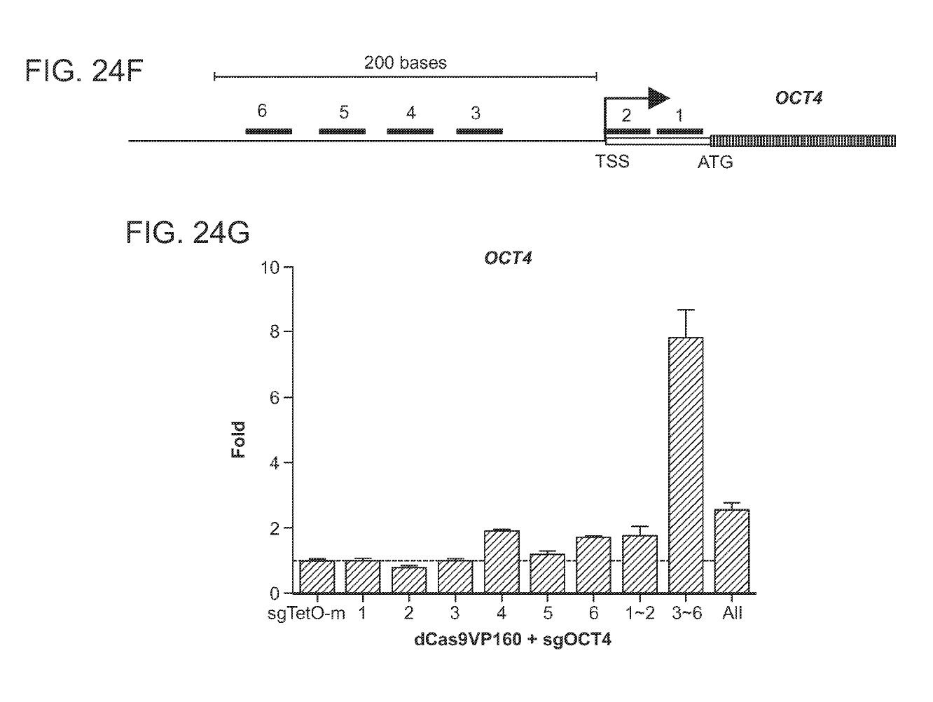

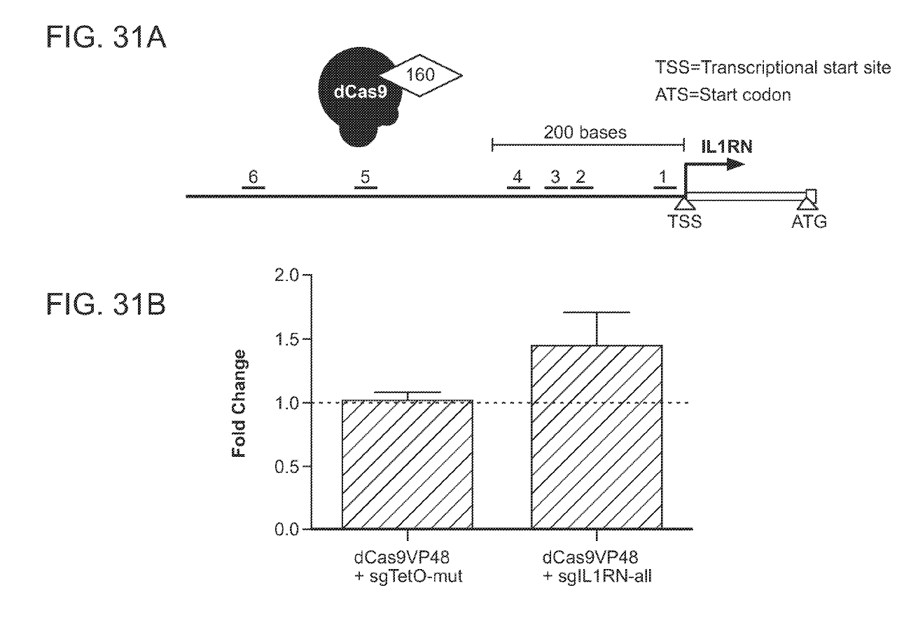

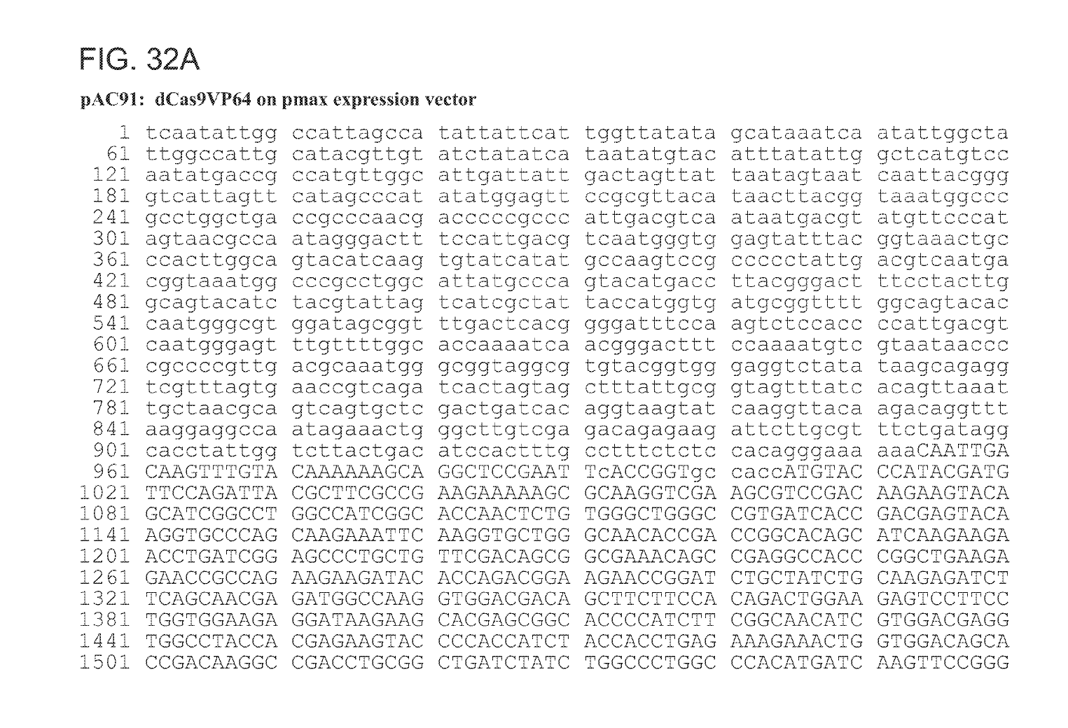

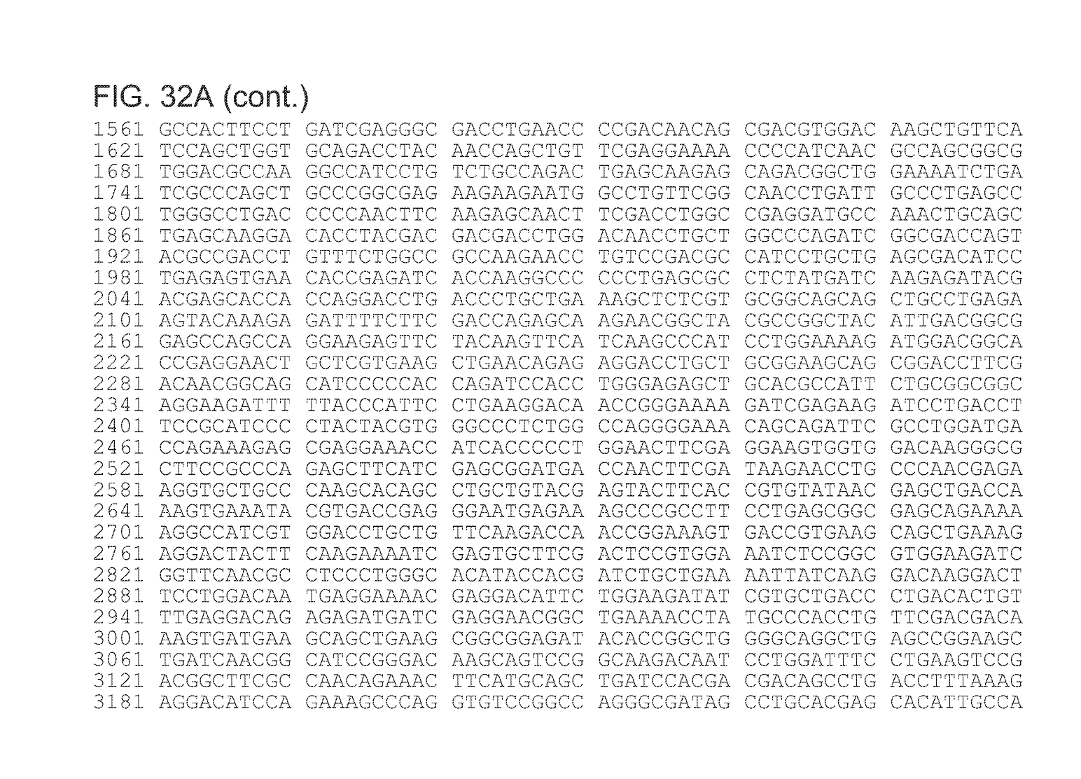

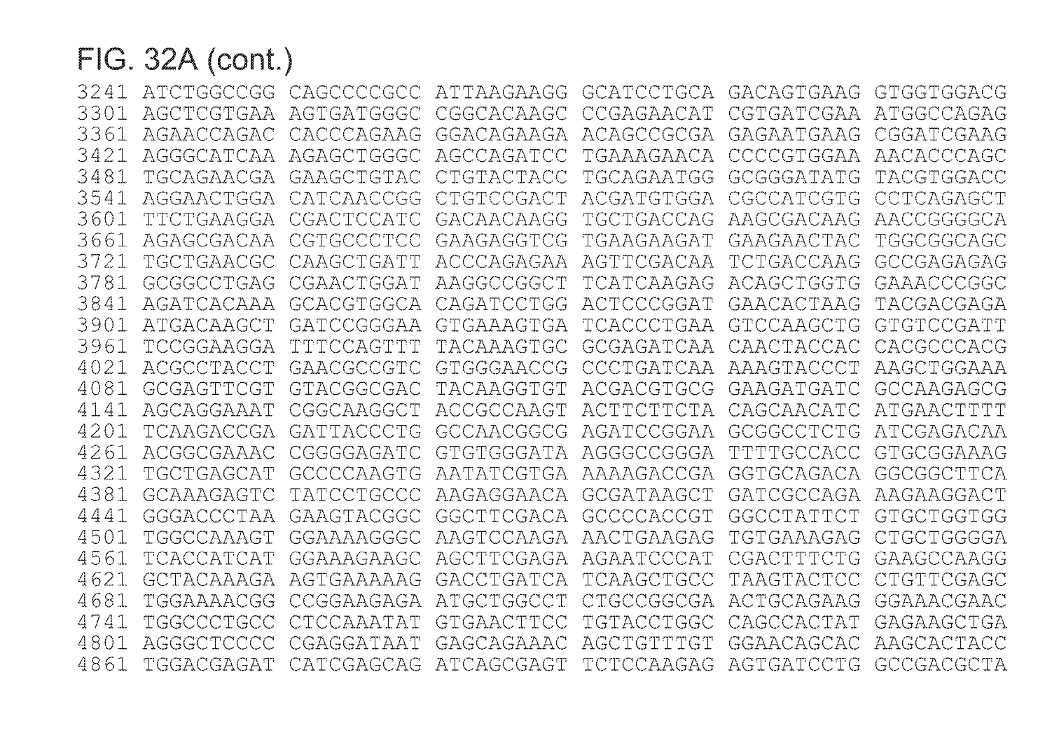

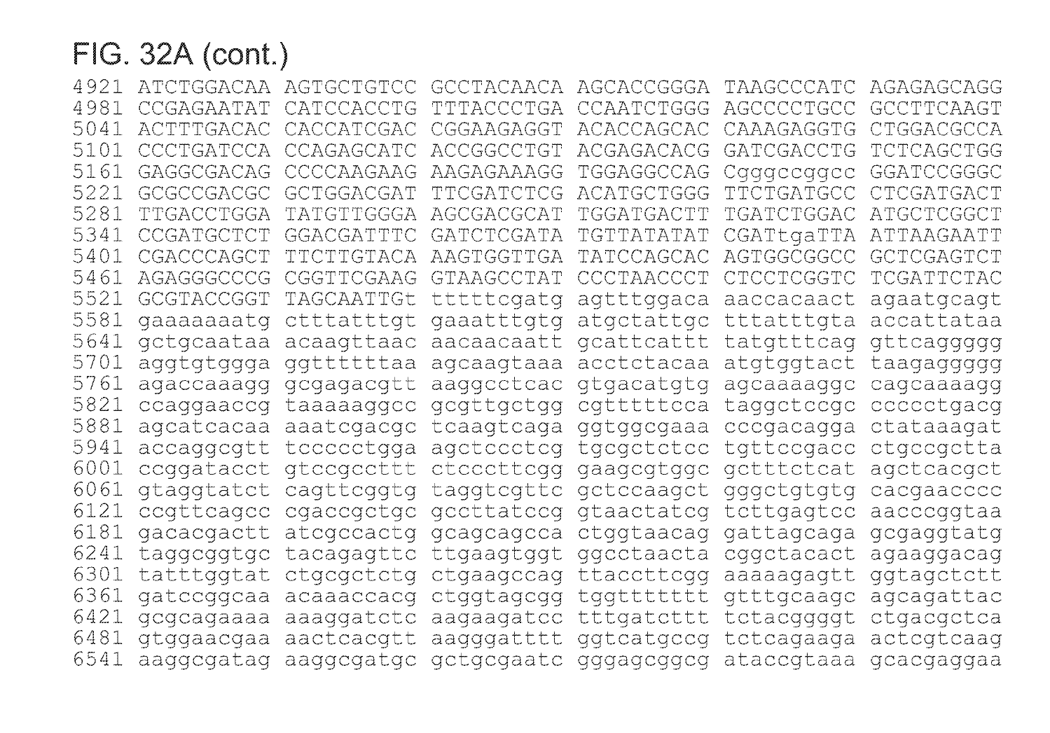

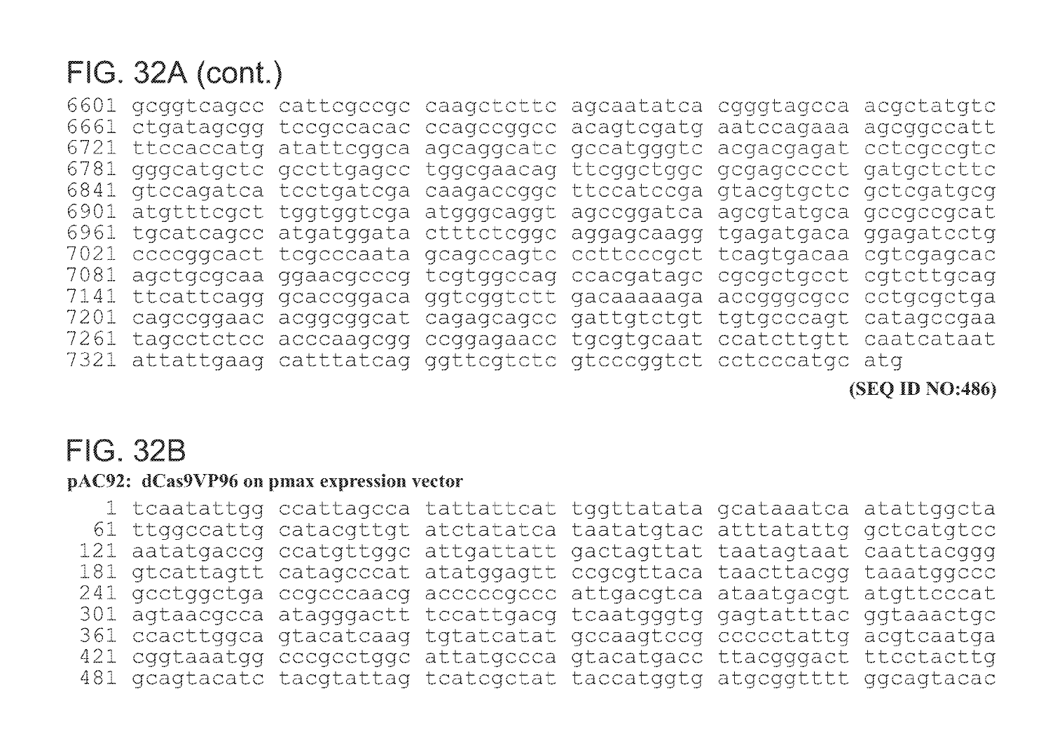

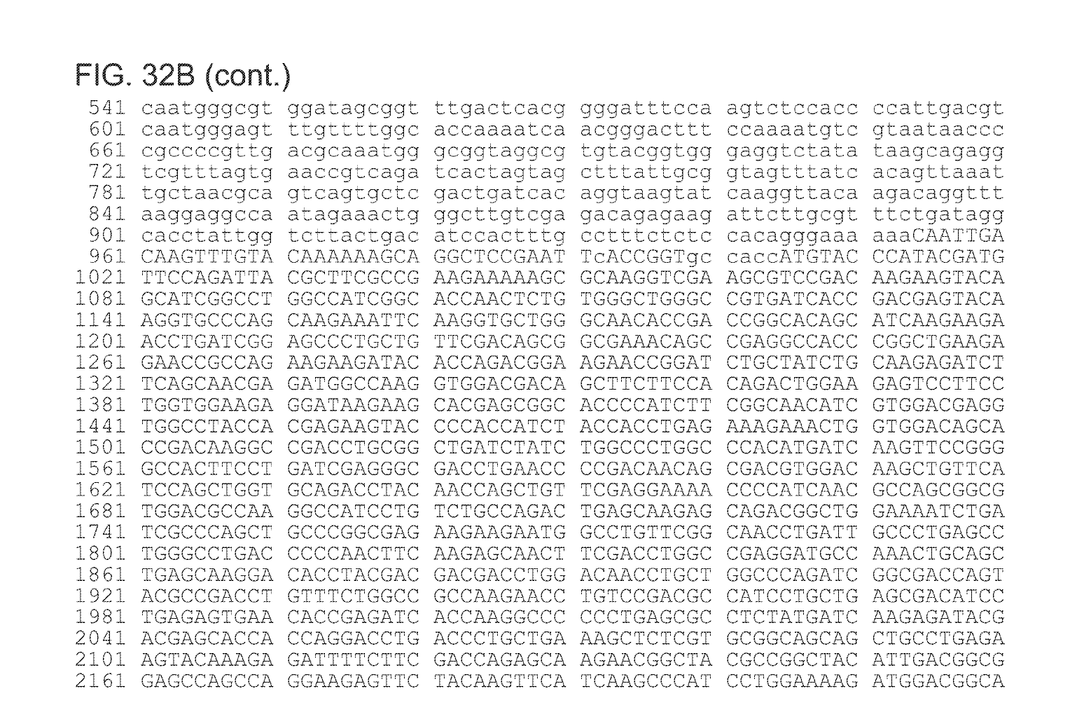

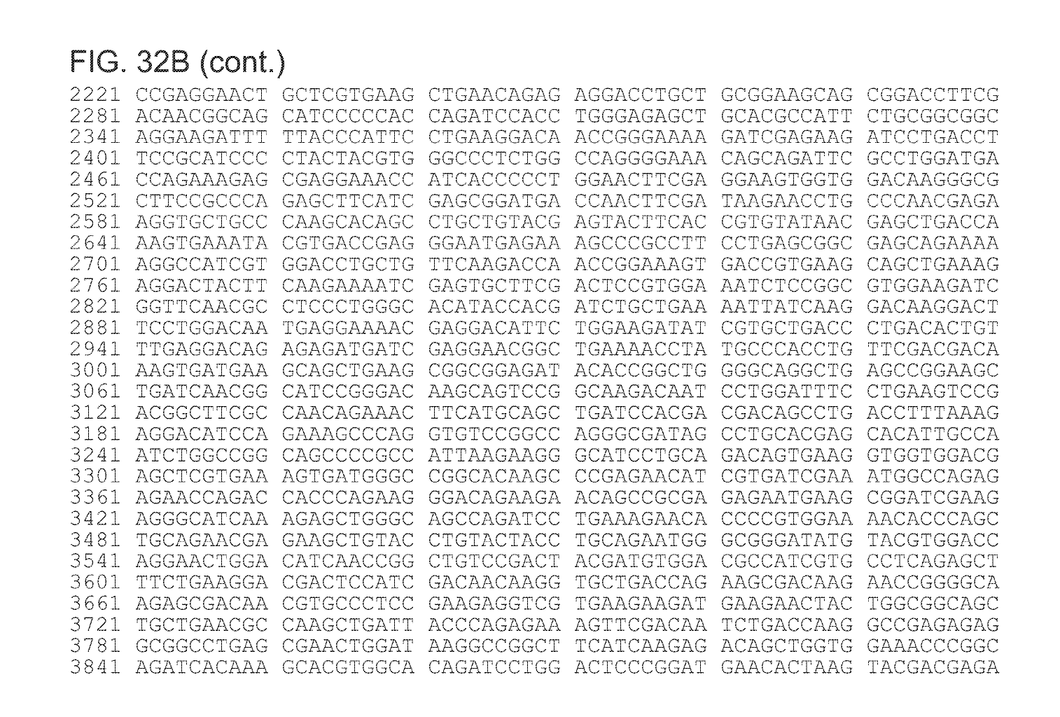

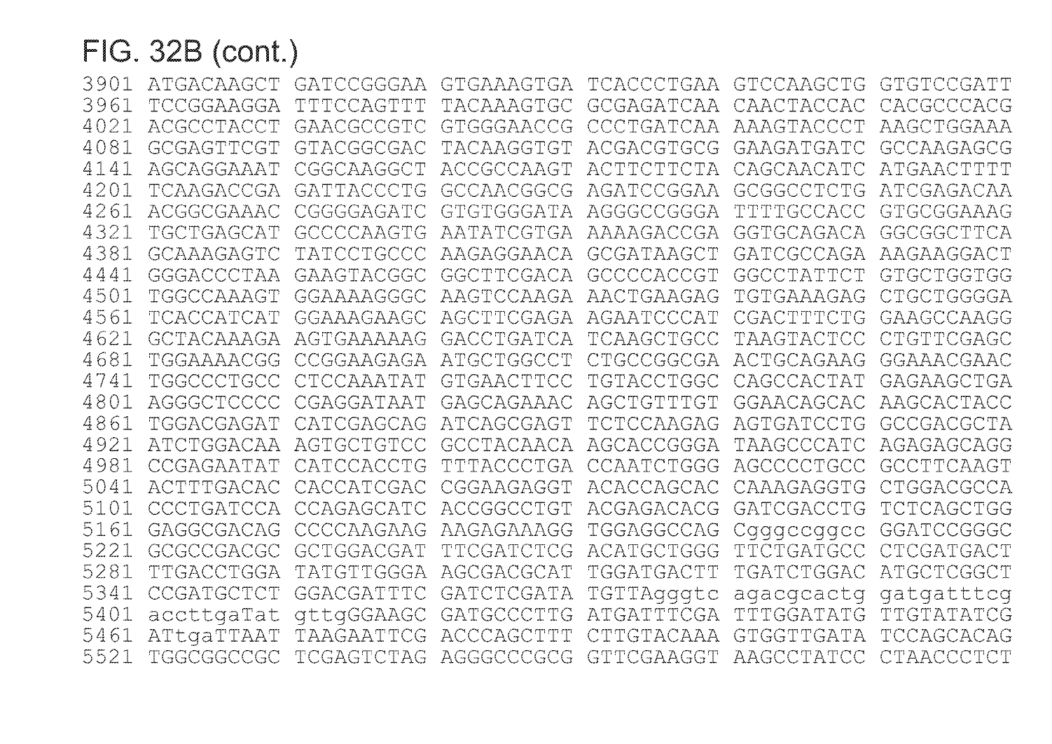

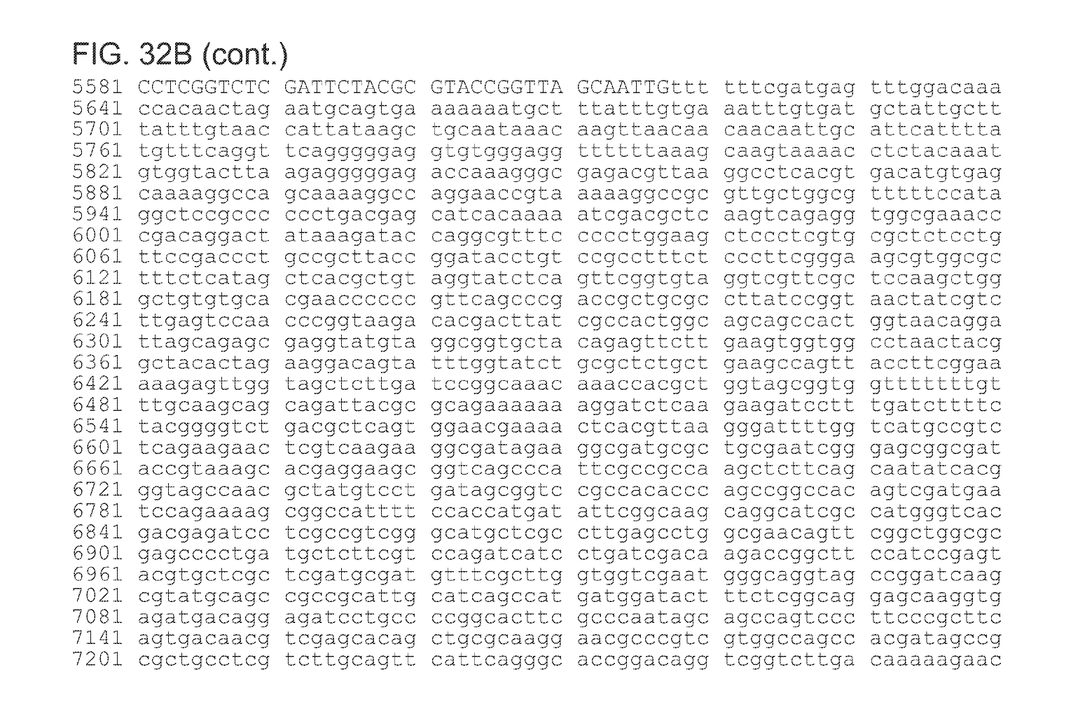

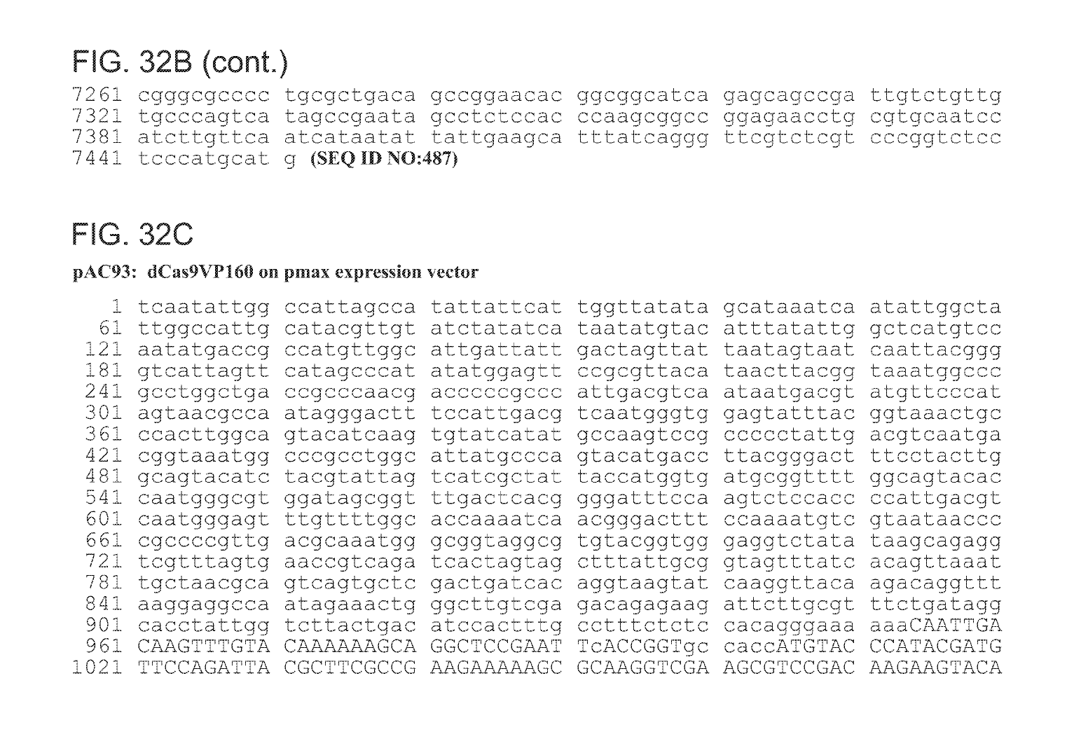

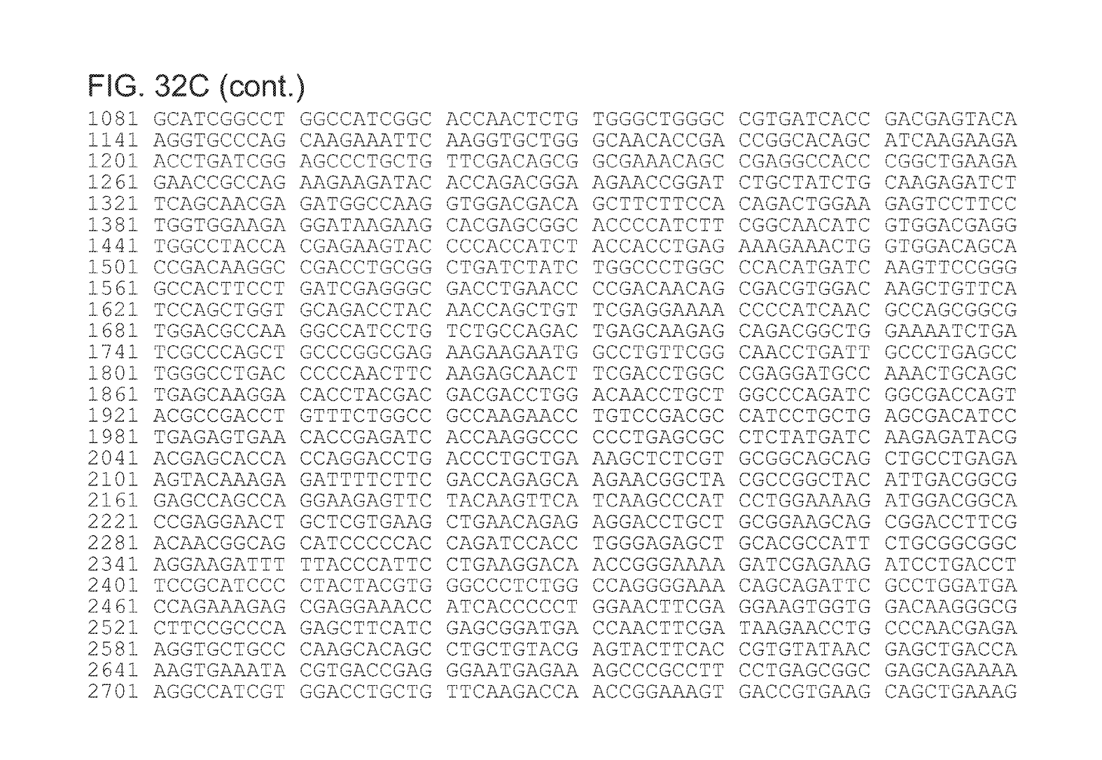

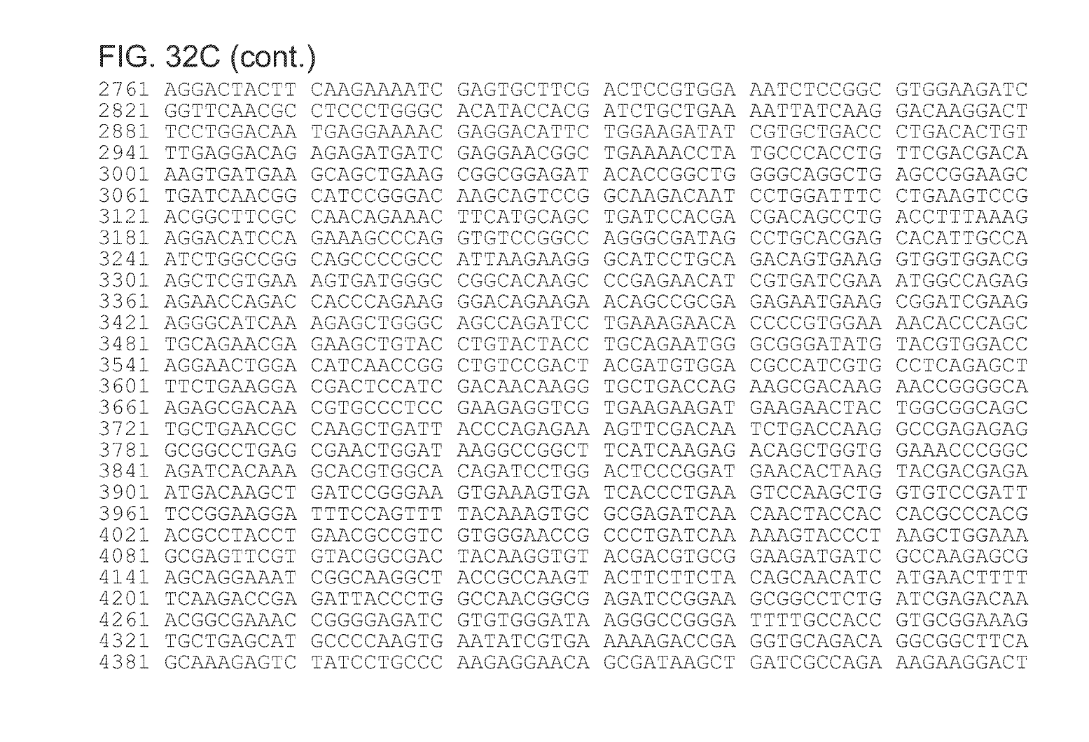

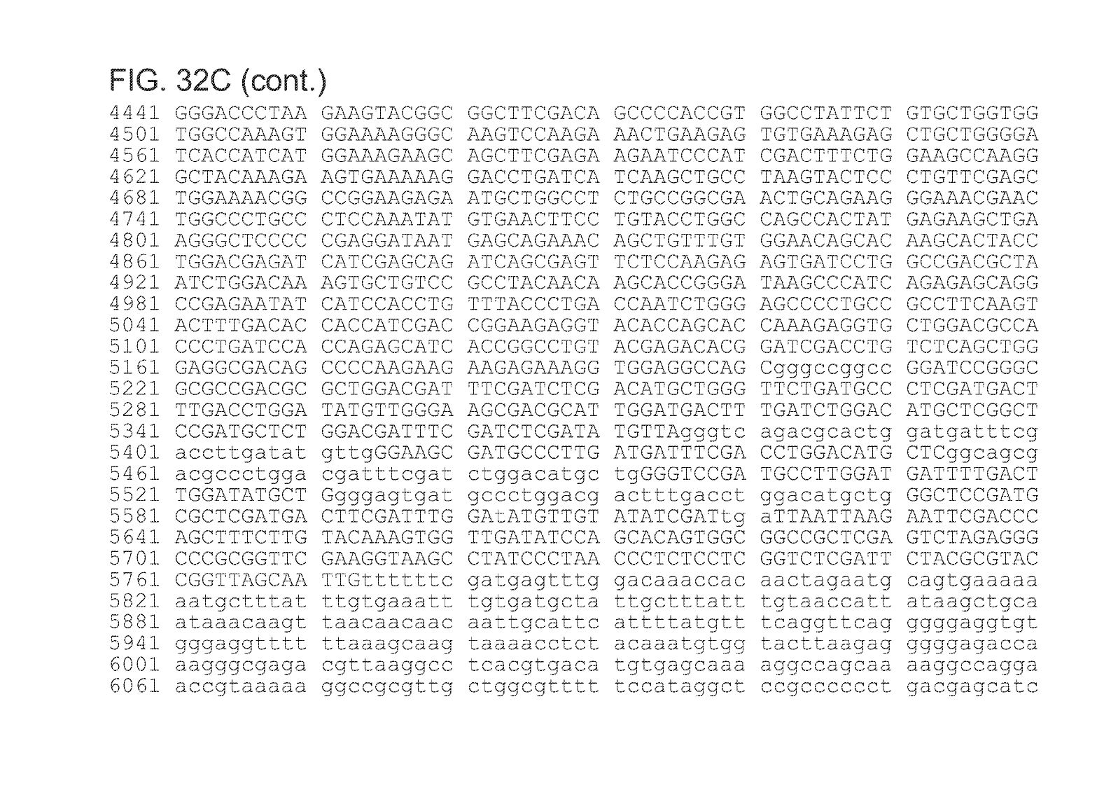

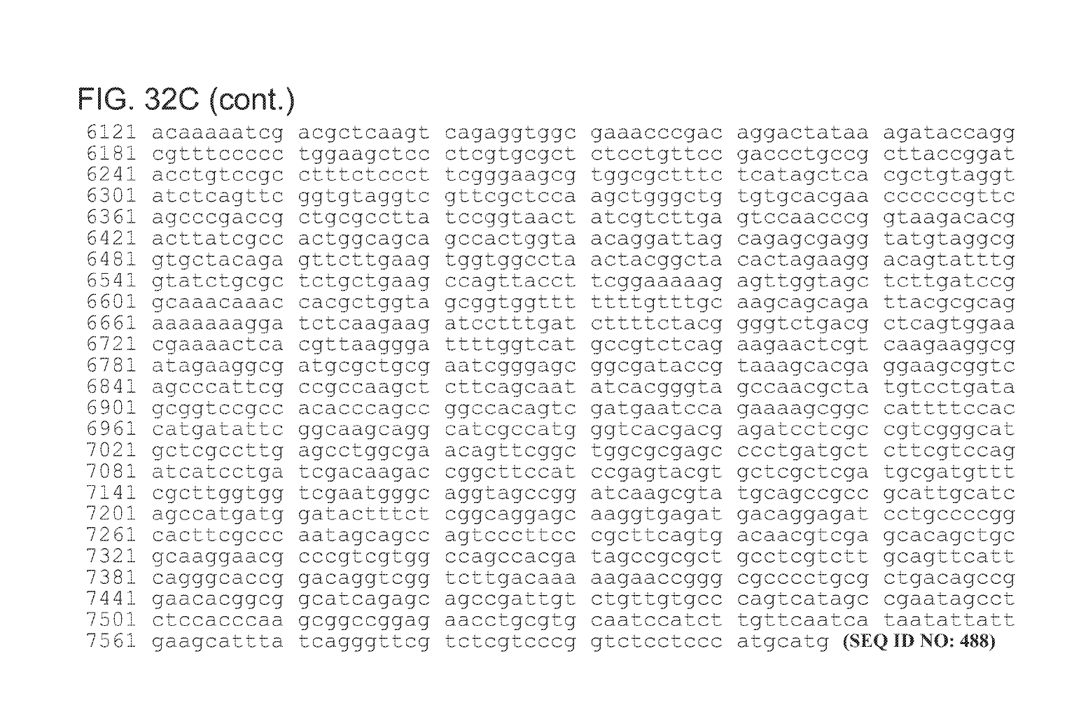

Having established that the CRSIPR-on system can activate reporter transgenes, sgRNAs targeting the endogenous human IL1RN gene were designed and tested their transactivation activity in HEK293T cells. To identify the binding sites most efficient for gene induction, six sgRNAs were designed to span the 1 kb IL1RN promoter (FIGS. 31A-31B). Initially, dCas9VP48 was transfected with all 6 sgRNAs, but failed to induce IL1RN gene expression (FIGS. 31A-31B). To test whether a stronger activation domain can activate IL1RN, a VP160 domain containing 10 tandem copies of VP16 motifs was fused with dCas9 to generate dCas9VP160 (FIG. 24A). When co-transfected with multiple but not single sgRNAs, dCas9VP160 readily activated IL1RN (FIGS. 24B and 24C). Transduction of three proximal sgRNAs (sgIL1RN1˜3) activated IL1RN approximately 6 fold, whereas the three distal sgRNAs (sgIL1RN4˜6) did not induce robust induction. Addition of sgRNA4-6 to the proximal sgRNAs (sgIL1RN1˜3) did not significantly augment the expression (FIG. 24C). These data suggest that gene activation is synergistically promoted by multiple dCas9VP160/sgRNA binding events at the proximal region of the IL1RN promoter.

A similar result was obtained with 10 sgRNAs spanning the SOX2 promoter (FIGS. 24D and 24E). As for IL1RN, expression of single sgRNAs did not yield strong activation of SOX2, while the triple sgRNAs (3˜5, 4˜6, 5˜7, 8˜10) activated SOX2 more than 4 fold. Seven fold activation was achieved with sgSOX2-4˜6 and sgSOX2-5˜7, while further distal sgRNAs (sgSOX2-8˜10) or those downstream of TSS (sgSOX2-1˜2) were less potent. Quintuple sgSOX2-1˜5 had a lower activity than triple sgSOX2 3˜5 suggesting that sgRNAs downstream of TSS (sgSOX2-1˜2) may be detrimental to activation. Binding of dCas9VP160 downstream of transcriptional start sites may sterically hinder transcription by blocking polymerase, consistent with a previously report on CRISPRi (Qi et al., Cell; 152 (5):1173-1183 (2013)). To further confirm this observation, six sgRNAs were designed spanning Oct4 promoter, including two targeting downstream of TSS (sgOCT4-1˜2) (FIG. 24F). An eight fold activation was achieved with sgOCT4-3˜6, albeit all six sgOCT4-1˜6 had a much lower activity than sgOCT4-3˜6 confirming that sgRNAs downstream of TSS (sgSOX2-1˜2) have a negative effect on gene activation (FIG. 24G). Therefore, in IL1RN, SOX2, and OCT4 promoters, three to five dCas9VP160/sgRNAs binding within 300 bp upstream TSS induced the most efficient gene activation.

Multiple Exogenous and Endogenous Genes can be Simultaneously Activated by CRISPR-On

Single, double and triple activation of a TetO::tdTomato transgene and the endogenous SOX2 and IL1RN genes (FIG. 3A) were tested in HEK293T cells carrying the stably integrated TetO::tdTomato transgene (HEK293T/TetO::tdTomato). Transfection of sgRNAs targeting the individual promoters (sgTetO for TetO::tdTomato, sgSOX2-1˜10 for SOX2 or sgIL1RN1˜6 for IL1RN) activated the respective genes (TetO: 6.6×; SOX2: 3.5×; IL1RN: 10.7×) while not affecting expression of the other two genes (FIG. 25A). Simultaneous transfection of sgRNAs targeting two or three promoters activated the corresponding sets of genes (FIG. 25A).

To test whether the system allows the activation of three different endogenous genes in a dose dependent manner, HEK293T cells were co-transfected with dCas9VP160 and the most efficient sgRNAs targeting all three genes (sgIL1RN1˜3 for IL1RN, sgSOX2-5˜7 for targeting SOX2, and sgOCT4-1˜3 for OCT4) in different ratios (FIG. 25B). When sgRNAs targeting one gene, or two genes were used, only the respective genes were activated. When all sgRNAs targeting three genes were transfected, albeit in different ratios, robust activation of all three genes was observed (FIG. 25B). More significantly, when different ratios of sgRNAs were used targeting SOX2 and IL1RN while maintaining the OCT4 sgRNAs constant, the predicted change of the ratio of SOX2 and IL1RN expression levels, and the OCT4 expression remained stable (FIG. 25B). These results demonstrate that the CRISPR-on system can be robustly used for multiplexed activation of endogenous genes.

CRISPR-On is Highly Specific

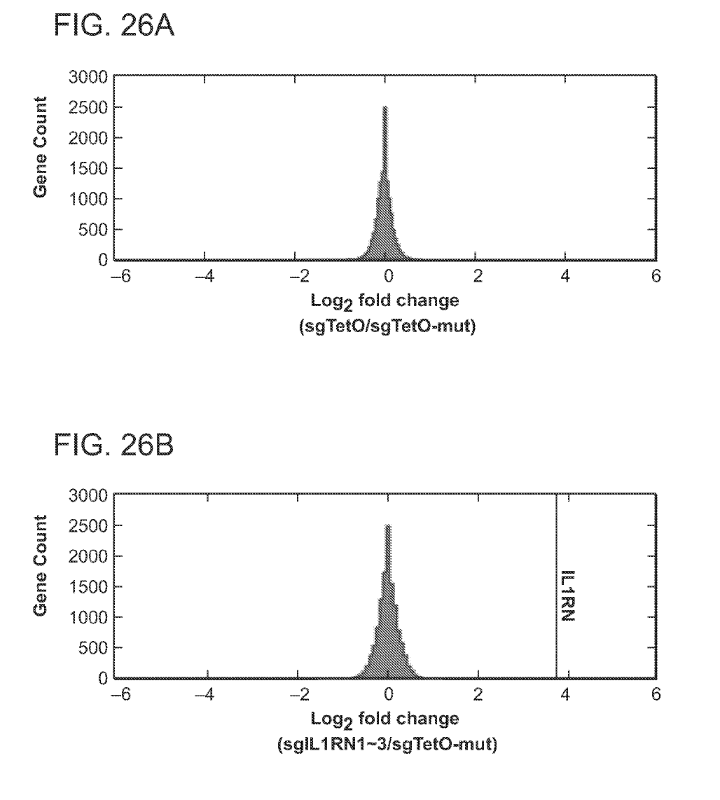

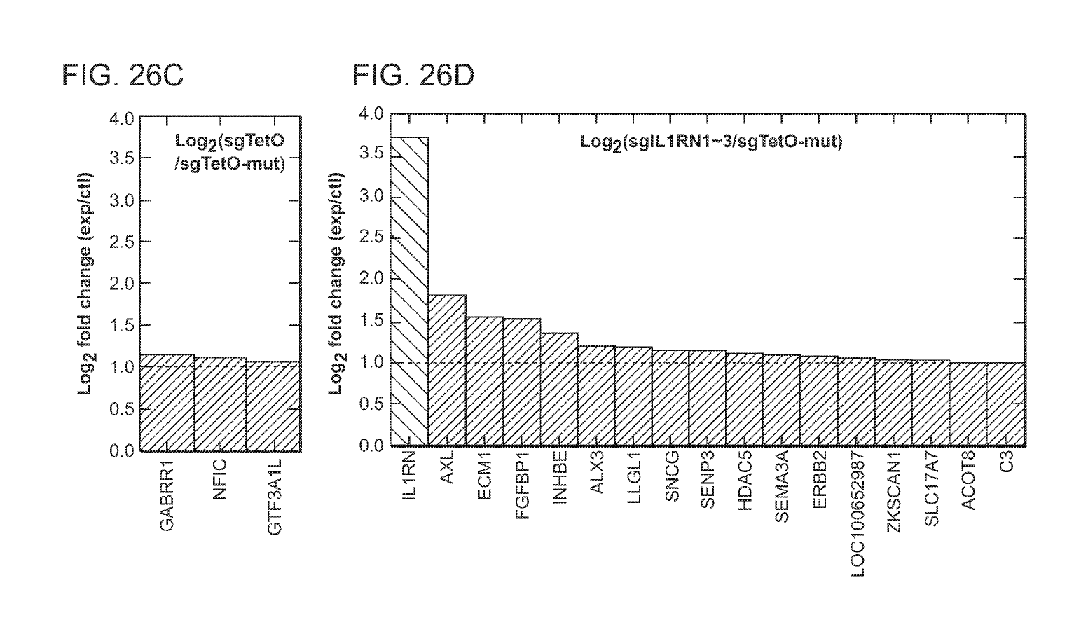

To test the specificity of CRISPR-on-mediated gene activation, microarray experiments were conducted to compare genome-wide gene expression profiles of cells transfected with dCas9VP160 and specific sgRNAs to cells transfected with dCas9VP160 and sgTetO-mut control sgRNA (FIGS. 26A-26D). While efficiently activating target genes, CRISPR-on did not cause major perturbations in the transcriptome (FIGS. 26A and 26B) as only three genes showed an over two fold up regulation upon transduction of dCas9VP160/sgTetO (FIG. 26C). While CRISPR-on mediated activation of IL1RN induced the IL1RN target gene 13 fold, only 16 other genes showed an about twofold increase in expression (FIG. 26D). The minor upregulation of these genes may be secondary due to the over-expression of tdTomato or IL1RN.

Discussion

Artificial transcription factors (ATFs) are valuable tools for studying gene functions and transcriptional networks. Zinc-fingers and TALE transcription factors have been developed over the recent decades and show promises in both bioengineering and therapeutic applications (Sera T., Adv Drug Deliv Rev; 61 (7-8):513-526 (2009); Perez-Pinera et al., Nat Methods; 10 (3):239-242 (2013); Maeder et al., Nature methods 2013; 10 (3):243-245 (2013)). Here, CRISPR-on was established as a novel class of artificial transcription factors based on the CRISPR/Cas system. A major advantage of this system is that only one Cas9 protein is required to activate multiple genes individually or simultaneously and that its DNA binding specificity is determined by sgRNAs, which are designed based on simple RNA/DNA complementarity.

Using CRISPR-on, robust activation was demonstrated of exogenous reporter genes in both human and mouse transformed cells as well as in ES cells. When the system was introduced into one-cell mouse embryos, efficient reporter gene activation occurred. This system can be used to manipulate transcriptional networks in early embryos.

Robust endogenous gene activation was achieved using the stronger activation domain VP160. Further optimization of activation domains, such as using different linker sequences, may improve the CRISPR-on activation efficiency even further. The promoter scanning experiments demonstrated that efficient activation of endogenous genes could be achieved by three to five sgRNAs binding within 300 bp region upstream of transcription start sites. Using additional sgRNAs targeting further upstream or downstream regions did not significantly improve the level of induction. This data suggest that only a small number of sgRNAs targeting the proximal promoter are sufficient to activate endogenous genes.

It is shown here that the CRISPR-on system can be used for the simultaneous induction of at least three different endogenous genes. More significantly, the stoichiometry of gene induction of multiple genes can be tuned by adjusting the relative amount of their cognate sgRNAs. Simultaneous activation of multiple endogenous genes with defined stoichiometry opens up novel opportunities for systems biology as it allows for the predictable manipulation of transcriptional networks.

Finally, with the ease of design and synthesis, a library of sgRNAs could be generated. When introduced into a cell line constitutively expressing dCas9 protein, gene activation screens mediated by RNA (RNAa) could be achieved. Since the specificity components (sgRNA) can be separately designed and constructed from the effector component (Cas protein), the same library of sgRNAs could be used with different dCas9 fusions (e.g., VP160 domain for transactivation, KRAB domain for transcriptional repression, chromatin modifier domains for specific histone modification) to exert different functions at particular genomic loci.

```
CRISPR dCas9 fusion peptides sequences
dCas9VP160-2A-puro
  (SEQ ID NO: 478)
MYPYDVPDYASPKKKRKVEASDKKYSIGLAIGTNSVGWAVITDEYKVPSKKFKVLGNTD
R HSIKKNLIGALLFDSGETAEATRLKRTARRRYTRRKNRICYLQEIFSNEMAKVDDSFEHR
LEESFLVEEDKKHERHPIFGNIVDEVAYHEKYPTIYHLRKKLVDSTDKADLRLIYLALAH
MIKFRGHFLIEGDLNPDNSDVDKLFIQLVQTYNQLFEENPINASGVDAKAILSARLSKSR
RLENLIAQLPGEKKNGLFGNLIALSLGLTPNEKSNEDLAEDAKLQLSKDTYDDDLDNLLA
QIGDQYADLFLAAKNLSDAILLSDILRVNTEITKAPLSASMIKRYDEHHQDLTLLKALVR
QQLPEKYKEIFEDQSKNGYAGYIDGGASQEEFYKEIKPILEKMDGTEELLVKLNREDLLR
KQRTFDNGSIPHQIHLGELHAILRRQEDFYPFLKDNREKIEKILTFRIPYYVGPLARGNS
RFAWMTRKSEETITPWNFEEVVDKGASAQSFIERMTNFDKNLPNEKVLPKHSLLYEYFTV
YNELTKVKYVTEGMRKPAELSGEQKKAIVDLLEKTNRKVTVKQLKEDYFKKIECEDSVEI
SGVEDRFNASLGTYHDLLKIIKDKDFLDNEENEDILEDIVLTLTLFEDREMIEERLKTYA
HLFDDKVMKQLKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNKLYLYYLQNGR
DMYVDQELDINRLSDYDVDAIVPQSFLKDDSIDNKVLTRSDKNRGKSDNVPSEEVVKKMK
NYWRQLLNAKLITQRKFDNLTKAERGGLSELDKAGFIKRQLVETRQITKHVAQILDSRMN
TKYDENDKLIREVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEITLANGEIRKR
PLIETNGETGEIVWDKGRDFATVRKVLSMPQVNIVKKTEVQTGGFSKESILPKRNSDKLI
ARKKDWDPKKYGGFDSPTVAYSVLVVAKVEKGKSKKLKSVKELLGITIMERSSFEKNPID
FLEAKGYKEVKKDLIIKLPKYSLFELENGRKRMLASAGELQKGNELALPSKYVNFLYLAS
HYEKLKGSPEDNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQSITGLYETRI
DLSQLGGDSPKKKRKVEASGPAGSGRADALDDFDLDMLGSDALDDFDLDMLGSDALDDFD
LDMLGSDALDDFDLDMLGSDALDDFDLDMLGSDALDDFDLDMLGSDALDDFDLDMLGSDA
LDDFDLDMLGSDALDDFDLDMLGSDALDDFDLDMLYIDRSGSGEGRGSLLTCGDVEENPG
PRLEMTEYKPTVRLATRDDVPRAVRTLAAAFADYPATRHTVDPDRHIERVTELQELFLTR
VGLDIGKVWVADDGAAVAVWTTPESVEAGAVFAEIGPRMAELSGSRLAAQQQMEGLLAPH
RPKEPAWFLATVGVSPDHQGKGLGSAVVLPGVEAAERAGVPAFLETSAPRNLPFYERLGF
TVTADVEVPEGPRTWCMTRKPGA
dCas9VP160-2A-neo
  (SEQ ID NO: 479)
MYPYDVPDYASPKKKRKVEASDKKYSIGLAIGTNSVGWAVITDEYKVPSKKFKVLGNTD
R HSIKKNLIGALLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKADLRLIYLALAH
MIKFRGHFLIEGDLNPDNSDVDKLFIQLVQTYNQLFEENPINASGVDAKAILSARLSKSR
RLENLIAQLPGEKKNGLFGNLIALSLGLTPNFKSNFDLAEDAKLQLSKDTYDDDLDNLLA
QIGDQYADLFLAAKNLSDAILLSDILRVNTEITKAPLSASMIKRYDEHHQDLTLLKALVR
QQLPEKYKEIFFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPYYVGPLARGNS
RFAWMTRKSEETITPWNFEEVVDKGASAQSFIERMTNFDKNLPNEKVLPKHSLLYEYFTV
YNELTKVKYVTEGMRKPAFLSGEQKKAIVDLLFKTNRKVTVKQLKEDYFKKIECFDSVEI
SGVEDRFNASLGTYHDLLKIIKDKDFLDNEENEDILEDIVLTLTLFEDREMIEERLKTYA
HLFDDKVMKQLKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGR
DMYVDQELDINRLSDYDVDAIVPQSFLKDDSIDNKVLTRSDKNRGKSDNVPSEEVVKKMK
NYWRQLLNAKLITQRKFDNLTKAERGGLSELDKAGFIKRQLVETRQITKHVAQILDSRMN
TKYDENDKLIREVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEITLANGEIRKR
PLIETNGETGEIVWDKGRDFATVRKVLSMPQVNIVKKTEVQTGGFSKESILPKRNSDKLI
ARKKDWDPKKYGGFDSPTVAYSVLVVAKVEKGKSKKLKSVKELLGITIMERSSFEKNPID
FLEAKGYKEVKKDLIIKLPKYSLFELENGRKRMLASAGELQKGNELALPSKYVNFLYLAS
HYEKLKGSPEDNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQSITGLYETRI
DLSQLGGDSPKKKRKVEASGPAGSGRADALDDFDLDMLGSDALDDFDLDMLGSDALDDFD
LDMLGSDALDDFDLDMLGSDALDDFDLDMLGSDALDDFDLDMLGSDALDDFDLDMLGSDA
LDDFDLDMLGSDALDDFDLDMLGSDALDDFDLDMLYIDRSGSGEGRGSLLTCGDVEENPG
PRLETRMGSAIEQDGLHAGSPAAWVERLFGYDWAQQTIGCSDAAVFRLSAQGRPVLFVKT
DLSGALNELQDEAARLSWLATTGVPCAAVLDVVTEAGRDWLLLGEVPGQDLLSSHLAPAE
KVSIMADAMRRLHTLDPATCPFDHQAKHRIERARTRMEAGLVDQDDLDEEHQGLAPAELF
ARLKARMPDGEDLVVTHGDACLPNIMVENGRFSGFIDCGRLGVADRYQDIALATRDIAEE
LGGEWADRFLVLYGIAAPDSQRIAFYRLLDEFF
dCas9p65
  (SEQ ID NO: 480)
MYPYDVPDYASPKKKRKVEASDKKYSIGLAIGTNSVGWAVITDEYKVPSKKFKVLGNTD
R HSIKKNLIGALLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKADLRLIYLALAH
MIKFRGHFLIEGDLNPDNSDVDKLFIQLVQTYNQLFEENPINASGVDAKAILSARLSKSR
RLENLIAQLPGEKKNGLFGNLIALSLGLTPNFKSNFDLAEDAKLQLSKDTYDDDLDNLLA
QIGDQYADLFLAAKNLSDAILLSDILRVNTEITKAPLSASMIKRYDEHHQDLTLLKALVR
QQLPEKYKEIFFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPYYVGPLARGNS
RFAWMTRKSEETITPWNFEEVVDKGASAQSFIERMTNFDKNLPNEKVLPKHSLLYEYFTV
YNELTKVKYVTEGMRKPAFLSGEQKKAIVDLLFKTNRKVTVKQLKEDYFKKIECFDSVEI
SGVEDRFNASLGTYHDLLKIIKDKDFLDNEENEDILEDIVLTLTLFEDREMIEERLKTYA
HLFDDKVMKQLKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGR
DMYVDQELDINRLSDYDVDAIVPQSFLKDDSIDNKVLTRSDKNRGKSDNVPSEEVVKKMK
NYWRQLLNAKLITQRKFDNLTKAERGGLSELDKAGFIKRQLVETRQITKHVAQILDSRMN
TKYDENDKLIREVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEITLANGEIRKR
PLIETNGETGEIVWDKGRDFATVRKVLSMPQVNIVKKTEVQTGGFSKESILPKRNSDKLI
ARKKDWDPKKYGGFDSPTVAYSVLVVAKVEKGKSKKLKSVKELLGITIMERSSFEKNPID
FLEAKGYKEVKKDLIIKLPKYSLFELENGRKRMLASAGELQKGNELALPSKYVNFLYLAS
HYEKLKGSPEDNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQSITGLYETRI
DLSQLGGDSPKKKRKVEASGPASPMEFQYLPDTDDRHRIEEKRKRTYETFKSIMKKSPFS
GPTDPRPPPRRIAVPSRSSASVPKPAPQPYPFTSSLSTINYDEFPTMVFPSGQISQASAL
APAPPQVLPQAPAPAPAPAMVSALAQAPAPVPVLAPGPPQAVAPPAPKPTQAGEGTLSEA
LLQLQFDDEDLGALLGNSTDPAVFTDLASVDNSEFQQLLNQGIPVAPHTTEPMLMEYPEA
ITRLVTGAQRPPDPAPAPLGAPGLPNGLLSGDEDFSSIADMDFSALLSQISSID
dCas9KRAB
(SEQ ID NO: 481)
MYPYDVPDYASPKKKRKVEASDKKYSIGLAIGTNSVGWAVITDEYKVPSKKFKVLGNTD
RHSIKKNLIGALLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKADLRLIYLALAH
MIKFRGHFLIEGDLNPDNSDVDKLFIQLVQTYNQLFEENPINASGVDAKAILSARLSKSR
RLENLIAQLPGEKKNGLFGNLIALSLGLTPNFKSNFDLAEDAKLQLSKDTYDDDLDNLLA
QIGDQYADLFLAAKNLSDAILLSDILRVNTEITKAPLSASMIKRYDEHHQDLTLLKALVR
QQLPEKYKEIFFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPYYVGPLARGNS
RFAWMTRKSEETITPWNFEEVVDKGASAQSFIERMTNFDKNLPNEKVLPKHSLLYEYFTV
YNELTKVKYVTEGMRKPAFLSGEQKKAIVDLLFKTNRKVTVKQLKEDYFKKIECFDSVEI
SGVEDRFNASLGTYHDLLKIIKDKDFLDNEENEDILEDIVLTLTLFEDREMIEERLKTYA
HLFDDKVMKQLKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGR
DMYVDQELDINRLSDYDVDAIVPQSFLKDDSIDNKVLTRSDKNRGKSDNVPSEEVVKKMK
NYWRQLLNAKLITQRKFDNLTKAERGGLSELDKAGFIKRQLVETRQITKHVAQILDSRMN
TKYDENDKLIREVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEITLANGEIRKR
PLIETNGETGEIVWDKGRDFATVRKVLSMPQVNIVKKTEVQTGGFSKESILPKRNSDKLI
ARKKDWDPKKYGGFDSPTVAYSVLVVAKVEKGKSKKLKSVKELLGITIMERSSFEKNPID
FLEAKGYKEVKKDLIIKLPKYSLFELENGRKRMLASAGELQKGNELALPSKYVNFLYLAS
HYEKLKGSPEDNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQSITGLYETRI
DLSQLGGDSPKKKRKVEASGPAASPKKKRKVEASMDAKSLTAWSRTLVTFKDVFVDFTRE
EWKLLDTAQQIVYRNVMLENYKNLVSLGYQLTKPDVILRLEKGEEPWLVSRGSID
dCas9PCP
(SEQ ID NO: 482)
MYPYDVPDYASPKKKRKVEASDKKYSIGLAIGTNSVGWAVITDEYKVPSKKFKVLGNTD
R HSIKKNLIGALLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKADLRLIYLALAH
MIKFRGHFLIEGDLNPDNSDVDKLFIQLVQTYNQLFEENPINASGVDAKAILSARLSKSR
RLENLIAQLPGEKKNGLFGNLIALSLGLTPNFKSNFDLAEDAKLQLSKDTYDDDLDNLLA
QIGDQYADLFLAAKNLSDAILLSDILRVNTEITKAPLSASMIKRYDEHHQDLTLLKALVR
QQLPEKYKEIFFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPYYVGPLARGNS
RFAWMTRKSEETITPWNFEEVVDKGASAQSFIERMTNFDKNLPNEKVLPKHSLLYEYFTV
YNELTKVKYVTEGMRKPAFLSGEQKKAIVDLLFKTNRKVTVKQLKEDYFKKIECFDSVEI
SGVEDRFNASLGTYHDLLKIIKDKDFLDNEENEDILEDIVLTLTLFEDREMIEERLKTYA
HLFDDKVMKQLKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGR
DMYVDQELDINRLSDYDVDAIVPQSFLKDDSIDNKVLTRSDKNRGKSDNVPSEEVVKKMK
NYWRQLLNAKLITQRKFDNLTKAERGGLSELDKAGFIKRQLVETRQITKHVAQILDSRMN
TKYDENDKLIREVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEITLANGEIRKR
PLIETNGETGEIVWDKGRDFATVRKVLSMPQVNIVKKTEVQTGGFSKESILPKRNSDKLI
ARKKDWDPKKYGGFDSPTVAYSVLVVAKVEKGKSKKLKSVKELLGITIMERSSFEKNPID
FLEAKGYKEVKKDLIIKLPKYSLFELENGRKRMLASAGELQKGNELALPSKYVNFLYLAS
HYEKLKGSPEDNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQSITGLYETRI
DLSQLGGDSPKKKRKVEASGPAIDMSKTIVLSVGEATRTLTEIQSTADRQIFEEKVGPLV
GRLRLTASLRQNGAKTAYRVNLKLDQADVVDCSTSVCGELPKVRYTQVWSHDVTIVANST
EASRKSLYDLTKSLVATSQVEDLVVNLVPLGR
dCas9MS2
  (SEQ ID NO: 483)
MYPYDVPDYASPKKKRKVEASDKKYSIGLAIGTNSVGWAVITDEYKVPSKKFKVLGNTD
R HSIKKNLIGALLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKADLRLIYLALAH
MIKFRGHFLIEGDLNPDNSDVDKLFIQLVQTYNQLFEENPINASGVDAKAILSARLSKSR
RLENLIAQLPGEKKNGLFGNLIALSLGLTPNFKSNFDLAEDAKLQLSKDTYDDDLDNLLA
QIGDQYADLFLAAKNLSDAILLSDILRVNTEITKAPLSASMIKRYDEHHQDLTLLKALVR
QQLPEKYKEIFFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPYYVGPLARGNS
RFAWMTRKSEETITPWNFEEVVDKGASAQSFIERMTNFDKNLPNEKVLPKHSLLYEYFTV
YNELTKVKYVTEGMRKPAFLSGEQKKAIVDLLFKTNRKVTVKQLKEDYFKKIECFDSVEI
SGVEDRFNASLGTYHDLLKIIKDKDFLDNEENEDILEDIVLTLTLFEDREMIEERLKTYA
HLFDDKVMKQLKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGR
DMYVDQELDINRLSDYDVDAIVPQSFLKDDSIDNKVLTRSDKNRGKSDNVPSEEVVKKMK
NYWRQLLNAKLITQRKFDNLTKAERGGLSELDKAGFIKRQLVETRQITKHVAQILDSRMN
TKYDENDKLIREVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEITLANGEIRKR
PLIETNGETGEIVWDKGRDFATVRKVLSMPQVNIVKKTEVQTGGFSKESILPKRNSDKLI
ARKKDWDPKKYGGFDSPTVAYSVLVVAKVEKGKSKKLKSVKELLGITIMERSSFEKNPID
FLEAKGYKEVKKDLIIKLPKYSLFELENGRKRMLASAGELQKGNELALPSKYVNFLYLAS
HYEKLKGSPEDNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQSITGLYETRI
DLSQLGGDSPKKKRKVEASGPAMASNFTQFVLVDNGGTGDVTVAPSNFANGIAEWISSNS
RSQAYKVTCSVRQSSAQNRKYTIKVEVPKGAWRSYLNMELTIPIFATNSDCELIVKAMQG
LLKDGNPIPSAIAANSGIYA
dCas9VP160ER
  (SEQ ID NO: 484)
MYPYDVPDYASPKKKRKVEASDKKYSIGLAIGTNSVGWAVITDEYKVPSKKFKVLGNTD
R HSIKKNLIGALLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKADLRLIYLALAH
MIKFRGHFLIEGDLNPDNSDVDKLFIQLVQTYNQLFEENPINASGVDAKAILSARLSKSR
RLENLIAQLPGEKKNGLFGNLIALSLGLTPNFKSNFDLAEDAKLQLSKDTYDDDLDNLLA
QIGDQYADLFLAAKNLSDAILLSDILRVNTEITKAPLSASMIKRYDEHHQDLTLLKALVR
QQLPEKYKEIFFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPYYVGPLARGNS
RFAWMTRKSEETITPWNFEEVVDKGASAQSFIERMTNFDKNLPNEKVLPKHSLLYEYFTV
YNELTKVKYVTEGMRKPAFLSGEQKKAIVDLLFKTNRKVTVKQLKEDYFKKIECFDSVEI
SGVEDRFNASLGTYHDLLKIIKDKDFLDNEENEDILEDIVLTLTLFEDREMIEERLKTYA
HLFDDKVMKQLKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGR
DMYVDQELDINRLSDYDVDAIVPQSFLKDDSIDNKVLTRSDKNRGKSDNVPSEEVVKKMK
NYWRQLLNAKLITQRKFDNLTKAERGGLSELDKAGFIKRQLVETRQITKHVAQILDSRMN
TKYDENDKLIREVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEITLANGEIRKR
PLIETNGETGEIVWDKGRDFATVRKVLSMPQVNIVKKTEVQTGGFSKESILPKRNSDKLI
ARKKDWDPKKYGGFDSPTVAYSVLVVAKVEKGKSKKLKSVKELLGITIMERSSFEKNPID
FLEAKGYKEVKKDLIIKLPKYSLFELENGRKRMLASAGELQKGNELALPSKYVNFLYLAS
HYEKLKGSPEDNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQSITGLYETRI
DLSQLGGDSPKKKRKVEASGPAGSGRADALDDFDLDMLGSDALDDFDLDMLGSDALDDFD
LDMLGSDALDDFDLDMLGSDALDDFDLDMLGSDALDDFDLDMLGSDALDDFDLDMLGSDA
LDDFDLDMLGSDALDDFDLDMLGSDALDDFDLDMLYIDSAGDMRAANLWPSPLMIKRSKK
NSLALSLTADQMVSALLDAEPPILYSEYDPTRPFSEASMMGLLTNLADRELVHMINWAKR
VPGFVDLTLHDQVHLLECAWLEILMIGLVWRSMEHPVKLLFAPNLLLDRNQGKCVEGMVE
IFDMLLATSSRFRMMNLQGEEFVCLKSIILLNSGVYTFLSSTLKSLEEKDHIHRVLDKIT
DTLIHLMAKAGLTLQQQHQRLAQLLLILSHIRHMSNKGMEHLYSMKCKNVVPLYDLLLEA
ADAHRLHAPTSRGGASVEETDQSHLATAGSTSSHSLQKYYITGEAEGFPATV
```

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

1. A method of mutating one or more target nucleic acid sequences in a stem cell or zygote comprising:
(a) introducing into the stem cell or zygote
(i) one or more ribonucleic acid (RNA) sequences that comprise a portion that is complementary to a portion of each of the one or more target nucleic acid sequences and comprise a binding site for a CRISPR associated (Cas) protein; and
ii) a Cas nucleic acid sequence or a variant thereof that encodes a Cas protein having nuclease activity; and
(b) maintaining the cell or zygote under conditions in which the one or more RNA sequences hybridize to the portion of each of the one or more target nucleic acid sequences, and the Cas protein cleaves each of the one or more target nucleic acid sequences upon hybridization of the one or more RNA sequences to the portion of the target nucleic acid sequence;
thereby mutating one or more target nucleic acid sequences in the stem cell or zygote.

2.-37. (canceled)

38. A method of producing a nonhuman mammal carrying mutations in one or more target nucleic acid sequences comprising:
(a) introducing into a zygote or an embryo
(i) one or more ribonucleic acid (RNA) sequences that comprise a portion that is complementary to a portion of each of the one or more target nucleic acid sequences and comprise a binding site for a CRISPR associated (Cas) protein; and
ii) a Cas nucleic acid sequence or a variant thereof that encodes a Cas protein having nuclease activity; and
(b) maintaining the zygote or the embryo under conditions in which RNA hybridizes to the portion of each of the one or more target nucleic acid sequences, and the Cas protein cleaves each of the one or more target nucleic acid sequences upon hybridization of the RNA to the portion of the target nucleic acid sequence, thereby producing an embryo having one or more mutated nucleic acid sequences;
(c) introducing the embryo having one or more mutated nucleic acid sequences into a foster nonhuman mammalian mother; and
(d) maintaining the foster nonhuman mammalian mother under conditions in which one or more offspring carrying the one or more mutated nucleic acid sequences are produced, thereby producing a nonhuman mammal carrying mutations in one or more target nucleic acid sequences.

39. The method of claim 38 wherein the Cas protein is Cas9.

40. The method of claim 38 wherein the Cas protein cleaves both strands of one or more of the target nucleic acid sequences.

41. (canceled)

42. The method of claim 38 wherein the RNA sequence is from about 10 base pairs to about 150 base pairs in length.

43. The method of claim 38 wherein the nonhuman mammal is a rodent, a nonhuman primate, a canine, a feline, a bovine, a porcine, an equine, or a caprine.

44. The method of claim 43 wherein the rodent is a mouse.

45. (canceled)

46. The method of claim 38 wherein one or more of the target nucleic sequences are a gene.

47. The method of claim 38 wherein both copies of one or more of the target nucleic acid sequences in the zygote or the embryo are mutated.

48. The method of claim 38 wherein the one or more target nucleic acid sequences are endogenous to the zygote or the embryo.

49. (canceled)

50. The method of claim 38 further comprising introducing into the zygote or the embryo one or more nucleic acid sequences that are complementary to a portion of the one or more target nucleic acid sequences cleaved by the Cas protein.

51. The method of claim 50 wherein the one or more nucleic acid sequences are a single stranded DNA oligonucleotide, a double stranded DNA oligonucleotide, a plasmid, a cDNA, a gene block or a PCR product.

52. The method of claim 50 wherein the one or more nucleic acid sequences replace one or more nucleotides, introduce one or more additional nucleotides, delete one or more nucleotides or a combination thereof in the one or more target nucleic acid sequences.

53. (canceled)

54. (canceled)

55. The method of claim 50 wherein the one or more nucleic acid sequences is from about 10 nucleotides to about 1000 nucleotides.

56.-59. (canceled)

60. The method of claim 38 wherein at least two of the target nucleic acid sequences are endogenous nucleic acid sequences.

61.-63. (canceled)

64. The method of claim 38 wherein at least one mutation comprises an insertion of a tag, a transgene, or an insertion of a site recognized by a recombinase.

65. The method of claim 38 wherein at least one mutation renders expression of an endogenous gene conditional.

66.-69. (canceled)

70. A non-human mammal produced by the method of claim 38.

71. A method of modulating the expression and/or activity of one or more target nucleic acid sequences in a cell comprising:
(a) introducing into the cell
(i) one or more ribonucleic acid (RNA) sequences that comprise a portion that is complementary to each of the one or more target nucleic acid sequences and comprise a binding site for a CRISPR associate (Cas) protein;
(ii) a Cas nucleic acid sequence or a variant thereof that encodes the Cas protein that targets but does not cleave the target nucleic acid sequence; and
(iii) an effector domain;
(b) maintaining the cell under conditions in which the one or more RNA sequences hybridize to the portion of each of the one or more target nucleic acid sequences, the Cas protein binds to each of the one or more RNA sequences and the effector domain modulates the expression and/or activity of the target nucleic acid,
thereby modulating the expression and/or activity of one or more target nucleic acid sequences in the cell.

72.-140. (canceled)
